(19) United States
(12) Patent Application Publication
(10) Pub. No.: US 2024/0141287 A1
(43) Pub. Date: May 2, 2024

(54) BACTERIAL STRAINS HAVING PESTICIDAL ACTIVITY AND USE THEREOF

(71) Applicant: LAVIE BIO LTD., Rehovot (IL)

(72) Inventors: Michal Shoresh, Holon (IL); Dhritiman Ghosh, Saint Peters, MO (US); James Presnail, Minneapolis, MN (US); Amir Bercovitz, Raanana (IL); Yossi Singer, Hashmonaim (IL); Michael Ionescu, Mazkeret-Batya (IL)

(21) Appl. No.: 18/546,967

(22) PCT Filed: Feb. 21, 2022
(86) PCT No.: PCT/IL2022/050200

§ 371 Date: Aug. 17, 2023

Related U.S. Application Data
(60) Provisional application No. 63/151,736, filed on Feb. 21, 2021.

(51) Int. Cl. C12N 1/20; A01N 63/20; A01N 63/27; A01P 7/02; A01P 7/04

(52) U.S. Cl. CPC C12N 1/205; A01N 63/20; A01N 63/27; A01P 7/02; A01P 7/04; C12R 2001/38

(57) ABSTRACT

The present invention is in the field of biocontrol of plant insect and/or mite pests. Particularly, the present invention discloses bacterial strains effective in inhibiting the development of or in killing insects and/or mites, and/or in protecting plants from deleterious effects of insects and/or mites, and use thereof as bio-pesticides.

### FIELD OF THE INVENTION

The present invention is in the field of biocontrol of plant insect and/or mite pests. Particularly, the present invention discloses bacterial strains effective in killing or inhibiting the development of insects and/or mites, and/or in protecting plants from deleterious effects of insects and/or mites, and use thereof for reducing deleterious effects of said pests on plants.

### BACKGROUND OF THE INVENTION

Pests including insects and mites are a major factor in the loss of agricultural crops worldwide. Insects destroy economically valuable plants (including crops, trees and ornamental plants) by feeding on the plants either while in larval stages or as adults. Plant insect pests are of a variety of forms, belonging to different orders including Lepidoptera, Coleoptera, Hemiptera and more.

For example, the Lepidopteran species Fall armyworm and European corn borer inflict damage that can be economically devastating to maize producers. Fall armyworm (Spodoptera frupperda) is a species in the order of Lepidoptera, of the Noctuidae family, and is the larval stage of a fall armyworm moth. The fall armyworm mainly attacks maize crops, and is capable of completely destroying maize fields. The fall armyworm is active in the late summer in the southern part of the United States, and during early fall in the northern regions. Insect pest-related crop loss as a result of European corn borer attacks on sweet corn fields alone has reached about one billion dollars a year in damage and control expenses.

Other insect pests of the Lepidopteran family Noctuidae include the Cabbage looper (Trichoplusia ni), Soybean looper (Chrysodeixis includens) and Black cutworm (Agrotis ipsilon). The Cabbage looper is a destructive crop pest in North America. During the larval stage, the pest eats three times its body weight in plant material a day. Thus, once established in a crop field, the cabbage looper is difficult to control. The soybean looper (Chrysodeixis includens) is widely spread from Southern Quebec and Southern Ontario through the eastern and southern part of the United States to Central America and South America, the Antilles and the Galapagos Islands. The larvae feed on a wide range of plants of the families Asteraceae, Brassicaceae, Commelinaceae, Euphorbiaceae, Fabaceae, Geraniaceae, Lamiaceae, Lauraceae, Malvaceae, Solanaceae, and Verbenaceae, and particularly on Medicago sativa, Phaseolus polystachios, Glycine max, Gossypium herbaceum, Nicotiana tabacum, Lycopersicum esculentum, Brassica and Lactuca sativa. Black cutworms attack corn in the Midwest USA. The moths are attracted to early spring vegetation, are active at night and prefer to deposit their eggs on low-growing, dense vegetation. It is noted that a single black cutworm larva is capable of cutting approximately four corn plants during its lifetime, depending on the size of the plants.

An additional major Lepidopteran plant insect pest is Corn earworm (Helicoverpa zea), also referred to as cotton bollworm and tomato fruitworm, which feeds on many different plants and crops (polyphagous). The species is widely distributed across the Americas with the exception of northern Canada and Alaska. It migrates seasonally, at night, and can be carried downwind up to 400 km. Pupae can make use of diapause to wait out adverse environmental conditions, especially at high latitudes and under drought conditions. The corn earworm has become resistant to many pesticides, and current techniques attempting to control this species include deep ploughing, trap crops, chemical control using mineral oil, and biological controls.

The Coleopteran species Western corn rootworm (Diabrotica virgifera virgifera) is one of the most devastating corn rootworm species in North America and belongs to the family Chrysomehdae. Corn rootworm larvae can destroy significant percentages of corn if left untreated. In the United States, current estimates show that 30 million acres of corn are potentially infested with corn rootworm each year, causing about 1 billion USD in lost revenue. Several other members of the genus Diabrotica are also important pests including Diabrotica barberi, Diabrotica undecimpunctata, and Diabrotica speciosa. In addition, other plant pests belonging to the family Chrysomehdae include flea beetles, the Colorado potato beetle, and bean beetles.

The Hemipteran species Nezara viridula, belonging to the family Pentatomidae (stinkbugs), commonly known as the Southern green stink bug (USA), Southern green shield bug (UK) or Green vegetable bug (Australia and New Zealand), is a plant-feeding stink bug which can be found around the world. Because of its preference for certain species of legumes, such as beans and soybeans, it has a significant economic effect on the growth of such crops. Nezara viridula reproduces throughout the year in tropic areas. In temperate zones this species presents a reproductive winter diapause, associated with a reversible change of body coloration from green to brown or russet. Numerous other hemipteran species both within the family Pentatomidae and belonging to other families within the order are important plant pests, including vectors of some of the most important plant viral diseases.

The Spider mite Tetranychus urticae is a member of the class Acari, and attacks a wide range of plants, including peppers, tomatoes, potatoes, beans, corn, cannabis, and strawberries, and has a global distribution. Spider mites injure plants by piercing cells and sucking out their contents, causing damage to leaves which then lose photosynthetic surface as feeding continues. Water loss can get out of control and both photosynthetic rate and leaf water status decline with increasing levels of spider mite injury. Spider mites are well-adapted and quick to develop pesticide resistance, often within a growing season. In addition, pesticides worsen the problem by destroying the predatory mite populations, thus eliminating the natural controls on the spider mite species and allowing them even more opportunity to explode in population numbers. Altogether, this raises the need of an alternative method of control. Many other members of the class Acari within and beyond the spider mite family (Tetranychidae) are serious pests in a wide range of crops.

Most widely used insecticides are chemical compounds, at a cost of millions of dollars every year in the US only. In recent years, awareness of the hazardous effect of chemical pesticides on the environment and on human and animal health brought regulators to ban or limit the use of some of the more hazardous pesticides that were traditionally employed in plant fields. In addition, emerging insect resistance issues stimulated the research for and development of alternative systems and compounds for controlling the damage caused by plant insect pest.

One approach taken to replace chemical insecticides is to genetically modify crop plants to express proteins enhancing plant tolerance and/or resistance to the pests. Widely used are genes encoding pesticidal proteins from Bacillus thuringiensis. Genetically engineered crops carrying B. thuringiensis pesticidal proteins are now widely used in American agriculture and have provided producers with an environmentally friendly alternative to traditional insect-control methods. For example, in 2012, 26.1 million hectares were planted with transgenic crops expressing Bt toxins (James, C. “Global Status of Commercialized Biotech/GM Crops: 2012”. ISAAA Brief No. 44). However, while they have proven to be very successful commercially and have been widely adopted due to their efficacy, these genetically engineered, insect-resistant (or insect-protected) crop plants typically provide resistance to only a narrow range of economically important pests. In addition, the broad use of transgenic insect-protected crops in some geographies and the limited variety of insecticidal proteins used in these crops has created a selection pressure for existing insect alleles that impart resistance to the currently utilized insecticidal proteins.

Another approach uses microbial biocontrol agents (microbials), such as bacteria, or one or more products thereof, to combat the damages caused by insect pests. These biocontrol agents are applied to the plant or to a part thereof, or to the plant habitat, to control the pathogen. Microbials are good candidates to replace hazardous pesticides. The most commonly used insecticidal bacteria today are Bacillus species, since it was discovered that Bacillus thuringiensis (Bt) strains show a high toxicity against specific insects. Bt strains produce delta-endotoxins that are localized within parasporal crystalline inclusion bodies at the onset of sporulation and during the stationary growth phase, and are also known to produce secreted insecticidal proteins. Upon ingestion by a susceptible insect, delta-endotoxins as well as secreted toxins exert their effects at the surface of the insect midgut epithelium, disrupting the cell membrane, leading to cell disruption and death. Genes encoding insecticidal proteins have also been identified in bacterial species other than Bt, including other Bacillus spp. and a diversity of other bacterial species, such as Brevibacillus laterosporus, Lysinibacillus sphaericus and Paenibacillus popilliae. Insect pathogenicity has also been attributed to strains of B. larvae, B. lentimorbus, B. sphaericus and B. cereus. However, due to intensive use of Bt and equivalent strains, insect resistance to these toxins is developing.

There is a continuous need for, and it would be highly advantageous to have, additional, diverse biocontrol agents, including alternate bacterial agents, to be used against a wide range of plant pests including insects and mites.

### SUMMARY OF THE INVENTION

The present invention answers the need for safe biocontrol agents that can protect plants, particularly crop plants, from a broad spectrum of insect and/or mite pests, particularly plant insect and/or mite pests. The present invention provides bacterial strains showing unexpectedly effective activity in inhibiting or killing insects/mites that have deleterious effects on plant growth and development (plant insect/mite pests), and/or in protecting plants from those deleterious effects of insects and/or mites. The present invention further provides preparations of the bacterial strains, lysates, extracts, compositions comprising same and uses thereof. In certain aspects the present invention provides methods for conferring or enhancing the resistance of plants towards a broad spectrum of insect and/or mite pests, comprising contacting the plants, or the plants' immediate surroundings, with the bacterial strain, combinations of bacterial strains and/or preparations thereof.

According to certain aspects, the present invention provides an isolated bacterial strain or a functional homologue thereof, wherein the isolated bacterial strain is selected from the group consisting of:
strain LAV61269, the strain being selected from the group consisting of (a) a strain deposited under Accession Number 43908 at NCIMB; (b) a strain comprising a 16S-rRNA sequence comprising the nucleic acid sequence set forth in SEQ ID NO:8; (c) a strain comprising at least one genomic marker comprising the nucleic acid sequence set forth in any one of SEQ ID NOs:73-75, and any combination thereof;
strain LAV41249, the strain being selected from the group consisting of (a) a strain deposited under Accession Number 43914 at NCIMB; (b) a strain comprising a 16S-rRNA sequence comprising the nucleic acid sequence set forth in SEQ ID NO:1; (c) a strain comprising at least one genomic marker comprising the nucleic acid sequence set forth in any one of SEQ ID NOs:52-55, and any combination thereof;
strain LAV61293, the strain being selected from the group consisting of (a) a strain deposited under Accession Number 43913 at NCIMB; (b) a strain comprising a 16S-rRNA sequence comprising the nucleic acid sequence set forth in any one of SEQ ID NO:11 and SEQ ID NO:12; (c) a strain comprising at least one genomic marker comprising the nucleic acid sequence set forth in any one of SEQ ID NOs:79-81, and any combination thereof;
strain LAV61301, the strain being selected from the group consisting of (a) a strain deposited under Accession Number 43910 at NCIMB; (b) a strain comprising a 16S-rRNA sequence comprising the nucleic acid sequence set forth in SEQ ID NO:17; (c) a strain comprising at least one genomic marker comprising the nucleic acid sequence set forth in any one of SEQ ID NOs:91-92, and any combination thereof;
strain LAV104746, the strain being selected from the group consisting of (a) a strain deposited under Accession Number 43911 at NCIMB; (b) a strain comprising a 16S-rRNA sequence comprising the nucleic acid sequence set forth in SEQ ID NO:26; (c) a strain comprising at least one genomic marker comprising the nucleic acid sequence set forth in any one of SEQ ID NOs:112-113, and any combination thereof;
strain LAV104743, the strain being selected from the group consisting of (a) a strain deposited under Accession Number 43903 at NCIMB; (b) a strain comprising a 16S-rRNA sequence comprising the nucleic acid sequence set forth in any one of SEQ ID NO:21 and SEQ ID NO:22; (c) a strain comprising at least one genomic marker comprising the nucleic acid sequence set forth in any one of SEQ ID NOs:104-107, and any combination thereof;
strain LAV61243, the strain being selected from the group consisting of (a) a strain deposited under Accession Number 43905 at NCIMB; (b) a strain comprising a 16S-rRNA sequence comprising the nucleic acid sequence set forth in SEQ ID NO:4; (c) a strain comprising at least one genomic marker comprising the nucleic acid sequence set forth in any one of SEQ ID NOs:62-64, and any combination thereof;
strain LAV106494, the strain being selected from the group consisting of (a) a strain deposited under Accession Number 43915 at NCIMB; (b) a strain comprising a 16S-rRNA sequence comprising the nucleic acid sequence set forth in SEQ ID NO:46; (c) a strain comprising at least one genomic marker comprising the nucleic acid sequence set forth in any one of SEQ ID NOs:144-145, and any combination thereof;
strain LAV1000771, the strain being selected from the group consisting of (a) a strain deposited under Accession Number 43912 at NCIMB; (b) a strain comprising a 16S-rRNA sequence comprising the nucleic acid sequence set forth in SEQ ID NO:49; (c) a strain comprising at least one genomic marker comprising the nucleic acid sequence set forth in any one of SEQ ID NOs:164-165, and any combination thereof;

strain LAV1000775, the strain the strain being selected from the group consisting of (a) a strain deposited under Accession Number 43904 at NCIMB; (b) a strain comprising a 16S-rRNA sequence comprising the nucleic acid sequence set forth in SEQ ID NO:50; (c) a strain comprising at least one genomic marker comprising the nucleic acid sequence set forth in any one of SEQ ID NOs:166-170, and any combination thereof;
strain LAV1000731, the strain being selected from the group consisting of (a) a strain deposited under Accession Number 43906 at NCIMB; (b) a strain comprising a 16S-rRNA sequence comprising the nucleic acid sequence set forth in SEQ ID NO:48; (c) a strain comprising at least one genomic marker comprising the nucleic acid sequence set forth in any one of SEQ ID NOs:159-163, and any combination thereof;
strain LAV104934, the strain being selected from the group consisting of (a) a strain deposited under Accession Number 43902 at NCIMB; (b) a strain comprising a 16S-rRNA sequence comprising the nucleic acid sequence set forth in SEQ ID NO:40; (c) a strain comprising at least one genomic marker comprising the nucleic acid sequence set forth in any one of SEQ ID NOs:137-139, and any combination thereof; and
strain LAV104944, the strain being selected from the group consisting of (a) a strain deposited under Accession Number 43907 at NCIMB; (b) a strain comprising a 16S-rRNA sequence comprising the nucleic acid sequence set forth in SEQ ID NO:45; (c) a strain comprising at least one genomic marker comprising the nucleic acid sequence set forth in any one of SEQ ID NOs:140-143, and any combination thereof
strain LAV104753, the strain being selected from the group consisting of (a) a strain comprising a 16S-rRNA sequence comprising the nucleic acid sequence set forth in SEQ ID NO:30; (b) a strain comprising at least one genomic marker comprising the nucleic acid sequence set forth in any one of SEQ ID NOs:119-122, and a combination thereof;
strain LAV106564, the strain being selected from the group consisting of (a) a strain comprising a 16S-rRNA sequence comprising the nucleic acid sequence set forth in any one of SEQ ID NO:41 and SEQ ID NO:42; (b) a strain comprising at least one genomic marker comprising the nucleic acid sequence set forth in any one of SEQ ID NOs:146-148, and a combination thereof;
strain LAV100189, the strain being selected from the group consisting of (a) a strain comprising a 16S-rRNA sequence comprising the nucleic acid sequence set forth in SEQ ID NO:20; (b) a strain comprising at least one genomic marker comprising the nucleic acid sequence set forth in any one of SEQ ID NOs:100-103, and a combination thereof;
strain LAV1000686, the strain being selected from the group consisting of (a) a strain comprising a 16S-rRNA sequence comprising the nucleic acid sequence set forth in SEQ ID NO:47; (b) a strain comprising at least one genomic marker comprising the nucleic acid sequence set forth in any one of SEQ ID NOs:156-158, and a combination thereof;

Each possibility represents a separate embodiment of the present invention.

According to certain embodiments, strain LAV61269, strain LAV104944, and strain LAV104753 each is of the genus Pseudomonas. According to certain embodiments, strain LAV41249, strain LAV61293, strain LAV1000771, and strain LAV104746 each is of the genus Serratia. According to certain embodiments, strain LAV61301 is of the genus Rahnella. According to certain embodiments, strains LAV104743 and LAV106564 each is of the genus Carnobacterium. According to certain embodiments, strain LAV61243 is of the genus Microbacterium. According to certain embodiments, strains LAV106494 and strain LAV100189 each is of the genus Stenotrophomonas. According to certain embodiments, strain LAV1000775 is of the genus Chryseobacterium. According to certain embodiments, strain LAV1000731 is of the genus Paraburkholderia. According to certain embodiments, strain LAV104934 and strain LAV1000686 each is of the genus Acinetobacter.

It is to be explicitly understood that the genus and/or the species of a bacterial strain of the invention as described herein is classified to the closest known species according to said genus/species characterization as are known in the art.

According to certain embodiments, the functional homolog comprises a 16S-rRNA sequence at least 97% identical to any one of SEQ ID NOs:1, 4, 11, 12, 17, 20, 21-22, 26, 30, 41, 42, 46, 47, 48, 49, or 50. Each possibility represents a separate embodiment of the present invention.

According to certain embodiments, the functional homolog comprises a 16S-rRNA sequence at least 98% identical to any one of SEQ ID NOs:8, 40 and 45.

According to certain additional or alternative embodiments, the functional homologs are characterized by at least one genomic marker having at least 99% local identity over 97% coverage to a nucleic acid sequence of the genomic markers of the isolated strains of the invention.

According to certain embodiments, the functional homolog of bacterial strain LAV61269 comprises a 16S-rRNA sequence at least 98% identical to SEQ ID NO:8; at least one genomic marker having at least 99% local identity to the nucleic acid sequence of any one of SEQ ID NOs:73-75 over 97% coverage; or a combination thereof.

According to some embodiments, the functional homolog of bacterial strain LAV41249 comprises a 16S-rRNA sequence at least 97% identical to SEQ ID NO:1; at least one genomic marker having at least 99% local identity to the nucleic acid sequence of any one of SEQ ID NOs:52-55 over 97% coverage; or a combination thereof.

According to some exemplary embodiments, the functional homolog of strain LAV41249 is strain LAV43112, comprising a 16S -rRNA sequence comprising the nucleic acid sequence set forth in SEQ ID NO:2, at least one genomic marker comprising the nucleic acid sequence set forth in any one of SEQ ID NOs:52, and 54-56, or a combination thereof. According to further certain exemplary embodiments, the functional homolog of strain LAV41249 is strain LAV104746. According to yet additional exemplary embodiments, the functional homolog of strain LAV41249 is strain LAV1000771.

According to certain embodiments, the functional homolog of bacterial strain LAV61293 comprises a 16S-rRNA sequence at least 97% identical to any one of SEQ ID NO:11 and SEQ ID NO:12; at least one genomic marker having at least 99% local identity to the nucleic acid sequence of any one of SEQ ID NOs:79-81 over 97% coverage; or a combination thereof. According to certain exemplary embodiments, the functional homolog of strain LAV61293 is strain LAV61300, comprising a 16S-rRNA sequence comprising the nucleic acid sequence set forth in SEQ ID NO:16, at least one genomic marker comprising the nucleic acid sequence set forth in any one of SEQ ID NOs:88-90, or a combination thereof.

According to certain embodiments, the functional homolog of bacterial strain LAV61301 comprises a 16S-rRNA sequence at least 97% identical to SEQ ID NO:17; at least one genomic marker having at least 99% local identity to the nucleic acid sequence of any one of SEQ ID NOs:91-92 over 97% coverage; or a combination thereof. According to further certain exemplary embodiments, the functional homolog of strain LAV61301 is strain LAV106657, comprising a 16S-rRNA sequence comprising the nucleic acid sequence set forth in SEQ ID NO:44, at least one genomic marker comprising the nucleic acid sequence set forth in any one of SEQ ID NOs:154-155, or a combination thereof.

According to certain embodiments, the functional homolog of bacterial strain LAV104746 comprises a 16S-rRNA sequence at least 97% identical to SEQ ID NO:26; at least one genomic marker having at least 99% local identity to the nucleic acid sequence of any one of SEQ ID NOs:112-113 over 97% coverage; or a combination thereof.

According to certain embodiments, the functional homolog of bacterial strain LAV104743 comprises a 16S-rRNA sequence at least 97% identical to any one of SEQ ID NO:21 and SEQ ID NO:22; at least one genomic marker having at least 99% local identity to the nucleic acid sequence of any one of SEQ ID NOs:104-107 over 97% coverage; or a combination thereof. According to certain exemplary embodiments, the functional homolog of strain LAV104743 is strain LAV106564.

According to certain embodiments, the functional homolog of bacterial strain LAV61243 comprises a 16S-rRNA sequence at least 97% identical to SEQ ID NO:4; at least one genomic marker having at least 99% local identity to the nucleic acid sequence of any one of SEQ ID NOs:62-64 over 97% coverage; or a combination thereof.

According to certain embodiments, the functional homolog of bacterial strain LAV106494 comprises a 16S-rRNA sequence at least 97% identical to SEQ ID NO:46; at least one genomic marker having at least 99% local identity to the nucleic acid sequence of any one of SEQ ID NOs:144-145 over 97% coverage; or a combination thereof.

According to certain embodiments, the functional homolog of bacterial strain LAV1000771 comprises a 16S-rRNA sequence at least 97% identical to SEQ ID NO:49; at least one genomic marker having at least 99% local identity to the nucleic acid sequence of any one of SEQ ID NOs:164-165 over 97% coverage; or a combination thereof. According to certain exemplary embodiments, the functional homolog of strain LAV1000771 is strain LAV41249. According to certain exemplary embodiments, the functional homolog of strain LAV1000771 is strain LAV104746. According to additional certain exemplary embodiments, the functional homolog of strain LAV1000771 is strain LAV43112.

According to certain embodiments, the functional homolog of bacterial strain LAV1000775 comprises a 16S-rRNA sequence at least 97% identical to SEQ ID NO:50; at least one genomic marker having at least 99% local identity to the nucleic acid sequence of any one of SEQ ID NOs:166-170 over 97% coverage; or a combination thereof.

According to certain embodiments, the functional homolog of bacterial strain LAV1000731 comprises a 16S-rRNA sequence at least 97% identical to SEQ ID NO:48; at least one genomic marker having at least 99% local identity to the nucleic acid sequence of any one of SEQ ID NOs:159-163 over 97% coverage; or a combination thereof.

According to certain embodiments, the functional homolog of bacterial strain LAV104753 comprises a 16S-rRNA sequence at least 97% identical to SEQ ID NO:30; at least one genomic marker having at least 99% local identity to the nucleic acid sequence of any one of SEQ ID NOs:119-122 over 97% coverage; or a combination thereof.

According to certain embodiments, the functional homolog of bacterial strain LAV106564 comprises a 16S-rRNA sequence at least 97% identical to any one of SEQ ID NOs:41-42; at least one genomic marker having at least 99% local identity to the nucleic acid sequence of any one of SEQ ID NOs:146-148 over 97% coverage; or a combination thereof. According to certain exemplary embodiments, the functional homolog of strain LAV106564 is strain LAV104743.

According to certain embodiments, the functional homolog of bacterial strain LAV100189 comprises a 16S-rRNA sequence at least 97% identical to SEQ ID NO:20; at least one genomic marker having at least 99% local identity to the nucleic acid sequence of any one of SEQ ID NOs:100-103 over 97% coverage; or a combination thereof.

According to certain embodiments, the functional homolog of bacterial strain LAV1000686 comprises a 16S-rRNA sequence at least 97% identical to SEQ ID NO:47; at least one genomic marker having at least 99% local identity to the nucleic acid sequence of any one of SEQ ID NOs:156-158 over 97% coverage; or a combination thereof.

According to certain embodiments, the functional homolog of bacterial strain LAV104934 comprises a 16S-rRNA sequence at least 98% identical to SEQ ID NO:40; at least one genomic marker having at least 99% local identity to the nucleic acid sequence of any one of SEQ ID NOs:137-139 over 97% coverage; or a combination thereof.

According to certain embodiments, the functional homolog of bacterial strain LAV104944 comprises a 16S-rRNA sequence at least 98% identical to SEQ ID NO:45; at least one genomic marker having at least 99% local identity to the nucleic acid sequence of any one of SEQ ID NOs:140-143 over 97% coverage; or a combination thereof.

According to certain embodiments, a functional homolog of a bacterial strain of the invention is characterized by having substantially the same coding and/or non-coding sequence orientation as that of the bacterial strain of the invention.

It is to be explicitly understood that the present invention encompasses a bacterium of the bacterial strains or the functional homolog strains thereof as well as a bacterium derivable from bacterial strains or the functional homolog strains thereof

According to certain currently exemplary embodiments, the bacterial strains, functional homologous bacterial strains, and bacterium derived therefrom are characterized by a capability to inhibit the growth and/or the development and/or the activity of, or to kill, at least one plant insect and/or mite pest, thereby protecting the plant from, or reducing the impact of the harmful effects of, the at least one insect and/or mite pest. The inhibitory activity or killing the pest can be exerted directly by the bacterial strain, or be mediated by the plant, part thereof or the plant habitat affected by the bacterial strain.

According to another aspect, the present invention provides a bacterial preparation comprising a plurality of bacteria of at least one bacterial strain selected from the group consisting of LAV61269, LAV41249, LAV61243, LAV61293, LAV61301, LAV104743, LAV104746, LAV104934, LAV104944, LAV106494, LAV1000771, LAV1000775, LAV1000731, LAV104753, LAV106564, LAV100189, LAV1000686, and functional homologs thereof, wherein the bacterial strain or functional homolog thereof is effective in inhibiting or preventing the growth and/or the development and/or the activity of at least one plant insect and/or mite pest, thereby reducing or preventing the damage caused to a plant by the insect and/or mite pest.

According to certain embodiments, the bacterial preparation comprises a culture medium. Culture media suitable for the growth of the various bacterial genera and species according to the teachings of the present invention are known to a person skilled in the Art.

According to certain embodiments, the bacterial strain or functional homolog thereof is present in the preparation at a concentration which exceeds that found in nature.

According to certain embodiments, the bacterial preparation comprises viable bacterial cells. According to some embodiments, the viable cells are in a dormant form. According to other embodiments, the bacterial preparation comprises non-viable cells.

According to certain additional aspects, the present invention provides a lysate of at least one bacterial strain of the invention or the functional homologs thereof. According to some embodiments, the lysate comprises a whole cell lysate of the bacterial cells. According to some embodiments, the lysate comprises a soluble fraction of the bacterial cells. According to some embodiments, the lysate comprises inclusion bodies of a bacterial preparation.

According to certain further aspects, the present invention provides a cell extract of at least one bacterial strain of the invention or the functional homologs thereof.

According to yet certain additional aspects, the present invention provides a whole cell broth collected from fermentation of at least one bacterial strain of the invention.

According to certain embodiments, the lysate, extract or broth is obtained from a plurality of the bacterial cells. According to certain embodiments, the lysate, extract or broth is of bacterial cells of the same bacterial species and/or strain. According to certain embodiments, the lysate, extract, or broth is of bacterial cells of different species and/or strains.

The bacterial strain and functional homologs thereof are as described hereinabove.

According to further certain aspects, the present invention provides an agricultural composition comprising a plurality of bacteria of at least one bacterial strain selected from the group consisting of LAV61269, LAV41249, LAV61243, LAV61293, LAV61301, LAV104743, LAV104746, LAV104934, LAV104944, LAV106494, LAV1000771, LAV1000775, LAV1000731, LAV104753, LAV106564, LAV100189, LAV1000686, and functional homologs thereof; a preparation of same, a lysate, a broth or an extract thereof. Each possibility represents a separate embodiment of the present invention. According to certain currently exemplary embodiments, the agricultural composition further comprises an agriculturally acceptable diluent(s) or carrier(s).

According to yet certain additional aspects, the present invention provides use of a plurality of at least one bacterial strain selected from the group consisting of LAV61269, LAV41249, LAV61243, LAV61293 LAV61301, LAV104743, LAV104746, LAV104934, LAV104944, LAV106494, LAV1000771, LAV1000775, LAV1000731, LAV104753, LAV106564, LAV100189, LAV1000686, and functional homologs thereof; a preparation of same, a lysate, a broth or an extract thereof, for the production of an agricultural composition. Each possibility represents a separate embodiment of the present invention.

According to certain embodiments, the agricultural composition comprises a combination of at least two, at least three, at least four, at least five or more distinct bacterial strains and/or functional homologs thereof. According to some embodiments, the agricultural composition comprises a lysate, an extract or a broth obtained from a combination of at least two, at least three, at least four, at least five or more distinct bacterial strains and/or functional homologs thereof.

According to certain embodiments, the agricultural composition is a plant protection product effective in reducing or preventing a damage caused to the plant by an insect and/or mite pest.

According to certain embodiments, the agricultural composition further comprises at least one additional active agent selected from the group consisting of a fertilizer, an acaricide, a bactericide, a fungicide, an additional insecticide, a microbicide, a nematicide, a pesticide, a plant growth regulator, a rodenticide, a nutrient and any combination thereof. Each possibility represents a separate embodiment of the present invention. According to certain embodiments, the at least one additional active agent is a synthetic agent.

The agricultural composition can be formulated in any form suitable for applying the composition to a plant or a part thereof or to the plant habitat as is known in the art. According to certain embodiments, the agricultural composition is formulated in a form selected from the group consisting of an emulsion, a colloid, a dust, a granule, a pellet, a powder, a spray, pressurized form, a pressurizable form, and a solution. Each possibility represents a separate embodiment of the present invention.

According to certain embodiments, the formulation further comprises at least one of a stabilizer, a tackifier, a preservative, a carrier, a surfactant, and a combination thereof. Each possibility represents a separate embodiment of the present invention.

According to certain embodiments, the formulation is substantially stable for at least 30 days at a temperature range of from about 4° C. to about 37° C. According to certain exemplary embodiments, the formulation is substantially stable at a temperature range of from about 20° C. to 25° C. for more than 30 days. According to certain additional exemplary embodiments, the formulation is substantially stable at a temperature range of from about 2-8° C., typically at 4° C., for at least 30 days.

According to some embodiments of the invention, the formulation is in a form selected from the group consisting of liquid, solid, semi-solid, gel or powder. Each possibility represents a separate embodiment of the present invention.

According to certain embodiments, the carrier is a plant seed. According to these embodiments, the present invention provides an agricultural composition comprising at least one plant seed and at least one bacterial strain selected from the group consisting of LAV61269, LAV41249, LAV61243, LAV61293, LAV61301, LAV104743, LAV104746, LAV104934, LAV104944, LAV106494, LAV1000771, LAV1000775, LAV1000731, LAV104753, LAV106564, LAV100189, LAV1000686, and functional homologs thereof a preparation of same, a lysate, a broth or an extract thereof.

According to certain currently exemplary embodiments, the agricultural composition is in a form of seed coating. According to these embodiments, the seed coating formulation further comprises at least one agent selected from the group consisting of a binding agent and a wetting agent. According to certain exemplary embodiments, the binding agent is carboxymethyl cellulose (CMC).

According to yet additional certain aspects, the present invention provides a seed coated with an agricultural composition comprising at least one bacterial strain, a lysate, an extract or a broth thereof as described herein.

According to certain aspects, the present invention provides a container adapted for a watering system of a plant field, comprising an agricultural composition comprising at least one bacterial strain, a lysate, an extract or a broth thereof as described herein.

According to certain aspects, the present invention provides a kit comprising (i) an agricultural composition comprising at least one bacterial strain, a lysate, an extract or a broth thereof as described herein; optionally (ii) a delivery system for applying the agricultural composition to a plant or a part thereof or to the plant growth medium and (iii) instructions for using the agricultural composition.

According to certain embodiments, the instructions for using the agricultural composition comprise instructions for the amounts and frequency of applying the agricultural composition as to reduce or prevent damage caused to the plant by an insect and/or mite pest.

According to further certain aspects, the present invention provides a method for inhibiting or preventing the growth and/or the development of at least one insect and/or mite pest present in or on a plant or a part thereof or a plant habitat, comprising contacting the plant or part thereof or plant habitat with at least one bacterial strain or a functional homologue thereof, wherein the isolated bacterial strain is selected from the group consisting of:
strain LAV61269, the strain being selected from the group consisting of (a) a strain deposited under Accession Number 43908 at NCIMB; (b) a strain comprising a 16S-rRNA sequence comprising the nucleic acid sequence set forth in SEQ ID NO:8; (c) a strain comprising at least one genomic marker comprising the nucleic acid sequence set forth in any one of SEQ ID NOs:73-75 and any combination thereof;
strain LAV41249, the strain being selected from the group consisting of (a) a strain deposited under Accession Number 43914 at NCIMB; (b) a strain comprising a 16S-rRNA sequence comprising the nucleic acid sequence set forth in SEQ ID NO:1; (c) a strain comprising at least one genomic marker comprising the nucleic acid sequence set forth in any one of SEQ ID NOs:52-55 and any combination thereof;
strain LAV61243, the strain being selected from the group consisting of (a) a strain deposited under Accession Number 43905 at NCIMB; (b) a strain comprising a 16S-rRNA sequence comprising the nucleic acid sequence set forth in SEQ ID NO:4; (c) a strain comprising at least one genomic marker comprising the nucleic acid sequence set forth in any one of SEQ ID NOs:62-64 and any combination thereof;
strain LAV61293, the strain being selected from the group consisting of (a) a strain deposited under Accession Number 43913 at NCIMB; (b) a strain comprising a 16S-rRNA sequence comprising the nucleic acid sequence set forth in any one of SEQ ID NO:11 and SEQ ID NO:12; (c) a strain comprising at least one genomic marker comprising the nucleic acid sequence set forth in any one of SEQ ID NOs:79-81 and any combination thereof;
strain LAV61301, the strain being selected from the group consisting of (a) a strain deposited under Accession Number 43913 at NCIMB; (b) a strain comprising a 16S-rRNA sequence comprising the nucleic acid sequence set forth in SEQ ID NO:17; (c) a strain comprising at least one genomic marker comprising the nucleic acid sequence set forth in any one of SEQ ID NOs:91-92 and any combination thereof;
strain LAV104743, the strain being selected from the group consisting of (a) a strain deposited under Accession Number 43903 at NCIMB; (b) a strain comprising a 16S-rRNA sequence comprising the nucleic acid sequence set forth in any one of SEQ ID NO:21 and SEQ ID NO:22; (c) a strain comprising at least one genomic marker comprising the nucleic acid sequence set forth in any one of SEQ ID NOs:104-107 and any combination thereof;
strain LAV104746, the strain being selected from the group consisting of (a) a strain deposited under Accession Number 43911 at NCIMB; (b) a strain comprising a 16S-rRNA sequence comprising the nucleic acid sequence set forth in SEQ ID NO:26; (c) a strain comprising at least one genomic marker comprising the nucleic acid sequence set forth in any one of SEQ ID NOs:112-113 and any combination thereof;
strain LAV104934, the strain being selected from the group consisting of (a) a strain deposited under Accession Number 43902 at NCIMB; (b) a strain comprising a 16S-rRNA sequence comprising the nucleic acid sequence set forth in SEQ ID NO:40; (c) a strain comprising at least one genomic marker comprising the nucleic acid sequence set forth in any one of SEQ ID NOs:137-139 and any combination thereof;
strain LAV104944, the strain being selected from the group consisting of (a) a strain deposited under Accession Number 43907 at NCIMB; (b) a strain comprising a 16S-rRNA sequence comprising the nucleic acid sequence set forth in SEQ ID NO:45; (c) a strain comprising at least one genomic marker comprising the nucleic acid sequence set forth in any one of SEQ ID NOs:140-143 and any combination thereof;
strain LAV106494, the strain being selected from the group consisting of (a) a strain deposited under Accession Number 43915 at NCIMB; (b) a strain comprising a 16S-rRNA sequence comprising the nucleic acid sequence set forth in SEQ ID NO:46; (c) a strain comprising at least one genomic marker comprising the nucleic acid sequence set forth in any one of SEQ ID NOs:144-145 and any combination thereof;
strain LAV1000771, the strain being selected from the group consisting of (a) a strain deposited under Accession Number 43912 at NCIMB; (b) a strain comprising a 16S-rRNA sequence comprising the nucleic acid sequence set forth in SEQ ID NO:49; (c) a strain comprising at least one genomic marker comprising the nucleic acid sequence set forth in any one of SEQ ID NOs:164-165 and any combination thereof;
strain LAV1000775, the strain the strain being selected from the group consisting of (a) a strain deposited under Accession Number 43904 at NCIMB; (b) a strain comprising a 16S-rRNA sequence comprising the nucleic acid sequence set forth in SEQ ID NO:50; (c) a strain comprising at least one genomic marker comprising the nucleic acid sequence set forth in any one of SEQ ID NOs:166-170 and any combination thereof;
strain LAV1000731, the strain being selected from the group consisting of (a) a strain deposited under Accession Number 43906 at NCIMB; (b) a strain comprising a 16S-rRNA sequence comprising the nucleic acid sequence set forth SEQ ID NO:48; (c) a strain comprising at least one genomic marker comprising the nucleic acid sequence set forth in any one of SEQ ID NOs:159-163 and any combination thereof;
strain LAV100189, the strain being selected from the group consisting of (a) a strain comprising a 16S-rRNA sequence comprising the nucleic acid sequence set forth in SEQ ID NO:20; (b) a strain comprising at least one genomic marker comprising the nucleic acid sequence set forth in any one of SEQ ID NOs:100-103 and a combination thereof;
strain LAV104753, the strain being selected from the group consisting of (a) a strain comprising a 16S-rRNA sequence comprising the nucleic acid sequence set forth in SEQ ID NO:30; (b) a strain comprising at least one genomic marker comprising the nucleic acid sequence set forth in any one of SEQ ID NOs:119-122 and a combination thereof;
strain LAV106564, the strain being selected from the group consisting of (a) a strain comprising a 16S-rRNA sequence comprising the nucleic acid sequence set forth in any one of SEQ ID NO:41 and SEQ ID NO:42; (b) a strain comprising at least one genomic marker comprising the nucleic acid sequence set forth in any one of SEQ ID NOs:146-148 and a combination thereof;
strain LAV1000686, the strain being selected from the group consisting of (a) a strain comprising a 16S-rRNA sequence comprising the nucleic acid sequence set forth SEQ ID NO:47 (b) a strain comprising at least one genomic marker comprising the nucleic acid sequence set forth in any one of SEQ ID NOs:156-158 and a combination thereof;
strain LAV104744, the strain being selected from the group consisting of (a) a strain deposited under Accession Number 43901 at NCIMB; (b) a strain comprising a 16S-rRNA sequence comprising the nucleic acid sequence set forth in SEQ ID NO:23; (c) a strain comprising at least one genomic marker comprising the nucleic acid sequence set forth in any one of SEQ ID NOs:108-111, and any combination thereof;
strain LAV104747, the strain being selected from the group consisting of (a) a strain deposited under Accession Number 43909 at NCIMB; (b) a strain comprising a 16S-rRNA sequence comprising the nucleic acid sequence set forth in any one of SEQ ID NO:27 and SEQ ID NO:28; (c) a strain comprising at least one genomic marker comprising the nucleic acid sequence set forth in any one of SEQ ID NOs:114-116 and any combination thereof;
strain LAV1000939, the strain being selected from the group consisting of (a) a strain deposited under Accession Number 43916 at NCIMB; (b) a strain comprising a 16S-rRNA sequence comprising the nucleic acid sequence set forth in SEQ ID NO:51; (c) a strain comprising at least one genomic marker comprising the nucleic acid sequence set forth in any one of SEQ ID NOs:171-175, and any combination thereof;
strain LAV61288, the strain being selected from the group consisting of (a) a strain comprising a 16S-rRNA sequence comprising the nucleic acid sequence set forth in any one of SEQ ID NO:9 and SEQ ID NO:10; (b) a strain comprising at least one genomic marker comprising the nucleic acid sequence set forth in any one of SEQ ID NOs:76-78, and a combination thereof;
a preparation of same, a lysate thereof, an extract thereof, a broth obtained therefrom or a composition comprising same.

The functional homologs of strains LAV61269, LAV41249, LAV61243, LAV61293, LAV61301, LAV104743, LAV104934, LAV104944, LAV104746,

LAV106494, LAV1000771, LAV1000775, LAV1000731, LAV100189, LAV104753, LAV106564, and strain LAV1000686 are as described hereinabove.

According to certain embodiments, the functional homolog of bacterial strain LAV104744 comprises a 16S-rRNA sequence at least 97% identical to SEQ ID NO:23; at least one genomic marker having at least 99% local identity to the nucleic acid sequence of any one of SEQ ID NOs:108-111 over 97% coverage; or a combination thereof.

According to certain embodiments, the functional homolog of bacterial strain LAV104747 comprises a 16S-rRNA sequence at least 97% identical to any one of SEQ ID NO:27 and SEQ ID NO:28; at least one genomic marker having at least 99% local identity to the nucleic acid sequence of any one of SEQ ID NOs:114-116 over 97% coverage; or a combination thereof.

According to certain embodiments, the functional homolog of bacterial strain LAV1000939 comprises a 16S-rRNA sequence at least 97% identical to SEQ ID NO:51; at least one genomic marker having at least 99% local identity to the nucleic acid sequence of any one of SEQ ID NOs:171-175 over 97% coverage; or a combination thereof.

According to certain embodiments, the functional homolog of bacterial strain LAV61288 comprises a 16S-rRNA sequence at least 97% identical to any one of SEQ ID NO:9 and SEQ ID NO:10; at least one genomic marker having at least 99% local identity to the nucleic acid sequence of any one of SEQ ID NOs:76-78 over 97% coverage; or a combination thereof.

According to certain aspects, the present invention provides a method for protecting a plant from a deleterious effect of at least one pest present in or on the plant or part thereof, comprising contacting said plant or part thereof or the plant habitat with at least one bacterial strain selected from the group consisting LAV61269, LAV41249, LAV61243, LAV61293, LAV61301, LAV104743, LAV104746, LAV106494, LAV1000771, LAV1000775, LAV1000731, LAV100189, LAV104753, LAV106564, LAV1000686, LAV104744, LAV104747, LAV104934, LAV104944, LAV1000939, LAV61288, and functional homologs thereof; a preparation of same, a lysate thereof, an extract thereof, a broth obtained therefrom or a composition comprising same.

According to certain embodiments, the plant or part thereof comprises the at least one insect and/or mite pest in or on at least one cell or tissue of said plant or part thereof. According to some embodiments, the plant or part thereof comprising the at least one insect and/or mite pest is affected with symptoms associated with the presence of said at least one insect and/or mite pest.

The bacterial strains, homologs, lysates, extracts, broth and compositions are as described hereinabove.

According to certain embodiments, the plant part is selected from the group consisting of a seed, a root, a shoot, a leaf, a branch, a flower, a fruit and any combination thereof. Each possibility represents a separate embodiment of the present invention.

Any method as is known in the art for contacting bacterial strains with a plant, a part thereof or the plant habitat can be used according to the teachings of the present invention. Typically, the plant or part thereof is contacted with a composition comprising the bacterial strains, functional homologs thereof or preparations of same. According to some embodiments, the plant can be contacted directly with the composition. According to some embodiments, the composition may be applied to the plant habitat. According to certain embodiments, the plant habitat is a growth medium, which can be a solid or a liquid growth medium. According to certain exemplary embodiments, the solid growth medium is soil.

According to certain embodiments, the composition is formulated in a liquid form. According to these embodiments, the plant or part thereof may be contacted with the composition by a method selected from the group consisting of infiltration, immersion/dipping, incubation, spraying, and any combination thereof. Each possibility represents a separate embodiment of the present invention.

According to certain exemplary embodiments, the plant part is a seed. According to certain embodiments, the at least one bacterial strain according to the teachings of the invention is applied to the seed via seed coating.

According to certain additional or alternative exemplary embodiments, the plant part is a leaf. According to some embodiments, the at least one bacterial strain is applied to the leaf by spraying a bacterial preparation or a composition comprising same according to the teachings of the present invention.

According to certain exemplary embodiments, the bacterial strain is applied at a concentration range of from about 10² CFU/ml to about 10⁸ CFU/ml of the bacterial preparation or the agricultural composition. According to some embodiments, the bacterial strain is applied to seeds at a concentration range of from about 10² CFU/seed to about 10⁷ CFU/seed.

According to certain embodiments, the pest is an insect of an order selected from the group consisting of Lepidoptera, Coleoptera, and Hemiptera. Each possibility represents a separate embodiment of the present invention. According to certain embodiments, the pest is of the order Acari (mites).

According to some embodiments of the invention, the insect is selected from the group consisting of Western corn rootworm (WCRW, Diabrotica virgifera virgifera, of the order Coleoptera), Southern green stink bug (STK, Nezara viridula of the order Hemiptera), Fall armyworm (FAW, Spodoptera frugiperda of the order Lepidoptera), Beet Armyworm (BAW, Spodoptera exigua of the order Lepidoptera), Western tarnished plant bug (Lygus hesperus of the order Hemiptera), Cabbage Looper (Trichoplusia ni of the order Lepidoptera), Diamondback Moth (Plutella xylostella of the order Lepidoptera), Green Peach Aphid (Myzus persicae of the order Hemiptera), and Soybean Looper (Chrysodeixis includens of the order Lepidoptera). According to certain exemplary embodiments, the mite of the order Acari is Twospotted spider mite (Tetranychus urticae). Each possibility represents a separate embodiment of the present invention.

According to some embodiments, the insect is Western corn rootworm (Diabrotica virgifera virgifera, WCRW) and the plant is from a plant family selected from the group consisting of: Poaceae, Asteraceae, Cucurbitaceae, and Fabaceae. Each possibility represents a separate embodiment of the present invention.

According to some embodiments the insect is Fall armyworm (Spodoptera frugiperda, FAW) and the plant is from a plant family selected from the group consisting of: Amaranthaceae, Apiaceae, Apocynaceae, Asteraceae, Brassicaceae, Caryophyllaceae, Chenopodiaceae, Convolvulaceae, Cucurbitaceae, Cyperaceae, Euphorbiaceae, Fabaceae, Geraniaceae, Iridaceae, Juglandaceae, Liliaceae, Malvaceae, Musaceae, Platanaceae, Poaceae, Poaceae, Polygonaceae, Portulacaceae, Rosaceae, Rutaceae, Solanaceae, Ericaceae, Violaceae, Vitaceae, and Zingiberaceae. Each possibility represents a separate embodiment of the present invention.

According to some embodiments, the insect is Southern green stink bug (Nezara viridula, STK) and the plant is from a plant family selected from the group consisting of: Anacardiaceae, Asteraceae, Brassicaceae, Caprifoliaceae, Chenopodiaceae, Convolvulaceae, Cucurbitaceae, Euphorbiaceae, Fabaceae, Juglandaceae, Lauraceae, Magnoliaceae, Malvaceae, Oleaceae, Passifloraceae, Pedaliaceae, Poaceae, Proteaceae, Rosaceae, Rutaceae, Scrophulariaceae, Solanaceae, and Sterculiaceae. Each possibility represents a separate embodiment of the present invention.

According to some embodiments, the insect is Beet Armyworm (Spodoptera exigua) and the plant is from a plant family selected from the group consisting of: Poaceae, Malvaceae, Liliaceae, Amaranthaceae, Fabaceae, Solanaceae, Chenopodiaceae, Brassicaceae, Solanaceae, Cyperaceae, Juglandaceae, Asteraceae, Cucurbitaceae, Rutaceae, Euphorbiaceae, Convolvulaceae, Caryophyllaceae, Apiaceae, Polygonaceae, Rosaceae, Iridaceae, Musaceae, Geraniaceae, Platanaceae, Apocynaceae, Portulacaceae, Rosaceae, Ericaceae, Violaceae, Vitaceae, and Zingiberaceae. Each possibility represents a separate embodiment of the present invention.

According to some embodiments, the insect is Cabbage Looper (Trichoplusia ni) and the plant is from a plant family selected from the group consisting of: crucifers, beet, cantaloupe, celery, cucumber, lima bean, lettuce, parsnip, pea, pepper, potato, snap bean, spinach, squash, sweet potato, tomato, watermelon, chrysanthemum, hollyhock, snapdragon, sweet pea, cotton, tobacco, Chenopodium album, Lactuca spp. (wild lettuce), Taraxacum officinale (dandelion), and Rumex crispus (curly dock). Each possibility represents a separate embodiment of the present invention.

According to some embodiments, the insect is Diamondback Moth (Plutella xylostella) and the plant is from a plant family selected from the group consisting of: Malvaceae, Brassicaceae, Capparaceae, Asteraceae and Fabaceae. Each possibility represents a separate embodiment of the present invention.

According to some embodiments, the insect is Green Peach Aphid (Myzus persicae) and the plant is from a plant family selected from the group consisting of: Malvaceae, Euphorbiaceae, Aloaceae, Boraginaceae, Apiaceae, Scrophulariaceae, Araceae, Fabaceae, Brassicaceae, Asteraceae, Liliaceae, Chenopodiaceae, Solanaceae, Caricaceae, Apocynaceae, Cucurbitaceae, Rutaceae, Convolvulaceae, Iridaceae, Rosaceae, Caryophyllaceae, Euphorbiaceae, Iridaceae, Malvaceae, Poaceae, Cannabaceae, Balsaminaceae, Convolvulaceae, Poaceae, Lamiaceae, Papaveraceae, Lauraceae, Myrtaceae, Punicaceae, Anacardiaceae, Polygonaceae, and Pedaliaceae. Each possibility represents a separate embodiment of the present invention.

According to some embodiments, the insect is Soybean Looper (Chrysodeixis includens) and the plant is from a plant family selected from the group consisting of: Amaranthaceae, Apiaceae, Araceae, Araliaceae, Asteraceae, Begoniaceae, Brassicaceae, Caryophyllaceae, Chenopodiaceae, Convolvulaceae, Cucurbitaceae, Euphorbiaceae, Fabaceae, Geraniaceae, Gesneriaceae, Hydrangeaceae, Lamiaceae, Lauraceae, Liliaceae, Malvaceae, Passifloraceae, Piperaceae, Poaceae, Polygonaceae, Portulacaceae, Rubiaceae, and Solanaceae. Each possibility represents a separate embodiment of the present invention.

According to some embodiments, the insect is Western tarnished plant bug (Lygus hesperus) and the plant is from a plant family selected from the group consisting of: Cruciferae, Fabaceae, Malvaceae, Rosaceae, and Umbelliferae. Each possibility represents a separate embodiment of the present invention.

According to some embodiments, the plant pest is Twospotted spider mite (Tetranychus urticae) and the plant is from a family selected from the group consisting of Malvaceae, Asteraceae, Actinidiaceae, Liliaceae, Fabaceae, Apiaceae, Oxalidaceae, Chenopodiaceae, Theaceae, Solanaceae, Caricaceae, Apocynaceae, Cucurbitaceae, Rutaceae, Convolvulaceae, Betulaceae, Orchidaceae, Caryophyllaceae, Ebenaceae, Zingiberaceae, Salacia, Euphorbiaceae, Moraceae, Rosaceae, Iridaceae, Araliaceae, Cannabaceae, quifoliaceae, Balsaminaceae, Lamiaceae, Poaceae, Papaveraceae, Geraniaceae, Arecaceae, Ericaceae, Grossulariaceae, Pedaliaceae, Combretaceae, Tiliaceae, Violaceae, and Vitaceae . Each possibility represents a separate embodiment of the present invention.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention provides isolated microbial strains, particularly biologically pure isolated bacterial strains having unexpected activity against a variety of insects and/or mites, particularly insects and/or mites the presence of which in/on a plant has deleterious effects on the plant growth, health and typically yield (plant pests). The isolated bacterial strains of the invention can be used in various forms (preparations comprising a plurality of bacteria from the same or different species, lysates, extracts, whole cell broth and the like) as biocontrol agents protecting the plant from the insect and/or mite pests, by killing or at least inhibiting the development and/or activity of the insects and/or mites, or otherwise protecting the plant from a deleterious effect of at least one insect and/or mite pest.

### Definitions

The terms “comprise”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. As used herein, the term “plurality” refers to “at least two”.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween.

As used herein, the term “method” refers to manners, means, techniques and procedures for accomplishing a given task, including, but not limited to, those manners, means, techniques and procedures either known to, or readily developed from known manners, means, techniques and procedures by practitioners of the agricultural, chemical, pharmacological, biological, biochemical and medical arts, particularly of the agricultural and chemical arts.

When reference is made to particular sequence listings, such reference is to be understood to also encompass sequences that substantially correspond to its complementary sequence, and as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the terms “sequence identity” or “identity” or grammatical equivalents, in the context of two nucleic acid or polypeptide sequences, includes reference to the residues in the two sequences which are the same when aligned. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity), and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG (Amino acid substitution matrices from protein blocks. Proc. Natl. Acad. Sci. U.S.A. 1992, 89(22): 10915-9).

Identity can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof. According to some embodiments, the identity is partial identity, i.e., an identity over certain percentages of the length of the amino acid or nucleic acid sequences as described herein. According to certain exemplary embodiments, the identity between 16S rRNA sequences is at least 97% local identity over 98%, or 99% query coverage.

As used herein, the term “query coverage” refers to a percentage that describes how much of the query sequence is included in the region aligned to the target sequence.

As used herein, the terms “marker”, “genomic marker” and “sub-genomic sequence” are used herein interchangeably and refer to a DNA (deoxyribonucleic acid) sequence present within the genome of a microbial strain.

According to certain exemplary embodiments, identity of a genomic marker sequence is defined as at least 97% query coverage with at least 99% identity, such as further described herein.

The terms “microbial strain(s)” and “bacterial strain(s)” are used herein interchangeably and refer to the bacterial strains of the invention as defined herein.

The terms “functional homolog”, “functionally homologous”, “variant” and grammatical equivalents are used herein interchangeably and refer to a modification (i.e., mutant, at least one mutation) of the bacterial strains of the invention resulting in a microbial strain that is endowed with substantially the same ensemble of biological activities, particularly the capability to kill or inhibit the development and/or activity of at least one type of insect and/or mite, or the ability to protect plants from a deleterious effect of at least one insect and/or mite pest, (+/−10%, 20%, 40%, 50%, or 60% when tested under the same conditions) as that of the strain of the invention and can be classified to the same species or strain based on known methods of species/strain classifications and as described herein. The modification can be man-made or evolutionary, e.g., during propagation with or without selection.

The terms “isolated” and “biologically pure” with reference to a bacterial strain of the invention relate to bacterium or bacteria of the same species at least partially separated from the natural environment e.g., from the microbial strain habitat or from one or more constituents thereof, cellular or otherwise, with which it may be associated if found in nature. According to certain embodiments, the bacterial strains of the invention are isolated from the plant (the strain being part of the plant microbiome), from the plant habitat (including the plant rhizoplane and rhizosphere), or from the insect (the insect microbiome).

As used herein, the term “rhizoplane” refers to the external surface of roots together with closely adhering soil particles and debris.

As used herein, the term “rhizosphere” refers to the region of soil in the vicinity of plant roots, in which the soil chemistry and microbiology is influenced by the plant root growth, respiration, and nutrient exchange.

As used herein, the term “phyllosphere” refers to the total above-ground portions of a plant, which are a habitat for microorganisms. The phyllosphere can be further subdivided into the caulosphere (stems), phylloplane (leaves), anthosphere (flowers), and carposphere (fruit).

The terms “whole cell broth” and “whole cell culture” are used herein interchangeably and refer to a liquid culture containing both cells and a liquid medium. In particular embodiments, the terms relate to liquid medium comprising at least one bacterial strain of the invention and fermentation products thereof.

In the course of the present invention, selected strains were isolated and screened according to their ability to kill an insect pest/mite and/or to decrease deleterious effects of the insect/mite on the plant growth and/or yield. The isolated bacterial strains are described in Tables 1-3, and the strains' insecticidal activities are described in Tables 4-8 hereinbelow. Also contemplated are functional homologs of these strains, as defined and described herein.

According to certain embodiments, the bacterial strain or functional homolog thereof interacting with the host plant is present in the plant habitat, particularly in the rhizosphere (soil around root). According to certain embodiments, the bacterial strain or functional homolog thereof interacting with the host plant is present on or inside a plant tissue, including, but not limited to, the rhizoplane (root surface), root endosphere (inside the root), stem endosphere (inside the stem), leaf endosphere (inside the leaf), phyllosphere (on the shoot, stem and leaf surface), seed surface and seed endosphere (inside the seed). Each possibility represents a separate embodiment of the present invention.

According to one aspect, the present invention provides an isolated bacterial strain or a functional homologue thereof, wherein the isolated bacterial strain is selected from the group consisting of:
strain LAV61269, the strain being selected from the group consisting of (a) a strain deposited under Accession Number 43908 at NCIMB; (b) a strain comprising a 16S-rRNA sequence comprising the nucleic acid sequence set forth in SEQ ID NO:8; (c) a strain comprising at least one genomic marker comprising the nucleic acid sequence set forth in any one of SEQ ID NOs:73-75 and any combination thereof;
strain LAV41249, the strain being selected from the group consisting of (a) a strain deposited under Accession Number 43914 at NCIMB; (b) a strain comprising a 16S-rRNA sequence comprising the nucleic acid sequence set forth in SEQ ID NO:1; (c) a strain comprising at least one genomic marker comprising the nucleic acid sequence set forth in any one of SEQ ID NOs:52-55, and any combination thereof;
strain LAV61293, the strain being selected from the group consisting of (a) a strain deposited under Accession Number 43913 at NCIMB; (b) a strain comprising a 16S-rRNA sequence comprising the nucleic acid sequence set forth in any one of SEQ ID NO:11 and SEQ ID NO:12; (c) a strain comprising at least one genomic marker comprising the nucleic acid sequence set forth in any one of SEQ ID NOs:79-81, and any combination thereof;
strain LAV61301, the strain being selected from the group consisting of (a) a strain deposited under Accession Number 43910 at NCIMB; (b) a strain comprising a 16S-rRNA sequence comprising the nucleic acid sequence set forth in SEQ ID NO:17; (c) a strain comprising at least one genomic marker comprising the nucleic acid sequence set forth in any one of SEQ ID NOs:91-92, and any combination thereof;
strain LAV104746, the strain being selected from the group consisting of (a) a strain deposited under Accession Number 43911 at NCIMB; (b) a strain comprising a 16S-rRNA sequence comprising the nucleic acid sequence set forth in SEQ ID NO:26; (c) a strain comprising at least one genomic marker comprising the nucleic acid sequence set forth in any one of SEQ ID NOs:112-113, and any combination thereof;
strain LAV104743, the strain being selected from the group consisting of (a) a strain deposited under Accession Number 43903 at NCIMB; (b) a strain comprising a 16S-rRNA sequence comprising the nucleic acid sequence set forth in any one of SEQ ID NO:21 and SEQ ID NO:22; (c) a strain comprising at least one genomic marker comprising the nucleic acid sequence set forth in any one of SEQ ID NOs:104-107, and any combination thereof;
strain LAV61243, the strain being selected from the group consisting of (a) a strain deposited under Accession Number 43905 at NCIMB; (b) a strain comprising a 16S-rRNA sequence comprising the nucleic acid sequence set forth in SEQ ID NO:4; (c) a strain comprising at least one genomic marker comprising the nucleic acid sequence set forth in any one of SEQ ID NOs:62-64, and any combination thereof;
strain LAV106494, the strain being selected from the group consisting of (a) a strain deposited under Accession Number 43915 at NCIMB; (b) a strain comprising a 16S-rRNA sequence comprising the nucleic acid sequence set forth in SEQ ID NO:46; (c) a strain comprising at least one genomic marker comprising the nucleic acid sequence set forth in any one of SEQ ID NOs:144-145, and any combination thereof;
strain LAV1000771, the strain being selected from the group consisting of (a) a strain deposited under Accession Number 43912 at NCIMB; (b) a strain comprising a 16S-rRNA sequence comprising the nucleic acid sequence set forth in SEQ ID NO:49; (c) a strain comprising at least one genomic marker comprising the nucleic acid sequence set forth in any one of SEQ ID NOs:164-165, and any combination thereof;
strain LAV1000775, the strain being selected from the group consisting of (a) a strain deposited under Accession Number 43904 at NCIMB; (b) a strain comprising a 16S-rRNA sequence comprising the nucleic acid sequence set forth in SEQ ID NO:50; (c) a strain comprising at least one genomic marker comprising the nucleic acid sequence set forth in any one of SEQ ID NOs:166-170, and any combination thereof;
strain LAV1000731, the strain being selected from the group consisting of (a) a strain deposited under Accession Number 43906 at NCIMB; (b) a strain comprising a 16S-rRNA sequence comprising the nucleic acid sequence set forth in SEQ ID NO:48; (c) a strain comprising at least one genomic marker comprising the nucleic acid sequence set forth in any one of SEQ ID NOs:159-163, and any combination thereof;
strain LAV104934, the strain being selected from the group consisting of (a) a strain deposited under Accession Number 43902 at NCIMB; (b) a strain comprising a 16S-rRNA sequence comprising the nucleic acid sequence set forth in SEQ ID NO:40; (c) a strain comprising at least one genomic marker comprising the nucleic acid sequence set forth in any one of SEQ ID NOs:137-139, and any combination thereof;
strain LAV104944, the strain being selected from the group consisting of (a) a strain deposited under Accession Number 43907 at NCIMB; (b) a strain comprising a 16S-rRNA sequence comprising the nucleic acid sequence set forth in SEQ ID NO:45; (c) a strain comprising at least one genomic marker comprising the nucleic acid sequence set forth in any one of SEQ ID NOs:140-143, and any combination thereof.
strain LAV104753, the strain being selected from the group consisting of (a) a strain comprising a 16S-rRNA sequence comprising the nucleic acid sequence set forth in SEQ ID NO:30; (b) a strain comprising at least one genomic marker comprising the nucleic acid sequence set forth in any one of SEQ ID NOs:119-122, and a combination thereof;
strain LAV106564, the strain being selected from the group consisting of (a) a strain comprising a 16S-rRNA sequence comprising the nucleic acid sequence set forth in any one of SEQ ID NO:41 and SEQ ID NO:42; (b) a strain comprising at least one genomic marker comprising the nucleic acid sequence set forth in any one of SEQ ID NOs:146-148, and a combination thereof;
strain LAV100189, the strain being selected from the group consisting of (a) a strain comprising a 16S-rRNA sequence comprising the nucleic acid sequence set forth in SEQ ID NO:20; (b) a strain comprising at least one genomic marker comprising the nucleic acid sequence set forth in any one of SEQ ID NOs:100-103, and a combination thereof;
strain LAV1000686, the strain being selected from the group consisting of (a) a strain comprising a 16S-rRNA sequence comprising the nucleic acid sequence set forth in SEQ ID NO:47; (b) a strain comprising at least one genomic marker comprising the nucleic acid sequence set forth in any one of SEQ ID NOs:156-158, and a combination thereof;
strain LAV61251, the strain being selected from the group consisting of (a) strain comprising a 16S-rRNA sequence comprising the nucleic acid sequence set forth in SEQ ID NO:6; (b) a strain comprising at least one genomic marker comprising the nucleic acid sequence set forth in any one of SEQ ID NOs:68-70, and a combination thereof;
strain LAV61249, the strain being selected from the group consisting of (a) strain comprising a 16S-rRNA sequence comprising the nucleic acid sequence set forth in SEQ ID NO:5; (b) a strain comprising at least one genomic marker comprising the nucleic acid sequence set forth in any one of SEQ ID NOs:65-67, and a combination thereof;
strain LAV61268, the strain being selected from the group consisting of (a) a strain comprising a 16S-rRNA sequence comprising the nucleic acid sequence set forth in SEQ ID NO:7; (b) a strain comprising at least one genomic marker comprising the nucleic acid sequence set forth in any one of SEQ ID NOs:71-72, and a combination thereof;
strain LAV61295, the strain being selected from the group consisting of (a) a strain comprising a 16S-rRNA sequence comprising the nucleic acid sequence set forth in SEQ ID NO:13; (b) a strain comprising at least one genomic marker comprising the nucleic acid sequence set forth SEQ ID NO:82, and a combination thereof;
strain LAV61300, the strain being selected from the group consisting of (a) a strain comprising a 16S-rRNA sequence comprising the nucleic acid sequence set forth in SEQ ID NO:16; (b) a strain comprising at least one genomic marker comprising the nucleic acid sequence set forth in any one of SEQ ID NOs:88-90, and a combination thereof;
strain LAV61303, the strain being selected from the group consisting of (a) a strain comprising a 16S-rRNA sequence comprising the nucleic acid sequence set forth in SEQ ID NO:18; (b) a strain comprising at least one genomic marker comprising the nucleic acid sequence set forth in any one of SEQ ID NOs:93-97, and a combination thereof;
strain LAV104751, the strain being selected from the group consisting of (a) a strain comprising a 16S-rRNA sequence comprising the nucleic acid sequence set forth in SEQ ID NO:29; (b) a strain comprising at least one genomic marker comprising the nucleic acid sequence set forth in any one of SEQ ID NOs:117-118, and a combination thereof;
strain LAV104758, the strain being selected from the group consisting of (a) a strain comprising a 16S-rRNA sequence comprising the nucleic acid sequence set forth in any one of SEQ ID NO:35 and SEQ ID NO:36; (b) a strain comprising at least one genomic marker comprising the nucleic acid sequence set forth in any one of SEQ ID NOs: 128-131, and a combination thereof;
strain LAV104759, the strain being selected from the group consisting of (a) a strain comprising a 16S-rRNA sequence comprising the nucleic acid sequence set forth in any one of SEQ ID NO:37 and SEQ ID NO:38; (b) a strain comprising at least one genomic marker comprising the nucleic acid sequence set forth in any one of SEQ ID NOs:132-133, and a combination thereof;
strain LAV104761, the strain being selected from the group consisting of (a) a strain comprising a 16S-rRNA sequence comprising the nucleic acid sequence set forth in SEQ ID NO:39; (b) a strain comprising at least one genomic marker comprising the nucleic acid sequence set forth in any one of SEQ ID NOs:134-136, and a combination thereof;
strain LAV106654, the strain being selected from the group consisting of (a) a strain comprising a 16S-rRNA sequence comprising the nucleic acid sequence set forth in SEQ ID NO:43; (b) a strain comprising at least one genomic marker comprising the nucleic acid sequence set forth in any one of SEQ ID NOs:149-153, and a combination thereof;
strain LAV106657, the strain being selected from the group consisting of (a) a strain comprising a 16S-rRNA sequence comprising the nucleic acid sequence set forth in SEQ ID NO:44; (b) a strain comprising at least one genomic marker comprising the nucleic acid sequence set forth in any one of SEQ ID NOs:154-155, and a combination thereof; and
strain LAV61308, the strain being selected from the group consisting of (a) a strain comprising a 16S-rRNA sequence comprising the nucleic acid sequence set forth in SEQ ID NO:19; (b) a strain comprising at least one genomic marker comprising the nucleic acid sequence set forth in any one of SEQ ID NOs:98-99, and a combination thereof.

Each possibility represents a separate embodiment of the present invention.

Bacterial strains of Accession numbers 43901, 43902, 43903, 43905, 43907, 43908, 20 43909, 43910 and 43913 were deposited at NCIMB Ltd., Ferguson Building, Craibstone Estate, Bucksburn, Aberdeen, AB21 9YA Scotland, United Kingdon, on Jan. 19, 2022; Bacterial strains of Accession numbers 43904, 43906, 43911, 43912, 43914, 43915 and 43916 were deposited at NCIMB Ltd., Ferguson Building, Craibstone Estate, Bucksburn, Aberdeen, AB21 9YA Scotland, United Kingdon, on Jan. 25, 2022.

According to certain embodiments, the strain of the invention and the functional homolog belong to the same operational taxonomic units (OTU).

An “OTU″” (or plural, “OTUs”) refers to a terminal leaf in a phylogenetic tree and is defined by a nucleic acid sequence, e.g., the entire genome or a specific genetic sequence, and all sequences that share sequence identity to this nucleic acid sequence at the level of species. According to certain exemplary embodiments, the specific genetic sequence may be the 16S-rRNA sequence or a portion of the 16S-rRNA (also referred to herein as “16S”) sequence, or other functionally conserved sequences as listed below. According to additional exemplary embodiments, the entire genomes of two entities are sequenced and compared. In another embodiment, selected regions such as multilocus sequence tags (MLST), specific genes, or sets of genes may be genetically compared. For 16S-rRNA sequences, OTUs that share at least 97% average nucleotide identity across the entire 16S or some variable region of the 16S are considered the same OTU (see e.g., Claesson M J, et al. 2010. Nucleic Acids Res 38:e200; Konstantinidis K T, et al. 2006. Philos Trans R Soc Lond B Biol Sci 361:1929-1940). In embodiments involving the complete genome, MLSTs, specific genes, or sets of genes, OTUs that share at least 95% average nucleotide identity are considered the same OTU (see e.g., Achtman M, and Wagner M. 2008. Nat. Rev. Microbiol. 6:431-440; Konstantinidis et al. 2006, ibid). OTUs are frequently defined by comparing sequences between organisms. Such characterization employs, e.g., whole genome sequencing (WGS) data.

According to certain embodiments, the functional homolog comprises a functionally conserved gene or a fragment thereof. According to certain embodiments, the functionally conserved gene is a house-keeping gene selected from the group consisting of, but not limited to, 16S-rRNA, cysS, EngA, gyrB, recA, RimM, RsmD, ruvA, ruvB, TilS_N, uvrB, uvrC, YchF, IMPD, IF2, hemN, murC, holA, glnII, atpD, gap, glnA, gltA, pnp, rpoB, thrC and dnaK, that is at least about 99%, at least about 99.1%, at least about 99.2%, at least about 99.3%, at least about 99.4%, at least about 99.5%, at least about 99.6%, at least about 99.7%, at least about 99.8% or at least about 99.9%, or identical, over 97% coverage, to the sequence of the gene of a strain of the invention as described herein. Each possibility represents a separate embodiment of the present invention.

According to certain exemplary embodiments, the functionally conserved gene is a house-keeping gene selected from the group consisting of cysS, EngA, gyrB, recA, RimM, RsmD, ruvB, TilS_N, uvrB, uvrC, YchF, IMPD, ruvA, IF2, hemN, murC, holA, and a combination thereof, wherein each gene is at least about 99%, at least about 99.1%, at least about 99.2%, at least about 99.3%, at least about 99.4%, at least about 99.5%, at least about 99.6%, at least about 99.7%, at least about 99.8% or at least about 99.9%, or identical, over 97% coverage, to the sequence of the gene of a strain of the invention as described herein. Each possibility represents a separate embodiment of the present invention.

As mentioned, and according to a specific additional or an alternative embodiment, a functional homolog can also be determined on the basis of a multilocus sequence analysis (MLSA) determination of various functionally conserved genes or fragments thereof, e.g., at least one, 2, 3, 4, 5, 6, 7, 8, 9, 10, 11, 12, 13, 14, 15 or more functionally conserved genes or fragments thereof, such as of e.g., 16S, cysS, EngA, gyrB, recA, RimM, RsmD, ruvA, ruvB, TilS_N, uvrB, uvrC, YchF, IMPD, IF2, hemN, murC, holA, glnIII, atpD, gap, glnA, gltA, pnp, rpoB, thrC and dnaK.

According to certain exemplary embodiments, the house-keeping gene is 16S ribosomal RNA (16S-rRNA).

According to certain exemplary embodiments, the identity of the 16S sequence is defined as at least 99% query coverage with at least 97% identity, at least 97.5% identity, at least 98% identity, at least 98.5% identity, at least 99% identity, at least 99.5% identity or more.

According to these embodiments, the functional homolog bacterial strain comprises a 16S-rRNA sequence at least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homologous, or identical to the 16S-rRNA sequence of a strain of the invention, said 16S-rRNA sequence of the strain of the invention comprising a nucleic acid sequence selected from the group consisting of SEQ ID NOs:1, 4, 5, 6, 7, 11-13, 16-22, 26, 29-30, 35-39, 41-44, 46-50. Each possibility represents a separate embodiment of the present invention.

According to further embodiments, the functional homolog bacterial strain comprises a 16S-rRNA sequence at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homologous, or identical to the 16S-rRNA sequence of a strain of the invention, said 16S-rRNA sequence of the strain of the invention comprising a nucleic acid sequence selected from the group consisting of SEQ ID NOs:8, 40, and 45. Each possibility represents a separate embodiment of the present invention.

According to certain exemplary embodiments, the functional homolog bacterial strain comprises a 16S-rRNA sequence at least about 99%, at least about 99.1%, at least about 99.2%, at least about 99.3%, at least about 99.4%, at least about 99.5%, at least about 99.6%, at least about 99.7%, at least about 99.8%, at least about 99.9%or more homologous, or identical, to the 16S-rRNA sequence of a strain of the invention, said 16S-rRNA sequence comprising a nucleic acid sequence selected from the group consisting of SEQ ID NOs:1-51. According to certain exemplary embodiments, the functional homolog bacterial strain comprises a 16S-rRNA sequence at least about 99% or more identical to any one of SEQ ID NOs:8, 40 and 45. Each possibility represents a separate embodiment of the present invention.

According to certain exemplary embodiments, the bacterial strain comprises more than one 16S-rRNA.

According to certain embodiments, each of the isolated strains may be additionally or alternatively characterized by at least one strain-specific genomic marker.

According to certain embodiments, a functional homolog of the bacterial strains of the invention may be further or alternatively identified by comprising at least one genomic marker having at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homology, or identity, to a genomic marker of a strain of the present invention, over 97% coverage.

The functional homologs of strains LAV61269, LAV41249, LAV61293, LAV61301, LAV104746, LAV104743, LAV61243, LAV106494, LAV1000771, LAV1000775, LAV1000731, LAV104934, LAV104944, LAV104753, LAV106564, LAV100189, and LAV1000686 are as described hereinabove.

According to some exemplary embodiments, the functional homolog of strain LAV61251 comprises a 16S-rRNA sequence at least 97% identical to SEQ ID NO:6; at least one genomic marker having at least 99% local identity to the nucleic acid sequence of any one of SEQ ID NOs:68-70 over 97% coverage; or a combination thereof.

According to certain exemplary embodiments, the functional homolog of strain LAV61249, comprises a 16S-rRNA sequence at least 97% identical to SEQ ID NO:5; at least one genomic marker having at least 99% local identity to the nucleic acid sequence of any one of SEQ ID NOs:65-67; or a combination thereof;

According to some exemplary embodiments, the functional homolog of strain LAV61268 comprises a 16S-rRNA sequence at least 97% identical to SEQ ID NO:7; at least one genomic marker having at least 99% local identity to the nucleic acid sequence of any one of SEQ ID NOs:71-72 over 97% coverage; or a combination thereof.

According to some exemplary embodiments, the functional homolog of strain LAV61295 comprises a 16S-rRNA sequence at least 97% identical to SEQ ID NO:13; at least one genomic marker having at least 99% local identity to the nucleic acid sequence of SEQ ID NO:82 over 97% coverage; a combination thereof.

According to some exemplary embodiments, the functional homolog of strain LAV61300 comprises a 16S-rRNA sequence at least 97% identical to SEQ ID NO:16; at least one genomic marker having at least 99% local identity to the nucleic acid sequence of any one of SEQ ID NOs:88-90 over 97% coverage; combination thereof.

According to some exemplary embodiments, the functional homolog of strain LAV61303 comprises a 16S-rRNA sequence at least 97% identical to SEQ ID NO:18; at least one genomic marker having at least 99% local identity to the nucleic acid sequence of any one of SEQ ID NOs:93-97 over 97% coverage; or a combination thereof.

According to some exemplary embodiments, the functional homolog of strain LAV104751 comprises a 16S-rRNA sequence at least 97% identical to SEQ ID NO:29; at least one genomic marker having at least 99% local identity to the nucleic acid sequence of any one of SEQ ID NOs:117-118 over 97% coverage; or a combination thereof.

According to some exemplary embodiments, the functional homolog of strain LAV104758 comprises a 16S-rRNA sequence at least 97% identical to any one of SEQ ID NO:35 and SEQ ID NO:36; at least one genomic marker having at least 99% local identity to the nucleic acid sequence of any one of SEQ ID NOs:128-131 over 97% coverage; or a combination thereof.

According to some exemplary embodiments, the functional homolog of strain LAV104759 comprises a 16S-rRNA sequence at least 97% identical to any one of SEQ ID NO:37 and SEQ ID NO:38; at least one genomic marker having at least 99% local identity to the nucleic acid sequence of any one of SEQ ID NO:132-133 over 97% coverage; or a combination thereof.

According to some exemplary embodiments, the functional homolog of strain LAV104761 comprises a 16S-rRNA sequence at least 97% identical to SEQ ID NO:39; at least one genomic marker having at least 99% local identity to the nucleic acid sequence of any one of SEQ ID NO:134-136 over 97% coverage; or a combination thereof.

According to some exemplary embodiments, the functional homolog of strain LAV106654 comprises a 16S-rRNA sequence at least 97% identical to SEQ ID NO:43; at least one genomic marker having at least 99% local identity to the nucleic acid sequence of any one of SEQ ID NOs:149-153 over 97% coverage; or a combination thereof.

According to some exemplary embodiments, the functional homolog of strain LAV106657 comprises a 16S-rRNA sequence at least 97% identical to SEQ ID NO:44; at least one genomic marker having at least 99% local identity to the nucleic acid sequence of any one of SEQ ID NOs:154-155 over 97% coverage; or a combination thereof.

According to some exemplary embodiments, the functional homolog of strain LAV61308 comprises a 16S-rRNA sequence at least 97% identical to SEQ ID NO:19; at least one genomic marker having at least 99% local identity to the nucleic acid sequence of any one of SEQ ID NOs:98-99 over 97% coverage; or a combination thereof.

According to certain additional or alternative embodiments, the genomic nucleic acid sequences of the bacterial strain and the functional homolog thereof comprises at least one shared marker.

According to certain embodiments, the microbial strain of the present invention or the functional homolog thereof comprises at least two genomic markers, at least three genomic markers, at least four genomic markers, or at least five genomic markers. Each possibility represents a separate embodiment of the present invention.

According to certain exemplary embodiments, the functional homolog of a bacterial strain of the invention comprises at least one genomic marker, preferably at least two genomic markers selected from the group consisting of a marker having a nucleic acid sequence at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homologous, or identical to any one of SEQ ID NOs:52-175.

According to additional or alternative embodiments, the strain of the invention and the functional homolog are characterized by substantially the same (+/−about 10%, 20%, 40%, 50%, 60% when tested under the same conditions) biochemical profiling (e.g., biochemical fingerprinting) using for example, the GEN III redox chemistry (BIOLOG Inc. 21124 Cabot Blvd. Hayward CA, USA), which can analyze both Gram-negative and Gram-positive bacteria, for their ability to metabolize all major classes of biochemicals, in addition to determining other important physiological properties such as pH, salt, and lactic acid tolerance. Further details of biochemical profiling can be obtained in “Modern Phenotypic Microbial Identification”, Bochner B.R., Encyclopedia of Rapid Microbiological Methods, 2006, v.2, Ch. 3, pp. 55-73.

Genomic data can be obtained by methods which are well known in the art, e.g., DNA sequencing, bioinformatics, electrophoresis, enzyme-based mismatch detection assay and a hybridization assay; including, e.g., PCR, RT-PCR, Rnase protection, in-situ hybridization, primer extension, Southern blot, Northern Blot and dot blot analysis and the like. According to certain embodiments, the genomic markers of the functional homologs of the present invention were identified in the genomes of the microbial strains of the invention using the TIGRFAM annotation for protein families and screening the identified genes against the NCBI bacterial genome RefSeq database, as described in the Example section herein below.

According to certain exemplary embodiments, the functional homolog and the strain belong to the same genus.

According to certain embodiments, the strains of the present invention are of a genus selected from the group consisting of Pseudomonas, Aminobacter, Acinetobacter, Bacillus, Brevibacillus, Carnobacterium, Chitinophaga, Chryseobacterium, Ensifer, Microbacterium, Pantoea, Paraburkholderia, Rahnella, Rathayibacter, Serratia, Stenotrophomonas, Streptomyces and Variovorax. Each possibility represents a separate embodiment of the present invention.

According to certain exemplary embodiments, the strains of the present invention are of a genus selected from the group consisting of Pseudomonas, Serratia, Rahnella, Carnobacterium, Microbacterium, Stenotrophomonas, Chryseobacterium, Paraburkholderia, and Acinetobacter. Each possibility represents a separate embodiment of the present invention.

Bacteria are typically identified by morphological and biochemical tests as well as by molecular techniques. Specialized tests such as serotyping and antibiotic inhibition patterns can be also used. Based on such classification tests, the species of the strains of the invention are classified according to the closest known species.

According to certain embodiments, strain LAV61269, strain LAV104944, strain LAV104753 and strain LAV104761 each is of the genus Pseudomonass. According to certain embodiments, strain LAV41249, strain LAV61293, strain LAV1000771, strain LAV61300 and strain LAV104746 each is of the genus Serratia. According to certain embodiments, strains LAV61301 and LAV106657 each is of the genus Rahnella. According to certain embodiments, strains LAV104743 and LAV106564 each is of the genus Carnobacterium. According to certain embodiments, strains LAV61243 and

LAV104751 each is of the genus Microbacterium. According to certain embodiments, strains LAV61249, LAV100189 and LAV106494 each is of the genus Stenotrophomonas. According to certain embodiments, strain LAV1000775 is of the genus Chryseobacterium. According to certain embodiments, strain LAV1000731 is of the genus Paraburkholderia. According to certain embodiments, strain LAV104934 and strain LAV1000686 each is of the genus Acinetobacter. According to certain embodiments, strain LAV61251 is of the genus Rathayibacter. According to certain embodiments, strain LAV61268 and strain LAV61308 each is of the genus Bacillus. According to certain embodiments, strain LAV61303 is of the genus Aminobacter. According to certain embodiments, strain LAV104758 is of the genus Chitinophaga.

According to certain embodiments, strain LAV104759 is of the genus Brevibacillus. According to certain embodiments, strain LAV106654 and strain LAV61295 each is of the genus Ensifer.

According to certain exemplary embodiments, the functional homolog and the strain of the invention belong to the same genus/species.

According to certain exemplary embodiments, the functional homolog and the strain of the invention belong to the same genus, species or sub-species.

As used herein, the term “preparation” refers to an isolate of bacteria in which the prevalence (i.e., concentration and/or ratio) of the bacterial strain or functional homolog is enriched over that (exceeds that) found in nature. In nature, the bacterial strain is typically part of the plant microbiome, consisting of more than thousands of microbial species, whether in the phyllosphere, including endophytes, rhizoplane, and rhizosphere, or in any other plant compartment. The natural habitat of the bacterial strain can also be the insect or mite pests (insect/mite microbiome). According to some embodiments of the invention, the preparation comprises less than 50, 20, 10, 9, 8, 7, 6, 5, or 4 microbial species, e.g., bacteria and fungi, particularly bacteria other than bacterial strains of the invention.

According to certain embodiments, the microbial preparations comprise 1, 2, 3, 4, 5, 6, 7, 8, 9 or 10 microbial species. Each possibility represents a separate embodiment of the present invention.

According to certain exemplary embodiments, the microbial preparation comprises a single microbial species, i.e., a bacterial strain according to the teachings of the present invention.

According to certain exemplary embodiments, the preparation comprises a single strain of the present invention, wherein said preparation is devoid of other microbial species. According to certain further exemplary embodiments, the preparation comprises a plurality of strains of the present invention, wherein said preparation is devoid of other microbial species.

According to certain embodiments, the preparation comprises the bacterial strain of the invention at a level of purity of at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95% or more, say 100% pure. As used herein, the term “pure” or “purity” refers to the percentage of the bacterial strain of the invention out of the total number of microorganisms in the preparation.

According to certain exemplary embodiments, the preparation comprises the bacterial strain of the invention at a level of purity of at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.95%, at least about 99.99%, at least about 99.995%, at least about 99.999% or more, say 100% pure.

As used herein, the term “enriched” refers to 2-10×10⁶ -fold enrichment over that found in nature in an isolate of microbiota obtained from a plant phyllosphere and/or rhizosphere comprising a strain of the invention or a functional homolog of same.

According to certain embodiments, the bacterial preparation comprises viable bacterial cells (capable of replicating). According to some embodiments, the bacterial strain comprises sporulating bacteria. According to some embodiments, the viable bacterial cells are in a dormant state. According to some embodiments, the isolated bacterial strain is in a sporulated form.

According to some embodiments, the bacterial preparations of the invention comprise non-viable forms of the bacterial strains of the invention. According to certain embodiments, the non-viable form comprises non-sporulating bacteria. According to some embodiments, the non-viable form comprises dried bacterial strains.

A “spore” or “spores” refers to microbial structures that are generally viable, more resistant to environmental influences such as heat and bactericidal agents than other forms of the same microbial species, and typically capable of germination and out-growth.

Bacteria that are “capable of forming spores” are those bacteria comprising the genes and other necessary abilities to produce spores under suitable environmental conditions.

As used herein, the phrase “CFUs” or “Colony Forming Units” refers to the number of microbial cells in a defined sample (e.g., milliliter of liquid, square centimeter of surface, one seed of grain, etc.) that form colonies and are thereafter numbered, on a semi-solid bacteriological growth medium.

According to certain embodiments, the concentration in the composition e.g., preparation, formulation, coated seed etc. is 10² CFU to 10⁹ CFU/seed or 10² CFU-10⁹ CFU/gr powder or 10² CFU-10⁹ CFU/ml.

According to some embodiments, the bacterial strain is applied to seeds at a concentration range of from about 10² CFU/seed to about 10⁷ CFU/seed.

According to certain embodiments, the preparation is in a form selected from the group consisting of a liquid culture, a still (plate or non-shaking liquid) culture, whole culture stored stock of cells (particularly glycerol stocks), agar strip, stored agar plug in glycerol/water, freeze dried stock, and dried stocks such as lyophilizate dried onto filter paper or grain seed.

As used herein, the term “culture” refers to a fluid, pellet, scraping, dried sample, lyophilizate or a support, container, or medium such as a plate, paper, filter, matrix, straw, pipette or pipette tip, fiber, needle, gel, swab, tube, vial, particle, etc. that contains the deposited strain or the functional homolog thereof in an amount that exceeds that found in nature, as described hereinabove. In the present invention, an “isolated culture” of a microbial strain is a culture fluid or a scraping, pellet, dried preparation, lyophilizate, or a support, container, or medium that contains the deposited strain or the functional homolog thereof, in the absence of other microorganisms.

Cultures of the strains of the invention or of functional homologs thereof may be prepared for use according to the teachings of the invention using standard fermentation techniques known in the art. Growth is commonly performed in a bioreactor.

A bioreactor refers to any device or system that supports a biologically active environment. As described herein, a bioreactor is a vessel in which microorganisms, including the microorganism of the invention, can be grown. A bioreactor may be any appropriate shape or size for growing the microorganisms. A bioreactor may range in size and scale from 10 mL (e.g., small scale) to liters to cubic meters (e.g., large scale) and may be made of stainless steel, disposable material (e.g., nylon, plastic bags) or any other appropriate material as known and used in the art. The bioreactor may be a batch type bioreactor, a fed batch type or a continuous-type bioreactor (e.g., a continuous stirred reactor). For example, a bioreactor may be a chemostat as known and used in the art of microbiology for growing and harvesting microorganisms. A bioreactor may be obtained from any commercial supplier (See also Bioreactor System Design, Asenjo and Merchuk, CRC Press, 1995).

For small scale operations, a batch bioreactor may be used, for example, to test and develop new processes, and for processes that cannot be converted to continuous operations.

Microorganisms grown in a bioreactor may be suspended or immobilized. Growth in the bioreactor is generally under aerobic conditions at suitable temperatures and pH for growth. For the organisms of the invention, cell growth can be achieved at temperatures between 5-37° C., with an exemplary temperature range selected from 15 to 30° C., 15 to 28° C., 20 to 30° C., or 15 to 25° C. The pH of the nutrient medium can vary between 4.0 and 9.0. For example, the operating range can be usually slightly acidic to neutral at pH 4.0 to 7.0, or 4.5 to 6.5, or pH 5.0 to 6.0. Typically, maximal cell yield is obtained in 20-72 hours after inoculation.

Optimal conditions for the cultivation of the microorganisms of this invention will, of course, depend upon the particular strain and strain species. However, by virtue of the conditions applied in the selection process and general requirements of most microorganisms, a person of ordinary skill in the art would be able to determine essential nutrients and conditions. The microorganisms would typically be grown in aerobic liquid cultures on media which contain sources of carbon, nitrogen, and inorganic salts that can be assimilated by the microorganism and supportive of efficient cell growth. Exemplary carbon sources are hexoses such as glucose, but other sources that are readily assimilated, such as amino acids, may form a substitute. Many inorganic and proteinaceous materials may be used as nitrogen sources in the growth process. Exemplary nitrogen sources are amino acids and urea, but others include gaseous ammonia, inorganic salts of nitrate and ammonium, vitamins, purines, pyrimidines, yeast extract, beef extract, proteose peptone, soybean meal, hydrolysates of casein, and the like. Among the inorganic minerals that can be incorporated into the nutrient medium are the customary salts capable of yielding calcium, zinc, iron, manganese, magnesium, copper, cobalt, potassium, sodium, molybdate, phosphate, sulfate, chloride, borate, and like ions.

The culture can be a pure culture, whereby a single microbial strain is grown, or a mixed culture. A mixed culture can be prepared pending the compliance of the microbial strains to co-exist and proliferate under the same culturing conditions. When needed, an antibiotic or other growth-restricting conditions, e.g., temperature, essential nutrients and the like can be employed during culturing to restrict the growth of other microorganisms (contaminants) not desired in the culture/co-culture.

According to alternative or additional embodiments, a desired strain combination is produced following culturing. Typically, a strain combination is made after culturing when the microbial strains do not share the same or optimal culturing conditions.

When a combination of strains is to be used, the ratio of each type of microorganism in the final product will depend on the target pest to be eradicated.

It is to be explicitly understood that the present invention encompasses complete cultures comprising a growth medium and at least one bacterial strains or functional homologs of the invention, as well as growth medium obtained after removal of the bacterial strains. According to certain embodiments, the growth medium obtained after removal of the at least one bacterial strain encompasses fermentation products of said at least one bacterial cell.

According to a further aspect, the present invention provides a lysate of at least one bacterial strain or of a functional homolog thereof according to the teachings of the present invention.

According to the certain embodiments, the lysate is of a single strain. According to some embodiments, the lysate is of a plurality of strains. According to certain embodiments, the plurality of strains is of the same genus and/or species. According to some embodiments, the plurality of strains comprises a plurality of strain genera and/or species.

According to certain embodiments, the lysate comprises a whole cell lysate of the bacteria.

According to certain embodiments, the lysate comprises a soluble fraction of the bacterial cells.

According to a further aspect, the present invention provides a cell extract of at least one bacterial strain or a functional homolog thereof according to the teachings of the present invention. According to some embodiments, the cell extract is of a plurality of strains. According to certain embodiments, the plurality of strains is of the same genus and/or species. According to some embodiments, the plurality of strains comprises a plurality of strain genera and/or species.

According to an additional aspect, the present invention provides a composition comprising the bacterial preparation, culture, culture medium, lysate or extract as described herein, further comprising agriculturally acceptable carriers and/or diluents.

According to certain embodiments, the composition further comprises an agriculturally effective amount of an active agent selected from the group consisting of a fertilizer, an additional acaricide, a fungicide, an additional insecticide, a nematicide, a plant growth regulator, a rodenticide, and a nutrient.

Also provided is a formulation comprising the bacterial preparation or the composition comprising same as described herein.

Any carrier suitable for agricultural use can form part of the compositions and/or formulations of the present invention. The carrier may be any one or more of a number of carriers that confer a variety of properties, including increased stability, wettability, dispersability, etc. Wetting agents such as natural or synthetic surfactants, which can be nonionic or ionic surfactants, or a combination thereof can be included in a composition of the invention. Water-in-oil emulsions can also be used to formulate a composition that includes at least one isolated microorganism of the present invention (see, for example, U.S. Pat. No. 7,485,451). Suitable formulations that may be prepared include wettable powders, granules, gels, agar strips or pellets, and the like, microencapsulated particles, and the like, liquids such as aqueous flowables, aqueous suspensions, water-in-oil emulsions, etc. The formulation may include grain or legume products (e.g., ground grain or beans, broth or flour derived from grain or beans), starch, sugar, or oil. The carrier may be an agricultural carrier. In certain preferred embodiments, the carrier is a seed, and the composition may be applied or coated onto the seed or allowed to saturate the seed.

According to some embodiments, the agricultural carrier may be soil or plant growth medium. Other agricultural carriers that may be used include water, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, clay, etc. Formulations may include food sources for the cultured organisms, such as barley, rice, or other biological materials such as seed, plant parts, sugar cane bagasse, hulls or stalks from grain processing, ground plant material (“yard waste”) or wood from building site refuse, sawdust or small fibers from recycling of paper, fabric, or wood. Other suitable formulations will be known to those skilled in the art.

In the liquid form, e.g., solutions or suspensions, the microbial strain may be mixed or suspended in water or in aqueous solutions. Suitable liquid diluents or carriers include water, aqueous solutions, petroleum distillates, or other liquid carriers.

Solid compositions can be prepared by dispersing the microbial strain in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

According to certain embodiments, the bacterial strains of the present invention are applied within a “dehydrated microfermentor” as described in International (PCT) Application Publication No. WO2020021549. As used herein, this term refers to a dehydrated composition comprising a particle encapsulating one or more microorganisms, wherein the particle is composed of an inner core comprising the one or more microorganisms surrounded by an outer shell layer, wherein said outer shell layer is selectively permeable to a rehydrating fluid, and wherein upon fluid absorption, said outer shell layer degrades at a predetermined rate, thereby releasing a plurality of microorganisms to the surrounding environment in a controlled manner. According to certain embodiments, the encapsulated microorganisms, particularly the bacterial strains and functional homologs of the invention, are present in the dehydrated composition at an initial concentration of less than about 1×10³ CFU/particle, and, following fluid absorption, the concentration of the encapsulated microorganisms is increased by at least 10-fold before the microorganisms are released to the surrounding environment. According to certain exemplary embodiments, the concentration of the released microorganisms is at least 10⁵ CFU. In certain embodiments, the dehydrated composition comprises a plurality of particles.

Any fertilizer as is known in the art can be added to the compositions/formulations of the present invention, as long as the fertilizer does not interfere with the bacterial growth and activity. According to certain embodiments, the fertilizer is selected from the group consisting of chemical or biological fertilizer. The amount of the at least one chemical or biological fertilizer employed can vary depending on the final formulation as well as the size of the plant and/or seed to be treated.

A variety of chemical pesticides may be added to the compositions of the present invention. Exemplary chemical pesticides include acylalanines, butyrolactones, oxazolidinones, hydroxy-(2-amino-) pyrimidines, isothiazolones, isoxazoles, carboxylic acids, benzimidazoles, thiophanates, N-phenyl carbamates, toluamides, ethylamino-thiazole-carboxamide, phenylureas, pyridinylmethyl-benzamides, aminocyanoacrylates, benzophenone, benzoylpyridine, pyrazole-5-carboxamides, pyrimidinamines, quinazoline, N-methoxy-(phenyl-ethyl)-pyrazole-carboxamides, furan-carboxamides, oxathiin-carboxamides, phenyl-benzamides, phenyl-oxo-ethyl thiophene amide, pyrazole-4-carboxamides, N-cyclopropyl-N-benzyl-pyrazole-carboxamides, pyridine-carboxamides, pyridinyl-ethyl-benzamides, thiazole-carboxamides, pyrazine-carboxamides, benzyl-carbamates, dihydro-dioxazines, imidazolinones, methoxy-acetamide, methoxy-acrylates, methoxy-carbamates, oxazolidine-diones, oximino-acetamides, oximino-acetates, tetrazolinones, cyano-imidazole, sulfamoyl-triazole, picolinamides, dinitrophenyl crotonates, 2,6-dinitro-anilines, tri-phenyl tin compounds, thiophene-carboxamides, triazolo-pyrimidylamine, anilino-pyrimidines, enopyranuronic acid, hexopyranosyls, glucopyranosyls, tetracycline, aryloxyquinoline, quinazolinone, phenylpyrroles, dicarboximides, dithiolanes, phosphoro-thiolates, aromatic hydrocarbons, 1,2,4-thiadiazoles, carbamates, piperidinyl-thiazole-isoxazolines, imidazoles, piperazines, pyridines, pyrimidines, triazoles, triazolinthiones, morpholines, piperidines, spiroketal-amines, amino-pyrazolinone, hydroxyanilides, allylamines, thiocarbamates, peptidyl pyrimidine nucleoside, cinnamic acid amides, mandelic acid amides, valinamide carbamates, isobenzo-furanone, pyrrolo-quinolinone, triazolobenzo-thiazole, carboxamide, cyclopropane-carboxamide, propionamide, trifluoroethyl-carbamate, benzothiadiazole, thiadiazole-carboxamide, ethyl phosphonates, cyanoacetamide-oxime, phthalamic acids, benzotriazines, benzene-sulfonamides, pyridazinones, phenyl-acetamide, guanidines, cyano-methylene-thiazolidines, pyrimidinone-hydrazones, 4-quinolyl-acetates, tetrazolyloximes, dithiocarbamates, phthalimides, chloronitriles, sulfamides, bis-guanidines, triazines, quinones, quinoxalines, maleimide and phthalonitriles, as well as copper fungicides (acypetacs-copper, Bordeaux mixture, Burgundy mixture, Cheshunt mixture, copper acetate, basic copper carbonate, copper hydroxide, copper naphthenate, copper oleate, copper oxychloride, copper silicate, copper sulfate, basic copper sulfate, copper zinc chromate, cufraneb, cuprobam, cuprous oxide, mancopper, oxine-copper, saisentong, thiodiazole-copper) and other fungicides, including dimethomorph, fosetyl-Al, tetraconazole, azoxystrobin, propineb, pyraclostrobin, potassium phosphite, mefenoxam, and folpet.

The formulation as used herein can also refer to a customary formulation in an effective amount to be applied either to the soil (e.g., in-furrow), to a portion of the plant (e.g., drench) or on the seed before planting (e.g., seed coating or dressing). Customary formulations include solutions, emulsifiable concentrates, wettable powders, suspension concentrates, soluble powders, granules, suspension-emulsion concentrates, natural and synthetic materials impregnated with active compounds, and very fine controlled release capsules in polymeric substances. In certain embodiments of the present invention, the microbial strains are formulated in powders that are available in either a ready-to-use formulation or are otherwise mixed together at the time of use. In either embodiment, the powder may be admixed with the soil prior to or at the time of planting.

Depending on the final formulation, one or more suitable additives can also be introduced to the compositions of the present invention. Adhesives such as carboxymethylcellulose and natural and synthetic polymers in the form of powders, granules or latexes, such as gum arabic, chitin, polyvinyl alcohol and polyvinyl acetate, as well as natural phospholipids, such as cephalins and lecithins, and synthetic phospholipids, can be added to the compositions/formulation of the present invention.

According to certain embodiments, the bacterial strains are formulated in a single, stable solution, or emulsion, or suspension. For solutions, the chemical compounds are typically dissolved in solvents before the microbial strain is added. Suitable liquid solvents include petroleum-based aromatics, such as xylene, toluene or alkylnaphthalenes, aliphatic hydrocarbons, such as cyclohexane or paraffins, for example petroleum fractions, mineral and vegetable oils, alcohols, such as butanol or glycol as well as their ethers and esters, ketones, such as methyl ethyl ketone, methyl isobutyl ketone or cyclohexanone, strongly polar solvents, such as dimethylformamide and dimethyl sulphoxide. For emulsion or suspension, the liquid medium is water. In some embodiments, the chemical agent and the microbial strain are suspended in separate liquids and mixed at the time of application. In a preferred embodiment of suspension, the chemical agent and the microbial strain are combined in a ready-to-use formulation that exhibits a reasonably long shelf-life. In use, the liquid can be sprayed or can be applied to the plant foliage as an atomized spray or in-furrow at the time of planting the crop. The liquid composition can be introduced in an effective amount on the seed (i.e., seed coating or dressing) or to the soil (in-furrow) before germination of the seed or directly to the soil in contact with the roots by utilizing a variety of techniques known in the art including, but not limited to, drip irrigation, sprinklers, soil injection or soil drenching. Optionally, stabilizers and buffers can be added, including alkaline and alkaline earth metal salts and organic acids, such as citric acid and ascorbic acid, inorganic acids, such as hydrochloric acid or sulfuric acid.

The amount of the bacterial strain or functional homolog within the composition/formulation is sufficient to interact, colonize and/or localize in a cultivated plant treated with same.

One of ordinary skill in the art would know how to calculate the concentration of the microbial strain or functional homolog.

According to certain embodiments, the bacterial strain(s) is about 2% w/w to about 80% w/w of the entire formulation/composition. According to other embodiments, the bacterial strains(s) employed in the compositions is about 5% w/w to about 65% w/w or about 10% w/w to about 60% w/w by weight of the entire formulation/composition.

According to certain embodiments, the preparation/composition provided herein is formulated to provide stability for the bacterial strain or functional homolog. Optionally, a shelf-stable formulation is in a dry form, e.g., a powder formulation, or a lyophilized formulation. According to certain embodiments, the formulation is substantially stable at temperatures between about −20° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In other embodiments, the formulation is substantially stable at temperatures between about 4° C. and about 37° C. for at least about 5, 10, 15, 20, 25, 30 or for about 180 days and more. According to certain exemplary embodiments, the formulation is substantially stable at temperatures between about 4° C. and about 37° C. for at least about 30 days. According to certain exemplary embodiments, the microbial strain or functional homolog may be shelf-stable, wherein at least 0.01% of the CFU or spores are viable after storage in desiccated form (i.e., moisture content of 30% or less) for 1, 2, 3, 4, 5, 6, 7, 8, 9, 10 or more than 10 weeks at 4° C. or at room temperature.

The bacterial strains of the present invention and their functional homologs are effective in inhibiting the activity and/or growth and/or development, and/or in killing insect and/or mite pests, particularly plant insect and/or mite pests.

As used herein and in the claims section below, the phrases “inhibiting the activity and/or growth and/or development, and/or in killing” and “having insecticidal activity” and “having acaricidal activity” are used herein interchangeably and refer to an effective amount of the bacterial strain(s) and homolog(s) thereof according to the teachings of the invention which is capable of killing or inhibiting the development of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% of a population of the insect/mite as compared to the population of an insect/mite of the same species not exposed to/being in contact with/consuming the effective amount of the bacterial strain(s) or homolog(s) thereof when grown under the same (e.g., identical) growth conditions; and/or when compared to the initial population of the insect/mite prior to being exposed to/contacted with/fed with the agent of some embodiments of the invention.

Methods of qualifying insecticidal/acaricidal activity of an agent are known in the art (e.g., MacIntosh, Susan C., et al. “Specificity and efficacy of purified Bacillus thuringiensis proteins against agronomically important insects” Journal of invertebrate pathology 56(2) (1990): 258-266; 0′ Callahan M., et al. Bioassay of bacterial entomopathogenes against insect larvae. Lacey, Lawrence A., ed. Manual of techniques in invertebrate pathology. Academic Press, 2012. Chapter IV p:101-127), and are further described and exemplified hereinbelow. In addition, IC₅₀ values can be determined to qualify effective concentration of the agent resulting in inhibiting growth and development of at least 50% of the insect/mite population.

According to certain embodiments, the phrases “inhibitory activity” and/or “inhibiting the development of an insect/mite”, which are interchangeably used herein, refer to an activity which results in reducing the size and/or mass (e.g., stunting) of the insect/mite as compared to the size and/or mass of an insect/mite of the same species in the absence of the effective amount of the agent under the same (e.g., identical) growth conditions; and/or when compared to the size and/or mass of the insect/mite prior to being contacted with the bacterial strains of the invention. The inhibitory activity can be direct, i.e., resulting from consumption by the pest, or indirect, i.e., resulting from application to the plant of the bacterial strain(s), extract, lysate, whole cell broth and the like according to the teachings of the invention.

According to certain embodiments, a strain of the invention is examined for its capability to inhibit the activity and/or development of, or to kill, an insect and/or a mite, by assessing the symptoms associated with the presence of the insect/mite in or on a plant, a part thereof or its habitat in a control plant compared to a plant treated with the bacterial strain. The control plant is typically, but not necessarily, of the same species as the treated plant. According to some embodiments, the control plant is of the same species and has the same genetic background as the treated plant. The capability can be manifested as an increase of 0.1%, 0.2%, 0.3%, 0.5%, 0.75%, 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, 100%, or more in health, growth, multiplication, fertility, vigor, strength, or yield, as compared to a control plant. The capability can be a decrease of 0.1%, 0.2%, 0.3%, 0.5%, 0.75%, 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, or 100% in the symptoms associated with the presence of the insect or mite. According to certain exemplary embodiments, the treated plant and the control plant are grown under the same conditions.

Symptoms associated with the presence of an insect/mite in or on a plant or its habitat are known to a person skilled in the art. Typically, a score scale is set for a certain symptom associated with the presence of the insect/mite based on symptom description and/or severity. Optionally, a specific score scale is set for a combination of certain symptoms and a plant species. The use of score scale simplifies the comparison of symptoms in plants subjected to various treatments.

According to certain aspects, the present invention provides a method for inhibiting or preventing the growth and/or the development of at least one pest present in or on a plant or a part thereof, comprising contacting the plant or part thereof or the plant habitat with at least one bacterial strain selected from the group consisting of LAV61249, LAV41249, LAV61242, LAV61243, LAV61251, LAV61268, LAV61269, LAV61288, LAV61293, LAV61295, LAV61297, LAV61300, LAV61301, LAV61303, LAV61308, LAV100189, LAV104743, LAV104744, LAV104746, LAV104747, LAV104751, LAV104753, LAV104754, LAV104758, LAV104759, LAV104761, LAV104934, LAV104944, LAV106494, LAV106564, LAV106654, LAV106657, LAV1000686, LAV1000731, LAV1000771, LAV1000775, LAV and functional homologs thereof; a preparation of same, a lysate thereof, an extract thereof, a broth obtained therefrom or a composition comprising same. Each possibility represents a separate embodiment of the present invention.

According to additional aspects, the present invention provides a method for preventing and/or treating symptoms associated with the presence of at least one insect and/or mite in or on a plant or a part thereof, comprising contacting the plant or part thereof or the plant habitat with at least one bacterial strain selected from the group consisting of LAV61269, LAV41249, LAV61242, LAV61243, LAV61249, LAV61251, LAV61268, LAV61288, LAV61293, LAV61295, LAV61297, LAV61300, LAV61301, LAV61303, LAV61308, LAV100189, LAV104743, LAV104744, LAV104746, LAV104747, LAV104751, LAV104753, LAV104754, LAV104758, LAV104759, LAV104761, LAV104934, LAV104944, LAV106494, LAV106564, LAV106654, LAV106657, LAV1000686, LAV1000731, LAV1000771, LAV1000775, LAV and functional homologs thereof; a preparation of same, a lysate thereof, an extract thereof, a broth obtained therefrom or a composition comprising same. Each possibility represents a separate embodiment of the present invention.

According to certain aspects, the present invention provides a method for protecting a plant from a deleterious effect of at least one pest present in or on the plant or part thereof, comprising contacting said plant or part thereof or the plant habitat with at least one bacterial strain selected from the group consisting of LAV61269, LAV41249, LAV61242, LAV61243, LAV61249, LAV61251, LAV61268, LAV61288, LAV61293,

LAV61295, LAV61297, LAV61300, LAV61301, LAV61303, LAV61308, LAV100189, LAV104743, LAV104744, LAV104746, LAV104747, LAV104751, LAV104753, LAV104754, LAV104758, LAV104759, LAV104761, LAV104934, LAV104944, LAV106494, LAV106564, LAV106654, LAV106657, LAV1000686, LAV1000731, LAV1000771, LAV1000775, LAV1000939 and functional homologs thereof; a preparation of same, a lysate thereof, an extract thereof, a broth obtained therefrom or a composition comprising same. Each possibility represents a separate embodiment of the present invention.

According to certain embodiments, strain LAV61242 is selected from the group consisting of (a) a strain comprising a 16S-rRNA sequence comprising the nucleic acid sequence set forth in SEQ ID NO:3; (b) a strain comprising at least one genomic marker comprising the nucleic acid sequence set forth in any one of SEQ ID NOs:57-61, and a combination thereof.

According to certain embodiments, strain LAV61297 is selected from the group consisting of (a) a strain comprising a 16S-rRNA sequence comprising the nucleic acid sequence set forth in any one of SEQ ID NOs:14-15; (b) a strain comprising at least one genomic marker comprising the nucleic acid sequence set forth in any one of SEQ ID NOs:83-87, and a combination thereof.

According to certain embodiments, strain LAV104754 is selected from the group consisting of (a) a strain comprising a 16S-rRNA sequence comprising the nucleic acid sequence set forth in any one of SEQ ID NO:31-32; (b) a strain comprising at least one genomic marker comprising the nucleic acid sequence set forth in any one of SEQ ID NOs:123-124, and a combination thereof.

According to certain embodiments, strain LAV61242 is of the genus Pantoea; According to certain embodiments, strain LAV61297 is of the genus Streptomyces; According to certain embodiments, strain LAV104744 is of the genus Acinetobacter; According to certain embodiments, strain LAV61288, strain LAV104747 and strain LAV104754 each is of the genus Pseudomonas; and according to certain embodiments, strain LAV1000939 is of the genus Variovorax.

The additional bacterial strains and functional homologs thereof, as well as preparations of same, lysates thereof, extracts thereof, broth obtained therefrom or compositions comprising same, are as described hereinabove.

According to certain embodiments, the plant part is selected from the group consisting of a seed, a root, a shoot, a leaf, a branch, a flower, a fruit and any combination thereof. Each possibility represents a separate embodiment of the present invention.

Any method as is known in the art can be used to contact the plant or part thereof with the at least one bacterial strain or functional homolog thereof of the present invention. According to certain embodiments, the plant or part thereof is contacted with a preparation/composition comprising the at least one bacterial strain or functional homolog thereof by a method selected from the group consisting of, but not limited to, immersing, dipping, spraying, coating, dusting and any combination thereof.

According to other embodiments, the plant or part thereof is contacted with the at least one bacterial strain or functional homolog thereof through the plant rhizosphere. According to these embodiments, the preparation/composition is applied to the plant rhizosphere.

According to certain exemplary embodiments, the plant part is a seed and contacting is affected by seed coating.

According to certain exemplary embodiments, the plant part is a leaf and contacting is performed by spraying or dusting.

According to certain exemplary embodiments, the plant part is a root and contacting is performed by dipping or immersing.

According to certain exemplary embodiments, the plant part is a seed or a root, and contacting is performed by applying the bacterial strain or functional homolog thereof, preparations, extract, lysate, whole cell broth or a composition comprising same, to the seed or root growth medium.

According to certain embodiments, the plant or part thereof is surface sterilized prior to contacting with the bacterial preparation/composition/formulation, particularly for research applications.

Any means for applying the bacterial strains and/or functional homologs thereof, preparations, lysates, extracts, whole cell broth and/or compositions comprising same, as such means are known in the art, can be used with the teachings of the present invention.

According to some embodiments, wherein the application is to the aerial part of a plant, application may be mediated by beneficial insects, for example bees, exposed to the bacterial strains and/or their formulations. According to some embodiment, the bacterial strains and/or formulations comprising same are at least partially coating the beneficial insects.

Application to the plant aerial part may also be mediated by moving vehicles (cars or agricultural vehicles), flying devices (e.g., drones), and the like.

In some embodiments, the bacterial preparation, compositions and/or formulations comprising same can be contacted with the plant or part thereof, for example the plant seed and/or plant aerial parts, and successful colonization can be confirmed by detecting the presence of the microbial strain within the plant. For example, after applying the composition/formulation/preparation to the seeds, high titers of the microbial strain can be detected in the roots and shoots of the plants that germinate from the seeds. In addition, significant quantities of the microbial strain can be detected in the rhizosphere of the plants. Therefore, in some embodiments, the microbial strain is applied in an amount effective to colonize the plant. The microbial strain may be colonized on the surface of the plant or within the plant tissues as an endophyte. In some embodiments, colonization of the plant can be detected, for example, by detecting the presence of the microbial strain inside the plant. This can be accomplished by measuring the viability of the microbial strain after surface sterilization of the plant portion: microbial strain colonization results in an internal localization of the microbe, rendering it resistant to conditions of surface sterilization. According to some embodiments, the microbial strain is applied in an amount effective to colonize the plant rhizosphere. The presence and quantity of the microbial strain can also be established using other means known in the art, for example, immunofluorescence microscopy using microbe-specific antibodies, or fluorescence of in situ hybridization. Alternatively, specific nucleic acid probes recognizing conserved sequences from the colonized bacterial strain can be employed to amplify a region, for example by quantitative PCR, and correlated to CFUs by means of a standard curve.

According to certain exemplary embodiments, the bacterial strain can be detectable within a target tissue of the mature cultivated plant selected from a fruit, a seed, a leaf, or a root, a portion thereof and a combination thereof. It is to be explicitly understood that the bacterial strain or the functional homolog can colonize a plant part distinct from the plant part with which said bacterial strain or functional homolog was contacted.

According to certain embodiments, the bacterial strain contacted with the plant, part thereof or the plant rhizosphere is not detected in said plant, plant part thereof and rhizosphere prior to application of said bacterial strain. According to other embodiments, the bacterial strain is naturally present in the plant, part thereof, and/or plant rhizosphere. In any case, the amount of the bacterial strain in the plant, part thereof or plant rhizosphere is higher after contacting with the strain compared to its amount prior to contacting (application).

According to certain aspects, the present invention provides a modified bacterial strain having an improved insect and/or mite killing or inhibitory activity, or an improved ability to protect a plant from a deleterious effect of at least one pest, as compared to a corresponding bacterial strain of the same species according to some embodiments of the present invention. According to certain currently exemplary embodiments, the modified bacterial strain is a non-genetically modified organism (non-GMO).

According to certain further aspects, the present invention provides a method of obtaining a modified bacterial strain having an improved insect and/or mite killing or inhibitory activity, or an improved ability to protect a plant from a deleterious effect of at least one pest, as compared to a parent bacterial strain of the same species according to some embodiments of the invention, comprising:
(a) culturing the bacterial strain according to some embodiments of the invention under conditions suitable for expanding a population of the bacterial strain and allowing development of at least one bacterial mutant, and
(b) selecting the at least one bacterial mutant resultant of step (a) for an improved insect and/or mite killing or inhibitory activity or for an improved protection of a plant from the deleterious effects of at least one insect and/or mite,
thereby obtaining the modified bacterial strain having the improved insect and/or mite killing or inhibitory activity, or the improved ability to protect a plant from a deleterious effect of at least one pest as compared to the parent bacterial strain of the same species according to some embodiments of the invention.

Insect pests include insects selected from the orders Lepidoptera, Coleoptera, Diptera, Hemiptera, Hymenoptera, Mallophaga, Homoptera, Orthroptera, Thysanoptera, Dermaptera, Isoptera, Anoplura, Siphonaptera, Trichoptera and the like.

According to certain exemplary embodiments, the insect is of an order selected from the group consisting of Lepidoptera, Coleoptera, Hemiptera. Each possibility represents a separate embodiment of the present invention.

According to certain embodiments the pest is a mite of the of the order Acari.

The order Lepidoptera includes several families such as Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Non-limiting examples of insects of the order Lepidoptera include, but are not limited to, armyworms, cutworms, loopers, and heliothines in the Family Noctuidae, e.g., Fall armyworm (Spodoptera frugiperda), Beet armyworm (Spodoptera exigua), Black armyworm (Spodoptera exempta), Southern armyworm (Spodoptera eridania), bertha armyworm (Mamestra configurata), black cutworm (Agrotis ipsilon), cabbage looper (Trichoplusia ni), soybean looper (Pseudoplusia includens), velvetbean caterpillar (Anticarsia gemmatalis), green cloverworm (Hypena scabra), tobacco budworm (Hehothis virescens), granulate cutworm (Agrotis subterranea), armyworm (Pseudaletia unipuncta), Western cutworm (Agrotis orthogonia); borers, casebearers, webworms, coneworms, cabbageworms and skeletonizers from the Family Pyrandae, e.g., European corn borer (Ostrinia nubilalis), navel orangeworm (Amyelois transitella), corn root webworm (Crambus caliginosellus), sod webworm (Herpetogramma licarsisalis), sunflower moth (Homoeosoma electellum), lesser cornstalk borer (Elasmopalpus hgnosellus); leafrollers, budworms, seed worms, and fruit worms in the Family Tortricidae, e.g., codling moth (Cydia pomonella), grape berry moth (Endopiza viteana), oriental fruit moth (Graphohta molesta), sunflower bud moth (Suleima hehanthana); and many other economically important Lepidoptera, e.g., diamondback moth (Plutella xylostella), pink bollworm (Pectinophora gossypiella), and gypsy moth (Lymantria dispar). Other insect pests of order Lepidoptera include, e.g., cotton leaf worm (Alabama argillacea), fruit tree leaf roller (Archips argyrospila), European leafroller (Archips rosana) and other Archips species, (Chilo suppressalis, Asiatic rice borer, or rice stem borer), rice leaf roller (Cnaphalocrocis medinalis), corn root webworm (Crambus caliginosellus), bluegrass webworm (Crambus teterrellus), southwestern corn borer (Diatraea grandiosella), surgarcane borer (Diatraea saccharalis), spiny bollworm (Earias insulana), spotted bollworm (Earias vittella), American bollworm (Helicoverpa armigera), corn earworm (Helicoverpa zea, also known as soybean podworm and cotton bollworm), tobacco budworm (Heliothis virescens), soy webworm (Herpetogramma licarsisalis), Western bean cutworm (Striacosta albicosta), European grape vine moth (Lobesia botrana), citrus leafminer (Phyllocnistis citrella), large white butterfly (Pieris brassicae), small white butterfly (Pieris rapae, also known as imported cabbageworm), beet armyworm (Spodoptera exigua), tobacco cutworm (Spodoptera litura, also known as cluster caterpillar), and tomato leafminer (Tuta absoluta).

The order Coleoptera includes the suborders Adephaga and Polyphaga. Suborder Adephaga includes the superfamilies Caraboidea and Gyrinoidea, while suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae; Superfamily Chrysomeloidea includes the family Chrysomelidae. The genus Diabrotica and the species Western corn rootworm (Diabrotica virgifera virgifera) are included within the family Chrysomelidae.

According to some embodiments of the invention, the insect from the order Coleoptera is the Western corn rootworm (WCRW, Diabrotica virgifera virgifera).

The order Hemiptera includes, but is not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schaffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper); Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Adelphocoris rapidus Say (rapid plant bug); Poecilocapsus lineatus Fabricius (four-lined plant bug); Nysius ericae Schilling (false chinch bug); Nysius raphanus Howard (false chinch bug); Eurygaster spp.; Coreidae spp.; Pyrrhocoridae spp.; Tinidae spp.; Blostomatidae spp.; Reduviidae spp.; Cimicidae spp; and Green Peach Aphids (Myzus persicae).

According to some embodiments of the invention, the insect from the order Hemiptera is the Southern green stink bug (STK, Nezara viridula).

According to some embodiments of the invention, the insect is of the genera Spodoptera, Helicoverpa, Chrysodeixis, Trichoplusia, Ostrinia and Agrotis. Examples include but are not limited to, the species Spodoptera exigua, Spodoptera littoralis and Spodoptera frugiperda, Helicoverpa zea and Helicoverpa armigera, Chrysodeixis includens, Chrysodeixis celebensis, Chrysodeixis eriosoma, Chrysodeixis argitifera, Chrysodeixis acuta illuminata, Chrysodeixis minutus and Chrysodeixis chalcites, Trichoplusia ni, Ostrinia nubilalis or Agrotis ipsilon.

According to some embodiments of the invention, the insect is of the genus Diabrotica. Examples include, but are not limited to, Diabrotica speciosa, Diabrotica barberi, Diabrotica balteata, Diabrotica undecimpunctata, and Diabrotica virgifera.

The order Diptera includes the Suborders Nematocera, Brachycera, and Cyclorrhapha. Suborder Nematocera includes the families Tipulidae, Psychodidae, Culicidae, Ceratopogonidae, Chironomidae, Simuliidae, Bibionidae, and Cecidomyiidae. Suborder Brachycera includes the families Stratiomyidae, Tabanidae, Therevidae, Asilidae, Mydidae, Bombyliidae, and Dolichopodidae . Suborder Cyclorrhapha includes the Divisions Aschiza and Aschiza. Division Aschiza includes the families Phoridae, Syrphidae, and Conopidae . Division Aschiza includes the Sections Acalyptratae and Calyptratae. Section Acalyptratae includes the families Otitidae, Tephritidae, Agromyzidae, and Drosophilidae. Section Calyptratae includes the families Hippoboscidae, Oestridae, Tachinidae, Anthomyiidae, Muscidae, Calliphoridae, and Sarcophagidae.

The term “plant” as used herein encompasses a whole plant, a grafted plant, ancestor(s) and progeny of the plants and plant parts, including seeds, shoots, stems, roots (including tubers), rootstock, scion, and plant cells, tissues and organs. The plant or part thereof may be in any form, including suspension cultures, embryos, meristematic regions, callus tissue, leaves, gametophytes, sporophytes, pollen, and microspores. Plants to be treated according to the methods of the invention include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp (peanut), Areca catechu, Astelia fragrans, Astragalus cicer, Baikiaea plurijuga, Betula spp., Brassica spp. (broccoli, Brussels sprouts, collard greens, cabbage, rapeseed/oilseed rape/canola, cauliflower, kale), Bruguiera gymnorrhiza, Burkea africana, Butea frondosa, Cadaba farinosa, Calliandra spp, Camellia sinensis (tea), Canna indica, Capsicum spp. (pepper), Cassia spp., Centroema pubescens, Chacoomeles spp., Cinnamomum cassia, Coffea arabica, Colophospermum mopane, Coronillia varia, Cotoneaster serotina, Crataegus spp., Cucumis spp. (squash), Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp. (eucalyptus), Euclea schimperi, Eulalia vi/losa, Pagopyrum spp., Feijoa sellowlana, Fragaria spp. (strawberry), Flemingia spp, Freycinetia banksli, Geranium thunbergii, Ginkgo biloba, Glycine max (soybean), Gliricidia spp, Gossypium hirsutum (cotton), Grevillea spp., Guibourtia coleosperma, Hedysarum spp., Hemaffhia altissima, Heteropogon contoffus, Hordeum vulgare (barley), Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago sativa (alfalfa), Metasequoia glyptostroboides, Musa sapientum, Nicotianum spp. (tobacco), Onobrychis spp., Ornithopus spp., Oryza spp. (rice), Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum (pea), Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Solanum spp. (potato, tomato, eggplant), Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp. (wheat), Tsuga heterophylla, Vaccinium spp., Vicia spp., Vitis vinifera (grape), Watsonia pyramidata, Zantedeschia aethiopica, Zea mays (maize), amaranth, artichoke, asparagus, carrot, celery, flax, lentil, okra, onion, sugar beet, sugar cane, sunflower, rye, oat, sunflower, a tree, an ornamental plant, a perennial grass and a forage crop.

The insects/mites to be controlled are pests of major crops, such as Maize, Sorghum, Wheat, Sunflower, Cotton, Rice, Soybean, Barley and Oil Seed Rape.

According to some embodiments, the insect is Western corn rootworm (Diabrotica virgifera virgifera, WCRW) and the plant is of a plant family selected from the group consisting of: Poaceae, Asteraceae, Cucurbitaceae, and Fabaceae.

According to some embodiments the insect is Fall armyworm (Spodoptera frugiperda, FAW) and the plant is of a plant family selected from the group consisting of: Amaranthaceae, Apiaceae, Apocynaceae, Asteraceae, Brassicaceae, Caryophyllaceae, Chenopodiaceae, Convolvulaceae, Cucurbitaceae, Cyperaceae, Euphorbiaceae, Fabaceae, Geraniaceae, Iridaceae, Juglandaceae, Liliaceae, Malvaceae, Musaceae, Platanaceae, Poaceae, Poaceae, Polygonaceae, Portulacaceae, Rosaceae, Rutaceae, Solanaceae, Ericaceae, Violaceae, Vitaceae, and Zingiberaceae.

According to some embodiments, the insect is Southern green stink bug (Nezara viridula, STK) and the plant is of a plant family selected from the group consisting of: Anacardiaceae, Asteraceae, Brassicaceae, Caprifoliaceae, Chenopodiaceae, Convolvulaceae, Cucurbitaceae, Euphorbiaceae, Fabaceae, Juglandaceae, Lauraceae, Magnoliaceae, Malvaceae, Oleaceae, Passifloraceae, Pedaliaceae, Poaceae, Proteaceae, Rosaceae, Rutaceae, Scrophulariaceae, Solanaceae, and Sterculiaceae.

According to some embodiments, the insect is Beet Armyworm (Spodoptera exigua) and the plant is of a plant family selected from the group consisting of: Poaceae, Malvaceae, Liliaceae, Amaranthaceae, Fabaceae, Solanaceae, Chenopodiaceae, Brassicaceae, Solanaceae, Cyperaceae, Juglandaceae, Asteraceae, Cucurbitaceae, Rutaceae, Euphorbiaceae, Convolvulaceae, Caryophyllaceae, Apiaceae, Polygonaceae, Rosaceae, Iridaceae, Musaceae, Geraniaceae, Platanaceae, Apocynaceae, Portulacaceae, Rosaceae, Ericaceae, Violaceae, Vitaceae, and Zingiberaceae.

According to some embodiments, the insect is Cabbage Looper (Trichoplusia ni) and the plant is of a plant family selected from the group consisting of: crucifers, beet, cantaloupe, celery, cucumber, lima bean, lettuce, parsnip, pea, pepper, potato, snap bean, spinach, squash, sweet potato, tomato, watermelon, chrysanthemum, hollyhock, snapdragon, sweetpea, cotton, tobacco, Chenopodium album, Lactuca spp. (wild lettuce), Taraxacum officinale (dandelion), and Rumex crispus (curly dock).

According to some embodiments, the insect is Diamondback Moth (Plutella xylostella) and the plant is of a plant family selected from the group consisting of: Malvaceae, Brassicaceae, Capparaceae, Asteraceae and Fabaceae.

According to some embodiments, the insect is Green Peach Aphid (Myzus persicae) and the plant is of a plant family selected from the group consisting of: Malvaceae, Euphorbiaceae, Aloaceae, Boraginaceae, Apiaceae, Scrophulariaceae, Araceae, Fabaceae, Brassicaceae, Asteraceae, Liliaceae, Chenopodiaceae, Solanaceae, Caricaceae, Apocynaceae, Cucurbitaceae, Rutaceae, Convolvulaceae, Iridaceae, Rosaceae, Caryophyllaceae, Euphorbiaceae, Iridaceae, Malvaceae, Poaceae, Cannabaceae, Balsaminaceae, Convolvulaceae, Poaceae, Lamiaceae, Papaveraceae, Lauraceae, Myrtaceae, Punicaceae, Anacardiaceae, Polygonaceae, and Pedaliaceae.

According to some embodiments, the insect is Soybean Looper (Chrysodeixis includens) and the plant is of a plant family selected from the group consisting of: Amaranthaceae, Apiaceae, Araceae, Araliaceae, Asteraceae, Begoniaceae, Brassicaceae, Caryophyllaceae, Chenopodiaceae, Convolvulaceae, Cucurbitaceae, Euphorbiaceae, Fabaceae, Geraniaceae, Gesneriaceae, Hydrangeaceae, Lamiaceae, Lauraceae, Liliaceae, Malvaceae, Passifloraceae, Piperaceae, Poaceae, Polygonaceae, Portulacaceae, Rubiaceae, and Solanaceae.

According to some embodiments, the insect is Western tarnished plant bug (Lygus hesperus) and the plant is of a plant family selected from the group consisting of: Cruciferae, Fabaceae, Malvaceae, Rosaceae, and Umbelliferae.

According to some embodiments the plant pest is Twospotted spider mite (Tetranychus urticae) and the plant is of a family selected from the group consisting of Malvaceae, Asteraceae, Actinidiaceae, Liliaceae, Fabaceae, Apiaceae, Oxalidaceae, Chenopodiaceae, Theaceae, Solanaceae, Caricaceae, Apocynaceae, Cucurbitaceae, Rutaceae, Convolvulaceae, Betulaceae, Orchidaceae, Caryophyllaceae, Ebenaceae, Zingiberaceae, Salacia, Euphorbiaceae, Moraceae, Rosaceae, Iridaceae, Araliaceae, Cannabaceae, quifoliaceae, Balsaminaceae, Lamiaceae, Poaceae, Papaveraceae, Geraniaceae, Arecaceae, Ericaceae, Grossulariaceae, Pedaliaceae, Combretaceae, Tiliaceae, Violaceae, and Vitaceae.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Sourcing of Microbial Strains with Potential Bio-pesticidal Activity

This example describes the source from which the microbial strains were isolated prior to screening for bio-insecticidal activity. Table 1 hereinbelow presents the microbial strains described in this invention and their origin.

Sampling was carried out during the years 2017-2019. Source plants were sampled from various relevant habitats across Israel and USA. Plant organs (e.g., rhizospheres, roots) used as bacterial source were detached from the plants and collected. In the laboratory, the detached organs were immersed in sterile 1% potassium citrate in phosphate buffered saline [PBS; per liter: 8 gr sodium chloride (NaCl), 0.2 gr potassium chloride (KCl), 1.42 gr disodium phosphate (Na₂HPO₄) and 0.24 gr potassium phosphate (KH₂PO₄), pH7.4], 10 mM EDTA, and 0.1% Tween 80, and shaken for 1 h at 200 revolutions per minute (RPM). Collected larvae of WCRW or Maladera insanabilis used as bacterial sources were crushed in lml of sterile 1% potassium citrate in PBS.

Thereafter, the plant organs or larvae were transferred carefully to a new 50 ml Falcon tube. Microbes released from the plant organs into the medium were serially diluted and the selected dilutions were plated onto several bacteriological growth media including, but not limited to, R2G [per liter: 0.5 g proteose peptone, 0.5 g casamino acids, 0.5 g yeast extract, 0.5 g dextrose, 0.5 g soluble starch, 0.3 g dipotassium phosphate (K₂HPO₄), 0.05 g, magnesium sulfate (MgSO₄·7H₂O), 0.3 g sodium pyruvate and 8 g gelrite as a gelling agent, R2G adjusted to PH 5.0]; or VL55 [per liter: 3.9 g MES, 20 ml of 20 mM MgSO₄, 20 ml of 30 mM CaCl₂, 20 ml of (NH₄)₂HPO₄, 2 ml Se/W, 2 ml Trace element solution SL10, 1.5 ml of Vitamin10×, 0.2M trehalose, adjusted to pH5.5, 1.5% gelrite].

Isolated colonies that appeared on plates after 24-72 hours of growth at 28° C. in the dark, were further picked and re-isolated on a new R2G plate, before storage in an R2A broth [per liter: 0.5 g proteose peptone, 0.5 g casamino acids, 0.5 g yeast extract, 0.5 g dextrose, 0.5 g soluble starch, 0.3 g dipotassium phosphate (K₂HPO₄), 0.05 g magnesium sulfate (MgSO₄·7H₂O), 0.3 g sodium pyruvate], supplemented with 25% glycerol, at minus 80° C. Isolates were identified to the strain level by whole genome sequencing using an Illumina MiSeq sequencing platform, or to the species level by Sanger sequencing of the 16S-rRNA gene with the universal primers 16S_27F and 16S_1492R (see Example 2 hereinbelow).

TABLE 1
Microbial strains described according to some embodiments of the invention

| Microbial | Locale of | Plant/insect | | |
|---|---|---|---|---|
| strain name | Origin | source | Source species | Source tissue |
| LAV41249 | Missouri | insect | WCRW | insect gut fluid |
| LAV43112 | Missouri | plant | Zea mays | rhizospheric soil |
| LAV61242 | Israel | plant | Citrullus melo | leaf endosphere |
| LAV61243 | Israel | plant | Vitis vinifera | leaf |
| | | | | phyllosphere |
| LAV61249 | Israel | plant | Piptatherum miliaceum | rhizoplane |
| LAV61251 | Israel | plant | Vitis vinifera | leaf |
| | | | | phyllosphere |
| LAV61268 | Israel | plant | Zea mays | leaf endosphere |
| LAV61269 | Israel | plant | Sorghum halepense | rhizosphere |
| LAV61288 | Israel | plant | Imperata cylindrica | root |
| LAV61293 | Israel | insect | Maladera insanabilis | insect gut fluid |
| LAV61295 | Israel | plant | Zea mays | rhizosphere |
| LAV61297 | Israel | plant | Zea mays | rhizosphere |
| LAV61300 | Israel | plant | Vicia peregrina | root |
| LAV61301 | Israel | plant | Lathyrus lentiformis | root endosphere |
| LAV61303 | Israel | plant | Zea mays | root rhizoplane |
| LAV61308 | Israel | plant | Zea mays | leaf |
| | | | | phyllosphere |
| LAV100189 | Illinois | plant | Zea mays | root rhizosphere |
| LAV104743 | Missouri | plant | Zea mays | rhizospheric soil |
| LAV104744 | Missouri | insect | WCRW | insect gut fluid |
| LAV104745 | Missouri | plant | Zea mays | rhizospheric soil |
| LAV104746 | Missouri | insect | WCRW | insect gut fluid |
| LAV104747 | Missouri | plant | Zea mays | rhizosphere |
| LAV104751 | Missouri | insect | WCRW | insect gut fluid |
| LAV104753 | Missouri | insect | WCRW | insect gut fluid |
| LAV104754 | Missouri | insect | WCRW | insect gut fluid |
| LAV104755 | Missouri | insect | WCRW | insect gut fluid |
| LAV104758 | Illinois | — | — | field soil |
| LAV104759 | Indiana | | — | field soil |
| LAV104761 | Illinois | — | — | field soil |
| LAV104934 | Missouri | insect | WCRW | insect gut fluid |
| LAV104944 | Missouri | insect | WCRW | insect gut fluid |
| LAV106494 | California | — | — | field soil |
| LAV106564 | Missouri | insect | WCRW | insect gut fluid |
| LAV106654 | Illinois | — | — | field soil |
| LAV106657 | Indiana | — | — | field soil |
| LAV1000686 | Missouri | plant | Zea mays | rhizospheric soil |
| LAV1000731 | Israel | plant | Nepenthes ventricosa | leaf |
| | | | | phyllosphere |
| LAV1000737 | Israel | plant | Nepenthes miranda | leaf |
| | | | | phyllosphere |
| LAV1000771 | Missouri | plant | Zea mays | rhizospheric soil |
| LAV1000775 | Missouri | plant | Zea mays | rhizospheric soil |
| LAV1000939 | Indiana | — | — | field soil |

### Example 2: Microbial Strain Characterization by 16S-rRNA

The microbial strains of the present invention have been further characterized based on their 16S-rRNA sequence. Without wishing to be bound by any theory or mechanism of action, any strain with a significantly homologous 16S-rRNA sequence is likely to exhibit the same functional properties and therefore can be used as an insecticide as described in this invention.

16S-rRNA sequences were obtained by either:
1) Polymerase Chain Reaction (PCR; U.S. Pat. No. 4,683,195) using the universal primers 16S_27F (AGAGTTTGATCMTGGCTCAG, SEQ ID NO:176) and 16S_1492R (TACGGYTACCTTGTTACGACTT, SEQ ID NO:177) (Eden, P.A., et al. 1991. Int. J. Syst. Bacteriol. 41:324-325) followed by Sanger sequencing (Sanger, F., and Coulson, A.R. 1975. J. Mol. Biol. 94:441-448) of the amplified fragments using the primers 16S_27F and 16S-1492R, and the additional primers 16S_5151F (GTGCCAGCMGCCGCGGTAA, SEQ ID NO:178) and 16S_970R (CCGTCAATTCMTTTRAGTTT, SEQ ID NO: 179).
2) Extraction of 16S-rRNA sequences from the assembly of microbial strains genome sequences (Applicant proprietary pipeline) using the MOTHUR tool (a bioinformatics tool for analyzing 16S-rRNA gene sequences; Schloss, P.D. et al. 2009. Appl. Environ. Microbiol. 75:7537-7541) and SILVA database (a comprehensive on-line resource for quality checked and aligned ribosomal RNA sequence data; Pruesse, E. et al. 2007. Nucl. Acids Res. 35:7188-7196) as a reference.

Obtained 16S-RNA sequences were clustered (grouped) to Operational Taxonomic Units (OTUs) using nucleotide-based local alignment search tool (BLASTN; Altschul, S. et al. J. of Mol. Biol. 215:403-410). Table 2 below presents a list of bacterial strains of the invention and their respective 16S-rRNA sequence SEQ ID NOs. as disclosed in the Sequence Listing herein. Some 16S-rRNA sequences showed homology higher than 97% global identity to sequences of other strains of the invention, as detailed in the rightmost column.

TABLE 2
Bacterial strain organism and 16S rRNA SEQ ID NOs.

| | | 16S rRNA | Homologous |
|---|---|---|---|
| Isolate ID | | SEQ ID | sequences |
| Number | Organism | NOs. | (Over 97%) |
| LAV41249 | Serratia sp. | 1 | 2, 26 |
| LAV43112 | Serratia sp. | 2 | 1, 26, 49 |
| LAV61242 | Pantoea sp. | 3 | |
| LAV61243 | Microbacterium sp. | 4 | 29 |
| LAV61249 | Stenotrophomonas sp. | 5 | 20, 33 |
| LAV61251 | Rathayibacter sp. | 6 | |
| LAV61268 | Bacillus sp. | 7 | 24, 25 |
| LAV61269 | Pseudomonas sp. | 8 | 9, 10 |
| LAV61288 | Pseudomonas sp. | 9-10 | 8, 27, 28, 32 |
| LAV61293 | Serratia sp. | 11-12 | |
| LAV61295 | Ensifer sp. | 13 | 43 |
| LAV61297 | Streptomyces sp. | 14-15 | |
| LAV61300 | Serratia sp. | 16 | 11, 12 |
| LAV61301 | Rahnella sp. | 17 | 44 |
| LAV61303 | Aminobacter sp. | 18 | |
| LAV61308 | Bacillus sp. | 19 | |
| LAV100189 | Stenotrophomonas sp. | 20 | 5, 34 |
| LAV104743 | Carnobacterium sp. | 21-22 | 41, 42 |
| LAV104744 | Acinetobacter sp. | 23 | 40, 47 |
| LAV104745 | Bacillus sp. | 24-25 | 7 |
| LAV104746 | Serratia sp. | 26 | 1, 2, 49 |
| LAV104747 | Pseudomonas sp. | 27-28 | 9, 10, 31, 32, 45 |
| LAV104751 | Microbacterium sp. | 29 | |
| LAV104753 | Pseudomonas sp. | 30 | 5, 20, 33, 34, 46 |
| LAV104754 | Pseudomonas sp. | 31-32 | 10, 27 |
| LAV104755 | Stenotrophomonas sp. | 33-34 | 5, 20, 30, 46 |
| LAV104758 | Chitinophaga sp. | 35-36 | |
| LAV104759 | Brevibacillus sp. | 37-38 | |
| LAV104761 | Pseudomonas sp. | 39 | |
| LAV104934 | Acinetobacter sp. | 40 | 23, 47 |
| LAV104944 | Pseudomonas sp. | 45 | 27 |
| LAV106494 | Stenotrophomonas sp. | 46 | 30, 33 |
| LAV106564 | Carnobacterium sp. | 41-42 | 21 |
| LAV106654 | Ensifer sp. | 43 | |
| LAV106657 | Rahnella sp. | 44 | 17 |
| LAV1000686 | Acinetobacter sp. | 47 | 23, 40 |
| LAV1000731 | Paraburkholderia sp. | 48 | |
| LAV1000771 | Serratia sp. | 49 | 2, 26 |
| LAV1000775 | Chryseobacterium sp. | 50 | |
| LAV1000939 | Variovorax sp. | 51 | |

Table 2: List of bacterial strains of the invention, and their respective 16S-rRNA sequences as disclosed in the Sequence Listing herein. Some 16S-rRNA sequences showed homology higher than 97% global identity to sequences of other strains of the invention, as detailed in the rightmost column.

### Example 3: Clustering of Microbial Strains Using Strain-Specific Genomic Markers

Encoded protein sequences for the following housekeeping genes: cysS, EngA, gyrB, recA, RimM, RsmD, ruvB, TilS_N, uvrB, uvrC, YchF, IMPD, ruvA, IF2, hemN, murC, holA (accessions TIGR00435, TIGR03594, TIGRO1059, TIGRO2012, TIGR02273, TIGR00095, TIGR00635, TIGR02432, TIGRO0631, TIGRO0194, TIGR00092, TIGR1032, TIGR00084, TIGR00487, TIGR00539, TIGRO1082, TIGRO1128, respectively) were identified in the genomes of the microbial strains of the invention using the TIGRFAM annotation for protein families, which is based on sequence alignments and hidden Markov models (HMIs). The genomic sequences of these identified genes were then screened against the NCBI bacterial genome RefSeq database, using NCBI local alignment tool TBLASTN (NCBI-blast-2.10.0+). Criteria for declaring a microbial strain-specific marker were no hits over 99% local protein identity over 97% coverage of the gene. Up to 5 genomic markers, being significantly microbial strain-specific, were selected for each microbial strain described in this invention.

TABLE 3
SEQ ID NOs of genomic markers of each strain

| | | Number of | |
|---|---|---|---|
| | Genomic | Microbial | Genomic Marker Sequences |
| Microbial | Marker | strain- | length (amino acids), |
| strain | SEQ ID | specific | concordant with Marker |
| number | NOs. | markers | SEQ ID NOs. |
| LAV41249 | 52-55 | 4 | 1091, 1064, 1331, 1490 |
| LAV43112 | 52, 54-56 | 4 | 1091, 1331, 1490, 1064 |
| LAV61242 | 57-61 | 5 | 1091, 1832, 2021, 1004, 1067 |
| LAV61243 | 62-64 | 3 | 1073, 617, 992 |
| LAV61249 | 65-67 | 3 | 1844, 1040, 1397 |
| LAV61251 | 68-70 | 3 | 2066, 2099, 1088 |
| LAV61268 | 71-72 | 2 | 1100, 1784 |
| LAV61269 | 73-75 | 3 | 2015, 2417, 1052 |
| LAV61288 | 76-78 | 3 | 1100, 2015, 1469 |
| LAV61293 | 79-81 | 3 | 2030, 2414, 1064 |
| LAV61295 | 82 | 1 | 1430 |
| LAV61297 | 83-87 | 5 | 1088, 2138, 2030, 1133, 1448 |
| LAV61300 | 88-90 | 3 | 2012, 2414, 1064 |
| LAV61301 | 91-92 | 2 | 1391, 1007 |
| LAV61303 | 93-97 | 5 | 1103, 3053, 2447, 1049, 1436 |
| LAV61308 | 98-99 | 2 | 1985, 1046 |
| LAV100189 | 100-103 | 4 | 2024, 512, 1328, 1397 |
| LAV104743 | 104-107 | 4 | 2462, 1148, 1331, 1481 |
| LAV104744 | 108-111 | 4 | 1091, 2021, 2468, 1409 |
| LAV104745 | NA | 0 | NA |
| LAV104746 | 112-113 | 2 | 2012, 2414 |
| LAV104747 | 114-116 | 3 | 2015, 2417, 1058 |
| LAV104751 | 117-118 | 2 | 617, 1025 |
| LAV104753 | 119-122 | 4 | 1091, 578, 1844, 1397 |
| LAV104754 | 123-124 | 2 | 2015, 5626 |
| LAV104755 | 125-127 | 3 | 1091, 578, 1685 |
| LAV104758 | 128-131 | 4 | 2054, 1982, 1010, 1313 |
| LAV104759 | 132-133 | 2 | 1922, 521 |
| LAV104761 | 134-136 | 3 | 1100, 1382, 1316 |
| LAV104934 | 137-139 | 3 | 2021, 2468, 1046 |
| LAV104944 | 140-143 | 4 | 1823, 1382, 2015, 1328 |
| LAV106494 | 144-145 | 2 | 2024, 1100 |
| LAV106564 | 146-148 | 3 | 572, 1409, 515 |
| LAV106654 | 149-153 | 5 | 1103, 2942, 2435, 1091, 1433 |
| LAV106657 | 154-155 | 2 | 1343, 1484 |
| LAV1000686 | 156-158 | 3 | 2021, 2468, 1046 |
| LAV1000731 | 159-163 | 5 | 1094, 2090, 2471, 1073, 1337 |
| LAV1000771 | 164-165 | 2 | 611, 1034 |
| LAV1000775 | 166-170 | 5 | 1091, 2012, 1934, 1001, 1310 |
| LAV1000939 | 171-175 | 5 | 1091, 2117, 2606, 1106, 1343 |

Table 3: *NA—Not Available

### Example 4: In Vitro Feeding Bioassay Against Coleoptera & Lepidoptera Target Pests

Microbial strains were grown on R2A 90 mm plates. Single colonies were then scraped and bacteria were suspended in 250-300 μl of appropriate insect liquid diet. (Coleoptera liquid diet: 30% glucose, 4% fructose, 4% sucrose, 1% linoleic acid, 2.5% blue food coloring suspended in sterile water; Lepidoptera liquid diet: 20% sucrose, 2.5% blue food coloring suspended in sterile water). Bacteria were fed in the liquid diets to a plurality of Western corn rootworm (WCRW; Coleoptera) and Fall armyworm (FAW; Lepidoptera) neonates for 5-30 min. E. coli bacteria were added to the liquid diets as a negative control.

The appropriate artificial insect diet, without bacteria, was then also dispensed into each well of a standard 96-well plate. A single first instar larva from the plurality fed previously with the bacterial solution was then deposited into each well of a 96 well plate. Mortality of insects was scored after 96 hours of incubating them in the artificial diet plate.

Table 4 shows the mortality percentage of insects fed with the various bacterial isolates. For comparison, mortality of the negative control larvae fed with E. coli was 4% on average, and never greater than 10% in any experiment.

TABLE 4
Insecticidal activity of bacterial isolates
in an in vitro feeding bioassay

| Bacterial | Target Insect | AVG. | |
|---|---|---|---|
| isolate | (WCRW/FAW) | Mortality | P-value |
| LAV41249 | WCRW | 66.7% | L |
| LAV43112 | WCRW | 25.0% | 0.02 |
| LAV61249 | WCRW | 45.5% | L |
| LAV61251 | WCRW | 8.3%* | 0.04 |
| LAV61268 | WCRW | 45.5% | 0.02 |
| LAV61268 | FAW | 16.7% | 0.01 |
| LAV61269 | WCRW | 58.3% | L |
| LAV61288 | WCRW | 33.3% | 0.05 |
| LAV61293 | WCRW | 83.3% | L |
| LAV61295 | WCRW | 66.7% | L |
| LAV61297 | WCRW | 58.3% | L |
| LAV61300 | WCRW | 72.7% | L |
| LAV61301 | WCRW | 50.0% | L |
| LAV61308 | WCRW | 27.3% | 0.02 |
| LAV100189 | WCRW | 41.7% | 0.09 |
| LAV104743 | WCRW | 66.7% | 0.01 |
| LAV104744 | WCRW | 75.0% | L |
| LAV104745 | WCRW | 72.7% | L |
| LAV104746 | WCRW | 83.3% | L |
| LAV104747 | WCRW | 75.0% | L |
| LAV104753 | WCRW | 9.1% | L |
| LAV104755 | WCRW | 50.0% | L |
| LAV104758 | WCRW | 36.4% | 0.05 |
| LAV104759 | WCRW | 66.7% | 0.02 |
| LAV104761 | WCRW | 54.5% | L |
| LAV104934 | WCRW | 41.7% | 0.02 |
| LAV104944 | WCRW | 66.7% | 0.01 |
| LAV106494 | WCRW | 58.3% | L |
| LAV106564 | WCRW | 36.4% | L |
| LAV106654 | WCRW | 36.4% | 0.07 |
| LAV106657 | WCRW | 75.0% | L |
| LAV1000686 | WCRW | 41.7% | 0.11 |
| LAV1000771 | WCRW | 41.7% | 0.07 |
| LAV1000775 | WCRW | 50.0% | 0.03 |

Table 4. Mortality of insects treated in a feeding assay in vitro was calculated as the percentage of dead and/or severely stunted insects from the total in each plate. P-value was calculated using Fisher's test. “AVG.” = average. “FAW” = Fall Armyworm (Spodoptera frugiperda); “WCRW” = Western corn rootworm (Diabrotica virgifera virgifera). “L” = P-value < 0.01.
*Mortality in this experiment was low, but 67% of the larvae were stunted.

### Example 5: In Planta Tri Trophic Bioassay Against Western Corn Rootworm

Microbial strains were grown as a lawn on five R2G plates each, for 2 days at 28° C. in the dark. The bacteria were then collected and suspended in 20 ml sterile R2A broth (with gelrite) supplemented with 25% glycerol and kept at −80° C. until use. Two weeks before a planned sowing of maize seeds, each strain was cultured on 25 R2G plates and regrown for 2 days at 28° C. in the dark. Cells were then harvested and suspended in 50 ml tap water supplemented with 2% Carboxy Methyl Cellulose (CMC) serving as a gluing agent. This suspension was mixed with maize seeds at a ratio of 30 ml of suspension for every 1000 g of seeds. As a control, seeds were incubated with water-CMC solution only.

The mixture was shaken vigorously for 10 min and thereafter the seeds were separated and dried on a paper towel in a biological cabinet for a few hours. The average number of CFUs on seeds was measured 3 days before sowing to ensure a titer of >10⁵ CFUs/seed by vigorously mixing five coated seeds in PBS to release bacteria from coat, serially diluting the sample, plating the dilutions on R2G plates and enumerating CFUs after 2 days.

Drench was performed by dispensing 1 ml bacterial suspension on each seed immediately before planting. Bacterial suspension for drench was prepared by scraping the bacterial lawn grown on R2A 90 mm plates and suspending in 10 ml 0.1×PBS.

Corn seeds (Zea mays) treated by seed coating or drench were planted in 16-oz. pots and placed to grow in a plant growth chamber (Conviron®, 25° C., 14 hours light: 10 hours dark). After 10 days, each pot was infested with 30 neonates of WCRW and returned to the growth chamber for further growth. After an additional 12 days, the height of the plants and the width of the stem were recorded and the larvae developed in the soil were extracted using Berlese apparatus, and live insects were counted.

The results presented in Table 5 show improvement of plant growth and increased mortality of insects of treated plants relative to the control.

TABLE 5
Activity of bacterial isolates against WCRW in corn tri trophic bioassay

| Bacterial | | Plant height | | Stem width | | Mortality | |
|---|---|---|---|---|---|---|---|
| isolate | Treatment | IMP. | P-value | IMP. | P-value | IMP. | P-value |
| LAV41249 | D | 0% | N.S. | 11% | 0.05 | 24% | 0.20 |
| LAV43112 | D | 8% | N.S. | 20% | L | 42% | 0.02 |
| LAV43112 | ST | 6.2% | N.S. | −1.6% | N.S. | 76.7% | 0.00 |
| LAV106564 | D | −0.2% | N.S. | 1.4% | N.S. | 87.8% | L |
| LAV6106564 | ST | 12.0% | N.S. | 6.0% | N.S. | 87.2% | L |
| LAV61242 | ST | 4.2% | N.S. | 4.4% | N.S. | 80.3% | L |
| LAV61242 | ST | −0.5% | N.S. | −7.3% | N.S. | 47.4% | 0.01 |
| LAV61243 | D | 20.2% | L | 13.4% | 0.03 | 57.4% | 0.06 |
| LAV61243 | ST | 16.1% | N.S. | 15.4% | L | 48.8% | 0.03 |
| LAV61249 | D | 13.0% | 0.07 | 0.0% | N.S. | 70.2% | 0.02 |
| LAV61249 | ST | 26.9% | 0.06 | 18.0% | 0.14 | 36.9% | N.S. |
| LAV61251 | D | 11.5% | 0.12 | −1.1% | N.S. | 80.9% | L |
| LAV61251 | ST | −5% | N.S. | 4% | N.S. | 60% | 0.10 |
| LAV61268 | D | 8.6% | N.S. | 16.3% | 0.03 | 67.4% | L |
| LAV61268 | ST | 19.7% | 0.10 | 12.8% | 0.02 | 82.4% | L |
| LAV61268 | ST | 30.7% | 0.03 | 19.4% | 0.09 | 76.9% | L |
| LAV61269 | D | 2.1% | N.S. | 10.7% | N.S. | 46.5% | 0.06 |
| LAV61269 | ST | 27.7% | L | 13.5% | L | 80.3% | L |
| LAV61288 | ST | 7.5% | N.S. | 6.5% | N.S. | 44.9% | L |
| LAV61288 | ST | −3.2% | N.S. | −10.1% | N.S. | 36.8% | 0.08 |
| LAV61293 | D | −1.5% | N.S. | −2.4% | N.S. | 87.5% | L |
| LAV61293 | ST | −8.76% | N.S. | −2.08% | N.S. | 94.1% | L |
| LAV61295 | D | 4.3% | N.S. | 4.7% | N.S. | 75.0% | L |
| LAV61295 | ST | 30.1% | 0.09 | 24.0% | L | 84.6% | L |
| LAV61297 | D | −5% | N.S. | −1% | N.S. | 76.2% | L |
| LAV61297 | ST | 5.7% | N.S. | 7.3% | N.S. | 86.9% | L |
| LAV61300 | D | 85.0% | L | 111.5% | L | 74.0% | L |
| LAV61300 | ST | 4.12% | N.S. | 6.13% | N.S. | 97.9% | L |
| LAV61301 | D | 7.2% | N.S. | 5.6% | N.S. | 70.0% | L |
| LAV61301 | ST | 15.87% | N.S. | 6.47% | N.S. | 92.7% | L |
| LAV61301 | ST | 1.1% | N.S. | 4.1% | N.S. | 77.0% | L |
| LAV61303 | D | 6.1% | N.S. | 4.7% | N.S. | 62.5% | 0.02 |
| LAV61303 | ST | 35.1% | 0.03 | 26.0% | L | 82.1% | L |
| LAV61303 | ST | 2.0% | N.S. | 4.7% | N.S. | 86.9% | L |
| LAV61308 | D | 51.0% | L | 27.0% | 0.03 | 50.9% | N.S. |
| LAV61308 | ST | 10.1% | N.S. | 9.7% | 0.09 | 17.2% | N.S. |
| LAV100189 | D | 9.9% | 0.11 | 12.1% | 0.17 | 62.0% | L |
| LAV104743 | D | −7% | N.S. | 2% | N.S. | 80.6% | 0.02 |
| LAV104743 | ST | 3.59% | N.S. | 6.60% | N.S. | 80.5% | L |
| LAV104743 | ST | 17.2% | 0.04 | 18.7% | 0.04 | 67.3% | L |
| LAV104744 | ST | −16% | N.S. | −6% | N.S. | 64% | 0.07 |
| LAV104745 | D | 39% | L | 59% | L | 50% | 0.03 |
| LAV104745 | ST | 24% | 0.12 | 7% | N.S. | 46% | N.S. |
| LAV104746 | D | 27% | 0.05 | 17% | N.S. | 47% | L |
| LAV104746 | ST | 5% | N.S. | −4% | N.S. | 64% | 0.07 |
| LAV104747 | D | 8.2% | 0.05 | 3.3% | 0.20 | 48.7% | 0.05 |
| LAV104747 | D | 74.3% | 0.05 | 55.1% | 0.05 | 50.7% | 0.05 |
| LAV104751 | D | −11% | N.S. | −3% | N.S. | 72.2% | 0.04 |
| LAV104751 | ST | 18.8% | N.S. | 14.1% | N.S. | 72.3% | 0.01 |
| LAV104753 | D | 8.4% | N.S. | 4.9% | N.S. | 52.5% | 0.06 |
| LAV104753 | ST | 15.5% | 0.20 | 6.5% | N.S. | 86.9% | L |
| LAV104754 | D | −12.9% | N.S. | −0.7% | N.S. | 95.1% | L |
| LAV104754 | ST | 4.8% | N.S. | 2.6% | N.S. | 79.5% | L |
| LAV104755 | D | 3.3% | N.S. | 1.8% | N.S. | 62.5% | 0.02 |
| LAV104755 | ST | 41.7% | L | 27.2% | L | 76.9% | L |
| LAV104759 | D | ND | ND | ND | ND | 85.1% | L |
| LAV104761 | D | ND | ND | ND | ND | 78.7% | L |
| LAV104761 | ST | −2.9% | N.S. | −1.7% | N.S. | 54.5% | 0.08 |
| LAV104761 | ST | 11.9% | N.S. | 1.9% | N.S. | 78.3% | L |
| LAV104934 | ST | 15.4% | 0.13 | 8.8% | N.S. | 30.2% | 0.08 |
| LAV104934 | ST | 16.1% | 0.19 | 9.6% | N.S. | 31.4% | 0.13 |
| LAV104944 | ST | ND | ND | ND | ND | 80.6% | L |
| LAV104944 | ST | 8.9% | N.S. | 13.0% | 0.19 | 38.2% | N.S. |
| LAV104944 | ST | 14.5% | N.S. | 9.4% | N.S. | 57.4% | 0.01 |
| LAV106494 | ST | −3.1% | N.S. | −3.8% | N.S. | 36.8% | 0.10 |
| LAV106494 | ST | 22.2% | 0.09 | 16.8% | 0.08 | 50.0% | 0.02 |
| LAV106654 | ST | 18.8% | 0.11 | 11.2% | 0.17 | 25.3% | N.S. |
| LAV106657 | D | ND | ND | ND | ND | 42.3% | N.S. |
| LAV106657 | ST | 16.6% | 0.19 | 13.5% | 0.06 | 40.2% | 0.08 |
| LAV106657 | ST | −6.2% | N.S. | 0.3% | N.S. | 47.1% | 0.06 |
| LAV1000686 | ST | 16.7% | 0.10 | 8.4% | N.S. | 31.5% | N.S. |
| LAV1000686 | ST | 29.1% | 0.03 | 22.6% | 0.09 | −6.0% | N.S. |
| LAV1000731 | ST | 12.1% | N.S. | −10.5% | N.S. | 54.9% | 0.08 |
| LAV1000771 | ST | 5.7% | N.S. | 4.4% | N.S. | 52.4% | 0.01 |
| LAV1000771 | ST | 15.4% | 0.03 | 10.5% | 0.05 | 34.8% | 0.04 |
| LAV1000775 | ST | 27.4% | 0.01 | 25.8% | 0.01 | −4.3% | N.S. |
| LAV1000775 | ST | 15.5% | 0.08 | 9.7% | 0.19 | 2.0% | N.S. |
| LAV1000939 | ST | 16.0% | N.S. | 9.7% | N.S. | 39.2% | 0.16 |
| LAV1000939 | ST | 12.9% | 0.19 | 18.4% | 0.02 | 32.7% | N.S. |
| LAV1000939 | ST | 12.5% | N.S. | 30.2% | L | −10.4% | N.S. |

Table 5: Stem width, plant height and insect mortality (calculated as in Example 4 hereinabove) were measured in treated and control plants. P-value was calculated using Dunnett's test. IMP. = % improvement compared to control. ST = seed treatment; D = Drench treatment; ND = Not determined; L = P-value < 0.01; N.S. = Not significant (P-value > 0.2).

### Example 6: In Planta Tri Trophic Bioassay Against Stink bug (STK)

Soybean seeds were planted in plant inserts (2⅜ in.×2⅜ in.×2⅛ in. deep) and grown under light racks until they reached their 2-leaf stage. Each plant was dipped into a bacterial suspension (prepared as in Example 4 hereinabove) in a tube and let stand for one hour. After the plants were dried off, 25 2^{nd} instar STK nymphs were infested at the base of each plant and each insert was wrapped up with Micro-Perforated Gusseted Transparent French Bread Bags. After 96 hours incubation under the light rack, each wrap was opened and the height of the plants and the width of the stem were recorded. Number of live and dead STKs were also counted.

Table 6 summarizes the results of plant growth improvement and percentage of live insects in treated plants relative to the control.

TABLE 6
Activity of bacterial isolates against STK in soybean leaf bioassay

| Bacterial | Plant height | | Stem width | | Mortality | |
|---|---|---|---|---|---|---|
| isolate | IMP. | P-value | IMP. | P-value | IMP. | P-value |
| LAV41249 | −12.4% | N.S. | ND | ND | 40.9% | 0.18 |
| LAV100189 | 5.0% | N.S. | ND | ND | 74.2% | 0.01 |
| LAV104745 | −16.2% | L | ND | ND | 56.8% | L |
| LAV104746 | 9.7% | N.S. | 1.8% | N.S. | 47.0% | 0.01 |

Table 6: Stem width, plant height and insect mortality (calculated as in Example 4 hereinabove) were measured in treated and control leaves. P-value was calculated using Dunnett's test. IMP. = % improvement compared to control; ND = Not determined; L = P-value < 0.01; N.S. = Not significant (P-value > 0.2).

### Example 7: In Vitro Feeding Bioassay Against Stinkbug

Second instar STK nymphs were infested in a 1 oz. cup and were fed a bacterial suspension (prepared as in Example 4 hereinabove) combined at a ratio of 1:4 with STK-specific artificial diet (suspension prepared by dissolving 39.07 g of dry mix Lygus hesperus diet purchased from Frontier in 172.5 ml of deionized water). This diet was layered in between two parafilm sheets making a sandwich and covered on top of the 1 oz. cup. Nymphs were incubated for 4 days and were able to ingest the diet by poking their mouthparts through the parafilm. Mortality of insects was scored after 4 days.

TABLE 7
Mortality of STK consuming diet containing
bacterial isolates of the invention

| Bacterial isolate | IMP. Mortality | P-value |
|---|---|---|
| LAV41249 | 89.5% | L |
| LAV43112 | 87.2% | L |
| LAV104745 | 71.1% | L |
| LAV104746 | 95.2% | L |

Table 7: Mortality of insects treated in a feeding assay in vitro was calculated as the percentage of dead and/or severely stunted insects from the total in each plate. P-value was calculated using a 2-tail T-test. L = P-value < 0.01. IMP. = % improvement compared to control; L = P-value < 0.01.

### Example 8: In Planta Tri Trophic Field Bioassay Against Western Corn Rootworm

Maize seeds were coated with bacterial strains of the invention as described in Example 5 hereinabove. Coated maize seeds (with bacteria/negative control) were sown in the field using a manual seed planter. Untreated seeds of plants expressing the Bacillus thuringiensis Cry insecticidal protein (“Cry trait-plants”) that were not treated with bacteria, nor with water-CMC solution (negative control) were used as a second (positive) control. The planter box was cleaned between seed batches using 70% ethanol and rinsing with tap water to eliminate ethanol traces. Each combination of seed with microbial strain coat, or control, was sown in six replicate plots (n=6) in a “flower” statistical design. Each plot was 6.1 m long and 3.0 m wide, and seeds were sown at a density of 200 seeds per plot. The plots were located in open fields with a high natural infestation of WCRW. In order to discover microbial strains that reduce pest symptoms, vegetative plant responses (numbers per plot of live plants, healthy plants, small plants and lodged plants) and Node injury score (NIS; for method see: J. of Economic Entomology, 98(1):1-8 (2005). doi.org/10.1603/0022-0493-98.1.1) were measured during and at the end of the experiment, respectively.

The improvement in NIS (NIS reduction) compared to the untreated control was calculated according to the following formula: % improvement=(NIS in treatment−MS in negative control)*100/(NIS in positive control−NIS in negative control), wherein plants grown from untreated seeds carrying the Cry trait served as the positive control and plants grown from seeds treated with water-CMC solution without any biological treatment served as the negative control.

TABLE 8
Activity of bacterial isolates against WCRW in tri trophic corn field bioassay

| | number of | | | | | | Node injury | | | |
|---|---|---|---|---|---|---|---|---|---|---|
| | live plants | | healthy plants | | small plants | | score | | | |
| | per plot | | per plot | | per plot | | reduction | | Yield | |
| Isolate | | P- | | P- | | P- | | P- | | P- |
| name | IMP. | value | IMP. | value | IMP. | value | IMP. | value | IMP. | value |
| LAV61242 | 7.4% | N.S. | 12.8% | 0.16 | 215.1% | 0.02 | −9.7% | N.S. | 31.6% | L |
| LAV61303 | 10.8% | 0.06 | 8.2% | N.S. | 45.7% | N.S. | 55.0% | 0.05 | 26.0% | 0.02 |
| LAV100189 | −3.1% | N.S. | −4.2% | N.S. | 201.6% | 0.02 | 22.7% | N.S. | 9.8% | N.S. |
| LAV104744 | −4.5% | N.S. | −5.6% | N.S. | 138.2% | 0.13 | 31.4% | N.S. | −23.0% | 0.04 |
| LAV104759 | 1.3% | N.S. | −1.9% | N.S. | 72.6% | N.S. | 20.9% | N.S. | 20.4% | 0.10 |
| LAV106654 | 9.8% | 0.05 | 13.7% | 0.03 | 53.5% | N.S. | 29.4% | N.S. | 11.0% | N.S. |
| LAV106657 | 2.5% | N.S. | 5.3% | N.S. | 232% | 0.01 | −1.3% | N.S. | 19.9% | 0.07 |

Table 8: Measurements of treated and control plants were compared. P-value was calculated using a one-sample t-test comparing the least square means of the (normalized) difference between control and treatment plots to zero. IMP. = % improvement compared to control; L = P-value < 0.01; N.S. = Not significant (P-value > 0.2).

### Example 9: Clustering, of Bacterial Isolates Having Pesticidal Activity based on Enzymatic Activity

Pesticidal activity of a microbial strain according to certain embodiments of the present invention is indicative of specific functional properties of the microbial strains. These functional properties contribute to plant resistance against an insect pathogen when the microbial strains are present in/on the plant. Microorganisms are known to produce lytic enzymes, such as chitinases, which hydrolyze chitin, a major component of insect/mite cuticles. Bacteria producing lytic enzymes or otherwise able to metabolize components of insect/mite anatomy or physiology, and/or bacteria producing signal compounds which attract or repel insects are therefore an alternative biocontrol strategy for controlling plant insect/mite pathogens. Such bacteria may be clustered according to their lytic properties.

### Degradation of Insect Cuticle

The ability of bacterial stains to degrade insect cuticle is assayed as described by Zhang et al 2019 (Zhang Q et al., 2019. PNAS 116(16):7982-7989; doi.org/10.1073/pnas.1816430116). In short, bacterial cells are collected and resuspended in basal salt solution plus 1% insect cuticle as a sole carbon and nitrogen source, and incubated in a shaking incubator at 220 rpm for 12 h at 28° C. The supernatant is collected on ice by filtration, lyophilized, and the residue dissolved in sterile water with protease inhibitor cocktail (Sigma, USA, or similar). The amino acids are quantified with an amino acid quantification kit (Solarbio, Shanghai, or similar) and proteins are quantified using the Bradford Assay kit as described (Bio-Rad, USA, or similar). Degradation of insect cuticle is determined by the ratio of amino acids to protein in the supernatant.

### Extracellular Protease Activity

The activity of total extracellular proteases in the supernatant collected as described above is assayed using Azocasein according to the manufacturer's instruction (Sigma, USA, or similar). One unit of proteolytic activity is defined as an increase of 0.01 absorbance at 440 nm after lh incubation at 28° C.

### Chitinolytic Activity

Ability of bacterial strains to degrade chitin is assayed as described by Zhang et al 2019 (ibid). Supernatants collected as described above are concentrated 20-fold by lyophilization, and assayed for chitinase activity using a Chitinase Assay Test Kit (Solarbio Life Sciences, Beijing, China, or similar). One unit of chitinase activity is defined as the amount of enzyme that releases 1 mg of N-acetylglucosamine per minute at 37° C.

### Neonicotinoid Production

Supernatants collected as described above are tested for Neonicotinoids using an Imidacloprid ELISA Kit (CDcreative Diagnostics, or eurofins, or similar).

### Siderophore Production

Supernatants collected as described above are tested for Siderophores content using a colorimetric test (SideroTec Kit, emergenbio, or similar).

### Utilization of Carbon Sources

The ability of bacterial isolates of the invention to utilize various carbon sources is assayed using a PM2A or PM1 MicroPlate™ Carbon Sources kit (Biolog).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A bacterial preparation comprising a plurality of at least one isolated bacterial strain or a functional homologue thereof at a concentration which exceeds that found in nature, wherein the isolated bacterial strain is selected from the group consisting of:
strain LAV61269, the strain being selected from the group consisting of (a) a strain deposited under Accession Number 43908 at NCIMB; (b) a strain comprising a 16S-rRNA sequence comprising the nucleic acid sequence set forth in SEQ ID NO:8; (c) a strain comprising at least one genomic marker comprising the nucleic acid sequence set forth in any one of SEQ ID NOs:73-75, and any combination thereof;
strain LAV41249, the strain being selected from the group consisting of (a) a strain deposited under Accession Number 43914 at NCIMB; (b) a strain comprising a 16S-rRNA sequence comprising the nucleic acid sequence set forth in SEQ ID NO:1; (c) a strain comprising at least one genomic marker comprising the nucleic acid sequence set forth in any one of SEQ ID NOs:52-55, and any combination thereof;
strain LAV61293, the strain being selected from the group consisting of (a) a strain deposited under Accession Number 43913 at NCIMB; (b) a strain comprising a 16S-rRNA sequence comprising the nucleic acid sequence set forth in any one of SEQ ID NO:11 and SEQ ID NO:12; (c) a strain comprising at least one genomic marker comprising the nucleic acid sequence set forth in any one of SEQ ID NOs:79-81, and any combination thereof;
strain LAV61301, the strain being selected from the group consisting of (a) a strain deposited under Accession Number 43910 at NCIMB; (b) a strain comprising a 16S-rRNA sequence comprising the nucleic acid sequence set forth in SEQ ID NO:17; (c) a strain comprising at least one genomic marker comprising the nucleic acid sequence set forth in any one of SEQ ID NOs:91-92, and any combination thereof;
strain LAV104746, the strain being selected from the group consisting of (a) a strain deposited under Accession Number 43911 at NCIMB; (b) a strain comprising a 16S-rRNA sequence comprising the nucleic acid sequence set forth in SEQ ID NO:26; (c) a strain comprising at least one genomic marker comprising the nucleic acid sequence set forth in any one of SEQ ID NOs:112-113, and any combination thereof;
strain LAV104743, the strain being selected from the group consisting of (a) a strain deposited under Accession Number 43903 at NCIMB; (b) a strain comprising a 16S-rRNA sequence comprising the nucleic acid sequence set forth in any one of SEQ ID NO:21 and SEQ ID NO:22; c) a strain comprising at least one genomic marker comprising the nucleic acid sequence set forth in any one of SEQ ID NOs:104-107, and any combination thereof;
strain LAV61243, the strain being selected from the group consisting of (a) a strain deposited under Accession Number 43905 at NCIMB; (b) a strain comprising a 16S-rRNA sequence comprising the nucleic acid sequence set forth in SEQ ID NO:4; (c) a strain comprising at least one genomic marker comprising the nucleic acid sequence set forth in any one of SEQ ID NOs:62-64, and any combination thereof;
strain LAV106494, the strain being selected from the group consisting of (a) a strain deposited under Accession Number 43915 at NCIMB; (b) a strain comprising a 16S-rRNA sequence comprising the nucleic acid sequence set forth in SEQ ID NO:46; (c) a strain comprising at least one genomic marker comprising the nucleic acid sequence set forth in any one of SEQ ID NOs:144-145, and any combination thereof;
strain LAV1000771, the strain being selected from the group consisting of (a) a strain deposited under Accession Number 43912 at NCIMB; (b) a strain comprising a 16S-rRNA sequence comprising the nucleic acid sequence set forth in SEQ ID NO:49; (c) a strain comprising at least one genomic marker comprising the nucleic acid sequence set forth in any one of SEQ ID NOs:164-165 and any combination thereof;
strain LAV1000775, the strain the strain being selected from the group consisting of (a) a strain deposited under Accession Number 43904 at NCIMB; (b) a strain comprising a 16S-rRNA sequence comprising the nucleic acid sequence set forth in SEQ ID NO:50; (c) a strain comprising at least one genomic marker comprising the nucleic acid sequence set forth in any one of SEQ ID NOs:166-170, and any combination thereof;
strain LAV1000731, the strain being selected from the group consisting of (a) a strain deposited under Accession Number 43906 at NCIMB; (b) a strain comprising a 16S-rRNA sequence comprising the nucleic acid sequence set forth in SEQ ID NO:48; (c) a strain comprising at least one genomic marker comprising the nucleic acid sequence set forth in any one of SEQ ID NOs:159-163, and any combination thereof;
strain LAV104934, the strain being selected from the group consisting of (a) a strain deposited under Accession Number 43902 at NCIMB; (b) a strain comprising a 16S-rRNA sequence comprising the nucleic acid sequence set forth in SEQ ID NO:40; (c) a strain comprising at least one genomic marker comprising the nucleic acid sequence set forth in any one of SEQ ID NOs:137-139, and any combination thereof;
strain LAV104944, the strain being selected from the group consisting of (a) a strain deposited under Accession Number 43907 at NCIMB; (b) a strain comprising a 16S-rRNA sequence comprising the nucleic acid sequence set forth in SEQ ID NO:45; (c) a strain comprising at least one genomic marker comprising the nucleic acid sequence set forth in any one of SEQ ID NOs:140-143, and any combination thereof. strain LAV104753, the strain being selected from the group consisting of (a) a strain comprising a 16S-rRNA sequence comprising the nucleic acid sequence set forth in SEQ ID NO:30; (b) a strain comprising at least one genomic marker comprising the nucleic acid sequence set forth in any one of SEQ ID NOs:119-122, and a combination thereof;
strain LAV106564, the strain being selected from the group consisting of (a) a strain comprising a 16S-rRNA sequence comprising the nucleic acid sequence set forth in any one of SEQ ID NO:41 and SEQ ID NO:42; (b) a strain comprising at least one genomic marker comprising the nucleic acid sequence set forth in any one of SEQ ID NOs:146-148, and a combination thereof;
strain LAV100189, the strain being selected from the group consisting of (a) a strain comprising a 16S-rRNA sequence comprising the nucleic acid sequence set forth in SEQ ID NO:20; (b) a strain comprising at least one genomic marker comprising the nucleic acid sequence set forth in any one of SEQ ID NOs:100-103, and a combination thereof; and
strain LAV1000686, the strain being selected from the group consisting of (a) a strain comprising a 16S-rRNA sequence comprising the nucleic acid sequence set forth in SEQ ID NO:47; (b) a strain comprising at least one genomic marker comprising the nucleic acid sequence set forth in any one of SEQ ID NOs:156-158, and a combination thereof.

2. The bacterial preparation of claim 1, wherein:
strain LAV61269, strain LAV104944, and strain LAV104753 each is of the genus Pseudomonas; strain LAV41249, strain LAV61293, strain LAV1000771, and strain LAV104746 each is of the genus Serratia; strain LAV61301 is of the genus Rahnella; strain LAV104743 and strain LAV106564 each is of the genus Carnobacterium; strain LAV61243 is of the genus Microbacterium; strain LAV106494 and strain LAV100189 each is of the genus Stenotrophomonas; strain LAV1000775 is of the genus Chryseobacterium; strain LAV1000731 is of the genus Paraburkholderia; and strain LAV104934 and strain LAV1000686 each is of the genus Acinetobacter.

3. The bacterial preparation of claim 1, wherein:
the functional homolog of bacterial strain LAV61269 comprises a 16S-rRNA sequence at least 98% identical to SEQ ID NO:8; at least one genomic marker having at least 99% local identity to the nucleic acid sequence of any one of SEQ ID NOs:73-75 over 97% coverage; or a combination thereof;
the functional homolog of bacterial strain LAV41249 comprises a 16S-rRNA sequence at least 97% identical to SEQ ID NO:1; at least one genomic marker having at least 99% local identity to the nucleic acid sequence of any one of SEQ ID NOs:52-55 over 97% coverage; or a combination thereof;
the functional homolog of strain LAV41249 is strain LAV43112, comprising a 16S-rRNA sequence comprising the nucleic acid sequence set forth in SEQ ID NO:2, at least one genomic marker comprising the nucleic acid sequence set forth in any one of SEQ ID NOs:52, and 54-56, or any combination thereof;
the functional homolog of bacterial strain LAV61293 comprises a 16S-rRNA sequence at least 97% identical to any one of SEQ ID NO:11 and SEQ ID NO:12; at least one genomic marker having at least 99% local identity to the nucleic acid sequence of any one of SEQ ID NOs:79-81 over 97% coverage; or a combination thereof;
the functional homolog of bacterial strain LAV61301 comprises a 16S-rRNA sequence at least 97% identical to SEQ ID NO:17; at least one genomic marker having at least 99% local identity to the nucleic acid sequence of any one of SEQ ID NOs:91-92 over 97% coverage; or a combination thereof;
the functional homolog of bacterial strain LAV104746 comprises a 16S-rRNA sequence at least 97% identical to SEQ ID NO:26; at least one genomic marker having at least 99% local identity to the nucleic acid sequence of any one of SEQ ID NOs:112-113 over 97% coverage; or a combination thereof;
the functional homolog of bacterial strain LAV104743 comprises a 16S-rRNA sequence at least 97% identical to any one of SEQ ID NO:21 and SEQ ID NO:22; at least one genomic marker having at least 99% local identity to the nucleic acid sequence of any one of SEQ ID NOs:104-107 over 97% coverage; or a combination thereof;
the functional homolog of bacterial strain LAV61243 comprises a 16S-rRNA sequence at least 97% identical to SEQ ID NO:4; at least one genomic marker having at least 99% local identity to the nucleic acid sequence of any one of SEQ ID NOs:62-64 over 97% coverage; or a combination thereof;
the functional homolog of bacterial strain LAV106494 comprises a 16S-rRNA sequence at least 97% identical to SEQ ID NO:46; at least one genomic marker having at least 99% local identity to the nucleic acid sequence of any one of SEQ ID NOs:144-145 over 97% coverage; or a combination thereof;
the functional homolog of bacterial strain LAV1000771 comprises a 16S-rRNA sequence at least 97% identical to SEQ ID NO:49; at least one genomic marker having at least 99% local identity to the nucleic acid sequence of any one of SEQ ID NOs:164-165 over 97% coverage; or a combination thereof;
the functional homolog of bacterial strain LAV1000775 comprises a 16S-rRNA sequence at least 97% identical to SEQ ID NO:50; at least one genomic marker having at least 99% local identity to the nucleic acid sequence of any one of SEQ ID NOs:166-170 over 97% coverage; or a combination thereof;
the functional homolog of bacterial strain LAV1000731 comprises a 16S-rRNA sequence at least 97% identical to SEQ ID NO:48; at least one genomic marker having at least 99% local identity to the nucleic acid sequence of any one of SEQ ID NOs:159-163 over 97% coverage; or a combination thereof;
the functional homolog of bacterial strain LAV104753 comprises a 16S-rRNA sequence at least 97% identical to SEQ ID NO:30; at least one genomic marker having at least 99% local identity to the nucleic acid sequence of any one of SEQ ID NOs:119-122 over 97% coverage; or a combination thereof;
the functional homolog of bacterial strain LAV106564 comprises a 16S-rRNA sequence at least 97% identical to any one of SEQ ID NOs:41-42; at least one genomic marker having at least 99% local identity to the nucleic acid sequence of any one of SEQ ID NOs:146-148 over 97% coverage; or a combination thereof;
the functional homolog of bacterial strain LAV100189 comprises a 16S-rRNA sequence at least 97% identical to SEQ ID NO:20; at least one genomic marker having at least 99% local identity to the nucleic acid sequence of any one of SEQ ID NOs:100-103 over 97% coverage; or a combination thereof;
the functional homolog of bacterial strain LAV1000686 comprises a 16S-rRNA sequence at least 97% identical to SEQ ID NO:47; at least one genomic marker having at least 99% local identity to the nucleic acid sequence of any one of SEQ ID NOs:156-158 over 97% coverage; or a combination thereof;
the functional homolog of bacterial strain LAV104934 comprises a 16S-rRNA sequence at least 98% identical to SEQ ID NO:40; at least one genomic marker having at least 99% local identity to the nucleic acid sequence of any one of SEQ ID NOs:137-139 over 97% coverage; or a combination thereof; and
the functional homolog of bacterial strain LAV104944 comprises a 16S-rRNA sequence at least 98% identical to SEQ ID NO:45; at least one genomic marker having at least 99% local identity to the nucleic acid sequence of any one of SEQ ID NOs:140-143 over 97% coverage; or a combination thereof

4. The bacterial preparation of claim 1, wherein the bacterial strain or functional homolog is characterized by a capability to inhibit the growth and/or the development and/or the activity of, or to kill, at least one plant insect and/or mite pest, thereby protecting the plant from, or reducing the impact of the harmful effects of, the at least one insect and/or mite pest.

5-6. (canceled)

7. The bacterial preparation of claim 1, wherein said bacterial preparation comprises the plurality of bacteria of at least one bacterial strain or functional homolog thereof in a form selected from the group consisting of viable form and non-viable form.

8. The bacterial preparation of claim 1, wherein said bacterial preparation is substantially stable for at least 30 days at a temperature range of from about 4° C. to about 37° C.

9. A lysate of the bacterial preparation of claim 1, wherein the lysate is selected from the group consisting of a whole cell lysate of the at least one bacterial strain or functional homolog thereof, and a soluble fraction of the at least one bacterial strain or functional homolog thereof.

10. (canceled)

11. An extract of the bacterial preparation of claim 1.

12. A whole cell broth collected from fermentation of the bacterial preparation of claim 1.

13. An agricultural composition comprising the bacterial preparation of claim 1, a lysate, an extract, or a whole cell broth thereof, further comprising agriculturally acceptable diluents or carriers.

14-16. (canceled)

17. The agricultural composition of claim 13, wherein the carrier is a plant seed.

18. A seed coated with the agricultural composition of claim 13.

19-20. (canceled)

21. A method for at least one of protecting a plant from at least one insect and/or mite pest and inhibiting or preventing the growth and/or the development of the at least one insect and/or mite pest present in or on a plant or a part thereof, comprising contacting the plant, part or habitat thereof with at least one bacterial strain or functional homolog thereof, wherein the bacterial strain is selected from the group consisting of:
strain LAV61269, the strain being selected from the group consisting of (a) a strain deposited under Accession Number 43908 at NCIMB; (b) a strain comprising a 16S-rRNA sequence comprising the nucleic acid sequence set forth in SEQ ID NO:8; (c) a strain comprising at least one genomic marker comprising the nucleic acid sequence set forth in any one of SEQ ID NOs:73-75, and any combination thereof;
strain LAV41249, the strain being selected from the group consisting of (a) a strain deposited under Accession Number 43914 at NCIMB; (b) a strain comprising a 16S-rRNA sequence comprising the nucleic acid sequence set forth in SEQ ID NO:1; (c) a strain comprising at least one genomic marker comprising the nucleic acid sequence set forth in any one of SEQ ID NOs:52-55, and any combination thereof;
strain LAV61293, the strain being selected from the group consisting of (a) a strain deposited under Accession Number 43913 at NCIMB; (b) a strain comprising a 16S-rRNA sequence comprising the nucleic acid sequence set forth in any one of SEQ ID NO:11 and SEQ ID NO:12; (c) a strain comprising at least one genomic marker comprising the nucleic acid sequence set forth in any one of SEQ ID NOs:79-81, and any combination thereof;
strain LAV61301, the strain being selected from the group consisting of (a) a strain deposited under Accession Number 43910 at NCIMB; (b) a strain comprising a 16S-rRNA sequence comprising the nucleic acid sequence set forth in SEQ ID NO:17; (c) a strain comprising at least one genomic marker comprising the nucleic acid sequence set forth in any one of SEQ ID NOs:91-92, and any combination thereof;
strain LAV104746, the strain being selected from the group consisting of (a) a strain deposited under Accession Number 43911 at NCIMB; (b) a strain comprising a 16S-rRNA sequence comprising the nucleic acid sequence set forth in SEQ ID NO:26; (c) a strain comprising at least one genomic marker comprising the nucleic acid sequence set forth in any one of SEQ ID NOs:112-113, and any combination thereof;
strain LAV104743, the strain being selected from the group consisting of (a) a strain deposited under Accession Number 43903 at NCIMB; (b) a strain comprising a 16S-rRNA sequence comprising the nucleic acid sequence set forth in any one of SEQ ID NO:21 and SEQ ID NO:22; c) a strain comprising at least one genomic marker comprising the nucleic acid sequence set forth in any one of SEQ ID NOs:104-107, and any combination thereof;
strain LAV61243, the strain being selected from the group consisting of (a) a strain deposited under Accession Number 43905 at NCIMB; (b) a strain comprising a 16S-rRNA sequence comprising the nucleic acid sequence set forth in SEQ ID NO:4; (c) a strain comprising at least one genomic marker comprising the nucleic acid sequence set forth in any one of SEQ ID NOs:62-64, and any combination thereof;
strain LAV106494, the strain being selected from the group consisting of (a) a strain deposited under Accession Number 43915 at NCIMB; (b) a strain comprising a 16S-rRNA sequence comprising the nucleic acid sequence set forth in SEQ ID NO:46; (c) a strain comprising at least one genomic marker comprising the nucleic acid sequence set forth in any one of SEQ ID NOs:144-145, and any combination thereof;
strain LAV1000771, the strain being selected from the group consisting of (a) a strain deposited under Accession Number 43912 at NCIMB; (b) a strain comprising a 16S-rRNA sequence comprising the nucleic acid sequence set forth in SEQ ID NO:49; (c) a strain comprising at least one genomic marker comprising the nucleic acid sequence set forth in any one of SEQ ID NOs:164-165 and any combination thereof;
strain LAV1000775, the strain the strain being selected from the group consisting of (a) a strain deposited under Accession Number 43904 at NCIMB; (b) a strain comprising a 16S-rRNA sequence comprising the nucleic acid sequence set forth in SEQ ID NO:50; (c) a strain comprising at least one genomic marker comprising the nucleic acid sequence set forth in any one of SEQ ID NOs:166-170, and any combination thereof;
strain LAV1000731, the strain being selected from the group consisting of (a) a strain deposited under Accession Number 43906 at NCIMB; (b) a strain comprising a 16S-rRNA sequence comprising the nucleic acid sequence set forth in SEQ ID NO:48; (c) a strain comprising at least one genomic marker comprising the nucleic acid sequence set forth in any one of SEQ ID NOs:159-163, and any combination thereof;
strain LAV104934, the strain being selected from the group consisting of (a) a strain deposited under Accession Number 43902 at NCIMB; (b) a strain comprising a 16S-rRNA sequence comprising the nucleic acid sequence set forth in SEQ ID NO:40; (c) a strain comprising at least one genomic marker comprising the nucleic acid sequence set forth in any one of SEQ ID NOs:137-139, and any combination thereof;
strain LAV104944, the strain being selected from the group consisting of (a) a strain deposited under Accession Number 43907 at NCIMB; (b) a strain comprising a 16S-rRNA sequence comprising the nucleic acid sequence set forth in SEQ ID NO:45; (c) a strain comprising at least one genomic marker comprising the nucleic acid sequence set forth in any one of SEQ ID NOs:140-143, and any combination thereof. strain LAV104753, the strain being selected from the group consisting of (a) a strain comprising a 16S-rRNA sequence comprising the nucleic acid sequence set forth in SEQ ID NO:30; (b) a strain comprising at least one genomic marker comprising the nucleic acid sequence set forth in any one of SEQ ID NOs:119-122, and a combination thereof;
strain LAV106564, the strain being selected from the group consisting of (a) a strain comprising a 16S-rRNA sequence comprising the nucleic acid sequence set forth in any one of SEQ ID NO:41 and SEQ ID NO:42; (b) a strain comprising at least one genomic marker comprising the nucleic acid sequence set forth in any one of SEQ ID NOs:146-148, and a combination thereof;
strain LAV100189, the strain being selected from the group consisting of (a) a strain comprising a 16S-rRNA sequence comprising the nucleic acid sequence set forth in SEQ ID NO:20; (b) a strain comprising at least one genomic marker comprising the nucleic acid sequence set forth in any one of SEQ ID NOs:100-103, and a combination thereof; and
strain LAV1000686, the strain being selected from the group consisting of (a) a strain comprising a 16S-rRNA sequence comprising the nucleic acid sequence set forth in SEQ ID NO:47; (b) a strain comprising at least one genomic marker comprising the nucleic acid sequence set forth in any one of SEQ ID NOs:156-158, and a combination thereof;
strain LAV104744, the strain being selected from the group consisting of (a) a strain deposited under Accession Number 43901 at NCIMB; (b) a strain comprising a 16S-rRNA sequence comprising the nucleic acid sequence set forth in SEQ ID NO:23; (c) a strain comprising at least one genomic marker comprising the nucleic acid sequence set forth in any one of SEQ ID NOs:108-111, and any combination thereof;
strain LAV104747, the strain being selected from the group consisting of (a) a strain deposited under Accession Number 43909 at NCIMB; (b) a strain comprising a 16S-rRNA sequence comprising the nucleic acid sequence set forth in any one of SEQ ID NO:27 and SEQ ID NO:28; (c) a strain comprising at least one genomic marker comprising the nucleic acid sequence set forth in any one of SEQ ID NOs:114-116 and any combination thereof;
strain LAV1000939, the strain being selected from the group consisting of (a) a strain deposited under Accession Number 43916 at NCIMB; (b) a strain comprising a 16S-rRNA sequence comprising the nucleic acid sequence set forth in SEQ ID NO:51; (c) a strain comprising at least one genomic marker comprising the nucleic acid sequence set forth in any one of SEQ ID NOs:171-175, and any combination thereof; and
strain LAV61288, the strain being selected from the group consisting of (a) a strain comprising a 16S-rRNA sequence comprising the nucleic acid sequence set forth in any one of SEQ ID NO:9 and SEQ ID NO:10; (b) a strain comprising at least one genomic marker comprising the nucleic acid sequence set forth in any one of SEQ ID NOs:76-78, and a combination thereof;
a bacterial preparation of same, a lysate thereof, an extract thereof; a whole cell broth obtained therefrom; or an agricultural composition comprising same.

22. The method of claim 21, wherein the plant or part thereof comprises the at least one insect and/or mite pest in or on at least one cell or tissue of said plant or part thereof.

23-27. (canceled)

28. The method of claim 21, wherein the plant or part thereof is contacted with the at least one bacterial cell at a concentration of from about 10² CFU/ml to about 10⁸ CFU/ml.

29. (canceled)

30. The method of claim 21, wherein the insect is of an order selected from the group consisting of Lepidoptera, Coleoptera, Hemiptera.

31. The method of claim 30, wherein the insect is selected from the group consisting of Western corn rootworm (WCRW, Diabrotica virgifera virgifera), Southern green stink bug (STK, Nezara viridula), Fall armyworm (FAW, Spodoptera frugiperda), Beet Armyworm (BAW, Spodoptera exigua), Western tarnished plant bug (Lygus hesperus), Cabbage Loopers (Trichoplusia ni), Diamondback Moth (Plutella xylostella), Green Peach Aphids (Myzus persicae), and Soybean Looper (Chrysodeixis includes).

32. The method of claim 21, wherein the mite is of the order Acari.

33. The method of claim 32, wherein the mite is Twospotted spider mite (Tetranychus urticae).

34. The method of claim 21, wherein:
the functional homolog of bacterial strain LAV61269 comprises a 16S-rRNA sequence at least 98% identical to SEQ ID NO:8; at least one genomic marker having at least 99% local identity to the nucleic acid sequence of any one of SEQ ID NOs:73-75 over 97% coverage; or a combination thereof;
the functional homolog of bacterial strain LAV41249 comprises a 16S-rRNA sequence at least 97% identical to SEQ ID NO:1; at least one genomic marker having at least 99% local identity to the nucleic acid sequence of any one of SEQ ID NOs:52-55 over 97% coverage; or a combination thereof;
the functional homolog of strain LAV41249 is strain LAV43112, comprising a 16S-rRNA sequence comprising the nucleic acid sequence set forth in SEQ ID NO:2, at least one genomic marker comprising the nucleic acid sequence set forth in any one of SEQ ID NOs:52, and 54-56, or any combination thereof;
the functional homolog of bacterial strain LAV61293 comprises a 16S-rRNA sequence at least 97% identical to any one of SEQ ID NO:11 and SEQ ID NO:12; at least one genomic marker having at least 99% local identity to the nucleic acid sequence of any one of SEQ ID NOs:79-81 over 97% coverage; or a combination thereof;
the functional homolog of bacterial strain LAV61301 comprises a 16S-rRNA sequence at least 97% identical to SEQ ID NO:17; at least one genomic marker having at least 99% local identity to the nucleic acid sequence of any one of SEQ ID NOs:91-92 over 97% coverage; or a combination thereof;
the functional homolog of bacterial strain LAV104746 comprises a 16S-rRNA sequence at least 97% identical to SEQ ID NO:26; at least one genomic marker having at least 99% local identity to the nucleic acid sequence of any one of SEQ ID NOs:112-113 over 97% coverage; or a combination thereof;
the functional homolog of bacterial strain LAV104743 comprises a 16S-rRNA sequence at least 97% identical to any one of SEQ ID NO:21 and SEQ ID NO:22; at least one genomic marker having at least 99% local identity to the nucleic acid sequence of any one of SEQ ID NOs:104-107 over 97% coverage; or a combination thereof;
the functional homolog of bacterial strain LAV61243 comprises a 16S-rRNA sequence at least 97% identical to SEQ ID NO:4; at least one genomic marker having at least 99% local identity to the nucleic acid sequence of any one of SEQ ID NOs:62-64 over 97% coverage; or a combination thereof;
the functional homolog of bacterial strain LAV106494 comprises a 16S-rRNA sequence at least 97% identical to SEQ ID NO:46; at least one genomic marker having at least 99% local identity to the nucleic acid sequence of any one of SEQ ID NOs:144-145 over 97% coverage; or a combination thereof;
the functional homolog of bacterial strain LAV1000771 comprises a 16S-rRNA sequence at least 97% identical to SEQ ID NO:49; at least one genomic marker having at least 99% local identity to the nucleic acid sequence of any one of SEQ ID NOs:164-165 over 97% coverage; or a combination thereof;
the functional homolog of bacterial strain LAV1000775 comprises a 16S-rRNA sequence at least 97% identical to SEQ ID NO:50; at least one genomic marker having at least 99% local identity to the nucleic acid sequence of any one of SEQ ID NOs:166-170 over 97% coverage; or a combination thereof;
the functional homolog of bacterial strain LAV1000731 comprises a 16S-rRNA sequence at least 97% identical to SEQ ID NO:48; at least one genomic marker having at least 99% local identity to the nucleic acid sequence of any one of SEQ ID NOs:159-163 over 97% coverage; or a combination thereof;
the functional homolog of bacterial strain LAV104753 comprises a 16S-rRNA sequence at least 97% identical to SEQ ID NO:30; at least one genomic marker having at least 99% local identity to the nucleic acid sequence of any one of SEQ ID NOs:119-122 over 97% coverage; or a combination thereof;
the functional homolog of bacterial strain LAV106564 comprises a 16S-rRNA sequence at least 97% identical to any one of SEQ ID NOs:41-42; at least one genomic marker having at least 99% local identity to the nucleic acid sequence of any one of SEQ ID NOs:146-148 over 97% coverage; or a combination thereof;
the functional homolog of bacterial strain LAV100189 comprises a 16S-rRNA sequence at least 97% identical to SEQ ID NO:20; at least one genomic marker having at least 99% local identity to the nucleic acid sequence of any one of SEQ ID NOs:100-103 over 97% coverage; or a combination thereof;
the functional homolog of bacterial strain LAV1000686 comprises a 16S-rRNA sequence at least 97% identical to SEQ ID NO:47; at least one genomic marker having at least 99% local identity to the nucleic acid sequence of any one of SEQ ID NOs:156-158 over 97% coverage; or a combination thereof;
the functional homolog of bacterial strain LAV104934 comprises a 16S-rRNA sequence at least 98% identical to SEQ ID NO:40; at least one genomic marker having at least 99% local identity to the nucleic acid sequence of any one of SEQ ID NOs:137-139 over 97% coverage; or a combination thereof; and
the functional homolog of bacterial strain LAV104944 comprises a 16S-rRNA sequence at least 98% identical to SEQ ID NO:45; at least one genomic marker having at least 99% local identity to the nucleic acid sequence of any one of SEQ ID NOs:140-143 over 97% coverage; or a combination thereof.


SEQUENCE LISTING (179 sequences)

SEQ ID NO: 1 - DNA, 1434 bp, Serratia sp.
  ribosomal RNA nucleic acid sequence
agagtttgatcatggctcagattgaacgctggcggcaggcttaacacatgcaagtcgagcggtagcacaggggagcttgctccctgggtgacgagcggcggacgggtgagtaatgtctgggaaactgcctgatggagggggataactactggaaacggtagctaataccgcataatgtcgcaagaccaaagagggggaccttcgggcctcttgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaaggtggtgaacttaatacgttcatcaattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcgtatacaaagagaagcgacctcgcgagagcaagcggacctcataaagtacgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggta

SEQ ID NO: 2 - DNA, 1397 bp, Serratia sp.
  ribosomal RNA nucleic acid sequence
tgcagtcgagcggtagcacaggggagcttgctccctgggtgacgagcggcggacgggtgagtaatgtctgggaaactgcctgatggagggggataactactggaaacggtagctaataccgcataatgtcgcaagaccaaagagggggaccttcgggcctcttgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaaggtggtgaacttaatacgttcatcaattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcgtatacaaagagaagcgacctcgcgagagcaagcggacctcataaagtacgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcg

SEQ ID NO: 3 - DNA, 1484 bp, Pantoea sp.
  ribosomal RNA nucleic acid sequence
aggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgacggggttaataaccctgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttggcagagatgccttagtgccttcgggaaccgtgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaggg

SEQ ID NO: 4 - DNA, 1326 bp, Microbacterium sp.
  ribosomal RNA nucleic acid sequence
aacacgtgagcaacctgcccctgactctgggataagcgctggaaacggcgtctaatactggatatgtgacgtgaccgcatggtctgcgtctggaaagaatttcggttggggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgcaagcctgatgcagcaacgccgcgtgagggacgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatccggaggctcaacctccggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtccattccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgcgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcctaacccttgtggagg

SEQ ID NO: 5 - DNA, 1486 bp, Stenotrophomonas sp.
  misc_feature (62)..(62): n is a, c, g, or t
  misc_feature (444)..(444): n is a, c, g, or t
  misc_feature (1007)..(1007): n is a, c, g, or t
  ribosomal RNA nucleic acid sequence
tcatggctcagagtgaacgctggcggtaggcctaacacatgcaagtcgaacggcagcacagnagagcttgctctctgggtggcgagtggcggacgggtgaggaatacatcggaatctactctgtcgtgggggataacgtagggaaacttacgctaataccgcatacgacctacgggtgaaagcaggggatcttcggaccttgcgcgattgaatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaatccagcnggctaatacccggttgggatgacggtacccaaagaataagcaccggctaacttcgtgccagcagccgcggtaatacgaagggtgcaagcgttactcggaattactgggcgtaaagcgtgcgtaggtggtcgtttaagtccgttgtgaaagccctgggctcaacctgggaactgcagtggatactgggcgactagagtgtggtagagggtagcggaattcctggtgtagcagtgaaatgcgtagagatcaggaggaacatccatggcgaaggcagctacctggaccaacactgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtcgagaactttccagagatggatnggtgccttcgggaactcgaacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacggccagggctacacacgtactacaatggtagggacagagggctgcaagccggcgacggtaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacggtgtggccgatgactggggtgaagtcgta

SEQ ID NO: 6 - DNA, 1470 bp, Rathayibacter sp.
  ribosomal RNA nucleic acid sequence
tcatggctcaggacgaacgctggcggcgtgcttaacacatgcaagtcgaacgatgaagcccagcttgctgggtggattagtggcgaacgggtgagtaacacgtgagtaacctgcccttgactctgggataagcgctggaaacggcgtctaataccggatacgacctgccccggcatcgggtgcgggtggaaagtttttcggtcaaggatggactcgcggcctatcaggtagttggtgaggtaatggctcaccaagccgacgacgggtagccggcctgagagggtgaccggccacactgggactgagacacggcccagactcctacgggaggcagcagtggggaatattgcacaatgggcgaaagcctgatgcagcaacgccgcgtgggggatgacggccttcgggttgtaaacctcttttagtagggaagaagggcttcggcttgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaaacccgaggctcaacctcgggcctgcagtgggtacgggcagactagagtgcggtaggggagaatggaattcctggtgtagcggtggaatgcgcagatatcaggaggaacaccgatggcgaaggcagttctctgggccgttactgacgctgaggagcgaaagcgtggggagcgaacaggattagataccctggtagtccacgccgtaaacgttgggaactagatgtggggacctttccacggtctccgtgtcgcagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatataccggaaacttccagaaatggttgccccgcaaggtcggtatacaggtggtgcatggttgtcgtcagctcgtgtcgtgagatgttgggttaagtcccgcaacgagcgcaaccctcgttctatgttgccagcacgtcatggtgggaactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccagtggcctaaccgcaaggagggagctgtcgaaggtgggatcggtgattaggactaagtcgtaacaggg

SEQ ID NO: 7 - DNA, 1414 bp, Bacillus sp.
  ribosomal RNA nucleic acid sequence
ggttaccccaccgacttcgggtgttacaaactctcgtggtgtgacgggcggtgtgtacaaggcccgggaacgtattcaccgcggcatgctgatccgcgattactagcgattccagcttcatgtaggcgagttgcagcctacaatccgaactgagaacgggttttatgagattagctccacctcgcggtcttgcagctctttgtaccgtccattgtagcacgtgtgtagcccaggtcataaggggcatgatgatttgacgtcatccccaccttcctccggtttgtcaccggcagtcaccttagagtgcccaacttaatgatggcaactaagatcaagggttgcgctcgttgcgggacttaacccaacatctcacgacacgagctgacgacaaccatgcaccacctgtcactctgctcccgaaggagaagctctatctctagagttttcagaggatgtcaagacctggtaaggttcttcgcgttgcttcgaattaaaccacatgctccaccgcttgtgcggccccccgtcaattcctttgagtttcagccttgcggccgtactccccaggcggagtgcttaatgcgttaacttcagcactaaagggcggaaaccctctaacacttagcactcatcgtttacggcgtggactaccagggtatctaatcctgtttgctccccacgctttcgcgcctcagtgtcagttacagaccagaaagtcgccttcgccactggtgttcctccatatctctacgcatttcaccgctacacatggaattccactttcctcttctgcactcaagtctcccagtttccaatgaccctccacggttgagccgtgggctttcacatcagacttaagaaaccacctgcgcgcgctttacgcccaataattccggataacgcttgccacctacgtattaccgcggctgctggcacgtagttagccgtggctttctggttaggtaccgtcaaggtgccagcttattcaactagcacttgttcttccctaacaacagagttttacgacccgaaagccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgacgcgggtccatccataagtgacagccgaagccgcctttcaatttcgaactatgcagttcaaaatattatccggtattagccccggtttcccggagttatcccagtcttatgggtaggttacccacgtgttactcacccgtccgccgctaacttcataagagcaagctgcttaatccattcgctcgactgcagtattaggca

SEQ ID NO: 8 - DNA, 1489 bp, Pseudomonas sp.
  misc_feature (447)..(447): n is a, c, g, or t
  misc_feature (452)..(452): n is a, c, g, or t
  misc_feature (1146)..(1146): n is a, c, g, or t
  ribosomal RNA nucleic acid sequence
ggctcagattgaacgctggcggcaggcctaacacatgcaagtcgagcggtagagaggtgcttgcacctcttgagagcggcggacgggtgagtaatgcctaggaatctgcctggtagtgggggataacgctcggaaacggacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttngtgtnttgacgttaccgacagaataagcaccggctaactctgtgccagcagccgcggtaatacagagggtgcaagcgttaatcggaattactgggcgtaaagcgcgcgtaggtggttcgttaagttggatgtgaaagccccgggctcaacctgggaactgcattcaaaactgtcgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaanccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacagggtacccgtaa

SEQ ID NO: 9 - DNA, 1505 bp, Pseudomonas sp.
  ribosomal RNA nucleic acid sequence
ttacggttacccctgttacgacttcaccccagtcatgaatcacaccgtggtaaccgtcctcccgaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatctatctctagaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaaaagctcaaggcttccaacggctagttgacatcgtttacggcgtggactaccagggtatctaatcctgtttgctccccacgctttcgcacctcagtgtcagtattagtccaggtggtcgccttcgccactggtgttccttcctatatctacgcatttcaccgctacacaggaaattccaccaccctctaccatactctagtcagtcagttttgaatgcagttcccaggttgagcccggggatttcacatccaacttaacaaaccacctacgcgcgctttacgcccagtaattccgattaacgcttgcaccctctgtattaccgcggctgctggcacagagttagccggtgcttattctgtcggtaacgtcaaaattgcagagtattaatctacaacccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgtccgtttccgaacgttatcccccactaccaggcagattcctaggcattactcacccgtccgccgctctcaagagaagcaagcttctctctaccgctcgacttgcatgtgttaggcctgccgccagcgttcaatctgagccatgattcaaactcta

SEQ ID NO: 10 - DNA, 1520 bp, Pseudomonas sp.
  ribosomal RNA nucleic acid sequence
aagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctgga

SEQ ID NO: 11 - DNA, 1510 bp, Serratia sp.
  ribosomal RNA nucleic acid sequence
ttacggttacccctgttacgacttcaccccagtcatgaatcacaaagtggtaagcgccctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacatactttatgaggtccgcttgctctcgcgaggtcgcttctctttgtatatgccattgtagcacgtgtgtagccctactcgtaagggccatgatgacttgacgtcatccccacctacctcaaatttatcaccggcagtctcctttgagttcccgaccgaatcgctggcaacaaaggataagggttgcgctcgttgcgggacttaacccaacatttcacaacacgagctgacgacagccatgcagcacctgtctcagagttcccgaaggcaccaaagcatctctgctaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgatttaacgcgttagctccggaagccacgcctcaagggcacaacctccaaatcgacatcgtttacagcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagcctgccagtttcaaatgcagttcccaagttaagctcggggatttcacatctgacttaacagaccgcctgcgtgcgctttacgcccagtaattccgattaacgcttgcaccctccgtattaccgcggctgctggcacggagttagccggtgcttcttctgcgagtaacgtcaatgaacggcgctattaacaccgaacccttcctcctcgctgaaagtgctttacaacccgaaggccttcttcacacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccattaccccacctactagctaatcccatctgggcacatccgatggtgtgaggcccgaaggtcccccactttggtccgtagacgttatgcggtattagctaccgtttccagtagttatccccctccatcgggcagtttcccagacattactcacccgtccgccgctcgccggcaaagtagcaagctactttccgctgccgctcgacttgcatgtgttaggcctgccgccagcgttcaatctgagccatgattcaaactcta

SEQ ID NO: 12 - DNA, 1524 bp, Serratia sp.
  ribosomal RNA nucleic acid sequence
agagtttgatcatggctcagattgaacgctggcggcaggcctaacacatgcaagtcgagcggcagcggaaagtagcttgctactttgccggcgagcggcggacgggtgagtaatgtctgggaaactgcccgatggagggggataactactggaaacggtagctaataccgcataacgtctacggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaagggttcggtgttaatagcgccgttcattgacgttactcgcagaagaagcaccggctaactccgtgccagcagccgcggtaatacggagggtgcaagcgttaatcggaattactgggcgtaaagcgcacgcaggcggtctgttaagtcagatgtgaaatccccgagcttaacttgggaactgcatttgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttgga

SEQ ID NO: 13 - DNA, 1429 bp, Ensifer sp.
  ribosomal RNA nucleic acid sequence
tggctcagaacgaacgctggcggcaggcttaacacatgcaagtcgagcgccccgcaaggggagcggcagacgggtgagtaacgcgtgggaatctacccttttctacggaataacgcagggaaacttgtgctaataccgtatgagcccttcgggggaaagatttatcgggaaaggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggaattactgggcgtaaagcgcacgtaggcggattgttaagtgaggggtgaaatcccagggctcaaccctggaactgcctttcatactggcaatctagagtccagaagaggtgagtggaattccgagtgtagaggtgaaattcgtagatattcggaggaacaccagtggcgaaggcggctcactggtctggaactgacgctgaggtgcgaaagcgtggggagcaaacaggattagataccctggtagtccacgccgtaaacgatgaatgttagccgtcgggcagtttactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcccgatcgcggatacgagagatcgtatccttcagttcggctggatcggagacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttctacccgaaggtagtgcgctaaccgcaaggaggcagctaaccacggtagggtcagcgactggggtgaagtcgtaacaggg

SEQ ID NO: 14 - DNA, 1124 bp, Streptomyces sp.
  ribosomal RNA nucleic acid sequence
tcatggctcaggacgaacgctggcggcgtgcttaacacatgcaagtcgaacgatgaagcccttcggggtggattagtggcgaacgggtgagtaacacgtgggcaatctgcccttcactctgggacaagccctggaaacggggtctaataccggatacgagttcgggaggcatctcctggactggaaagctccggcggtgaaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcgggtgtgaaagcccggggcttaaccccgggtctgcatccgatacgggcaggctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcattagagatagtgccccccttgtggtcggtatacaggtggtgcatggctgtcgtcagctcgtgtcgtgagatgttgggttaagtcccgcaacgagcgcaacccttgtcctgtgttgccagcatgcccttcggggtgatggggactcacaggagaccg

SEQ ID NO: 15 - DNA, 1509 bp, Streptomyces sp.
  ribosomal RNA nucleic acid sequence
agagtttgatcctggctcaggacgaacgctggcggcgtgcttaacacatgcaagtcgaacgatgaagcccttcggggtggattagtggcgaacgggtgagtaacacgtgggcaatctgcccttcactctgggacaagccctggaaacggggtctaataccggatacgagttcgggaggcatctcctggactggaaagctccggcggtgaaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcgggtgtgaaagcccggggcttaaccccgggtctgcatccgatacgggcaggctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcattagagatagtgccccccttgtggtcggtatacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaaagagctgcgatgccgcgaggcggagcgaatctcaaaaagccggtctcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccggtggcccaaccccttgtgggagggagctgtcgaaggtgggactggcgattgggacgaagtcgtaacaaggtagccgtaccggaaggtgcggctggat

SEQ ID NO: 16 - DNA, 1417 bp, Serratia sp.
  misc_feature (1045)..(1046): n is a, c, g, or t
  misc_feature (1049)..(1049): n is a, c, g, or t
  misc_feature (1053)..(1054): n is a, c, g, or t
  misc_feature (1173)..(1173): n is a, c, g, or t
  ribosomal RNA nucleic acid sequence
tcctgggtgacgagcggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaagggtttggtgttaatagcaccattcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcnngtnatgnngggaactcaaaggagactgccggtgataaaccggaggaaggtggggatgacgtcaagtcatcatggcccttacgagtagggctacacacgtgctacaatggcatatacaaagagaagcnaactcgcgagagcaagcggacctcacaaagtatgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtacaggg

SEQ ID NO: 17 - DNA, 1510 bp, Rahnella sp.
  ribosomal RNA nucleic acid sequence
ttacggttacccctgttacgacttcaccccagtcatgaatcacaaagtggtaagcgccctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacatactttatgaggtccgcttgctctcgcgagttcgcttctctttgtatatgccattgtagcacgtgtgtagccctactcgtaagggccatgatgacttgacgtcatccccaccttcctccggtttatcaccggcagtctcctttgagttcccgacattactcgctggcaacaaaggataagggttgcgctcgttgcgggacttaacccaacatttcacaacacgagctgacgacagccatgcagcacctgtctcacagttcccgaaggcactaagccatctctggcgaattctgtggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacagcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagcttgccagtttcaaatgcagctcccacggtaagcgcggggatttcacatctgacttaacaaaccgcctgcgtgcgctttacgcccagtaattccgattaacgcttgcaccctccgtattaccgcggctgctggcacggagttagccggtgcttcttctgcgagtaacgtcaatcaccacacgtattaagtgtgatgccttcctcctcgctgaaagtgctttacaaccctaaggccttcttcacacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccattacctcacctactagctaatcccatctgggcacatccgatggcgtgaggtccgaagatcccccactttgctctttcgaggtcatgcggtattagctaccgtttccagtagttatccccctccatcaggcagtttcccagacattactcacccgtccgccgctcgccggcaaagtagcaagctacttcccgctgccgctcgacttgcatgtgttaggcctgccgccagcgttcaatctgagccatgattcaaactct

SEQ ID NO: 18 - DNA, 1438 bp, Aminobacter sp.
  ribosomal RNA nucleic acid sequence
tggctcagaacgaacgctggcggcaggcttaacacatgcaagtcgagcgccccgcaaggggagcggcagacgggtgagtaacgcgtgggaatctacccagctctacggaataactcagggaaacttgtgctaataccgtatacgtccgataggagaaagatttatcggagttggatgagcccgcgttggattagctagttggtggggtaatggcctaccaaggcgacgatccatagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggaattactgggcgtaaagcgcacgtaggcggatatttaagttaggggtgaaatcccagggctcaaccctggaactgcctttaatactgggtatctcgagtccgagagaggtgagtggaattccgagtgtagaggtgaaattcgtagatattcggaggaacaccagtggcgaaggcggctcactggctcggtactgacgctgaggtgcgaaagcgtggggagcaaacaggattagataccctggtagtccacgccgtaaactatgagagctagccgtcggcaagtttacttgtcggtggcgcagctaacgcattaagctctccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggattccttcagttcggctggaccggtgacaggtgctgcatggctgtcgtcagctcgtgtcgtgagatgttgggttaagtcccgcaacgagcgcaaccttcgcccttagttgccatcattaagttgggcactctaaggggactgccggtgataagccgcgaggaaggtgaagatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttttacccgaaggcactgtgctaaccgcaaggaggcaggtgaccacggtagggtcagcgactggggtgaagtcgtaacagggtaaccgtaa

SEQ ID NO: 19 - DNA, 1472 bp, Bacillus sp.
  ribosomal RNA nucleic acid sequence
ccttcggcggctggctcctaaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtaccgccctattcgaacggtacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgttgccttggtgagccgttacctcaccaactagctaatgcgccgcgggtccatctgtaagtggtagccgaagccaccttttatgtttgaaccatgcggttcaaacaagcatccggtattagccccggtttcccggagttatcccagtcttacaggcaggttacccacgtgttactcacccgtccgccgctaacatcagggagcaagctcccatctgtccgctcgacttgcatgtattaggcacgccggccagcgttcgtcctgagccatgattcaaactc

SEQ ID NO: 20 - DNA, 1437 bp, Stenotrophomonas sp.
  ribosomal RNA nucleic acid sequence
tggcggtaggcctaacacatgcaagtcgaacggcagcacaggagagcttgctctctgggtggcgagtggcggacgggtgaggaatacatcggaatctactctgtcgtgggggataacgtagggaaacttacgctaataccgcatacgacctacgggtgaaagcaggggatcttcggaccttgcgcgattgaatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaatccagccggctaatacctggttgggatgacggtacccaaagaataagcaccggctaacttcgtgccagcagccgcggtaatacgaagggtgcaagcgttactcggaattactgggcgtaaagcgtgcgtaggtggttatttaagtccgttgtgaaagccctgggctcaacctgggaactgcagtggatactggatgactagaatgtggtagagggtagcggaattcctggtgtagcagtgaaatgcgtagagatcaggaggaacatccatggcgaaggcagctacctggaccaacattgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtcgagaactttccagagatggattggtgccttcgggaactcgaacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacggccagggctacacacgtactacaatggtagggacagagggctgcaagccggcgacggtaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacg

SEQ ID NO: 21 - DNA, 1377 bp, Carnobacterium sp.
  ribosomal RNA nucleic acid sequence
tacctcaccggcttcgggtgttacaaactctcgtggtgtgacgggcggtgtgtacaagacccgggaacgtattcaccgcggcgttctgatccgcgattactagcgattccggcttcatgtaggcgagttgcagcctacaatccgaactgagaatggctttaagagattagcttggcctcgcgaccttgcgactcgttgtaccatccattgtagcacgtgtgtagcccaggtcataaggggcatgatgatttgacgtcatccccaccttcctccggtttgtcaccggcagtctcactagagtgcccaactaaatgctggcaactagtaataagggttgcgctcgttgcgggacttaacccaacatctcacgacacgagctgacgacaaccatgcaccacctgtcactttgtccccgaagggaaagctctatctctagagtggtcaaaggatgtcaagacctggtaaggttcttcgcgttgcttcgaattaaaccacatgctccaccgcttgtgcgggtccccgtcaattcctttgagtttcagccttgcggccgtactccccaggcggagtgcttaatgcgttagctgcagcactgaagggcggaaaccctccaacacttagcactcatcgtttacggcgtggactaccagggtatctaatcctgtttgctccccacgctttcgagcctcagcgtcagttacagaccagagagtcgccttcgccactggtgttcctccacatatctacgcatttcaccgctacacgtggaattccactctcctcttctgcactcaagttctccagtttccaatgaccctccccggttgagccgggggctttcacatcagacttaaagaaccgcctgcgctcgctttacgcccaataaatccggacaacgcttgccacctacgtattaccgcggctgctggcacgtagttagccgtggctttctggttaaataccgtcaggggatgagcagttactctcatccttgttcttctttaacaacagagttttacgatccgaaaaccttcttcactcacgcggcgttgctccgtcagactttcgtccattgcggaagattccctactgctgcctcccgtaggagtctgggccgtgtctcagtcccagtgtggccgatcaccctctcaggtcggctacgtatcattgccttggtgagccattacctcaccaactagctaatacgccgcgggtccatccattagtggtagccgaagccacctttccttcaagaatcatgcgattcctgaaactatgcggtattagcatccgtttccgaatgttatccccctctaatgggcaggttacccacgtgttactcacccgtccgccactcacttggttaaagagcaagct

SEQ ID NO: 22 - DNA, 1510 bp, Carnobacterium sp.
  ribosomal RNA nucleic acid sequence
ggagagtttgatcctggctcaggacgaacgctggcggcgtgcttaacacatgcaagtcgaacggtgacgctagagcttgctctggttgatcagtggcgaacgggtgagtaacacgtgagtaacctgcccttgactctgggataactccgggaaaccggggctaataccggatacgagacgcgatcgcatggtcggcgtctggaaagtttttcggtcaaggatggactcgcggcctatcagctagttggtgaggtaacggctcaccaaggcgtcgacgggtagccggcctgagagggcgaccggccacactgggactgagacacggcccagactcctacgggaggcagcagtggggaatattgcacaatgggcgaaagcctgatgcagcgacgccgcgtgagggatgaaggccttcgggttgtaaacctctttcagtagggaagaagcgaaagtgacggtacctgcagaagaagcgccggctaactacgtgccagcagccgcggtaatacgtagggcgcaagcgttgtccggaattattgggcgtaaagagctcgtaggcggtttgtcgcgtctggtgtgaaaactcaaggctcaaccttgagcttgcatcgggtacgggcagactagagtgtggtaggggtgactggaattcctggtgtagcggtggaatgcgcagatatcaggaggaacaccgatggcgaaggcaggtcactgggccactactgacgctgaggagcgaaagcatggggagcgaacaggattagataccctggtagtccatgccgtaaacgttgggcactaggtgtggggctcattccacgagttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacaccggaatcggccagagatggtcgcgtcttcggactggtgtacaggtggtgcatggttgtcgtcagctcgtgtcgtgagatgttgggttaagtcccgcaacgagcgcaaccctcgtcctatgttgccagcacgtcatggtggggactcataggagactgccggggtcaactcggaggaaggtggggatgacgtcaaatcatcatgccccttatgtcttgggcttcacgcatgctacaatggccggtacaaagggctgcgataccgcgaggtggagcgaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaaccccttgtgggatggagccgtcgaaggtgggattggcgattgggactaagtcgtaacaaggtagccgtaccggaaggtgcggctggatc

SEQ ID NO: 23 - DNA, 1403 bp, Acinetobacter sp.
  ribosomal RNA nucleic acid sequence
tgcagtcgagcggagtgatggtgcttgcactatcacttagcggcggacgggtgagtaatgcttaggaatctgcctattagtgggggacaacatttcgaaaggaatgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatgggcgcaagcctgatccagccatgccgcgtgtgtgaagaaggccttatggttgtaaagcactttaagcgaggaggaggctactgaagttaataccttcagatagtggacgttactcgcagaataagcaccggctaactctgtgccagcagccgcggtaatacagagggtgcaagcgttaatcggatttactgggcgtaaagcgcgcgtaggcggctaattaagtcaaatgtgaaatccccgagcttaacttgggaattgcattcgatactggttagctagagtgtgggagaggatggtagaattccaggtgtagcggtgaaatgcgtagagatctggaggaataccgatggcgaaggcagccatctggcctaacactgacgctgaggtgcgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgagtaatgtcgggaactttaaggatactgccagtgacaaactggaggaaggcggggacgacgtcaagtcatcatggcccttacggccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgcaaggaggacgct

SEQ ID NO: 24 - DNA, 1420 bp, Bacillus sp.
  ribosomal RNA nucleic acid sequence
tgcagtcgagcgaatggattgagagcttgctctcaagaagttagcggcggacgggtgagtaacacgtgggtaacctgcccataagactgggataactccgggaaaccggggctaataccggataacattttgaactgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtgcggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaaccttttcgagccagccgcct

SEQ ID NO: 25 - DNA, 1538 bp, Bacillus sp.
  ribosomal RNA nucleic acid sequence
ggagagtttgatcctggctcaggatgaacgctggcggcgtgcctaatacatgcaagtcgagcgaatggattgagagcttgctctcaagaagttagcggcggacgggtgagtaacacgtgggtaacctgcccataagactgggataactccgggaaaccggggctaataccggataacattttgaactgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaaggtgggacagatgattggggtgaagtcgtaacaaggtagccgtatcggaaggtgcggctggatc

SEQ ID NO: 26 - DNA, 1419 bp, Serratia sp.
  ribosomal RNA nucleic acid sequence
gcttaccatgcagtcgagcggtagcacaaggggagcttgctccctgggtgacgagcggcggacgggtgagtaatgtctgggaaactgcctgatggagggggataactactggaaacggtagctaataccgcataatgtcgcaagaccaaagagggggaccttcgggcctcttgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaaggtggtgaacttaatacgttcatcaattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcgtatacaaagagaagcgacctcgcgagagcaagcggacctcataaagtacgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgctacc

SEQ ID NO: 27 - DNA, 1386 bp, Pseudomonas sp.
  ribosomal RNA nucleic acid sequence
tgcagtcgagcggatgaagggagcttgctcctgaattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaaattaatactttgctgttttgacgttaccgacagaataagcaccggctaactctgtgccagcagccgcggtaatacagagggtgcaagcgttaatcggaattactgggcgtaaagcgcgcgtaggtggttcgttaagttggatgtgaaatccccgggctcaacctgggaactgcatccaaaactggcgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaa

SEQ ID NO: 28 - DNA, 1520 bp, Pseudomonas sp.
  ribosomal RNA nucleic acid sequence
aagagtttgatcatggctcagattgaacgctggcggcaggcctaacacatgcaagtcgagcggatgaagggagcttgctcctgaattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaaattaatactttgctgttttgacgttaccgacagaataagcaccggctaactctgtgccagcagccgcggtaatacagagggtgcaagcgttaatcggaattactgggcgtaaagcgcgcgtaggtggttcgttaagttggatgtgaaatccccgggctcaacctgggaactgcatccaaaactggcgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaacaaggtagccgtaggggaacctgcggctgga

SEQ ID NO: 29 - DNA, 1366 bp, Pseudomonas sp.
  misc_feature (11)..(12): n is a, c, g, or t
  ribosomal RNA nucleic acid sequence
agggttaggcnnccggcttcaggtgttaccgactttcatgacttgacgggcggtgtgtacaagacccgggaacgtattcaccgcagcgttgctgatctgcgattactagcgactccgacttcatgaggtcgagttgcagacctcaatccgaactgggaccggctttttgggattcgctccacctcgcggtattgcagccctttgtaccggccattgtagcatgcgtgaagcccaagacataaggggcatgatgatttgacgtcatccccaccttcctccgagttgaccccggcagtatcccatgagttcccaccattacgtgctggcaacatagaacgagggttgcgctcgttgcgggacttaacccaacatctcacgacacgagctgacgacaaccatgcaccacctgtttacgagtgtccaaagagttgaccatttctggcccgttctcgtatatgtcaagccttggtaaggttcttcgcgttgcatcgaattaatccgcatgctccgccgcttgtgcgggtccccgtcaattcctttgagttttagccttgcggccgtactccccaggcggggaacttaatgcgttagctgcgtcacggaatccgtggaatggaccccacaactagttcccaacgtttacggggtggactaccagggtatctaagcctgtttgctccccaccctttcgctcctcagcgtcagttacggcccagagatctgccttcgccatcggtgttcctcctgatatctgcgcattccaccgctacaccaggaattccaatctcccctaccgcactctagtctgcccgtacccactgcaggccggaggttgagcctccggatttcacagcagacgcgacaaaccgcctacgagctctttacgcccaataattccggataacgcttgcgccctacgtattaccgcggctgctggcacgtagttagccggcgctttttctgcaggtaccgtcactttcgcttcttccctgctaaaagaggtttacaacccgaaggccgtcgtccctcacgcggcgttgctgcatcaggcttgcgcccattgtgcaatattccccactgctgcctcccgtaggagtctgggccgtgtctcagtcccagtgtggccggtcaccctctcaggccggctacccgtcgacgccttggtgagccattacctcaccaacaagctgataggccgcgagcccatccccaaccgaaattctttccagacgcagaccatgcggtcacgtcacatatccagtattagacgccgtttccagcgcttatcccagagtcaggggcaggttgctcacgtgttactcacccgttcgccactgatcccacagagcaagctccgtgttcaccg

SEQ ID NO: 30 - DNA, 1373 bp, Pseudomonas sp.
  ribosomal RNA nucleic acid sequence
ggttaagctacctgcttctggtgcaacaaactcccatggtgtgacgggcggtgtgtacaaggcccgggaacgtattcaccgcagcaatgctgatctgcgattactagcgattccgacttcatggagtcgagttgcagactccaatccggactgagatagggtttctgggattggcttaccgtcgccggcttgcagccctctgtccctaccattgtagtacgtgtgtagccctggccgtaagggccatgatgacttgacgtcatccccaccttcctccggtttgtcaccggcggtctccttagagttcccaccattacgtgctggcaactaaggacaagggttgcgctcgttgcgggacttaacccaacatctcacgacacgagctgacgacagccatgcagcacctgtgttcgagttcccgaaggcacccatccatctctggaaagttctcgacatgtcaaggccaggtaaggttcttcgcgttgcatcgaattaaaccacatactccaccgcttgtgcgggcccccgtcaattcctttgagtttcagtcttgcgaccgtactccccaggcggcgaacttaacgcgttagcttcgatactgcgtgccaaattgcacccaacatccagttcgcatcgtttagggcgtggactaccagggtatctaatcctgtttgctccccacgctttcgtgcctcagtgtcagtgttggtccaggtagctgccttcgccatggatgttcctcctgatctctacgcatttcactgctacaccaggaattccgctaccctctaccacactctagtcgcccagtatccactgcagttcccaggttgagcccagggctttcacaacggacttaaacgaccacctacgcacgctttacgcccagtaattccgagtaacgcttgcacccttcgtattaccgcggctgctggcacgaagttagccggtgcttattctttgggtaccgtcatcccaaccgggtattagccagctggatttctttcccaacaaaagggctttacaacccgaaggccttcttcacccacgcggtatggctggatcaggcttgcgcccattgtccaatattccccactgctgcctcccgtaggagtctggaccgtgtctcagttccagtgtggctgatcatcctctcagaccagctacggatcgtcgccttggtgggcctttaccccgccaactagctaatccgacatcggctcattcaatcgcgcaaggcccgaaggtcccctgctttcacccgtaggtcgtatgcggtattagcgtaagtttccctacgttatcccccacgacagagtagattccgatgtattcctcacccgtccgccactcgccacccaaggagcaagctc

SEQ ID NO: 31 - DNA, 1389 bp, Pseudomonas sp.
  ribosomal RNA nucleic acid sequence
aggttagactagctacttctggagcaacccactcccatggtgtgacgggcggtgtgtacaaggcccgggaacgtattcaccgtgacattctgattcacgattactagcgattccgacttcacgcagtcgagttgcagactgcgatccggactacgatcggttttatgggattagctccacctcgcggcttggcaaccctttgtaccgaccattgtagcacgtgtgtagccctggccgtaagggccatgatgacttgacgtcatccccaccttcctccggtttgtcaccggcagtctccttagagtgcccaccattacgtgctggtaactaaggacaagggttgcgctcgttacgggacttaacccaacatctcacgacacgagctgacgacagccatgcagcacctgtgtctgagttcccgaaggcaccaatccatctctggaaagttctcagcatgtcaaggccaggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaagttctcaaggaacccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccgtactctagctcgccagttttggatgcagttcccaggttgagcccggggctttcacatccaacttaacgaaccacctacgcgcgctttacgcccagtaattccgattaacgcttgcacccttcgtattaccgcggctgctggcacgaagttagccggtgcttattctgtcggtaacgtcaaaacactaacgtattaggttaatgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgttcctttcggaacgttatcccccactaccaggcagattcctaggcattactcacccgtccgccgctaaatcaaggagcaagctcctctcatccgctcgactgca

SEQ ID NO: 32 - DNA, 1520 bp, Pseudomonas sp.
  ribosomal RNA nucleic acid sequence
aagagtttgatcatggctcagattgaacgctggcggcaggcctaacacatgcaagtcgagcggatgagaggagcttgctccttgatttagcggcggacgggtgagtaatgcctaggaatctgcctggtagtgggggataacgttccgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggttccttgagaacttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccttcggggggacggttaccacggagtgattcatgactggggtgaagtcgtaacaaggtagccgtaggggaacctgcggctgga

SEQ ID NO: 33 - DNA, 1363 bp, Stenotrophomonas sp.
  misc_feature (383)..(383): n is a, c, g, or t
  ribosomal RNA nucleic acid sequence
agcttgctctctgggtggcgagtggcggacgggtgaggaatacatcggaatctactttttcgtgggggataacgtagggaaacttacgctaataccgcatacgacctacgggtgaaagcaggggatcttcggaccttgcgcgattgaatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaatccagctggntaatacccggttgggatgacggtacccaaagaataagcaccggctaacttcgtgccagcagccgcggtaatacgaagggtgcaagcgttactcggaattactgggcgtaaagcgtgcgtaggtggtcgtttaagtccgttgtgaaagccctgggctcaacctgggaactgcagtggatactgggcgactagaatgtggtagagggtagcggaattcctggtgtagcagtgaaatgcgtagagatcaggaggaacatccatggcgaaggcagctacctggaccaacattgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtcgagaactttccagagatggattggtgccttcgggaactcgaacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacggccagggctacacacgtactacaatggtagggacagagggctgcaagccggcgacggtaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggt

SEQ ID NO: 34 - DNA, 1528 bp, Stenotrophomonas sp.
  ribosomal RNA nucleic acid sequence
aagagtttgatcctggctcagagtgaacgctggcggtaggcctaacacatgcaagtcgaacggcagcacaggagagcttgctctctgggtggcgagtggcggacgggtgaggaatacatcggaatctactttttcgtgggggataacgtagggaaacttacgctaataccgcatacgacctacgggtgaaagcaggggatcttcggaccttgcgcgattgaatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaatccagctggttaatacccggttgggatgacggtacccaaagaataagcaccggctaacttcgtgccagcagccgcggtaatacgaagggtgcaagcgttactcggaattactgggcgtaaagcgtgcgtaggtggtcgtttaagtccgttgtgaaagccctgggctcaacctgggaactgcagtggatactgggcgactagagtgtggtagagggtagcggaattcctggtgtagcagtgaaatgcgtagagatcaggaggaacatccatggcgaaggcagctacctggaccaacactgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtcgagaactttccagagatggattggtgccttcgggaactcgaacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacggccagggctacacacgtactacaatggtagggacagagggctgcaagccggcgacggtaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacggtgtggccgatgactggggtgaagtcgtaacaaggtagccgtatcggaaggtgcggctgga

SEQ ID NO: 35 - DNA, 1422 bp, Chitinophaga sp.
  ribosomal RNA nucleic acid sequence
gctcaggatgaacgctagcggcaggcctaatacatgcaagtcgaggggcagcgcaggtagcaataccgggcggcgaccggcaaacgggtgcggaacacgtacgcaaccttccttcaagcggggaatagcccagagaaatttggattaataccccataagaatgtggactggcatcagtcagcatttaaagatttatcacttgaagatgggcgtgcgtctgattaggtagttggtgaggtaacggctcaccaagccgacgatcagtaactggcgtgagagcgcgaccagtcacacgggcactgagacacgggcccgactcctacgggaggcagcagtaaggaatattggtcaatggacgaaagtctgaaccagccatgccgcgtggaggatgaaggccctctgggttgtaaacttcttttatttgggacgaaacacttcatttctatggagcttgacggtaccagatgaataagcaccggctaactccgtgccagcagccgcggtaatacggagggtgcaagcgttatccggattcactgggtttaaagggtgcgtaggcggacatgtaagtccgtggtgaaatctccgagcttaactcggaaactgccatggatactatatgtcttgaatgttgtggaggttagcggaatatgtcatgtagcggtgaaatgcatagatatgacatagaacaccaattgcgaaggcagctggctacacaaatattgacgctgaggcacgaaagcgtggggatcaaacaggattagataccctggtagtccacgccctaaacgatgattactcgacatacgcgatacacagtgtgtgtctgagcgaaagcattaagtaatccacctgggaagtacgaccgcaaggttgaaactcaaaggaattgacgggggtccgcacaagcggtggagcatgtggtttaattcgatgatacgcgaggaaccttacctgggctagaatgcagtctgaccgatgctgaaaggtgtctttgtagcaatacacagattgtaaggtgctgcatggctgtcgtcagctcgtgccgtgaggtgttgggttaagtcccgcaacgagcgcaacccctatcactagttgccagcacgtcaaggtgggaactctagtgaaactgccgtcgtaagacgcgaggaaggaggggatgatgtcaagtcatcatggcctttatgcccagggctacacacgtgctacaatggtgggaacaaagggctgctccctggtaacaggctgctaatctcaaaaatcccatctcagttcggattgaggtctgcaactcgacctcatgaagctggaatcgctagtaatcgtatatcagcaatgatacggtgaatacgttcccggaccttgtacacaccgcccgtcaagccatgaaagccggggggacctgaagtcggcaaccgcaag

SEQ ID NO: 36 - DNA, 1508 bp, Chitinophaga sp.
  ribosomal RNA nucleic acid sequence
agagtttgatcctggctcaggatgaacgctagcggcaggcctaatacatgcaagtcgaggggcagcgcaggtagcaataccgggcggcgaccggcaaacgggtgcggaacacgtacgcaaccttccttcaagcggggaatagcccagagaaatttggattaataccccataagaatgtggactggcatcagtcagcatttaaagatttatcacttgaagatgggcgtgcgtctgattaggtagttggtgaggtaacggctcaccaagccgacgatcagtaactggcgtgagagcgcgaccagtcacacgggcactgagacacgggcccgactcctacgggaggcagcagtaaggaatattggtcaatggacgaaagtctgaaccagccatgccgcgtggaggatgaaggccctctgggttgtaaacttcttttatttgggacgaaacacttcatttctatggagcttgacggtaccagatgaataagcaccggctaactccgtgccagcagccgcggtaatacggagggtgcaagcgttatccggattcactgggtttaaagggtgcgtaggcggacatgtaagtccgtggtgaaatctccgagcttaactcggaaactgccatggatactatatgtcttgaatgttgtggaggttagcggaatatgtcatgtagcggtgaaatgcatagatatgacatagaacaccaattgcgaaggcagctggctacacaaatattgacgctgaggcacgaaagcgtggggatcaaacaggattagataccctggtagtccacgccctaaacgatgattactcgacatacgcgatacacagtgtgtgtctgagcgaaagcattaagtaatccacctgggaagtacgaccgcaaggttgaaactcaaaggaattgacgggggtccgcacaagcggtggagcatgtggtttaattcgatgatacgcgaggaaccttacctgggctagaatgcagtctgaccgatgctgaaaggtgtctttgtagcaatacacagattgtaaggtgctgcatggctgtcgtcagctcgtgccgtgaggtgttgggttaagtcccgcaacgagcgcaacccctatcactagttgccagcacgtcaaggtgggaactctagtgaaactgccgtcgtaagacgcgaggaaggaggggatgatgtcaagtcatcatggcctttatgcccagggctacacacgtgctacaatggtgggaacaaagggctgctccctggtaacaggctgctaatctcaaaaatcccatctcagttcggattgaggtctgcaactcgacctcatgaagctggaatcgctagtaatcgtatatcagcaatgatacggtgaatacgttcccggaccttgtacacaccgcccgtcaagccatgaaagccggggggacctgaagtcggcaaccgcaaggaaccgcctagggtaaaatcggtaattggggctaagtcgtaacaaggtagccgtatcggaaggtgcggctgg

SEQ ID NO: 37 - DNA, 1384 bp, Brevibacillus sp.
  misc_feature (21)..(23): n is a, c, g, or t
  misc_feature (215)..(215): n is a, c, g, or t
  misc_feature (1376)..(1376): n is a, c, g, or t
  misc_feature (1378)..(1379): n is a, c, g, or t
  ribosomal RNA nucleic acid sequence
gcagtcgagcgagggtcttcnnnccctagcggcggacgggtgagtaacacgtaggcaacctgcctgtaagactgggataacatagggaaacttatgctaataccggatagggttttgcttcgcctgaagcgaaacggaaagatggcgcaagctatcacttacagatgggcctgcggcgcattagctagttggtgaggtaacggctcaccaaggcnacgatgcgtagccgacctgagagggtgaccggccacactgggactgagacacggcccagactcctacgggaggcagcagtagggaattttccacaatggacgaaagtctgatggagcaacgccgcgtgaacgatgaaggctttcgggtcgtaaagttctgttgttagggaagaaacagtgctatttaaataaggtagcaccttgacggtacctaacgagaaagccacggctaactacgtgccagcagccgcggtaatacgtaggtggcaagcgttgtccggaattattgggcgtaaagcgcgcgcaggtggctatgtaagtctgatgttaaagcccgaggctcaacctcggttcgcattggaaactgtgtagcttgagtgcaggagaggaaagtggtattccacgtgtagcggtgaaatgcgtagagatgtggaggaacaccagtggcgaaggcgactttctggcctgtaactgacactgaggcgcgaaagcgtggggagcaaacaggattagataccctggtagtccacgccgtaaacgatgagtgctaggtgttaggggtttcaatacccttagtgccgcagctaacgcaataagcactccgcctggggagtacgctcgcaagagtgaaactcaaaggaattgacgggggcccgcacaagcggtggagcatgtggtttaattcgaagcaacgcgaagaaccttaccaggtcttgacatcccactgaccgctctagagatagagcttcccttcggggcagtggtgacaggtggtgcatggttgtcgtcagctcgtgtcgtgagatgttgggttaagtcccgcaacgagcgcaacccttatctttagttgccagcattcagttgggcactctagagagactgccgtcgacaagacggaggaaggcggggatgacgtcaaatcatcatgccccttatgacctgggctacacacgtgctacaatggttggtacaacgggatgctacttcgcgagaagatgctaatctcttaaaaccaatctcagttcggattgtaggctgcaactcgcctacatgaagtcggaatcgctagtaatcgcggatcagcatgccgcggtgaatacgttcccgggccttgtacacaccgcccgtcacaccacgggagtttgcaacacccgaagtcgntnnaggta

SEQ ID NO: 38 - DNA, 1521 bp, Brevibacillus sp.
  ribosomal RNA nucleic acid sequence
gagagtttgatcctggctcaggacgaacgctggcggcgtgcctaatacatgcaagtcgagcgagggtcttcggaccctagcggcggacgggtgagtaacacgtaggcaacctgcctgtaagactgggataacatagggaaacttatgctaataccggatagggttttgcttcgcctgaagcgaaacggaaagatggcgcaagctatcacttacagatgggcctgcggcgcattagctagttggtgaggtaacggctcaccaaggcaacgatgcgtagccgacctgagagggtgaccggccacactgggactgagacacggcccagactcctacgggaggcagcagtagggaattttccacaatggacgaaagtctgatggagcaacgccgcgtgaacgatgaaggctttcgggtcgtaaagttctgttgttagggaagaaacagtgctatttaaataaggtagcaccttgacggtacctaacgagaaagccacggctaactacgtgccagcagccgcggtaatacgtaggtggcaagcgttgtccggaattattgggcgtaaagcgcgcgcaggtggctatgtaagtctgatgttaaagcccgaggctcaacctcggttcgcattggaaactgtgtagcttgagtgcaggagaggaaagtggtattccacgtgtagcggtgaaatgcgtagagatgtggaggaacaccagtggcgaaggcgactttctggcctgtaactgacactgaggcgcgaaagcgtggggagcaaacaggattagataccctggtagtccacgccgtaaacgatgagtgctaggtgttaggggtttcaatacccttagtgccgcagctaacgcaataagcactccgcctggggagtacgctcgcaagagtgaaactcaaaggaattgacgggggcccgcacaagcggtggagcatgtggtttaattcgaagcaacgcgaagaaccttaccaggtcttgacatcccactgaccgctctagagatagagcttcccttcggggcagtggtgacaggtggtgcatggttgtcgtcagctcgtgtcgtgagatgttgggttaagtcccgcaacgagcgcaacccttatctttagttgccagcattcagttgggcactctagagagactgccgtcgacaagacggaggaaggcggggatgacgtcaaatcatcatgccccttatgacctgggctacacacgtgctacaatggttggtacaacgggatgctacttcgcgagaagatgctaatctcttaaaaccaatctcagttcggattgtaggctgcaactcgcctacatgaagtcggaatcgctagtaatcgcggatcagcatgccgcggtgaatacgttcccgggccttgtacacaccgcccgtcacaccacgggagtttgcaacacccgaagtcggtgaggtaaccgtaaggagccagccgccgaaggtggggtagataactggggtgaagtcgtaacaaggtatccgtaccggaaggtgcggatggatc

SEQ ID NO: 39 - DNA, 1378 bp, Pseudomonas sp.
  ribosomal RNA nucleic acid sequence
ctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctttgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatctatctctagaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaagagctcaaggctcccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccatactctagcttgccagttttggatgcagttcccaggttgagcccggggctttcacatccaacttaacaaaccacctacgcgcgctttacgcccagtaattccgattaacgcttgcaccctctgtattaccgcggctgctggcacagagttagccggtgcttattctgtcggtaacgtcaaaacaatcacgtattaggtaactgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctaatagcgtgaggtccgaagatcccccactttctcccgtaggacgtatgcggtattagcgcccgtttccggacgttatcccccactactaggcagattcctaggcattactcacccgtccgccgctcgccaccaggtacaagtacccgtgctgccgctcgactgc

SEQ ID NO: 40 - DNA, 1400 bp, Acinetobacter sp.
  ribosomal RNA nucleic acid sequence
gccctctttgcagttaggctagctacttctggtgcaacaaactcccatggtgtgacgggcggtgtgtacaaggcccgggaacgtattcaccgcggcattctgatccgcgattactagcgattccgacttcatggagtcgagttgcagactccaatccggactacgatcggctttttgagattagcatcctatcgctaggtagcaaccctttgtaccgaccattgtagcacgtgtgtagccctggccgtaagggccatgatgacttgacgtcgtccccgccttcctccagtttgtcactggcagtatccttaaagttcccgacattactcgctggcaaataaggaaaagggttgcgctcgttgcgggacttaacccaacatctcacgacacgagctgacgacagccatgcagcacctgtatgtaagttcccgaaggcaccaatccatctctggaaagttcttactatgtcaaggccaggtaaggttcttcgcgttgcatcgaattaaaccacatgctccaccgcttgtgcgggcccccgtcaattcatttgagttttagtcttgcgaccgtactccccaggcggtctacttatcgcgttagctgcgccactaaagcctcaaaggccccaacggctagtagacatcgtttacggcatggactaccagggtatctaatcctgtttgctccccatgctttcgcacctcagcgtcagtgttaggccagatggctgccttcgccatcggtattcctccagatctctacgcatttcaccgctacacctggaattctaccatcctctcccacactctagctaaccagtatcgaatgcaattcccaagttaagctcggggatttcacatttgacttaattagccgcctacgcgcgctttacgcccagtaaatccgattaacgcttgcaccctctgtattaccgcggctgctggcacagagttagccggtgcttattctgcgagtaacgtccactatctctaggtattaactaaagtagcctcctcctcgcttaaagtgctttacaaccataaggccttcttcacacacgcggcatggctggatcaggcttccgcccattgtccaatattccccactgctgcctcccgtaggagtctgggccgtgtctcagtcccagtgtggcggatcatcctctcagacccgctacagatcgtcgccttggtaggcctttaccccaccaactagctaatccgacttaggctcatctattagcgcaaggtccgaagatcccctgctttctcccgtaggacgtatgcggtattagcattcctttcgaaatgttgtcccccactaataggcagattcctaagcattactcacccgtccgccgctaagatcagtagcaagctacctctctccgctcgactgca

SEQ ID NO: 41 - DNA, 1372 bp, Carnobacterium sp.
  misc_feature (1069)..(1069): n is a, c, g, or t
  ribosomal RNA nucleic acid sequence
gagcttgctctttaaccaagtgagtggcggacgggtgagtaacacgtgggtaacctgcccattagagggggataacattcggaaacggatgctaataccgcatagtttcaggaatcgcatgattcttgaaggaaaggtggcttcggctaccactaatggatggacccgcggcgtattagctagttggtgaggtaatggctcaccaaggcaatgatacgtagccgacctgagagggtgatcggccacactgggactgagacacggcccagactcctacgggaggcagcagtagggaatcttccgcaatggacgaaagtctgacggagcaacgccgcgtgagtgaagaaggttttcggatcgtaaaactctgttgttaaagaagaacaaggatgagagtaactgctcatcccctgacggtatttaaccagaaagccacggctaactacgtgccagcagccgcggtaatacgtaggtggcaagcgttgtccggatttattgggcgtaaagcgagcgcaggcggttctttaagtctgatgtgaaagcccccggctcaaccggggagggtcattggaaactggagaacttgagtgcagaagaggagagtggaattccacgtgtagcggtgaaatgcgtagatatgtggaggaacaccagtggcgaaggcgactctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacggccgcaaggctgaaactcaaaggaattgacggggacccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcattnagttgggcactctagtgagactgccggtgacaaaccggaggaaggtggggatgacgtcaaatcatcatgccccttatgacctgggctacacacgtgctacaatggatggtacaacgagtcgcaaggtcgcgaggccaagctaatctcttaaagccattctcagttcggattgtaggctgcaactcgcctacatgaagccggaatcgctagtaatcgcggatcagaacgccgcggtgaatacgttcccgggtcttgtacacaccgcccgtcacaccacgagagtttgtaacacccgaagccggt

SEQ ID NO: 42 - DNA, 1538 bp, Carnobacterium sp.
  ribosomal RNA nucleic acid sequence
gagagtttgatcctggctcaggacgaacgctggcggcgtgcctaatacatgcaagtcgaacgcacgaagttgaagagcttgctctttaaccaagtgagtggcggacgggtgagtaacacgtgggtaacctgcccattagagggggataacattcggaaacggatgctaataccgcatagtttcaggaatcgcatgattcttgaaggaaaggtggcttcggctaccactaatggatggacccgcggcgtattagctagttggtgaggtaatggctcaccaaggcaatgatacgtagccgacctgagagggtgatcggccacactgggactgagacacggcccagactcctacgggaggcagcagtagggaatcttccgcaatggacgaaagtctgacggagcaacgccgcgtgagtgaagaaggttttcggatcgtaaaactctgttgttaaagaagaacaaggatgagagtaactgctcatcccctgacggtatttaaccagaaagccacggctaactacgtgccagcagccgcggtaatacgtaggtggcaagcgttgtccggatttattgggcgtaaagcgagcgcaggcggttctttaagtctgatgtgaaagcccccggctcaaccggggagggtcattggaaactggagaacttgagtgcagaagaggagagtggaattccacgtgtagcggtgaaatgcgtagatatgtggaggaacaccagtggcgaaggcgactctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacggccgcaaggctgaaactcaaaggaattgacggggacccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggatgacgtcaaatcatcatgccccttatgacctgggctacacacgtgctacaatggatggtacaacgagtcgcaaggtcgcgaggccaagctaatctcttaaagccattctcagttcggattgtaggctgcaactcgcctacatgaagccggaatcgctagtaatcgcggatcagaacgccgcggtgaatacgttcccgggtcttgtacacaccgcccgtcacaccacgagagtttgtaacacccgaagccggtgaggtaaccttttaggagccagccgtctaaggtgggatagataattggggtgaagtcgtaacaaggtagccgtatcggaaggtgcggctgga

SEQ ID NO: 43 - DNA, 1466 bp, Ensifer sp.
  ribosomal RNA nucleic acid sequence
gagagtttgatcctggctcagaacgaacgctggcggcaggcttaacacatgcaagtcgagcgccccgcaaggggagcggcagacgggtgagtaacgcgtgggaatctacccttttctacggaataacgcagggaaacttgtgctaataccgtataagcccttcgggggaaagatttatcgggaaaggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggaattactgggcgtaaagcgcacgtaggcggacatttaagtcaggggtgaaatcccagagctcaactctggaactgcctttgatactgggtgtctagagtatggaagaggtgagtggaattccgagtgtagaggtgaaattcgtagatattcggaggaacaccagtggcgaaggcggctcactggtccattactgacgctgaggtgcgaaagcgtggggagcaaacaggattagataccctggtagtccacgccgtaaacgatgaatgttagccgtcgggcagtttactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcccgatcgcggattacagagatgttttccttcagttcggctggatcggagacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttctacccgaaggtagtgcgctaaccgcaaggaggcagctaaccacggtagggtcagcgactggggtgaagtcgtaacaaggtagccgtaggggaacctgcggctg

SEQ ID NO: 44 - DNA, 1072 bp, Rahnella sp.
  ribosomal RNA nucleic acid sequence
acttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttgga

SEQ ID NO: 45 - DNA, 1391 bp, Pseudomonas sp.
  ribosomal RNA nucleic acid sequence
gaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatccatctctggaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaagagctcaaggctcccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccatactctagctcgccagttttggatgcagttcccaggttgagcccggggatttcacatccaacttaacgaaccacctacgcgcgctttacgcccagtaattccgattaacgcttgcaccctctgtattaccgcggctgctggcacagagttagccggtgcttattctgtcggtaacgtcaaaatactcacgtattaggtaagtacccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgtccgtttccggacgttatcccccactaccaggcagattcctaggcattactcacccgtccgccgctctcaagagaagcaagcttctctctaccgctcgactgca

SEQ ID NO: 46 - DNA, 1384 bp, Stenotrophomonas sp.
  ribosomal RNA nucleic acid sequence
taagctacctgcttctggtgcaacaaactcccatggtgtgacgggcggtgtgtacaaggcccgggaacgtattcaccgcagcaatgctgatctgcgattactagcgattccgacttcatggagtcgagttgcagactccaatccggactgagatagggtttctgggattggcttaccgtcgccggcttgcagccctctgtccctaccattgtagtacgtgtgtagccctggccgtaagggccatgatgacttgacgtcatccccaccttcctccggtttgtcaccggcggtctccttagagttcccaccattacgtgctggcaactaaggacaagggttgcgctcgttgcgggacttaacccaacatctcacgacacgagctgacgacagccatgcagcacctgtgttcgagttcccgaaggcacccatccatctctggaaagttctcgacatgtcaaggccaggtaaggttcttcgcgttgcatcgaattaaaccacatactccaccgcttgtgcgggcccccgtcaattcctttgagtttcagtcttgcgaccgtactccccaggcggcgaacttaacgcgttagcttcgatactgcgtgccaaattgcacccaacatccagttcgcatcgtttagggcgtggactaccagggtatctaatcctgtttgctccccacgctttcgtgcctcagtgtcagtgttggtccaggtagctgccttcgccatggatgttcctcctgatctctacgcatttcactgctacaccaggaattccgctaccctctaccacactctagtcgtccagtatccactgcagttcccaggttgagcccagggctttcacaacggacttaaacgaccacctacgcacgctttacgcccagtaattccgagtaacgcttgcacccttcgtattaccgcggctgctggcacgaagttagccggtgcttattctttgggtaccgtcatcccaaccaggtattagccggctggatttctttcccaacaaaagggctttacaacccgaaggccttcttcacccacgcggtatggctggatcaggcttgcgcccattgtccaatattccccactgctgcctcccgtaggagtctggaccgtgtctcagttccagtgtggctgatcatcctctcagaccagctacggatcgtcgccttggtgggcctttaccccgccaactagctaatccgacatcggctcattcaatcgcgcaaggtccgaagatcccctgctttcacccgtaggtcgtatgcggtattagcgtaagtttccctacgttatcccccacgacagagtagattccgatgtattcctcacccgtccgccactcgccacccagagagcaagctctcctgtgctgccgt

SEQ ID NO: 47 - DNA, 1402 bp, Acinetobacter sp.
  ribosomal RNA nucleic acid sequence
agcgtcctccttgcggttagactacctacttctggtgcaacaaactcccatggtgtgacgggcggtgtgtacaaggcccgggaacgtattcaccgcggcattctgatccgcgattactagcgattccgacttcatggagtcgagttgcagactccaatccggactacgatcggctttttgagattagcatcctatcgctaggtagcaaccctttgtaccgaccattgtagcacgtgtgtagccctggccgtaagggccatgatgacttgacgtcgtccccgccttcctccagtttgtcactggcagtatccttaaagttcccgacattactcgctggcaaataaggaaaagggttgcgctcgttgcgggacttaacccaacatctcacgacacgagctgacgacagccatgcagcacctgtatgtaagttcccgaaggcaccaatccatctctggaaagttcttactatgtcaaggccaggtaaggttcttcgcgttgcatcgaattaaaccacatgctccaccgcttgtgcgggcccccgtcaattcatttgagttttagtcttgcgaccgtactccccaggcggtctacttatcgcgttagctgcgccactaaagcctcaaaggccccaacggctagtagacatcgtttacggcatggactaccagggtatctaatcctgtttgctccccatgctttcgcacctcagcgtcagtgttaggccagatggctgccttcgccatcggtattcctccagatctctacgcatttcaccgctacacctggaattctaccatcctctcccacactctagctaaccagtatcgaatgcaattcccaagttaagctcggggatttcacatttgacttaattagccgcctacgcgcgctttacgcccagtaaatccgattaacgcttgcaccctctgtattaccgcggctgctggcacagagttagccggtgcttattctgcgagtaacgtccactatctctaggtattaactaaagtagcctcctcctcgcttaaagtgctttacaaccataaggccttcttcacacacgcggcatggctggatcaggcttgcgcccattgtccaatattccccactgctgcctcccgtaggagtctgggccgtgtctcagtcccagtgtggcggatcatcctctcagacccgctacagatcgtcgccttggtaggcctttaccccaccaactagctaatccgacttaggctcatctattagcgcaaggtccgaagatcccctgctttctcccgtaggacgtatgcggtattagcattcctttcgaaatgttgtcccccactaataggcagattcctaagcattactcacccgtccgccgctaagatcagtagcaagctacctctctccgctcgactgc

SEQ ID NO: 48 - DNA, 1383 bp, Paraburkholderia sp.
  ribosomal RNA nucleic acid sequence
gtcctccttgcggttagactagccacttctggtaaaacccactcccatggtgtgacgggcggtgtgtacaagacccgggaacgtattcaccgcggcatgctgatccgcgattactagcgattccagcttcatgcactcgagttgcagagtgcaatccggactacgatcggttttctgggattggctccacctcgcggcttggcaaccctctgttccgaccattgtatgacgtgtgaagccctacccataagggccatgaggacttgacgtcatccccaccttcctccggtttgtcaccggcagtctccttagagtgctcttgcgtagcaactaaggacaagggttgcgctcgttgcgggacttaacccaacatctcacgacacgagctgacgacagccatgcagcacctgtgtatcggttctctttcgagcacccctagatctctccagggttccgaccatgtcaagggtaggtaaggtttttcgcgttgcatcgaattaatccacatcatccaccgcttgtgcgggtccccgtcaattcctttgagttttaatcttgcgaccgtactccccaggcggtcaacttcacgcgttagctacgttactaaggaaatgaatccccaacaactagttgacatcgtttagggcgtggactaccagggtatctaatcctgtttgctccccacgctttcgtgcatgagcgtcagtattggcccagggggctgccttcgccatcggtattcctccacatctctacgcatttcactgctacacgtggaattctacccccctctgccatactctagcccgccagtcaccaatgcagttcccaggttgagcccggggatttcacatcggtcttagcgaaccgcctgcgcacgctttacgcccagtaattccgattaacgctcgcaccctacgtattaccgcggctgctggcacgtagttagccggtgcttattcttccggtaccgtcatccaccccggatattagccaggatgatttctttccggacaaaagtgctttacaacccgaaggccttcttcacacacgcggcattgctggatcagggttgcccccattgtccaaaattccccactgctgcctcccgtaggagtctgggccgtgtctcagtcccagtgtggctggtcgtcctctcagaccagctactgatcgtcgccttggtaggcctttaccccaccaactagctaatcagccatcggccaaccctatagcgcgaggtcttgcgatcccccgctttcatccctagatcgtatgcggtattaatccggctttcgccgggctatcccccactacaggacatgttccgatgtattactcacccgttcgccactcgccaccagggttgcccccgtgctgccg

SEQ ID NO: 49 - DNA, 1405 bp, Serratia sp.
  ribosomal RNA nucleic acid sequence
gtagcgccctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacgtactttatgaggtccgcttgctctcgcgaggtcgcttctctttgtatacgccattgtagcacgtgtgtagccctactcgtaagggccatgatgacttgacgtcatccccaccttcctccagtttatcactggcagtctcctttgagttcccggccgaaccgctggcaacaaaggataagggttgcgctcgttgcgggacttaacccaacatttcacaacacgagctgacgacagccatgcagcacctgtctcagagttcccgaaggcaccaatccatctctggaaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgatttaacgcgttagctccggaagccacgcctcaagggcacaacctccaaatcgacatcgtttacagcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactctagcttgccagtttcaaatgcagttcccaggttgagcccggggatttcacatctgacttaacaaaccgcctgcgtgcgctttacgcccagtaattccgattaacgcttgcaccctccgtattaccgcggctgctggcacggagttagccggtgcttcttctgcgagtaacgtcaattgatgaacgtattaagttcaccaccttcctcctcgctgaaagtgctttacaacccgaaggccttcttcacacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccattaccccacctactagctaatcccatctgggcacatctgatggcaagaggcccgaaggtccccctctttggtcttgcgacgttatgcggtattagctaccgtttccagtagttatccccctccatcaggcagtttcccagacattactcacccgtccgccgctcgtcacccagggagcaagctcccctgtgctaccgctcgac

SEQ ID NO: 50 - DNA, 1381 bp, Chryseobacterium sp.
  ribosomal RNA nucleic acid sequence
gctcctgttacggtaaccgacttcaggtaccccatacttccatggcttgacgggcggtgtgtacaaggcccgggaacgtattcaccgcgccatggctgatgcgcgattactagcgattccagcttcatagagtcgagttgcagactccaatccgaactgagaccggctttcgagatttgcatcacatcgctgtgtagctgccctctgtaccggccattgtattacgtgtgtggcccaaggcgtaagggccgtgatgatttgacgtcatccccaccttcctctctacttgcgtaggcagtctcactagagtccccaacttaatgatggcaactagtgacaggggttgcgctcgttgcaggacttaacctaacacctcacggcacgagctgacgacaaccatgcagcaccttgaaaaatgtccgaagaaaagtctatttctaaacctgtcatttcccatttaagccttggtaaggttcctcgcgtatcatcgaattaaaccacataatccaccgcttgtgcgggcccccgtcaattcctttgagtttcattcttgcgaacgtactccccaggtggctaacttatcactttcgcttagtctctgaatccgaaaacccaaaaacgagttagcatcgtttacggcgtggactaccagggtatctaatcctgttcgctccccacgctttcgtccatcagcgtcagttgttgcttagtaacctgccttcgcaattggtgttctaagtaatatctatgcatttcaccgctacactacttattccagctacttcaataacactcaagacctgcagtatcaatggcagtttcacagttaagctgtgagatttcaccactgacttacagatccgcctacggaccctttaaacccaataaatccggataacgcttgcaccctccgtattaccgcggctgctggcacggagttagccggtgcttattcgtatagtaccttcagctactctcacgagagtaggtttatccctatacaaaagaagtttacaacccatagggccgtcgtccttcacgcgggatggctggatcaggctctcacccattgtccaatattcctcactgctgcctcccgtaggagtctggtccgtgtctcagtaccagtgtgggggatcaccctctcaggccccctaaagatcgtagacttggtgagccgttacctcaccaactatctaatcttgcgcgtgcccatctctatccaccggagttttcaatatccatcgatgccgacaaatatattatggggtattaatcttcctttcgaaaggctatcccccagataaaggcaggttgcacacgtgttccgcacccgtacgccgctctcaagatcccgaaagatctctaccgctcggctgca

SEQ ID NO: 51 - DNA, 1437 bp, Variovorax sp.
  ribosomal RNA nucleic acid sequence
atcgccctccttgcggttaagctaactacttctggcagaacccgctcccatggtgtgacgggcggtgtgtacaagacccgggaacgtattcaccgtgacattctgatccacgattactagcgattccgacttcacgcagtcgagttgcagactgcgatccggactacgactggttttatgggattagctccccctcgcgggttggcaaccctttgtaccagccattgtatgacgtgtgtagccccacctataagggccatgaggacttgacgtcatccccaccttcctccggtttgtcaccggcagtctcattagagtgcccaactgaatgtagcaactaatgacaagggttgcgctcgttgcgggacttaacccaacatctcacgacacgagctgacgacagccatgcagcacctgtgttacggttctctttcgagcactaagccatctctggcgaattccgtacatgtcaaaggtgggtaaggtttttcgcgttgcatcgaattaaaccacatcatccaccgcttgtgcgggtccccgtcaattcctttgagtttcaaccttgcggccgtactccccaggcggtcaacttcacgcgttagcttcgttactgagtcagtgaagacccaacaaccagttgacatcgtttagggcgtggactaccagggtatctaatcctgtttgctccccacgctttcgtgcatgagcgtcagtacaggtccaggggattgccttcgccatcggtgttcctccgcatatctacgcatttcactgctacacgcggaattccatccccctctaccgtactctagctatgcagtcacagatgcagttcccaggttgagcccggggatttcacaactgtcttacataaccgcctgcgcacgctttacgcccagtaattccgattaacgcttgcaccctacgtattaccgcggctgctggcacgtagttagccggtgcttattcttacggtaccgtcattagccctctttattagaaaaggccgtttcgttccgtacaaaagcagtttacaacccgaaggccttcatcctgcacgcggcatggctggatcaggctttcgcccattgtccaaaattccccactgctgcctcccgtaggagtctgggccgtgtctcagtcccagtgtggctggtcgtcctctcagaccagctacagatcgaaggcttggtgagcctttacctcaccaactacctaatctgccatcggccgctccattcgcgcaaggtcttgcgatcccctgctttcatccgtagatcgtatgcggtattagcacagctttcgctgcgttatcccccacgattgggcacgttccgatgtattactcacccgttcgccactcgccgccaggattgctcccgcgctgccgttcgacttgcatgtgtaaggcatgccgccagcgttcaatctgagcca

SEQ ID NO: 52 - DNA, 1091 bp, Serratia sp.
  Marker nucleic acid sequence
atgggattcaaatgcggtatcgtcggcctgcctaacgtaggcaaatccaccctgttcaacgcgttgaccaaagcgggcattgaagcagccaacttcccgttctgcaccattgaaccgaacaccggtgtggtgccgatgcccgatccgcgtctggataagctggccgagatcgtcaagccgcagcgcattctgccgaccaccatggaattcgtcgacatcgccggcctggtgaaaggcgcgtccaaaggtgaaggcctgggcaaccagttcctgaccaacatccgcgaaaccgaagccatcggccacgtagtgcgctgcttcgaaaacgacaacatcatccacgtcaacaacaaggtcgatccggccgaagacatcgacgtgatcaacaccgagctggcgctgtccgacctggacacctgcgagcgcgccatccaccgtgtgcagaagaaagccaaaggcggcgacaaagacgctaaagccgagctcgcggcgctggagaaatgcctgccgcacctggaaaacgccggcatgctgcgcgcgctggatctgaccgcggaagacaaggcggcgatcaaatacctgagcttcctgacgctgaagccgaccatgtacatcgccaacgtcaacgaagacggcttcgagaacaacccgtacctggacaccgtgcgtaaaatcgccgacgccgaaggctccgtagtggttgccgtgtgcgccgccgtggaatccgacatcgccgagctggaagacgaagagcgcgacgagttcatggccgagctgggcctggaagagccgggtctgaaccgcgtgatccgcgccggttatgaactgctgaacctgcaaacctacttcaccgccggcgtgaaagaagtgcgtgcctggaccatcccggtaggcgccactgccccgcaggcggccggcaagatccacaccgacttcgagaaaggctttatccgcgcccaaaccatctcctatgacgacttcatcacctacaagggtgagcaaggcgccaaagaagccggcaaaatgcgttccgaaggtaaagactacatcgtcaaagacggcgacgtgatgaacttcttgttcaacgtcta

SEQ ID NO: 53 - DNA, 1064 bp, Serratia sp.
  Marker nucleic acid sequence
atggctattgatgagaacaagcaaaaggcgttagctgcggcactgggccagattgagaaacagttcggcaaaggctccatcatgcgcctgggtgaagaccgctccatggacgtggaaacgatctccaccggctcactgtcactcgatatcgccctgggcgccggcggcctgccgatgggccgcatcgtagaaatctacggcccggaatcgtccggtaaaaccactttgacgctgcaggtgatcgccgctgcgcagcgcgaaggcaaaacctgtgcgttcatcgacgccgaacacgcgctggatcctatctatgcgaaaaagctgggcgtcgatatcgacaacctgctgtgctcccagccggacaccggcgagcaggcgctggaaatctgtgatgcgctgacccgctccggcgcggttgatgtcatcatcgtcgactccgtggcggcgctgacgccgaaggcggaaatcgaaggtgaaatcggcgattcgcacatgggcctggcggcgcgcatgatgagccaggcgatgcgtaaactggccggcaacctgaaaaacgccaataccctgctgatcttcatcaaccagatccgtatgaaaattggtgtgatgttcggcaacccggaaaccacgaccggcggtaacgccctgaagttctatgcttcggtgcgtctggatatccgccgcatcggcgccatcaaagaaggcgacgaagtggtgggcagcgaaacccgcgtgaaagtggtgaagaacaaaatcgctgcgccgttcaaacaggctgagttccaaatcatgtacggcgaaggcatcaacagccgcggcgaactggtcgatctgggcgtgaagcacaagatgatcgaaaaagccggcgcctggtacagctataacggcgagaagatcggtcagggcaaggcgaacgcctgtaacttcctgaaagaaaacccggctatcgccgccgagctggacaagaagctgcgcgacctgctgctgcacagcggcggtgagctggtggctgcttccggtgacgacttcgaagatgacgaagcggaaaccagcgagcagttcta

SEQ ID NO: 54 - DNA, 1331 bp, Serratia sp.
  Marker nucleic acid sequence
tggatactgatcaattaacgacgcaggtggcggaccagctgggcgcgcagcagcggctgctggtggcgttcagcggcggcctggattccagcgtgctgctgcatctgctggcggcgttgcgccgccaccgtccggcgctgcagctgcgtgcgctgcacgtgcatcatggcctgagcgttttcgccgaccgctgggttgagcattgccgcagccgatgtgccgactggcaggtgccgctgacggtgacgcacgtgcaggtggacgcccgccagggcggcatcgaagcggcggcgcgtgcggcgcgctatgccgccttcagcgccgccctggcggagggcgaagcgctgctcaccgctcaacaccttgacgatcaaagcgaaactttcctgttggcgctcaagcgcggcagcgggccggcggggctatcggcgatggcggcgcgcgccacgctgggcgagcatttgctgttgcggccgttgctgggctgctcgcgtcaaactctggaaagctacgcgcagcgccacgccttaagctggatagatgacgacagcaatcaggacacgcgcttcgatcgcaatttcctgcgtttgcaggttttgccgcagctcaaccagcgctggccgcacttcgcttcggcggtggcgcgcagcgccagcctgtgcgccgagcaggagcagctgctggacgaactgctggcggaaccgctgcaaagcctgttggcggcggaccgctcattggcgatcgacggcctgaccgactgttcgccggtgcggcgtttcgccttgttgcggcgctggatcgcgttgtttaacgtcactatgccggcgcgtgagcagctacagcgcctgtgggaggaggtggcgctcagccgggaagacgccgaaccgcagctgcagctcggcgcctatcagatccgccgattccgtggccgactgcacctgctgccgccgctggcggcgctgcgcggtgtgcatctgcactggcggcctgaccagccgctgacgttgccggacgggttggggcagttggttgccggtgagggcgatctggcgctacgcgcgccgcagccagcgcagcggatcagcattcgtttcggcggcgtgcagggcgccgtgcgtatcgtggggcgcgcccattcgcggccgataaaaaaactgtggcaggagctggggatccctccctggcagcgtgatcgcatcccgttgatttattatgatgagcagctgatcgctgctttgggcgcattcgtctgtgaggcggggcaggtgccggaaggcgaacgcccgtggcggctgcgctgggaaaaaaatcataatcgcgaggagcactga

SEQ ID NO: 55 - DNA, 1490 bp, Serratia sp.
  Marker nucleic acid sequence
atgatacctgtcgtcgcgctggtcgggcgcccgaatgtgggtaaatccaccttgttcaaccgtttaacccatacgcgcgatgcgctggtggcggatttcccggggctgacgcgtgaccgcaagtatggtcgtgctgaaatcgaaggcaatgaattcatcatcgtcgataccggcggcatcgacggcaccgaagacggcgtagaaacgcgcatggccggccagtcgctgctggcgatcgaagaagcggacatcgtgctgttcatggtcgacgcccgcgccggcctgatgccggcggatcagggcattgcccagcacctgcgcagccgccagaaggcgaccttcctggtggccaacaagaccgacggcctggatccggacaccgccaccgccgatttctactcgctcggtctgggtgaagtgttcgctatcgccgcttctcacggccgcggtgtgacccagctgattgaacacgtgctggtgccgttcgtgccggaaaaaccggaagacgtcgagctgaccgaggaagaggccaacgccgcttactgggccgagcaaaacggcgaaacgctggaaggcgaagaagacgaagagccggaagaagcgttcaatccgcaggatctgccgatcaagctggcgatcgtcggccgcccgaacgtcggtaagtctacgctcactaaccgcatcctcggcgaggagcgcgtggtggtgtatgacatgccgggcaccacccgcgacagcatctacattccgatggtgcgcgacgaacgcgaatacgtgctgatcgacaccgccggggtgcgcaaacgcggcaaggtgaccgaaactgtcgagaaattctcggtgatcaagactctgcaggcgattgaagacgccaacgtggtgctgctggtgatcgacgcgcgtgaaggcatttccgatcaggatctctctctgctcggctttatcctcaatagtgggcgctcactggtgattgcggtcaacaagtgggacggcatgagcgaagaagatcgcgagcacgtgaaagagatgctcgatctgcgtctgggcttcgtcgacttcgcgcgcgtgcacttcatttccgccctgcacggcagcggcgtcggcaacctgttcgaatcggtacaggaagcctacgagtgcgcaacccgccgcgtaaacacctcgatgctgaccaaaatcatgcagatggcggttgacgatcaccagccgccgttggtgcgcggccgccgcgtgaagctgaaatatgcccatgccggcggttacaacccgccgatcgtggtgatccacggcaaccaggtcagcgatctggccgattcgtacaaacgctatctgatgaactatttccgccgctcgctgaatgtgatggggaccccgatccgcatccagttcaaagagggggacaacccgtttgccggcaaacgcaacctgctgacgccgacccagatgcgtaagcgcaagcgcctgatgagccacctgaaaaagagcaagta

SEQ ID NO: 56 - DNA, 1064 bp, Serratia sp.
  Marker nucleic acid sequence
tggctattgatgagaacaagcaaaaggcgttagctgcggcactgggccagattgagaaacagttcggcaaaggctccatcatgcgcctgggtgaagaccgctccatggacgtggaaacgatctccaccggctcactgtcactcgatatcgccctgggcgccggcggcctgccgatgggccgcatcgtagaaatctacggcccggaatcgtccggtaaaaccactttgacgctgcaggtgatcgccgctgcgcagcgcgaaggcaaaacctgtgcgttcatcgacgccgaacacgcgctggatcctatctatgcgaaaaagctgggcgtcgatatcgacaacctgctgtgctcccagccggacaccggcgagcaggcgctggaaatctgtgatgcgctgacccgctccggcgcggttgatgtcatcatcgtcgactccgtggcggcgctgacgccgaaggcggaaatcgaaggtgaaatcggcgattcgcacatgggcctggcggcgcgcatgatgagccaggcgatgcgtaaactggccggcaacctgaaaaacgccaataccctgctgatcttcatcaaccagatccgtatgaaaattggtgtgatgttcggcaacccggaaaccacgaccggcggtaacgccctgaagttctatgcttcggtgcgtctggatatccgccgcatcggcgccatcaaagaaggcgacgaagtggtgggcagcgaaacccgcgtgaaagtggtgaagaacaaaatcgctgcgccgttcaaacaggctgagttccaaatcatgtacggcgaaggcatcaacagccgcggcgaactggtcgatctgggcgtgaagcacaagatgatcgaaaaagccggcgcctggtacagctataacggcgagaagatcggtcagggcaaggcgaacgcctgtaacttcctgaaagaaaacccggctatcgccgccgagctggacaagaagctgcgcgacctgctgctgcacagcggcggtgagctggtggctgcttccggtgacgacttcgaagatgacgaagcggaaaccagcgagcagttctaa

SEQ ID NO: 57 - DNA, 1091 bp, Pantoea sp.
  Marker nucleic acid sequence
atgggattcaaatgcggtatcgtgggcctgccgaacgtcggtaaatccactctgtttaacgcgctgactaaagcgggcatcgaagcggctaacttccccttctgcaccattgagccaaacacgggcgtggtgccgatgcctgacctgcgtctcgaccagctttctgagattgttaagccgcagcgcgtggtgccgactaccatggaattcgtcgatatcgccggtctggtaaaaggcgcgtcaaaaggtgaaggcctgggtaaccagttcctgaccaacatccgtgaaaccgaagcgatcggccacgtggtgcgctgctttgaaaacgacaacatcatccacgtggcgggtaaagtgaacccggctgaagatattgatgtgatcaacaccgagctggcgctgtccgatcttgaaacctgtgaacgtgcgattcagcgtgtgcagaagaaagccaaaggcggcgacaaagatgccaaagccgagctggctgcgctggagaagtgcctgccgcatctttctgaagcgggcatgctgcgttcactgaatctggatgaggacgacaaagcggcaatccgctatctgagcttcctgaccctgaaacccaccatgtacattgctaacgtcaatgaagatggtttcgagaacaacccgtacctggatcaggtgcgcgctatcgccgaagcggaaggttcagtcgtggtgcccgtgtgcgccgccgttgagtcggacattgctgagctggaagatgaagaccgtgacgagttcatggcagagctgggactggaagagccaggcctgaaccgcgttatccgcgccggttactccctgctgaacctgcagacctacttcaccgctggcgtgaaagaagtacgtgcctggaccatcccggttggcgcgacggctccacaggccgccggtaaaatccataccgacttcgagaaaggctttatccgcgcccagaccatcgcgtttgaagatttcatcgcttataaaggcgagcaaggtgctaaagaagccggcaagatgcgttcagaaggcaaagagtacatcgtgaaagatggcgatgtgatgaacttcctgttcaacgtctg

SEQ ID NO: 58 - DNA, 1832 bp, Pantoea sp.
  Marker nucleic acid sequence
gtgaatgacgtttttgattccaaagcctttctgagtacggtaaccagcaagcccggtgtttaccgcatgtatgacgcctcaggcaccgtcatctatgtaggcaaagccaaagacctgaagaagcgtctgaccagctacttccgcactcaggtcggcagccgcaaaaccgaagcgctggttagcaatatccagcacatcgatgtcaccgtgacccacaccgaaactgaagcgctgctgcttgagcataactacatcaagctctaccagccgcgctacaacgtgctgctgcgcgacgataaatcttacccctacatctttcttagcagcgatacccatccgcgactggcgatgcaccgtggtgcaaagcatgccaaaggcgaatactttggcccgttccccaacggctatgcggtgcgtgaaacgctgggattgctgcaaaaagtcttccctatccgccagtgcgaaaacagcgtctaccgcaatcgctcccggccctgtctgcaatatcagattggccgctgtctggggccctgtgtcgcggggctggtcagtgaggaggagtatgcacagcagaccgattacgtacgtctgttcctggcgggtaaagacgatcaggttctcaaccagctggtcaaacggatggaagaggcgagcacgggcctgcgctttgaagaggcggcacgcctgcgcgaccagattcaggctgtgcgacggatcaccgaaaagcagtttgtctcgaatcagggtgatgaccttgatgtgatgggtgtggcttatgaagcgggcatggcctgtctccacgtgctgtttatccgtcagggcaaagtgttgggcagccgcagctacttcccgaaagtgcccgtcgataccgatctggctgaggtcgttcagacctttgtcgggcagttctacctgcagggcagcgaggcacgcacgctgcctggcgatattctgctggacttcaccttgccagaacgtgaactgctggctgagtcgctcagtgagctggccgggcgtcgggtgagtattcagagcaagccgcgcggggatcgcgcccgttatctcaaactggcgcgcaccaacgccgcgacggcgttaaccacccggctgtcacagcactccacaattcatcagcggctgatggcgctggcggagtttcttgagctggacaaaattaaacgcatggagtgtttcgatatcagtcataccatgggtgagcagaccatcgcctcctgcgtagtgttcgaccagaatggtccgctgcgtgccgattatcgtcgctacaacatcagcggtattacgccgggggatgactacgccgccatgaatcaggtgctacgccgccgctatggtaaagcgattgaagaagataagattccggatgtgattctgatcgatggcggtaagggccagctgtctcaggcgaagcaggtcttcgcggaactggatgtgccctgggacaaaagccggccgattttactcggcgtagcgaaaggcagcgatcgtaaagccggactggagacgctgttctttgaagcggaaggtgagggcgtttcactgccacccgatgcgccggcgctgcatgtgatccagcatattcgtgatgacgctcacaatcatgccatttcaggccaccgtaaaaaacgggccaaagtaaagaacaccagcgcactggaaagcatcgaaggggtggggcctaaacggcgtcagcaactgcttaagtacatggggggcctgcaaccgctgatgaacgcgtcggtggaggagatcgccaatgtacccggtatctcacatgctctggcagaaaaaatatactactcgttgaaacacta

SEQ ID NO: 59 - DNA, 2021 bp, Pantoea sp.
  Marker nucleic acid sequence
atgagcaaagcctttaaattgaactccgcctttaaaccttcgggcgaccagcctgaggcgatacgtcgcctggaagaggggctggaggatggactggcccatcagaccttattaggcgtaaccggctctggtaaaacgtttaccgtcgccaacgtgattgccgacctcaaccgtccgaccatggtgctggcgcccaacaagacgctggccgcccaactctatggcgagatgaaagagtttttcccggataacgccgtcgagtttttcgtctcttactacgactattaccagcctgaagcttatgtgccgagctccgacacctttatcgaaaaggatgcgtcggtgaacgaacatatcgaacagatgcgtctgtcggccaccaaggcgctgctggaacggcgcgatgtcattgtggtggcgtcggtttcggctatctacggtctgggcgatcccgatctctatctgaaaatgatgctgcacctgacccgcggcatgattatcgatcagcgcagtattctgcgccgtctgtcagagctgcagtacacccgcaacgaccaggcgttccagcgcggtaccttccgggtgcgcggcgaagtgatcgatatcttcccggcggagtcggacgacattgcgctgcgcgtcgagctgtttgacgaagaggttgagcgtctgtcgctgtttgatccgctgaccggccagatcgattcggtcgtgccgcgctttaccatctaccccaagacgcactacgtgacgccgcgtgagcgtattctgcaggcgatggaagatatcaaagtcgagctggcggaccgccgccgggtgctgctggagaacaataagctgctggaagagcagcgcattacccagcgtacccagtttgacctggagatgatgagtgagctgggctactgctccggcatcgagaactactcacgctacctctccgcacgcggaccgggcgaagcgccaccgacgctgtttgactatctgcccgccgatggcctgctggtggtggatgagtcgcatgtgaccattccgcagattggcggcatgtataaaggcgaccgggcgcgtaaagagacgctggtggagtatggcttccgcctgccgtcggcgctggataaccgtccgatgaagtttgaagagtttgaagcactggccccgcagaccatctatgtttcggcgacgccgggcaaatatgagctggaaaagtcgggcgacgaagtgatcgatcaggttgtgcgtcctaccggtcttctggacccgattatcgaagtgcgtccggtcggtacccaggtcgacgatctgctgtcagaaatccgtcagcgcgtggcgatcaacgaacgtgtactggtgacggtgctgaccaaacgcatggcggaagatctcaccgaatatctgtctgagcatggtgagaaggtgcgttatctgcactctgatatcgacacggtagagcgtatggagattattcgcgacttacgtctgggcgagtttgatgtgctggtgggcattaacttactgcgtgaggggcttgatatgccggaagtgtcgctggtggcgattctcgatgcggataaagagggcttcctgcgctccgaacgctcgctgattcagaccattggccgtgcggcgcgtaacatcaatggtaaggcgattctctacggcgataaaatcacgccgtcgatggcgcgcgccattgaagagaccgagcgccgccgtgagaaacagcagctgcacaacgaagagaacggcatcgtgccgcagggcctgaataagaagatcaccgatattctggaactgggcaaaaacgtggtgaaaacgcgcggcaaagcgaaaccagcccgccctggcgaagcccaggcgctggccgatttccagctgttaacgccgcaggccctgcagaagaaaatccacgagctggaagggcagatgcagcagcatgcgcagaacctggagttcgaacaggcggccaacgtgcgcgatcagctgcatgaactgcgaacgctgtttattgcggcctcgta

SEQ ID NO: 60 - DNA, 1004 bp, Pantoea sp.
  Marker nucleic acid sequence
atgatagaagccgaccgcctgatctccgctgccagtatcagcgaagaagaggttattgaccgcgctattcgcccgaagatgctgaccgagtatgtcggtcagccgcaggtgcgagagcagatggagattttcatcaaggcagcgcaactgcgcggcgatgcgctcgatcatctgctgatcttcggtccgccgggactgggtaaaaccaccctggcgaacatcgtggctaacgagatgggcgttaatcttcgcactacctcaggtccggtgctggaaaaggcgggcgacctggcggcgatgctgaccaaccttgaaccgcatgacgtcctgtttatcgatgaaatccatcgcctctcaccggtggtggaagagatactctacccggcgatggaagattatcagctggatatcatgatcggcgaaggcccggctgcgcgctcaatcaagctcgatctgccgccgtttaccctgattggcgccaccacgcgtgctggctcgctgacttcgccgctgcgcgatcgttttggcattgtgcagcggcttgagttctaccaggttaaagatttgcagcacatcgtaggccgcagcgccgcctgtctgggtctggcgctgagtgacgaaggggcgttagagattgcccgtcgcgcacgcggcacaccgcgtatcgccaaccgcctgctgcgccgcgttcgtgacttcgccgaagtgcgggcaaacggtgatttgagcggtgaagtctcctgcagcgcgctggatatgcttaacgtagacagcgaaggattcgattatatggaccgtaaactgctgctggcaatcatcgacaaatttatgggtgggccggtggggctggataacctggctgcggctattggtgaagagcgcgaaaccatcgaagatgtgattgaacccttcctgattcagcagggcttcattcagcgcacaccgcgtggccggatggccacgcagcacgcttatcgtcacttcggcattacgcgtgaagagta

SEQ ID NO: 61 - DNA, 1067 bp, Pantoea sp.
  Marker nucleic acid sequence
tggcgattgatgaaaacaaacagaaggctttagctgccgcgcttggccagattgagaaacaatttggtaaaggctccatcatgcgcttgggtgaagaccgctcaatggatgtggaaaccatctctaccggctcgctgtcgctggatatcgcattaggcgcaggcggtctgccaatgggccgtatcgttgagatctacggacctgagtcttcaggtaaaaccacgctgactttgcaggttatcgcatctgcacagcgtaaaggtaaaacctgtgcctttatcgatgccgagcatgcgcttgatccggtttacgccaagaaactcggcgtagacattgataacctgctctgctctcagccagacactggtgagcaggcgctggaaatctgtgatgcgctggcgcgctctggtgccgttgacgtgatcatcgtcgactccgtcgcggcgctgacgcctaaagcggaaatcgaaggtgaaatcggtgactcacacatgggcctcgcggcacgtatgatgagccaggcgatgcgtaaactggccggtaacctgaagcagtccaataccctgctgattttcatcaaccagattcgaatgaaaattggtgtgatgtttggtaacccggaaaccaccactggtggtaacgcgctgaagttctacgcctctgtccgtcttgatatccgccgtatcggtgcaatcaaagagggcgataacgtggtcggcagtgagacccgcgttaaagtggtgaaaaacaaaatcgccgcgccattcaaacaggctgagttccagatcatgtacggcgaaggtatcaatacctttggtgagctggtcgacctgggcgtgaagcacaagctgattgaaaaagcgggtgcatggtacagctacaatggcgacaagattggtcagggtaaatcgaatgccggcaacttcctgaaagagaatgctgctgtggcaaacgagattgatctgaagttacgtgagatgctgctcaacggcgcggatgaaaaactcgcagcagccgacaaggctgctgagaaagaagacgcggccgaagcgaacgaagacttctaa

SEQ ID NO: 62 - DNA, 1073 bp, Microbacterium sp.
  Marker nucleic acid sequence
tggctctcactatcggtatcgtcggcctgcccaacgtcggcaagtccaccctcttcaacgcactcaccaagaacgacgtgctcgcggcgaactacccgttcgcgacgatcgagccgaacgtcggcgtggtgaacctgcccgatcctcgcctcgacaagctcgcggagatcttcggcagcgagcgcatcctgcccgcagcggtgtcgttcgtcgacatcgccggcatcgtgcgcggggcgagcgagggtgagggtctgggcaacaagttcctcgcgaacatccgtgaggccgacgccatcgctcaggtcgtgcgcggtttcgccgacgacgatgtcgtccacgtcgacggcacggttaaccctgcatccgacatggagaccatcaacgcggaactcatgctcgccgacctcgagaccgtcgacaaggcgatcgcgcggtacgagaaggaagtgcgcggcaagaagatcgagcccatcgtgctcgagaccgcgaacgcggcgaaggatgctctcgagcggggtgtgctgctctcggtcgccggcatcgacctgacgccgatccgggagctgggtctgctcaccgcgaagcccgtcatcttcgtcttcaacgtcgacgagtccgtgctgaccgacgacgcgcgcaaggcggagctcgccgccctcgtcgccccggcgaaggccatcttcctcgacgcgaagatcgagtccgagctcatcgacctcgacccggaagacgcagccgagctcctcgcctcgaccggccaggacgagtccggtctcgaccagctcgcccgcatcggcttcgacacgctcggcctgcagacgtacctgaccgccggtccgaaggaagcccgcgcctggaccatccccaaggggtcgaaggcgccacaggctgcgggcgtcatccacaccgacttcgagaagggcttcatcaaggccgagatcgtctcgttcgacgatctggtcgagacgggctccgtcgtcgaggcccgcgccaagggcaaggcccgcctcgagggcaaggactacgtcatgcaggacggcgacgtcgtggaatttcgatttaataactag

SEQ ID NO: 63 - DNA, 617 bp, Microbacterium sp.
  Marker nucleic acid sequence
gtgacgaggatcatcgcaggcagcgccagaggggcccggctcgaggttccgggctcgggaacccgacccacgagcgaccgtgtacgcgaatcgctgttcggcgcgctggagtccctgagcgccatcgccggcgcacgggtcctcgacctgtacgccggttcgggcgccctcggactcgagtcgctcagccgtggagcagcgagcgtcgagttcgtggaacgcggccgtcaggccgcagacatcgtccgacgcaacgcggaagtcgtcgcgaaggcggggagcctcccgctcgggcgcgttcacgagagcacggcgcatgccttcctgcatcgcgcagtcggaccctacgacctcgtgttcagcgatccgccctacgacctcgacgacgcggccatgacggccgacctcgtcgcgctcgcaccgctgctgtcctcggacgccgtggtggtggtggaacgagctcgtcggtccacgcctccggacttcggcgccgcgaacctggagcagttccgcgagaagtcgtacggcgacaccaccctgtggtgggccgagccggcgacggaggacgaggaccctggccccgtgggtcagcccgcggccgagtcccagtcccgata

SEQ ID NO: 64 - DNA, 992 bp, Microbacterium sp.
  Marker nucleic acid sequence
tgccgtcactcgctcccgccatcgccgagatccgcctcgccgtgcgcaccgcgctcgccgaccttcccgagggagcggccgtcatcgtcgccctgtccgggggtgcggattcgctcgcgctcgccgcggccgtcgcgttcgaagcgcccaagctcggacttcgtgtcgcgtccctgaccgtcgatcacggcctgcagcacgactcggcagaggtcgcatccgtggcggctcgtgcagccgccgacctcggcctcgacccgttgatcgtgcgcgtggacgtcgcaggggagggcggtcctgaggccgccgcccgagaggcgcgataccgggtgctgcgagacgcggctgccgacgtcggagccgccgccgtgcttctcgggcacacgctcgacgaccaggcggagaccgttctgctcggcctggctcgtggctccggggcggccagcctgcaggggatggctccggtccgtgaggatgaggacggtgtgcgatgggtgcgtccgctcctgggcgtgcgccgcgagacgacgcgcgctttctgcgtcgcctccgacctcatgccgtgggacgatccgcacaaccgcgacgaccgctacgccagggtccgggtgcgcgagcacgtgctcccggttctcgaagacgagctcggtcccggcatcgccgaggccctcgcccgcacggcggagcagctccgggaggacgccgaggcgttcgacgagatgatccacgagacgatcgaggacatcgtcgagcacgcggaagccgggatctccgtcagcgtggcggcgctcgccgcgaatcccgccgcactgcgcaaccggatcatccggctcgtcgtcgacagcgagttcggggtgagcctgaccaggtcgcagacgatcgaggtcgcgcggctggtcaccgactggaatgggcaggggccgatcgacctgccggagtgctcggccgtgcgccacggggggcgcatcgtcttcgccgcatccggtcgctga

SEQ ID NO: 65 - DNA, 1844 bp, Stenotrophomonas sp.
  Marker nucleic acid sequence
tgaccgacgttcccgccccggccttcgacggcaaggccttcgcggcccagctcagcaccgcccctggcgtgtaccgcatgtacgccgccgacgacaccctgctgtacgtgggcaaggcgcgcgcgctgcgcaaccgtgtcggcagctatttcaacggcagcccgaagaacgcgcggatcatgtcgatgatctcgcagatcacgcgcatggacgtgaccgtgacgcgctcggaagccgaggcgttgctgctggaaaaccagttgatcaagtcgctgtcgccgcgctacaacgtctcactgcgcgatgacaagacctatccgcatgtgctgctgacccgcgaggactggccacgcatcgcgctgcatcgtggtccgcgtgccattcctgggcgctacttcgggccgtatcccggcgtcaccgccgtgcgcgagacgctgaacctgatgcacaagctgttcaagctgcgcagctgcgaggacagcgtgttccgcaaccgttcgcgaccgtgcctgcaataccagatcgggcgttgcagcgcgccgtgcgtggagctggtcccggcgccggagtacgccgagtcggtgcgccgcgccgccctgttcctggaaggcaagagtgacgagctgacccgcgaactgggcgagcagatgcaggccgccagcgaggcgctggagttcgagcaggccgcgcgcctgcgcgacctgatctcctcgctgcgcagcatgcagacccgccagtacgtggatggccgcgccgcggacctcgatgtgctggccgtggccatgcagggctcgcaggcctgcgtgctgctgctggccttccgtgatggtcgcaacctcggcacccgcccgttcttcccgcgcaccaatggcgaggaaagcccggaagaggtgcttgccgccttcgtctcgcagtactacatcgaattcgagccgccgcgcgagatcctgctggaccgcgagattcccgacgccgacctgctggtcgccgcgctgtcggcctcggccgagcgcaaggtgcagctgaagtggaacgtgcgcggcgagcgcgccggctatgtggaactggccagccgcaacgcgcagctgaccctggccaccgaactcaacagccgcaatgcgcagcacgcgcgcagcgacgcgctgcgcgagatgctgggcctggccgagccggtcaagcgtgtcgagtgttttgatatcagccacaccctgggcgaggccaccgtagcctcgtgcgtggtgttcgacgccgccggcccggtgcgcgcgcagtaccgccgcttcaacatcagcggcatcgagccgggcgacgactacgccgccatgcgccaggccatcgaccgccgtttccgccgcgcagtggaagagcagggcgtgctgccggacgtgctgctgatcgacggtggcgccggccagctgtcgcaggcccaggccgcactggccgatctgggcgtggaaggcgtgctgctggtgggcgtggccaagggcgtggagcgccgtgccggccacgaagcgctggtgatgcccgatggccgcgagctgcgcccgggcgcggccagcccggcgctgcagttcatccagcaggtgcgcgacgaggcgcatcgcttcgccatcaccggccacagggggcgccgccagaaggcgcggatgaccagcaagctggaggatatccccggcatcgggccgcgccgccgcgccagcctgctcaagcatttcggcggcctggtgggcctgaaagccgctggcgaagccgagatcgccaaggtggaaggcatcaatgacgccctcgcggcacgtatctacgctaacctgcacgggttggccacgcctgatgcggccgagtag

SEQ ID NO: 66 - DNA, 1040 bp, Stenotrophomonas sp.
  Marker nucleic acid sequence
tgaccgacgaccgcatcatcggcgccggcgccacccgcgaggatgacgccgccgacgccagcatccgccccaggcgcttggccgactacctcggccaggccccggtgcgcgagcagctggagatctacatcgaagcggccaaggcgcgtggcgatgcgctggaccacgtgctgatcttcggcccgcccggcctgggcaagaccaccctcagccacgtcatcgccaacgaactgggcgtggccctgcgcgtgacctccggcccggtgatcgagaaggccggcgacctcgctgcgctgctgaccaacctgcagccgcatgacgtgctgttcgtggatgagatccaccgcctgtcgccggtggtcgaggaagtgctgtacccggcgatggaagacttccagatcgacatcatgatcggcgagggcccggcggcccgctcgatcaagatcgacctgccgccgttcaccctgatcggcgccaccacccgcgccggcctgctgaccgcgccgctgcgcgaccgcttcggcatcgtccagcgcctggagttctacagcgtcgaggagctgacccggatcgtgcgccgctcggccaccatcctcggcatcgactgcaccgccgacggcgccggcgagatcgcgcggcgcgcgcgtggcaccccgcgcatcgccaaccggctgctgcgccgcgtgcgcgactacgcgcaggtgaaggccggcggccacatcgacgaggccgtggcacaggccgcgatgaagatgctcaaggtcgatccggagggcttcgacgagctcgaccggcgcctgctcaagaccctggtcgactacttcgacggcggcccggtcggcatcgaatcgctggccgcggcgctgtccgaagagcgcggcacgctggaagacgtggtcgagccgtacctgatccagcagggcttcctggtgcgtaccgcgcgcggccgcatggccacccacaaggcttaccggcacatgggcctgaagccgaagaacccgccgcaggacctgttcgcggaggttcccgatgtcggttga

SEQ ID NO: 67 - DNA, 1397 bp, Stenotrophomonas sp.
  Marker nucleic acid sequence
tgctgcctttggtcgccctggttggacggccgaatgtcggcaagtcgactattttcaatgcgcttacccgcacccgtgacgcgctggtccatgaccagcccggcgtcacccgcgaccgcaactacggtgtctgccggctggacgaggacaatcatttcctcgtcgtggacaccggcggtatcgcggaagaggaagagggtctggcaggggctaccacccgccaggcccgcgccgctgcggcggaagccgacctgatcctgttcgtggtcgacgcccgtgagggtacctcggcgatggacgacgagatcctggcctggctgcgcaagctgtcgcgcccgacgctgctgctgatcaacaagatcgacggcaccgacgaggacaccgtgcgttcggagttcgcccgttacggcttcggcgagatgctgaccgtgtcggccgcgcatcgccagggcttggacgacctgctggacgaagtgatccagcgcctgccggaagaaggcagcggcgaagagctggacaacgatccgaaccgcatccgcatcgccttcgtcggccgcccgaacgtgggcaaatcgaccctggtcaaccgcatcctcggcgaggagcgcatgatcgcctccgacgtgccgggcaccacccgcgactcgatcgcggtggacctggagcgtgacggccgcgaataccgcctgatcgataccgccggcctgcgccgtcgctcgcgcgtggacgaggtcgtcgagaagttctcggtggtcaagaccatgcagtcgatcgagcagtgccaggtggccgtgctgatgctcgacgccaccgaaggcgtgaccgaccaggacgccaccgtgctcggtgccgtgctcgatgccggccgtgcgctggtgatcgccatcaacaagtgggatggcctgaccgagtaccagcgcgagcaggctgaaaccatgctgtcgctgaagctgggcttcgtgccgtgggccgagtcggtgcgcatctcggccaagcatggttccggcctgcgtgagctgttccgcgcggtgcaccgcgcgcacgaatcggcgaacaagaccttcaccaccagcgaagtgaacaaggcgctggaagtggcctacgagaccaatccgccgccgaccatccgcggccatgtctccaagctgcgttacgtgcacccggccggttccaacccgccgaccttcatcgtgcacggcacccgcctgaaggagctgcaggagtcatacaagcgctacctggagaacttcttccgcaagcgcttcaagctgatcggcacgccggtgagcttcatcttccgcgagggtaccaacccgtacgagggcaagaagaacgtcctcaccgagcggcaggtcaaggccaagcggcgcttgatgaagcacgtcaagggcaagtga

SEQ ID NO: 68 - DNA, 2066 bp, Rathayibacter sp.
  Marker nucleic acid sequence
tggagccaacccgtgccgtgcaccccttcgaggtcatcagcgaatacaagccgagcggtgaccagcccggagcgatcgccgatctgagcgcccggatcaacgccggtgagaccgatgtggtcctgctgggcgccaccggcaccggcaagtccgcgacgaccgcctggctggtcgaggccgtccagcggccgaccctcgtgctggcgcacaacaagacgctcgccgctcagctcgccaacgagttccgcgagctcttccccaacaacgccgtcgagtacttcgtctcgtactacgactactaccagcccgaggcctacgtcccgcagacggacaccttcatcgagaaggacagctcggtcaacgcggaggtcgagcgcctgcggcactccacgaccaactcgctgctcagccggcgcgacgtcatcgtcgtctcgaccgtctcctgcatctacggcctgggccagcccgagcagtacctcgaggcgatggtcgcgctccaggtcggccagcgagtcgaccgggaccgcctcatccgcaagttcgtctcgatgcagtacgcccgcaacgacgtcgacttcgcccgcggcaccttccgcgtccgcggcgacacgatcgagatcatcccgatgtacgaggagctggcgatccgcatcgagatgttcggcgacgagatcgaggcgctctacacgctccacccgctcacgggcgacgtcgtccgcaagctcgagtcggtctccgtcttccccgggtcgcactacgtcgcgagccaggacgtcatgcaccgggcgatcgacaccatccagcaggagctcgaggtgcgcctggccgacctcgagaagcagaacaagctcctcgaggcgcagcggctgcggatgcggaccaccttcgacctcgagatgatgcagcagatcggcttctgctccggcatcgagaactactcgcggcacatcgacgggcgcgagtccggcgaggctccgcactgcctgctcgactacttccccgacgacttcctcgtcgtgatcgacgagtcccacgtcaccgtgccccagatcggcgcgatgtacgagggcgacgcgtcgcgcaagcgcaccctggtcgagcacggcttccggctgccctcggcgatggacaaccgcccgctgaagtgggaggagttcaagggccgcgtcggccagacggtctacctctcggcgacgccgggccgctacgagatgggcatcgccgacggcatcgtcgagcagatcatccgccccaccggtctgatcgaccccgagatcgtggtcaagccgacgaagggccagatcgacgacctgctcgaggagatcaagctccggaccgagcgcgacgagcgcgtgctggtcaccacgctcaccaagcggatggcggaggagctcaccgacttcctcaccgaggccggtgtccgggtgcgctacctgcactcggacgtcgacacgctccgccgcgtcgagctgctgtccgagctgcgccagggcgtctacgacgtcctcgtcggcatcaacctcctccgcgagggcctcgacctgcccgaggtgtcgctcgtcgcgatcctcgacgccgacaaggagggcttcctccgctcttcgacctcgctcatccagaccatcggccgcgccgcgcgcaacgtctccggtgaggtgcacatgtacgcggacaagatgaccgactcgatgaagctcgcgatcgacgagaccgaccgccgccgcgagaagcaggtcgcctacaacctcgaccacggcatcgacccgaccccgctgcggaagaagatcgccgacatcacggacgcactcgcccgcgagggagccgacacggcgaagctcctggccggccgggacgcgaagaagaagagcccgacgccgaacctccgccgtcaggggctggccgctaacggcggcaacgacctcgaggcgatcatcgccgacctcaacggccagatgctccaggccgccggcgagctgaagttcgagctcgcggcccgactgcgcgacgaggtctccgagctcaagcgcgagctgcggcagatggagaaggccgggcacctgtcctga

SEQ ID NO: 69 - DNA, 2099 bp, Rathayibacter sp.
  Marker nucleic acid sequence
atgacacccgagccgaacgactccacgtcagcccccgcagcgaacgacctggacgcgaacggctccaccggccccgccgcctcgtccgaggccgcccctgccgccgccccgtcggagggcatgtcgacgaagtcctccggcgactacggcgcgaaccagatccaggtcctcgagggcctcgaggcggtccgcaagcgccccggcatgtacatcggatcgacgggcccgcgcggtctgcaccacctggtctacgagatcgtcgacaacgccgtcgacgaggcgctcgcgggctactgcgacaacatcgaggtcgtcatgcgcgccgacggcggtctgacggtcgtcgacaacggccgcggcatcccggtcgacatgcaccccaccgagggcatctccaccgtcgagctcgtgctgacggtgctgcacgccggcggcaagttcggcggtggcggctacgccgtctccggtggtctgcacggcgtcggctcgtccgtcgtcaacgcgctctccacccggctcgaggtcgaggtcaagcgccagggcgcctcctacagcatgaccttcgccgacggcgtcccggaggcgccgctcgccaagggcgagccgagcgacgagaccggcaccaccatcaccttctggccgaacgccgagatcttcgagaccgtcgagttcgactacgagaccctgcgcgcccggttccagcagatggccttcctcaacaagggtctccgcatcgcgctgaccgacgagaaggagatcgagttcgagggcgagggcgagaacgagacctccgggccccgcagcgacgtcttcctctacgagaagggcctcgtcgactacgtcgagtacatcaactccctcaagaagtccgatctggtgcactccgagatcatcgacttcgagtccgaggacaccgagcgccgcatctcgctcgaggtcgcgatgcagtggacgacggcgtacaccgagagcgtgcacacctacgcgaacacgatcaacacgcacgagggcggcacgcacgaggagggcttccgcgcggcgctgaccacgctcgtgaacaagtacgcgcgcgcgaacaacctgctgaaggacaaggacgagaacctctccggcgacgacatccgcgagggcctcaccgccgtcgtgtcgatcaagctcggcgagccgcagttcgagggccagaccaagaccaagctcggcaacaccgaggcgaaggccttcgtgcagcgggtcgccggcgagcagctcgccgactggttcgaccgcaatcccaaccaggccaaggacatcatccggaaggcgatccaggccgcctcggcccggatggcggcccgcaaggcccgcgagaccgcgcgccgcaagggcctcctcgagggcggcggaatgcccggcaagctcaaggactgccagtccaaggacccgtcgatctccgagatcttcatcgtcgagggcgattcggccggcggctccgccgtccagggccggaaccccgagacgcaggcgatcctcccgctgcgcggcaagatcctcaacgtcgagaaggcccgcctcgaccgggccctcggcaacaacgaggtccaggcgatgatcacggccttcggagccggcatcggcgaggacttcaaccccgacaaggcgcggtaccacaagatcatcctgatggccgacgccgacgtcgacggccagcacatcaccacgctgctgctgacgctgctgttccgctacatgcgcccgctgatcgacctcggctacgtctacctcgcgcagccgccgctctaccgcctcaagtggtcgaactcggctcacgagtacgtctacagcgaccgcgagcgcgacgcgctgctcgcgcacggcgccgcgtcgaacaagcgcatgcccaaggagaacgggatccagcgctacaagggtctcggcgagatggactacaaggagctgtgggagaccacgatggaccccgccacgcggaccctcctccaggtcaccctcgacgacgcggccgccgccgacgagatcttctcgaccctcatgggcgaggacgtcgagtcccgccgctccttcatccagaaaaatgcgaaggacgtcaggttcctcgacatcta

SEQ ID NO: 70 - DNA, 1088 bp, Rathayibacter sp.
  Marker nucleic acid sequence
tgcccagttcctcagaccgcgagaagtccctcgagaccgcactcgcccagatcgaccgccagttcggcaagggcgccgtcatgcgcctcggcagcgacgagcgcgctcccgtcgaggtgatccagaccggctcgatcgcgctggacgtcgcgctcggcatcggcgggctcccgcgcggccgcatcgtcgagatctacggccccgagtcctcgggaaagacgacgctcaccctgcacgcgatcgccaatgcgcagcgcaacggcggcatcgcggcgttcatcgacgcggagcacgctctcgaccccgagtacgcgaagaagctcggcgtcgacatcgactcgctcctcgtgtcgcagcccgacaccggtgagcaggctctcgagatcgccgacatgctggtgcgctcgggctccatcgacctcatcgtcatcgactccgtggcggccctcgtgccccgcgccgagatcgagggcgagatgggcgactcgcacgtcggcctccaggcccgcctcatgtcgcaggccctgcgcaagctcaccggtgggctcaacaccaccaacaccacgatgatcttcatcaaccagctccgcgagaagatcggcgtgttcttcggcagccccgagaccaccgcgggcggcaaggcgctcaagttctacgcctcggttcgcctcgacatccgccgcatcgagacgctgaaggacggcaccgacgcggtcggcaaccgcacccgcgtcaaggtcgtcaagaacaagatggcgccgccgttcaagcaggccgagttcgacatcctctacggcatcggcatctcccgcgagggcagcctgatcgacttcggtgtcgagcacgagatcgtccgcaagtcgggcgcctggtacacctacgacggcgaccagctcggccagggcaaggagaaggcgcgccagttcctcatcgacaaccccgacatcgcggccgagatcgagaagcgcatcctgatcaagctcggtgtcgtggccgaccccaacgcggtggccgacgcgaccccggtcgcgatcgagacgaagcagccggtccgcgagaccaaggcgcccgtccggaagggcgcgtag

SEQ ID NO: 71 - DNA, 1100 bp, Bacillus sp.
  Marker nucleic acid sequence
tgggattaacggctgggattgttggattacctaacgtaggtaagtccactttatttaatgcaattacacaagcaggagcagaatctgcgaactatccattctgtacaatcgatccaaacgtagggattgtagaagtaccagatgagcgtttaaataaattaacggaattagtagaaccgaaaaaaactgttccgactgtatttgaatttactgatattgcaggtatcgtaaaaggtgcgagtaaaggtgaagggttaggaaataaattcttatctcacattcgtcaagtagatgcaatttgccaagttgttcgttgttttgaagatgaaaatattacgcacgtttcaggaaaagtagatccaattgatgacattgaaacaattaacttagaactaatcttagcggatttggaatctgttgataagcgtatcgaacgagttgcgaagttagcaagacaaaaagataaagaagctgtatatgagcatgaaattttagttcgtttaaaagaagcttttgaagagggaaaaccagctcgtactgttgaatttacagaagagcaaatgaagattgttaaaggcctgcatttacttacaacgaaagaaatgctatatgtagcaaacgtaagtgaagatgatattatggatccttccgacaataaatatgtacaaatggtaaaagaatttgcagcaaatgaaaattcacaagtaattgttgtatgtgcaaaaatagaatcagaaattgctgagttagatgaggaagagaagaaagtattccttgaagaactaggtattgaagaatctggtttagatcaattaattcgtgctgcatatgacttattaggacttgctacttacttcacagctggtgtgcaagaagtacgtgcatggacgtttaaacaaggtatgaaagcaccacaatgtgctggtgttattcatacggactttgaacgtggatttattcgtgcggaaacagtttcttatactgatttaatgacaaatggctctatgacagctgcaaaagaagctggtaaagtacgtttagaaggaaaagagtatatcgtaaaagacggagatgttatgcacttccgctttaacgtttaa

SEQ ID NO: 72 - DNA, 1784 bp, Bacillus sp.
  Marker nucleic acid sequence
gtgcacgagcatttaaaagagaagttagcaattttgcccgatcaacctggttgttatttaatgaaggataagcagggaacggttatatatgtcggaaaagcaaaggtactcaaaaatcgtgtgcgctcgtactttactggttcgcatgatgggaaaacacttcggcttgttggagaaattgttgattttgaatatattgtgacttcctcaaatttggaggctcttattttagagttaaatttaataaaaaaatatgatccaaaatataatattcaattaaaagatgataaaacatatcctttcattaaaattacagctgaaaaacagccgcgcttactcattacgcgaaatgtaaaaaaggataaaggaaagtactttgggccttatccaaatgcgcaatcggcacatgaaacaaagaaattattagatcgtatgtatccacttcgtaaatgttcaaatatgccagataaagtttgcttgtattatcatatgggtcaatgtttagcaccttgtgtgaaagaagtaacggatgagcaaaacaaagaaattgttgatgaaattattaaattcttaaacggtgggcataaagaaattcgttcagaattagaaacgaagatgtatgaggcttcagaaaagctagaatttgaacgggcgaaagagttgcgagatcaaattgctcatattgatgcaattatggaaaagcaaaagatgattatgagtgatttagtggatcgtgatgtgtttggatatgcagttgataaagggtggatgtgtgttcaagttttcttcgttcgaaaaggaaagctaattgaacgtgatgtttctatgtttccgatttatgatgaaccagaagagggatttttaacttttatcggtcaattttatgaaaacagtagtcatttcaagccgaaggaaattgttgttccaggaagtatagattcagaattagtagagcgctttttagaagttgaagcgacacagccgaagcgtgggaagaaaaaagatcttgtggaattagcaaataaaaatgcaaaaattgcactagaagagaaatttcacttaattgaacgtgatgaagaacggacgattaaagctgttgataatctagggaagcaactagggattgaaacaccatatcgtattgaagcgtttgataattcaaatattcaaggaacaaatcctgtttcagcaatgattgcttttatcgatgggaaagcagcgaagaaagaatatcggaaatataaaattaaaacggttcaaggtcctgatgattacgaatctatgagggaagttgtaagacgccgttatacgagggcgcttaaagagaatttacctttaccagatttaatcattattgatggtggaaaaggccatctggccgctacaagtgatgttctggagaatgaactcggattatatattccgatggcagggcttgtaaaagatgacaaacataaaacatcacatttaatcattggagatccgcctgaacctgtgatgcttgagaggaatagccaagaattttatttattgcaacgcattcaagatgaagtgcatagatttgcaattacattccatcgccagttgcacgggaaatcggtcattcaatcagcattggatgatattccaggaatcggcgataaacgaaaaaaggtattgctaaaacattttggttcattaaaaaagatgaaagaagcttccatgtcggaatttgtcgaagcgggtatgccgaaaaatgtagcggagacaatttatacttatttaacagataagaagacgttgta

SEQ ID NO: 73 - DNA, 2015 bp, Pseudomonas sp.
  Marker nucleic acid sequence
tgtctgaattccagctagtcacccgcttccagcccgccggcgatcagccggaagccatccgcctgatggtcgagggtatcgaggccgggctggcgcaccagacgttgctcggtgtgaccggttcgggcaagactttcagcattgccaacgtgatcgcccaggtacagcgcccgaccctggtgctggcgccgaacaagaccctggcggcgcagctgtacggtgagttcaaggcgtttttcccgaataatgccgtcgagtacttcgtgtcctattacgactactaccaacccgaggcctacgtgccgtcgtcggatacgtttatcgagaaggacgcctcgatcaacgaccacattgaacagatgcgtttgtcggcgaccaaggcgttgctcgaacgcaaggacgcgatcatcgtcaccacggtgtcgtgcatctatggcttgggcagcccggaaacctacctgaagatggtgctgcacgtggaccgcggcgacaaactcgaccagcgcgccttgctccggcgcctggcggacttgcagtacacccgcaatgacatggatttcgcccgggcgacgttccgagtgcgcggcgatgtgatcgatatctacccggcggaatcggacctggaagcgatccgcatcgaactgttcgacgatgaagtggagagcatttcggcgttcgacccgctgaccggcgaagtgatccgcaagttgccacgcttcaccttctaccccaagagccactatgtgacgccccgggaaaccctgctggacgcgatcgaagggatcaaggtcgagttgcaggagcgcctggagtacctgcgttcgaataacaaattggtggaagcccaacgcctggagcaacggacccgcttcgacctggagatgatcctggagctgggctactgcaacggcatcgaaaactactcgcgctacctgtcgggtcgcccggcgggtgcgccgccgcccacgttgtatgactacctgccagccgatgccttgctggtgatcgacgagtcccacgtcagcgtgccccaggtcggcgccatgtacaaaggcgaccgttcgcgcaaggaaaccctggtggaatacggcttccgcctgccctcggccctggacaaccggccaatgcgcttcgacgaatgggaaagcgtcagcccgcagacaatttttgtctcggccacccccggcaactacgaggccgagcatgccgggcgggtggtcgagcaagtggtacggccgaccgggctggtggacccacaggtcgaggtgcgcccggcgttgacccaggtcgatgacctgctctctgaaatcaccaagcgcgtggcggtggaggagcgggtgctggtcaccacgttgaccaagcgcatggccgaagacctcaccgactacctggccgaccatggcgtgcgggtgcgttacttgcactcggacatcgacacggtggagcgggtcgagatcattcgcgacctgcgcctgggcaccttcgatgtgctggtgggcatcaacctgctgcgcgaaggcctggacatgccggaagtgtcgctggtggcgatcctcgacgctgacaaggaaggtttcctgcgttccgagcgctcgttgatccaaaccatcggtcgcgcggcgcgtaacctcaatggccgggcgatcctgtatgccgatcgcatcaccggctccatggaacgggccatcggcgagaccgagcgccgtcgcgacaagcagatcgccttcaacctggccaatggcatcacccccaagggtgtattcaaggacgtcgccgacatcatggaaggcgccaccgtgcccggctcgcgcagcaagaagcgcaagggtatggccaaggccgccgaagagagcgcccgctacgaggccgaactgcgctcgccaagcgagatcaccaagcgtatccggcagttggaagagaagatgtaccaactggcccgagacctggaattcgaagcggcggcgcagttgcgtgatgagattggcaagttgcgggagcggttgctgactgtctga

SEQ ID NO: 74 - DNA, 2417 bp, Pseudomonas sp.
  Marker nucleic acid sequence
tgagcgaagaaaatacgtacgactcaacgagcattaaagtgctaaaaggcctggatgccgtacgcaaacgtcccggtatgtacattggtgacaccgacgatggcagcggtctgcaccacatggtgttcgaggtggtcgacaactccatcgacgaagccctcgccggccattgcgacgacatcagcatcatcatccacccggatgagtccatcaccgttcgcgacaacggccgtggcatcccggtagacgtacacaaagaggaaggcgtttccgccgccgaggtcatcatgaccgtcctccacgcaggcggtaagttcgacgacaactcctacaaggtctccggcggtctgcacggcgtaggtgtctcggtggtgaacgcgctgtccgagcaactggtcctgaccgttcgccgtagcggcaagatctgggaacagacctacgtccacggcgtgcctcaggcaccgatggcgatcgttggcgacagcgagtccaccggcacccagattcacttcaaggcatcgagcgaaaccttcaagaatatccacttcagctgggacatcctggccaagcggattcgtgaactgtccttcctcaactccggtgtcggcatcgtcctcaaggacgagcgcagcggcaaggaagagctgttcaagtacgaaggtggcctgcgtgcgttcgttgagtatttgaacaccaacaagaccgcggtcaaccaggtgtttcacttcaatatccagcgtgaagacggcattggcgtggaaatcgccctgcagtggaacgacagcttcaacgagaacctgttgtgcttcaccaacaacattccgcagcgcgacggcgggacccacctggtgggcttccgttcggcgttgacccgtaacctgaacaactacatcgaacaggaaggcctggcgaagaagcataaagtcgccactaccggtgacgatgcccgtgaaggcctgacggcgatcatttcggtgaaggtgccggatccgaagttcagttcccagaccaaggacaagctggtgtcttccgaagtgaagaccgcggtcgaacaggaaatgggcaagtacttctccgacttcctgctggagaacccgaacgaagccaagctggtggtcggcaagatgatcgacgccgcccgcgcccgtgaagccgcgcgcaaggctcgtgagatgacccgccgcaaaggcgcgctggacatcgccggcctgccaggcaaactggctgactgccaggaaaaggaccccgccctgtccgagctgtacctcgtggaaggtgactctgctggtggttccgccaagcagggtcgcaaccgtcgcacccaggccatcctgccgctcaagggcaagatcctcaacgtggagaaggcgcgcttcgacaagatgatttcctctcaggaagtcggcaccttgatcaccgcgctgggctgcggcattggccgtgaagagtacaacatcgacaagctgcgctatcacaacatcatcatcatgaccgatgctgacgtcgacggctcgcacatccgtaccctgctgctgaccttcttcttccgtcagttgccagagctgatcgagcgcggctacatctacatcgcccagccgccgctgtacaaggtcaagaaaggcaagcaagagcaatacatcaaagacgacgacgccatggaagagtacatgacgcagtcggccctggaagacgcgagcctgcacctgaacgaagacgcgccgggcatttccggtgaagccctcgagcgcctggtgaatgacttccgcctggtgatgaagaccctcaagcgcctgtcgcgcctgtacccacaggaactgaccgagcacttcatctacctgccggccgtgagcctggagcaactgtccgatcacgcagcgatgcaggattggctggcccagtacgaagtacgcctgcgcaccgtggaaaaatccggcctggtctacaaagccagcctgcgcgaagaccgtgaacgtaacgtctggttgccagaggttgaactgatctcccacggcctgtcgaactacgtcaccttcaaccgcgacttctttggcagcaacgactacaagaccgtcgtatccctcggcgcgcagctgagcacgctgctggacgaaggtgcgtacatccaacgcggtgagcgcaagaagtccgtgaccgagttcaaggaagcccttgaatggctgatggctgaaagcaccaagcgccacactatccagcgatacaaaggtctgggcgagatgaacccggatcagctgtgggaaaccaccatggacccgggctcgcgccgcatgctgaaagtcaccatcgaagacgccattggcgcggaccagatcttcaacaccctgatgggtgatgcggtcgagcctcgtcgtgacttcatcgagagcaatgcattggcagtgtccaacctggatttctga

SEQ ID NO: 75 - DNA, 1052 bp, Pseudomonas sp.
  Marker nucleic acid sequence
tggacgacaacaagaagaaagccttggctgcggccttgggtcagatcgaacgtcaattcggcaagggtgccgtaatgcgtatgggcgatcaggaccgtcaggctattccttccatctccaccggctctctgggcctggacattgcgctaggcattggcggtctgccaaaaggccggatcgttgaaatctacggtcctgaatcgtccggtaaaaccacactgaccttgtccgtgatcgcccaggcccaaaaagccggcgcgacctgcgccttcgtcgacgccgaacacgccctggatccggaatacgccggcaaactgggcgtcaacgtcgacgacctgctggtttcccagccggacaccggcgagcaggccctggaaatcaccgacatgctggtgcgttccaacgcggttgacgtgatcatcgtcgactccgtggcggcactggtgccgaaggctgaaatcgaaggtgaaatgggcgacatgcacgtgggcctccaggctcgcctgatgtcccaggccctgcgcaagatcaccggtaacatcaagaatgccaactgccttgtgatcttcatcaaccagatccgtatgaagatcggcgtgatgttcggcagcccggaaaccaccaccggtggtaacgcactgaaattctacgcctcggttcgcctcgacattcgccgtactggcgcggtgaaggaaggtgacgaagtggtcggcagcgaaacccgcgtcaaggtcgtgaagaacaaggtcgcttcgccgttccgtcaggccgagttccagattctttatggcaagggcatctacctcaatggcgagatgatcgacctgggtgtgttgcacggttttgtcgagaagtcgggcgcctggtatgcctacaacggcaccaagatcggtcagggcaaggccaactcggccaagttcctggcggataacccggacgttgccgctgccctcgagaagcaactgcgtgacaagctgttgagcccggttgcagacgtcaagtccgtggctaaccgcgagaccgctgacgatctggctgacgctgacatctga

SEQ ID NO: 76 - DNA, 1100 bp, Pseudomonas sp.
  Marker nucleic acid sequence
atgggattcaattgcggcatcgtcggcctgcccaacgtcggcaagtccaccctgttcaacgccctgaccaagtccggtattgcggcggagaacttccccttctgcaccatcgaacccaacagcggcatcgtgccgatgccggacccgcgtcttgacgcactggcggccatcgtcaatcccaagcgcatcctgccgaccaccatggagttcgtcgacatcgcgggcctcgtcgccggcgcctcgaaaggtgaaggcctgggcaacaagttcctggccaacatccgcgaaaccgatgctatcgcccacgtggtccgctgctttgaagacgagaacgtgattcacgtctccaacagcgtcgaccccaagcgcgacatcgagatcatcgacctggaactgatcttcgccgacctcgacagctgtgaaaagcaactgcagaaagtcgcgcgtaacgccaagggcggcgacaaggatgcagtggtccagaagggcctgctggagcagctgatcgcgcacttcaccgaaggcaagcctgcgcgcagcctgatgaagaacatgggggccgatgagaaggccgtgatcaagggcttccacctgctgaccaccaagcctgtgatgtacatcgccaacgttgccgaagacggcttcgagaacaacccgctgctcgacgtggtcaaggctattgccgacgaagaaggcgccatcgttgttccggtgtgcaacaagatcgaagcggaaatcgccgagctcgatgacggcgaagaaaaggacatgttcctcgaggccctgggcctggaagagcctggcctgaaccgcgtgatccgcgcaggctacgaaatgctcaacctgcagacctacttcaccgccggtgtcgaagaagtccgcgcctggaccgtcaaggtcggcgccaccgcgccacaggctgctggcgtgatccacaccgactttgagaaaggctttatccgagctgaggtgatcgcctacaacgacttcatccagttcaagggtgaagccggtgccaaggaagccggtaaatggcgcctggaaggcaaggactacattgttaaagatggcgatgtgatgcacttccgctttaacgtgta

SEQ ID NO: 77 - DNA, 2015 bp, Pseudomonas sp.
  Marker nucleic acid sequence
atgtcggatttccagctcgttacccgttttgaacccgccggcgatcaaccggaagccattcgtcagatggtcgaaggcattgaagccggcctggcgcaccagacgctgcttggggtgaccgggtccggcaagaccttcagcatcgccaacgtgatcgcccagaccaaccgcccgaccctggtgctggcgccgaacaaaaccctggcggcgcagctctatggcgagttcaaggcgttcttcccgaacaacgcggtggagtatttcgtttcctactacgactactaccagcccgaagcctatgtgccgtcgtccgatacctttatcgagaaggatgcgtcgatcaacgaccacatcgagcagatgcgcctgtcggccaccaaggcactgctggagcgcaaagacgcgatcatcgtcaccacggtgtcgtgcatctacggcttgggcagcccggaaacctatctgaagatggtgctgcacgtcgaccgtggtgacaaactcgaccagcgcgaattgctgcggcgcctggcgagcctgcagtacacccgtaacgacatggattttgcccgcgccaccttccgggtgcgcggcgatgtgatcgacatctacccggcggaatccgacctggaagcgattcgcatcgagctgttcgatgatgaggtcgaaagcctgtcagccttcgacccgttgaccggtgaagtgatccgcaagctgccacgcttcaccttctaccccaaaagccattacgtgaccccacgcgaaaccttgatgggcgccatcgagggcatcaaggtcgagctggccgaacgcctggaatacttgcgctccaacaataagctggtggaagcccagcgcctggagcagcgcactcgcttcgacctggagatgatcctggaactgggttactgcaacggcatcgaaaactactcgcgttacctgtcgggccgtgactccggggcgccgccaccgacgctctatgactacctgccgccggacgccttgctggtgatcgacgaatcccacgtcagcgtgccgcaggtgggcgccatgtataagggcgaccgttcgcgcaaggaaaccctggtggaatatggtttccgcctgccgtcggcgctggacaaccggccgatgcgctttgacgagtgggaagccatcagcccgcagaccatcttcgtctccgctacgcccggcaactatgaagccgaacatgccggtcgcgtgatcgagcagttggtgcgaccgactggtttggtggacccggaaatcgaaatccgccccgcgctgacccaggtcgacgacttgctctcggaaatcaacaagcgtgtggccctggaagagcgggtgctggtcaccactctgaccaagcgcatgtccgaagacttgactgattacctggccgaccacggcgtgcgcgtgcgttacctgcactcggacatcgacaccgtggaacgcgtggaaatcatccgcgacctgcgcctgggcaccttcgatgtgctggtggggatcaacctgctgcgtgaaggcctggacatgccggaagtgtcgctggtggcgatcctggatgcggacaaggagggcttcctgcgctccgaacgctcgctgatccagaccatcggccgagcggcgcgtaacctcaatggacgcgcgatcctgtatgcggaccgcattaccggttccatggagcgggcgatcggcgagacccagcgtcgtcgcgacaagcagatcgcgttcaacctggaacacggcattacccccaaaggtgtgttcaaggacgttgccgacatcatggaaggcgccaccgtgccgggctcgcgcagcaagaagcgcaagggcatggccaaggctgccgaggaaagtgccaagtacgagaacgaactgcgctcgccgagtgagatcaccaagcggattcggcagttggaagaaaaaatgtaccagctggcgcgggatctggagtttgaagcggcggcgcagacgcgtgatgagattggcaagttgcgtgagcgtctgctggctgtttg

SEQ ID NO: 78 - DNA, 1469 bp, Pseudomonas sp.
  Marker nucleic acid sequence
tggttcccgtaatcgccctggtgggccgacctaacgtcggcaagtccaccctgttcaaccgcctgaccaggactcgcgacgccattgtcggcgacttgtccggtctgacccgtgatcgccaatacggtgaggcaaagtggcaagggcgctcctacattattgtcgacaccggtggtatctccggtgacgagcatggcatggacgaaaaaatggccgagcagtcactgctggccattgaagaagccgatgtcgtgttgttcctggtggacgcccgcgcgggctataccgctgccgaccagatgattggcgagcacctgcgcaagcgcaacaagcgctcctatctggtcgccaacaagatcgacaacatcgatcctgaggcagcccgtgccgaattcagcccgatgggcctgggcgatgcgatcccggtcgccggtgcccacggtcgcggcatcacccagatgctggaaatcgccctgcgcgacttccccaaggatgacgtcgaggaagaagagggcgaggaagagatcgtcgctgaaggtgaggaagccaagcgcattcctggcccgagcgaaaaagacggtatcaagatcgctatcatcggccgcccgaacgtcggcaagtcgaccctggtcaaccgtatgctcggtgaagaccgcgttatcgtctatgaccaacccggcaccacgcgcgacagtatttacatcccgtttgagcgtaacgacgagaagtacacgctgatcgacaccgccggtgtgcgcaagcgcggcaagatccacgaagaagttgaaaagttctccgtggtcaaaaccctgcaagcgatcaaagacgccaacgtggtgatcttcgtaatggacgcccgcgaaggcgtggtggaccacgacctcaacttgctgggctttgccctggaagccggtcgcgcccttgtgatcgcgatcaacaagtgggacggcatgacgccgagcgagcgcgatttcgtcaaaatcgagctgcagcgtcggttgttcttcgtcgagttcgccgatatccactttatctcggcactgcacggcaccggcgtgggcaacctctacgcgtccgtacagaactcgttcaagtccgcggttacccgctggccgaccaaccgcctgacccagatcctggaagacgcggtgggcgagcacgcgccgccgatggtcaacaaccgccggatcaaactgcgttacgcccacttgggtggtgccaacccgccgattatcgtgatccacggtaaccagatcgagaaggtgcccaagtcctacgtgcgttacctggaaaacacctaccgccgtgtcttgaaactggtcggtacgccgatccgtatcgagttcaagggtggcgagaacccatacgaaggcaacaagaacacactgactgaccgtcaggtgaacaagaagcgtcgtttgatgactcaccacaagaaagccgacaagaagcgccgcgacaagaaataa

SEQ ID NO: 79 - DNA, 2030 bp, Serratia sp.
  Marker nucleic acid sequence
ttgaaagatgaaggggatatgagcaaacttttcaaactgcattcagaattcaaaccggccggcgatcagccggaagccatccgcaagttggaagagggcttggaagacggcttggcgcatcaaacgctgctgggggtcaccggttcaggtaaaaccttcaccatcgccaatgtgatcgccgatctgaaccggccaaccatggtgctggcgcccaacaaaacgttggcggcgcagctctatggcgagatgaaagagttctttcctgagaatgcggtcgagtattttgtctcctactacgattactaccagccagaagcttacgtaccgagctccgatacctttatcgagaaagatgcttcggtaaacgaacatatcgaacagatgcgcctgtccgcgaccaaggcgctgcttgaacggcgcgatgtggtggtcgtggcgtcggtttccgcaatctacggcctgggtgatccggatctttatttgaaaatgatgctgcatctgaccaagggcatgatcatcgatcaacgttcgatcctgcgccgtctggccgagttgcaatatacccgtaacgatcaggcgtttcagcgagccactttccgcgttcgcggcgaagtgatcgacatctttccggcggaatcggatgaactggcgctgcgcgttgagctgttcgacgaagaagtcgaacgcctgtcgctgttcgatccgctcaccggccagattgaacagacggtgccgcgtttcaccatctaccccaaatctcactacgttaccccgcgtgaacgtattgtgcaggcaatggaagagatcaaggcggagctggccctacggcgtaaggtactgttggaaaacaacaagctgttggaagagcagcggctgtcgcagcgtacccagttcgatctggagatgatgaacgagttggggtactgctccggcattgaaaactattcgcgttacctctccggccgtggcgagggcgagccgccgccgaccttgttcgattacctgccggcagacggtttgctggtggtggatgagtctcacgttaccattccgcagattggcggcatgttcaaaggtgaccgttcacgtaaagaaacgctggtggaatacggtttccgcttgccttcggcgctggataaccggccattgcgctttgaagaatttgaagctttggcgccgcagactatttatgtgtccgccacgccgggcaaatatgagctggagaaatccggcggcgagattatcgatcaggttgtccgcccaaccggtctgctggatccgatcgtggaagtccgcccggtcacgacccaggttgacgatctgctttccgagatccgcaagcgcgtagccatcaacgaacgcgtgctggttactaccctgaccaaacgtatggcggaagatctgacggaatacctggaagaacacggtgagcgggttcgttacctgcactcggatatcgatacggttgagcgtgtcgaaatcattcgtgatttgcgtctcggcgagttcgatgtgctggtggggatcaacctgctgagagaggggctggacatgcctgaggtgtcgttggtggcgatcctggacgcagacaaagaaggtttcctgcgttccgaacgttcgctgatccagaccatcggccgtgcggcgcgcaacctcaatggtaaagcgattttgtacggcgacaggatcaccgactcgatggccagggcgatcggcgagacggaacgtcgccgcgccaagcagcaggcgtataacgaagccaatggtatcgtgcctaagggcctgaacaagaagatttccgatattctgcaacttgggcagccaagcacgcgcggcaggaacaaaggcaagggcaaagcggcagagagcgcggcgcaataccgccatctcacgccaaaagcgctggaacagaaaattcaggaactggaagccaaaatgtattcatatgcgcagaacctggagttcgaacaggctgccgctgtgcgtgatgaaattcaccagttgcgcgagcagtttatcgcgctgtcata

SEQ ID NO: 80 - DNA, 2414 bp, Serratia sp.
  Marker nucleic acid sequence
tgtcgaattcttatgactcctcaagtatcaaggtattaaaagggctggatgcggtgcgtaaacgcccgggtatgtatatcggcgatactgatgacggcactggtctgcaccacatggtattcgaggtcgtggacaacgctatcgacgaagcactcgctggccactgtagtgatattcaggtgactatccatgccgacaactccgtgtcggtgcaggatgatggccgtggtattccaaccggtatccacgaagaggaaggggtgtctgcggcgcaggtcatcatgaccgtgctgcatgccggcggtaaattcgacgacaactcctacaaggtttccggcggcctgcacggcgtgggcgtttccgttgttaacgccctgtcagagaagcttgagctggtgatccgccgtgaaggcaaagtgcatgagcaaacctacaagcacggcgaaccgcaaggcgcgctgaaagttgtgggcgaaaccgaacaaaccggcaccaccgtgcgcttctggcctagctaccagacctttaccaataataccgaatttgagtacgacattctggccaaacgcctgcgtgaactgtcgttccttaactctggcgtatccattcgtctgaaagacaaacgcaacgataaagaagaccacttccattatgaaggcggcatcaaggcgttcgttgaatacctgaacaagaacaagactccgatccacccaacggtgttctacttctcgaccataaaagacgatatcggtgtggaagtggcgctgcagtggaacgatggtttccaggaaaatatctattgcttcaccaacaacatcccgcagcgtgatggggggacccacctggccggattccgcgccgcgatgacccgtaccctgaacaactacatggataaagaaggctacagcaaaaagtccaaaatcagcgcgaccggtgacgatgcgcgtgaagggctgattgccgttgtctcggtgaaagtgccagatccgaagttctcttcacagaccaaagacaaactggtgtcttcagaagtgaaaacagcggttgaatcgttgatgaacgagaaactggtggattacctgatggaaaatccgaacgatgcgaaaatcgtcgtcggtaaaatcatcgatgcggcccgtgctcgtgaagcggcgcgtaaagcgcgcgaaatgacccgccgtaaaggcgcgttggatctggctggcttgccaggcaagctggcggattgtcaggaacgcgacccggcactgtctgaactgtatctggtggaaggggactccgcgggcggctccgcaaaacagggccgtaaccgcaaaaaccaggcgattttgccgttgaaaggtaaaatcctcaacgttgaaaaagcgcgcttcgacaagatgctttcgtctcaggaagtcgccacgctgatcaccgcgctgggatgcggtattggccgtgacgaatataacccggacaagctgcgctatcacagcatcatcatcatgaccgacgccgacgttgacggttcgcacattcgtacgttgctgctgaccttcttctatcgtcagatgccggaaatcattgagcgtggccacgtgtttatcgcacagccgccgctgtacaaggtgaaaaaaggcaagcaggaacagtacatcaaagacgatgaagcgatggatcaatatcagatctcgatcgctctcgatggtgcgaccctgcataccaatgccagcgcgccggccttggcgggtgaaccgctggagaaaatggtcgctgagcactacagcgtacagaagctgatcggtcgtatggagcgccgctatccacgcgcgttgctgaataacctgatctaccagccgaccttgaacgaagacgatctgagcgatcaggctaaggtgaaaacctggatcgagtcgctggtgaaattgctgaacgataacgagcagcatggcagcagctatgatttcgtgatccttgagaaccgtgaacgtcagatgtttgagcctgtgctgcgcattcgcacgcacggtgtggataccgactatcagctggacttcgatttcatccacggtggcgaatatcgcaagatctgccagcttggcgagaaactgcgtggcctgctggaagaagatgccttcatcgaacgtggcgagcgtcgtcagccggtagacagcttcgaacaggcgctggaatggctggtgaaagagtctcgtcgtggcttatcggtgcagcgttataaaggtctgggcgaaatgaacccagagcagctgtgggaaaccaccatggatccagagcagcgccgcatgctgcgtgtgacggtgaaagacgccattgcggccgatcagctgttcaccaccctgatgggcgacgccgttgaaccgcgccgcgcctttatcgaagaaaacgccctgaaagcggcgaatattgatatctga

SEQ ID NO: 81 - DNA, 1064 bp, Serratia sp.
  Marker nucleic acid sequence
atggctattgatgagaacaagcaaaaggcgttagctgcagcactgggccagattgagaaacaattcggcaaaggctccatcatgcgtctgggtgaagaccgctccatggacgtagaaacgatctccaccggctcgctgtcgcttgatatcgcactgggggccggcggtctgccaatgggccgtatcgtcgagatttatggcccggaatcctcgggcaaaacaaccttgaccctgcaggtaattgccgccgcccagcgcgaaggtaaaacctgcgcgttcatcgatgccgagcacgcgttggatcccatttacgccaaaaaactgggtgtcgatatcgacaacttgctgtgttctcagccggataccggcgagcaggcgttggaaatctgtgatgcattgactcgctctggcgctgttgacgtcatcatcgtcgactcagtagcggcgttgacgccgaaagcagaaatcgaaggtgaaatcggtgattcacacatggggctggcggcacgtatgatgagccaggcaatgcgtaaattggccggtaacctgaagaacgccaataccttgctgatcttcatcaaccagatccgtatgaagattggcgtgatgtttggtaacccggagaccaccaccggcggtaacgccctgaaattttacgcttcggttcgcctggatatccgccgtatcggcgcgatcaaagaaggtgacgaagtgatcggcagcgaaacccgcgtgaaagtggtgaagaacaagattgccgcgccgttcaaacaggcggagttccaaatcctgtatggcgaagggatcaacagccgtggcgaattggtcgatctgggcgtgaagcacaagatgatcgagaaagccggcgcttggtacagctataacggcgagaagatcggtcagggcaaatcaaatgcctgtaacttcttgaaagaaaatccggcaattgctgctgaactggataagaaactgcgtgacctgttgctgcacagtaatggcgaattggttgctgccggtgtcgaagatgcagacgataacgaagcagaaaccagcgaagagtttta

SEQ ID NO: 82 - DNA, 1430 bp, Ensifer sp.
  Marker nucleic acid sequence
atgagcttcaccgtcgccattatcgggcgccccaatgtcggcaagtccaccttgttcaaccgcctggttggcaagaagctggcgctcgtcgacgatacgccgggggtaacccgcgaccggcgtccgggcgacgcgaagctggtcgacctcaagttccgcatcatcgatacggccggtctcgaggaatcgtcacccgacagcctgcaggggcggatgtgggcgcagaccgaggcggccatcgacgaggcggatctgtcgctcttcgtcgtcgatgccaaggccggcctgacgccggcagatcagacgctcgccgaaatgctgcgccgccggggcaagcccgtcgtcgtcgtcgccaacaaatccgaggcgcgcggctccgagggcggcttctacgacgccttcacgctcggcctcggcgaaccctgcccgatctcggccgaacacggccagggcatgctcgatctgcgcgacgcgatcgtcgccgcactcggcgaggagagggcgttcccgccggcggaagacgttgccgagacgaatgtggatatccgccccgttgccggtgaaggtaccgaggacgaagaggtcgagcccgcctatgacgaaaccaagccgctccgggtggcgatcgtcggccggccgaatgccggaaagtcgactctgatcaaccggttccttggcgaagaccggctgctgacggggccggaggcgggcattacccgcgactccatctccgtcgaatgggactggcgcggccgcacgataaagatgttcgatacggccggcatgcgccgcaaggcgaaggtacaggagaagctcgaaaagctctcggttgcggatgcgctgcgggcgatccgcttcgccgagaccgtcgtcatcgtcttcgacgccacggtccctttcgagaagcaagacctgcagatcgtcgatctggtgctgcgcgaggggcgtgcagccgtgcttgccttcaacaaatgggatctcgtcgagaactggcaggcgctgcttgtagacttgcgcgaaaagaccgagcggctgttgccgcaggcgcgcggcatccgtgcagtgccgatttccgggcacaccggctatggcctcgaccggctcatgcaggccatcatcgaaacggacaaggtctggaaccggcgcatctccacggcgcgcctcaaccggtggctggaatcgcagcaggtgcagcatcccccgccggccgtctcgggccgccgcctcaagctcaaatacatgacgcaggtcaaggcgcggccaccgggtttcatgatttcctgcacacgccccgaggcggtgccggagtcctacacccgctacctcatcaacggtttgcgaaacgacttcgacctgccgggcgtgccgatccgcgtgcatttccgagcgtcggagaatcccttcgagtcgaaggcgcgcaaaaggcgctg

SEQ ID NO: 83 - DNA, 1088 bp, Streptomyces sp.
  Marker nucleic acid sequence
gtgtcgctcacgatcggaatcgtcggtctgccgaatgtcggcaagtcgaccctgttcaacgccctgaccaagaacgacgtgctggcggccaactacccgttcgccacgatcgagcccaacgtcggcgtcgtcggcgtccccgacgcccgcctcaccaagctggcggagatcttcggctcccagcgcatcctcccggcgacggtcgacttcgtcgacatcgccggcatcgtgcgcggcgcgagcgagggcgagggcctgggcaacaagttcctcgcgaacatccgcgagtcggacgcgatctgccaggtcatccgcgccttcaaggacgagaacgtcgtccacgtcgacggcaaggtctcgccgaaggacgacatcgagacgatcaacacggagctgatcctcgcggacctccagacgatcgagaaggtcctgccgcgcctccagaaggagatgcggatcaagaaggacacggcgccgaaggtcgccgcggtcgaggccgcccaggcgatcctggagaagggcgacacgctcttctcgcagggcatcgtccagggctcggagaaggcggagctcctccacgacctccacctcctcaccacgaagccgttcctctacgtcttcaacgtcgacgaggacgagctgacggacgacgccttcaaggccgagcagagcgccctggtcgccccggccgaggcgatcttcctcaacgcgaagctggagcaggacctcgcggagctcgacgaggaggacgccatggagctcctccagtccgtcggcgccgaggagcccggcctcgccaccctcgcccgcgtcggcttcgcgaccctcggcctccagacctacctgacggccggcccgaaggaatcccgcgcctggacgatcaagcagggcgcgacggcccccgaggcggccggtgtcatccacaccgacttccagaagggcttcatcaaggccgaggtcatctccttcgccgacctcgtcgaaaccggctcggtagccgacgcccgcgccgccggcaaggcccgcatggagggcaaggagtacgtgatgcaggacggcgacgtggtggagttccgcttcaacgtctg

SEQ ID NO: 84 - DNA, 2138 bp, Streptomyces sp.
  Marker nucleic acid sequence
tgcggcccgtttccaagatcgaacgttcggtggcgcccttcgaggtcgtcagctcctaccagcccagcggcgaccagcccacggccatcgccgagctcgagcggcgcatccgcgcgggcgagaaggacgtcgtgctcctcggcgccaccggcaccggcaagtcggccaccaccgcgtggatgatcgagaaactccagcgccccaccctcgtgatggcgccgaacaagacgctcgcggcccagctcgccaacgagttccgcgagctcctgccgaacaacgccgtcgagtacttcgtctcgtactacgactactaccagcccgaggcgtacgtcccccagtcggacacgtacatcgagaaggactcctcgatcaacgaggaggtggagcggctccgccactccgcgacgaactccctgctcacccgcagggacgtcgtcgtggtcgcctccgtctcctgcatctacggcctcggcacgccccaggagtacgtggacaggatggtcccgctcaaggtcggcgaggagatcgaccgcgaccagctgctccgccgcttcgtcgacatccagtacacccgcaacgacctggcgttcacccggggcaccttccgggtccgcggcgacaccatcgagatcttcccggtctacgaggagctggccgtccgcatcgagatgttcggcgacgagatcgaggccctctccaccctccatccgctcaccggcgaggtcatcagcgaggaccgcgagctgtacgtcttccccgccagccactacgtcgcgggccccgagcgcatggagcgcgcggtcaacgacatcgagcgggagctggagggccgcctcgccgaactggacaagcagggcaagcacctggagtcccagcggctgcgcatgcgcaccacctacgacatcgagatgatgcgccagatcggctcctgctccggcatcgagaactactcgatgcacttcgacggccgcgagcccggctccgctcccaacacgctcctcgactacttccccgaggacttcctgctcgtcatcgacgagtcgcacgtcaccgtgccgcagatcggcgccatgtacgagggcgacgcctcccgcaagcggaccctcgtcgaccacggcttccggctgccctccgccctcgacaaccgccccctgaagtgggaggagttccagcagcggatcggccagaccgtctatctctcggccacccccggcacgtacgaactctcccgcggcgacggcttcgtcgagcagatcatccggcccaccggcctcgtcgacccggaggtcgtcgtcaagccgaccgagggccagatcgacgacctggtccacgagatccggctgcgcaccgagaaggacgagcgcgtcctcgtcaccacgctcaccaagaagatggccgaggacctcaccgactacttcctcgaactcggcatccaggtgcgctacctgcacagcgacgtggacaccctgcgccgcatcgagctcctgcgcgaactgcgcgccggcgagtacgacgtcctcgtcggcatcaacctcctccgggagggcctcgacctccccgaggtgtcgctcgtcgccatcctcgacgccgacaagcagggcttcctgcgctccgggacctccctcatccagaccatcggccgcgccgcccgaaacgtctccggccaggtccacatgtacgccgacaccatcaccccggcgatggcgcaggccatcgacgagaccaaccggcgccgcgagaagcaggtcgcgtacaacacggcgcacggcatcgacccgcagccgctccgcaagaagatcaacgacatcgtcgcgacgatcgcgcgcgaggaggtcgacaccgaggagctgctgggcagcggctaccgccagggcaaggagggcaagggcgcgaaggcacccgtccccgcgctcgccgcgcacggcgtcacgagccgcggcaaggccgggaaggcgggctccaccgtcgaactcggcgaccgcccggccaccgaactggcggcgatcatcgaggagatgaccgaccggatgcgtgccgccgccgccgagctgcagttcgaggtggcggcccggctccgcgacgaagtgggagagctgaagaaggagttgcgccagatgaaggaggcgggtctcgcctga

SEQ ID NO: 85 - DNA, 2030 bp, Streptomyces sp.
  Marker nucleic acid sequence
tgctgtgccagaaagggcgcttcgtggccgattccggcaaccccaacgagaacaatcagtacaccgccagcaacatccaggtcctcgaaggcctggatgccgtgcgcaagcgtcctggcatgtacatcggctcgaccggtgagcgcggtctgcaccacatggtgcaggaggtcgtcgacaactccgtcgacgaggccctggcaggccacgcggacaccatcgacgtgacgatcctggccgacggcggcgtccgcgtcgtcgacaacggccgcggcattcccgtcggcatcgtcgagtccgagggcaagccggccgtcgaggtcgtgctgaccgtcctccacgcgggcggcaagttcggcggcggcggctacgccgtctccggcggtctgcacggcgtcggcgtctccgtcgtcaacgccctctccaccaaggtcgcggtggagatcaagaccgacggccaccgctggacgcaggactacaagatgggcgccccgaccgcgcccctcgcgaagcacgaggcgaccgacgagaccggcacctcggtcaccttctggtccgacccggacatcttcgagaccgtcgagtactccttcgagacgctctcgcgccgcttccaggagatggccttcctcaacaagggcctgacgatcacgctcaccgacgagcgcgagtccgccaaggccacggtcggcgccgacgacccggacgccgaggcctccgccgagcaggccgcgcgcacggtgacataccactacgagggcggcatcgtcgacttcgtgaagtacctcaactcgcgcaagggcgagctgatcaacccgaccgtcatcgacatcgaggccgaggacaaggagcgcctgctctccgccgagatcgcgatgcagtggaactcgtcgtacagcgagggcgtgtactccttcgccaacacgatccacacccacgagggcggtacccacgaggagggcttccgcgccgcgctgacgacgctcgtcaacaagtacgcgcgcgagaagaagctgctccgcgagaaggacgacaacctcacgggcgacgacgtccgcgagggtctgaccgcgatcatctcgatcaagatcggcgagccgcagttcgagggccagacgaagaccaagctcggcaacaccgaggcgaagaccttcgtccagaaggtcgtccacgagcacctgacggactggttcgaccgcaaccccaacgaggccgcggacatcatccgcaagggcatcaccgccgccaccgcgcgcgtcgccgcccgcaaggcgcgggacctcacccgccgcaagggcctcctggagtcggcctcgctgccgggcaagctctccgactgccagtcgaacgacccgtccaagtgcgagatcttcatcgtcgagggtgactccgccggcggctcggccaagtcgggccgcaacccgatgtaccaggccatcctgccgatccgcggcaagatcctgaacgtcgagaaggcccgtatcgacaagatcctgcagaacaccgaggtccaggcgctcatctccgccttcggcaccggtgtgcacgaggacttcgacatcgagaagctccgctatcacaagatcatcctgatggcggacgccgacgtcgacggtcagcacatcaacaccctgctgctgaccttcctcttccgcttcatgcggccgctggtcgaggccggtcacgtctacctctcgcgcccgccgctctacaagatcaagtggggccgggacgacttcgagtacgcgtactcggaccgcgagcgggacgcgctcgtcacgctgggcaagcagaacggcaagcggatcagggaagactcgatccagcgcttcaagggtctcggtgagatgaacgccgaggagctgcgcgtcaccaccatggacgtcgagcaccgcgtgctcggccaggtcaccctggacgacgcggcccaggccgacgacctgttctcggtgctgatgggcgaggacgtcgaggcgcggcgctcgttcatccagcggaacgccaaggacgttcgcttcctcgacatctga

SEQ ID NO: 86 - DNA, 1133 bp, Streptomyces sp.
  Marker nucleic acid sequence
atggcaggaaccgaccgcgagaaggcgctcgacgccgcgctcgcacagattgaacgccaattcggcaagggcgccgtgatgcgcatgggtgaccgtacccaggagccgatcgaggtgatctccaccggatcgaccgcgctcgacatcgccctcggcgtcggcgggctccctcggggccgtgtcgtggagatctacggaccggagtcctccggtaagacgactctgacgctgcacgccgtggccaacgcgcagaaggccggcggccaggtcgccttcgtcgacgcggagcacgccctcgaccccgagtacgcgaagaagctcggcgtcgacatcgacaacctcatcctgtcccagccggacaacggtgagcaggccctcgagatcgtcgacatgctggtccgctccggcgccctcgacctcatcgtcatcgactccgtcgccgcgctcgtcccgcgcgcggagatcgagggcgagatgggcgactcgcacgtcggtctccaggcccgactgatgagccaggcgctccggaagatcaccagcgcgctcaaccagtccaagaccaccgcgatcttcatcaaccagctccgcgagaagatcggcgtgatgttcggctcgccggagaccacgaccggtggccgcgccctgaagttctacgcctcggtgcgcctcgacatccgccgcatcgagaccctcaaggacggcacggacgcggtcggcaaccgcacccgcgtcaaggtcgtcaagaacaaggtcgcgcccccgttcaagcaggccgagttcgacatcctctacggccagggcatcagccgcgagggcggcctgatcgacatgggcgtggagcacggcttcgtgcgcaaggccggtgcctggtacacgtacgagggcgaccagctcggccagggcaaggagaacgcccgcaacttcctcaaggacaaccccgacctcgccgacgagatcgagaagaagatcaaggagaagctcggcgtcggcgtcaagccccaggcgtcggccgagccgggtgcggacgcggcggccggtgccgacgcggccggcgcggacgacgccgccaagaccgtcccggcaccggcgaccaagaccgcgaaggcgaccaaggccaccgcggccaagagctg

SEQ ID NO: 87 - DNA, 1448 bp, Streptomyces sp.
  Marker nucleic acid sequence
atgaacgaccagcacgaccacggagcgcttggcgacgccgagtacgcggagttcatggagctcgccgcggaagagggcttcgacgtcgaggaggtcgagggcgcgatcgaggaggccgggcacggcccgctccccgtcctcgccgtcgtcggccgtccgaacgtcggcaagtcgaccctggtgaaccgcatcatcggccgccgcgaggccgtcgtcgaggacaagccgggcgtcacccgcgaccgcgtcacctacgaggccgaatgggccggccgccgcttcaaggtcgtcgacaccggcggctgggagcaggacgtcctcggcatcgacgcctccgtcgccgcccaggccgagtacgccatcgaggccgccgacgcctgcctcttcgtcgtggacgccaccgtcggcgccaccgacaccgacgaggccgtcgtcaagctgctccgccgcgcgggcaagccggtcgtcctcgccgccaacaaggtcgacggccagtccggcgaggccgacgcggccacgctctggtccctgggcctcggcgagccgttccccgtctcctccctgcacggccgcggcaccggcgacctcctcgacgaggtccttcgcaagctccccgaggcccccgagcagcgcttcggcaacgccgtcggcggcccccgccgcatcgccctcatcggccgcccgaacgtcggcaagtcctccctgctcaacaaggtggccggcgaggaccgcgtggtcgtcaacgagctggccggcaccacccgcgacccggtcgacgagctcatcgagctcggcggcgtgacgtggaagttcgtcgacaccgccggcatccgcaagaaggtccacctccagtcgggcgcggactactacgcctcgctgcggaccgccgccgccatcgagaaggcggaggtggccgtcatcctcatcgacaccaccgacaagatctcggtccaggaccagcgcatcatcacgatggcggtcgagtcgggccgcgccatcgtcatcgcctacaacaagtgggacgagctcgacgaggagcgccgctactacctcgagcgcgagatcgagaccgagatgcagcaggtcgtctgggcgccccgggtcaacgtctccgcgaagaccggccgccacatggacaagctggtcccggccatcgagaccgccctcgccggctgggagacccgcgtccccaccggccggctgaacgccttcctcggtgagatcgtcgcggcccacccgcacccgatccgcggcggcaagcagccccgcatcctcttcggtacgcaggcgggcaccaagccgccccgcttcgtgctcttcgcctcgggcttcctggaggccggctaccgccgcttcatcgagcgccgtctgcgcgaggagttcggcttcgagggcaccccgatccacatctcggtgcgcgtgcgcgagaagcgcggccggaagaagta

SEQ ID NO: 88 - DNA, 2012 bp, Serratia sp.
  Marker nucleic acid sequence
atgagcaaacatttcaaactgcattccgaattcaaaccggcaggcgatcagccgcaagccatcaaaaagctggaagagggcctggaagacggcctggcgcatcaaacgctgctgggggttaccggttcgggcaaaacctttaccatcgccaacgtgatcgccgatctcaaccgacccaccatggtgctggcgcccaacaaaaccctggcggcgcagctgtatggcgagatgaaagagttcttccctgagaatgcagtggagtattttgtctcctactacgattactaccagccagaagcctatgtgccgagctccgatacctttatcgagaaagatgcctcgatcaacgaacatatcgaacagatgcgcctgtcggcaaccaaagcgctgcttgagcggcgtgacgtgatagtggtggcttcggtctccgccatttacggtctgggcgatccggatctgtacctgaaaatgatgctgcacctgaccaagggcatgatcatcgatcagcgttcaatcctgcgccgcctggccgaactgcaatatacccgcaacgatcaggccttccagcgtgccactttccgcgtgcgtggcgaagtgatcgacattttcccggcggaatcggatgagctggcgctgcgcgttgagctgttcgacgaagaggtcgagcgcctgtcgctgttcgatccgctaactggccagattgagcaggtcgttccgcgttttaccatctaccccaaatcccactacgtcaccccgcgcgagcgtatcgtgcaggcgatggaagatatcaaggtcgaactggcccagcggcgcaaggtgctgctggaaaacaacaagctgttggaagagcaacggctgtcgcagcgcacccagtttgatctggaaatgatgaacgaactgggctattgctcgggcattgaaaactactcgcgctacctttccgggcgcggtgagggtgaacccccgccaaccctgtttgactacctgccagccgatgggctgctggtggtcgacgaatctcacgtcaccatcccgcaaatcggcggtatgttcaaaggcgaccgttcgcgtaaagaaacgctggtagagtacggtttccgtctgccttcggcgctggataaccggccaatgcgctttgaagagtttgccgcagcggcgccgcagaccatctacgtgtctgctacaccgggcaaatatgagctggagaaatccggtggcgatatcatcgatcaggtggtgcgcccaaccggcctgctggacccgatcgtcgaagtgcgcccggtgactactcaggtggacgatctgctgtctgaaatccgcaaacgcgttgccatcaacgaacgcgtgctggtcaccacgctgaccaaacgtatggcggaagacctgacggaatacctggaagaacatggcgagcgggtgcgctatctgcactcggacatcgatacggtcgaacgtgtcgaaatcatccgtgatttgcgccttggcgagtttgacgtgctggtgggtatcaacctgttaagggaaggcctggacatgccagaggtgtcgctggtagccatcctggacgcagacaaagaaggcttcctgcgttccgaacgatcgttaatccagaccattggccgagcggcacgtaacctcaacggcaaagcgatcctctatggcgacaggatcaccgattccatggccaaggcgatcggcgaaacggaacgccgccgtgccaaacagcaggcgcataacgaagccaacggcattgtgccgaagggcctgaacaagaaggtctcggatattctgcagcttgggcagccaagcagccgtggcaagcacaaagggaagggcaaggcggcagagaacgcggcgcaatacaagctgctcacccctaaagcgctggagcaaaaaatccacgagctggaggcacagatgtattcccacgcgcagaatctggagttcgaacaggcggctgcggtgcgcgatgaaatccaccaactgcgcgaacagtttattgcgctatccta

SEQ ID NO: 89 - DNA, 2414 bp, Serratia sp.
  Marker nucleic acid sequence
tgtcgaattcttatgactcctcaagtatcaaggtattaaaagggctggatgcggtgcgtaagcgtccgggcatgtatatcggtgatactgatgacggcaccggtctgcaccacatggtattcgaggtcgtggacaacgctatcgacgaagcactcgctggccactgtagcgacattcaggtgacgatccacgccgacaactctgtctcggtgcaggatgatggccgtggtattccaaccggtatccacgaagaggaaggggtgtccgcggcgcaggtcatcatgaccgtactgcacgcaggcggtaagtttgacgacaactcctataaggtttctggtggtctgcacggcgtaggcgtttcggttgttaacgccctgtcagagaagctggagctggtgatccgccgcgaaggcaaggtgcatgagcaaacctacaagcatggcgaaccgcaagccccgctgaaagtggtaggtgaaaccgagctgaccggcaccaccctgcgcttctggcctagctacctgacctttaccaataacatcgagtttgagtacgacatcctggccaagcgcctgcgtgaactgtcgttcctcaactccggcgtgtctatccgcctgaaagacaaacgcaatgaccgagaagaccacttccattacgaaggcggtatccgggcgtttgtggaatacctgaacaagaacaagaccccgatccacccaacggtgttctacttctcaaccgtgaaagacgatatcggtgttgaagtggcattgcagtggaacgatggtttccaggaaaacatctactgcttcaccaacaacatcccgcagcgtgatggggggacacacctggccggtttccgtgccgcgatgacccgtaccctgaacacctacatggataaagaaggctacagcaaaaagtccaagatcagcgccaccggtgacgatgcgcgtgaaggtctgattgccgtggtttcggtgaaagtgccagaccctaaattctcttctcagaccaaagataaactggtgtcttctgaagtgaaaacagcggtcgaatcgctgatgaacgagaagctggtggattacctgatggaaaacccaggtgatgccaagatcgtcgtcggtaaaatcatcgatgcggcccgtgctcgtgaagcggcgcgtaaagcccgtgaaatgacgcgtcgtaaaggtgcgctggatttggctggcttgccaggcaaactggcggattgccaggaacgtgacccggcgctctccgaactctacttggtggaaggggactccgcgggcggctcagccaaacagggccgtaaccgtaagaatcaggcgatcttgccgttgaaagggaaaatcctcaacgttgagaaagcccgcttcgacaagatgctgtcttcacaggaagttgccacgctgatcactgcgctgggctgcggcattggccgtgacgaatacaacccggacaaactgcgttatcacagcatcatcatcatgaccgatgccgacgtcgatggctcgcacatccgtacgctgctgttgaccttcttctatcgtcagatgccggaaatcatcgagcgtggccacgtctatattgcccagccaccgttgtacaaggtgaagaaaggcaagcaggaacagtacatcaaagacgacgaagcgatggatcagtatcagatctccattgccctcgacggcgcgaccctgcacaccaacggttctgcaccagccttggcgggtgagccgttagagaagctggtggctgagcattacgccgtgcagaaacagattggccggatggaacgccgcttcccgcgtgcgttgctgaataacctgatctaccagccaaccctgaacgaagaagatctgggtgaccgtgccaaggtggaaggctggatcgaatcgctggtgaaattgctcaacgataacgagcagcacggcagcagctacagttctgtgctggttgaaaaccgtgagcgtcagatgttcgaaccggtgctgcgtattcgtacccacggcgtagataccgactatccgctggactttgaattcatccgcggtggcgaataccgcaggctctgcactctgggtgaaaaactgcgcggcctgttggaagaagatgcctatatcgaacgtggcgaacgccgcctgccggtagacagcttcgagcaggcgttggagtggctggtgaaagagtcacgccgcggcctgtcgatccagcgctataaagggttgggcgaaatgaacccggatcaactgtgggaaaccaccatggacccggatcagcgccgtatgctgcgcgtggcagtgaaagacgctatcgcagcggatcagctgttcaccaccctgatgggcgatgcagttgaaccacgccgtgcctttatcgaagaaaacgccctgaaagcggcgaacatcgatatctaa

SEQ ID NO: 90 - DNA, 1064 bp, Serratia sp.
  Marker nucleic acid sequence
atggctattgatgagaacaagcaaaaggcgttagctgcagcactgggccagatcgagaaacagttcggcaaaggctccatcatgcgcctgggtgaagaccgctccatggacgtagaaactatctccaccggctcgctgtcactcgatatcgcactgggcgcaggcggcttgccaatgggccgtatcgtcgaaatttatggcccggaatcctccggtaaaacaaccctgactctgcaagttattgcctcagcgcagcgtgaaggtaaaacctgtgcgtttatcgatgccgaacatgcgctggaccctatctacgccaagaaactgggcgtggatatcgataacctgctgtgttctcaaccggataccggcgagcaggcattggaaatctgtgatgcactgacccgctccggcgcggttgacgtcatcatcgtcgactccgttgcggcactgacgccaaaagcggaaatcgaaggcgaaatcggtgactcacacatggggctggcggcacgtatgatgagccaggccatgcgtaagctggccggtaacctgaagaacgccaacaccttgctgatcttcatcaaccagatccgtatgaaaattggcgtgatgttcggtaacccggaaaccaccaccggtggtaacgccctgaagttctacgcttctgtccgtctggatatccgccgtatcggcgcgatcaaagaaggcgatgaagtgatcggcagcgaaacccgcgtcaaagtggtgaagaacaagattgctgcaccgttcaaacaggcagagttccagatcctgtatggcgaagggatcaacagccgtggcgaactggtcgatctgggtgtgaaacataagatgatcgaaaaagcgggtgcctggtacagctacaacggcgagaagatcggccagggcaaatcaaacgcctgtaacttcctgaaagaaaacccggcaattgctgcggaactggacaagagactgcgtgacctgctgctgcacagcactggcgaagcggttgctgctggtgccgataaggccgatgacaacgaagcagaaaccagcgaagagtttta

SEQ ID NO: 91 - DNA, 1391 bp, Rahnella sp.
  Marker nucleic acid sequence
tgctgaagatttttaataccctgagtcgccaaaaagaggaattcaagccaattcatgccggtaaagtcgggatgtacgtgtgtgggatcactgtttacgatctgtgtcatatcggccacggtcgcacttttgtgtctttcgacgtggtatcccgttacctgcgctacctgggttatgacctgaaatatgtgcgtaacatcaccgatatcgacgacaaaatcattaaacgtgccgctgaaaatggcgaaacgtttatggcgctgaccgaccgtatgattggcgaaatgcacgctgattttgctgcactgaatattctgccacctgactctgaaccgcgcgccacgcagcacattcacgaaattatcgatatcgtgcagacgctgattgaccgtgaccacgcctatgtcgcgtcgaacggcgatgtgatgttctgggtggaaagtgatccggattacggactgttgtcccgtcaggatctggatcagttacaggcgggggcgcgcgtcgaagtcgaagccaacgtgaaacgcaatccgatggatttcgtactgtggaaaatgtccaagccgggcgaaccgagctggacatctccgtggggcgaaggtcgcccgggctggcatatcgaatgttccgcgatgaactgcaaacagctcggtgcgcatttcgacattcacggcggcggctctgacctgatgttcccgcaccatgaaaacgaaatcgcacagtcaagctgcgcacatgacggcccgtatgtgaatacctggatgcactccggcatggtgatgatcgaccgcgaaaagatgtccaaatcactggataacttcttcaccgtgcgggatgtgctgaaatattacgatgcggaaagcgtgcgttacttcctgatgtcgggtcactatcgcagccagctaaactacagcgaagagaacctgaaactggcgcgtacctcgatggagcgtctgtacaccgcactgcgcggcactgacgccgctgcaacgccagccggtggtgaggctttcgttacgcgcttccgtgcggctatggacgatgatttcaacacaccggaagcctattccgtgctgttcgatatggcacgtgacatcaaccgtctgaaaacggaagacctgtctgcggccaacggtctggcggcggaactgcggcaactggcaggcgtgcttggcctgctgaagcaggatccggagcagttcctgaaatccggagctcaggcggataacgatgacgaagtggctgaaatcgaagcgttgattaaacagcgtaacgatgcccgcgccagcaaagactggggcatggcggataaagcgcgcgatcgcctcaatgaaatgggtatcgtgttagaagacggaccggcaggcaccacctggcgtagaaaataa

SEQ ID NO: 92 - DNA, 1007 bp, Rahnella sp.
  Marker nucleic acid sequence
tgattgaagctgaccgcctgatttcttccgaagtcttcagtgaagaagaggtcattgaccgtgcgatccgcccgaagctgctcaacgaatacgtcggtcagcctcatgtgcgctcgcaaatggaaatctttattcaggcggctaagcaacgcaaagacgcgctcgatcacctgctgattttcgggccgccggggttgggtaaaaccacgctggctaacatcgtggcgaacgaaatgggcgtgaatttacgcaccacgtcgggtccggtgctggaaaaggccggcgatctggccgcgatgctgaccaatcttgagccgcacgatgtgttgttcattgatgaaatccaccgtctttcacccgtcgttgaagaaattctttatccggcgatggaagactatcagctggatattatgatcggcgaaggtcctgcggcgcgttccatcaaactcgatcttccgccgtttaccctgattggcgcgaccacccgcgccggttccctgacgtcacccttgcgtgaccgtttcggtatcgtgcagcgtctggagttttatcaggtagctgatttgcagcacattgtcgggcgcagcgcgtcctgcctcgggctggagttgtcggaagagggcgcgcatcagattgcccgccgttcacgcggaacgccgcgtattgccaaccgtctgttgcgccgtgtgcgtgactttgccgaagtcaaaggcgacggcaccattaacggtgatgtggctaacggcgcgctggatatgctggatgttgatgccgaaggcttcgattacatggaccgcaaactgctgctggcgattatcgacaagttcaccggcggaccggtcggactggataacttagcggcggcgattggtgaagagcgcgagaccatcgaagacgtgatcgaacccttccttatccagcagggctttattcagcgcacaccgcgcggtcgcatggcaaccaatcatgcgtataagcactttggtatcgcgagagagactgagtaa

SEQ ID NO: 93 - DNA, 1103 bp, Aminobacter sp.
  Marker nucleic acid sequence
tgggtttcaaatgcggcatcgttggcctgcccaacgttggcaaatcgacgcttttcaacgcgctgacgcgcacggctgccgcgcaggcggcgaactaccccttctgcaccatcgagccgaacaccggcgaggtcgcagtgccggacccgcgcctcaagcagctggccaatatcgccaagtcgaaggagatcattccgacccgcatctccttcgtcgacatcgccggcctggtgcgcggcgcttccaagggtgaaggtctgggcaaccagttcctcgccaatatccgcgaggtcgacgccatcgtgcatgtgctgcgctgtttcgaggatgacgacatcacccatgtcgagggccgtatcgacccggtcgccgacgccgagacggtcgagaccgagctgatgctcgccgaccttgaaagcctcgagcgccgcatcgtgcagttccgcaagcgcggcgccagcaaggacaaggaagcaaccaccgtgctgcccatgatggagcaggcgctcgcgctgctccaggacggcaaaccggcgcgcctgctgctcaagggcatcgctgccgaggacctcgccatcctgcagggcctgaaccttctgacttcgcatcctgtgctctatgtctgcaacgtcgccgaaagcgatgctgcaactggcaatgtgcacaccgcagcagtccagaagatggccgaggcgcagggcgcaggggtggtcatcatctcggccgcgatcgaggccgaagtggcgcaactgcctgacgaggaagcccaggaattcctggccgacatgggcctcgacgagccgggcctcgataagctcatccgcgccggctacgagctgttgcacctgatcacctacttcacagtcggtcctaaggagacgcgcgcctggaccgtgcacaagggcgccaaggcgcctcaggcggcgggtgtcatccacaccgacttcgaacgtggtttcatccgcgcccagaccatcgccttcaacgacttcgtcaccctcggcggcgaagttgcggccaaggaagccggcaaggcacgcgacgaaggcaaggaatatgtcgtccaggacggcgacgtgatgctgttcaagttcaacacctga

SEQ ID NO: 94 - DNA, 3053 bp, Aminobacter sp.
  Marker nucleic acid sequence
tggccaaatctgctgacaagaaatcgcccgcctcgggaagcgacaaccgtgccggaccgcgcaagcgcggcggcaacggcgtaaccgacttcctcgacgcctccgagcctttgcatcgccccggcttcggtgaggcgccgcaggccgcgttctcgggcgcgccgctgtcgggcacgatttcggattgggccgaggagatcgcgcgcgaggctgaaaagccccaacccaaggccaagaaaatcccggaaaaaagcgcggccccgacccgctcggcgcgtggcacctcgatgggaggtgctgcctccgccaaggagcgcgcggcggccggcctcaacccggtcgccggcctcgacatctcgctcgaggatgctgcatcgctgtccaattccggcgttactgccaccgtcgcggcgctctcggcgctgatcgagagcggcaacccgctgcacaagaacggcgagatctgggttccccatcgccccgcccggccggaaaagtccgagggcggcatacagatcgtcatgcaatcggagttcgagccggccggcgaccagccgaccgccatcaaggatctcgtcgagggcgtcggcaatgccgaccgcacccaggtgctgctcggtgtcaccggctccggcaagacgttcaccatggccaaggtgatcgagcagacgcagcgtccggcgctgatcctcgcgccgaacaagaccctggccgcccagctctatgccgagttcaagaagttcttccccgacaacgcggtcgagtatttcgtctcctattacgactactaccagcccgaggcctacgtcccgcgtaccgacacctacatcgagaaggaatcgaccatcaacgagcagatcgaccgcatgcgccactcggcgacgcgctcgctgctcgaacgcgacgacgtcatcatcgtcgcctcggtctcctgcatctacggtatcggctcggtcgagacctatacggcgatgaccttccagatgcaggtcggcgatcgcctcgaccagcgccaactgctcgccgacctcgtcgcccaacagtacaagcgccaggacatcaacttcgtccgcggctcgttccgcgtccgcggcgacaccatcgagatcttccccgcccacttggaagactcggcctggcgcatctcgctgttcggcgacgagatcgatgccatcaccgagttcgatccgctcaccggccagaagaccggcgacctcaaaagcgtcaagatctacgccaactcgcactacgtcacgccgcgcccgaccctgaatcaggccatcaagtcgatcaaggaagagctgcagcaccgcctgcaagagcttgaaaaggctggccgactgctcgaagcccagcgcctggagcagcgcacccgcttcgacctcgagatgctggaagccaccggctcctgcgccggcatcgagaactattcgcgttatctcaccggtcgtcgccctggcgatccgccgccgaccctgtttgagtacatccccgacaatgcgctggtcttcatcgacgaaagccacgtcaccgtgccgcagatcggcggcatgtaccggggcgacttccggcgcaaggcgacgctggccgagtacggcttccgcctgccatcctgcatggacaaccgtcccttgcgcttcgaggaatgggacgcaatgcgcccgctctcggtcgcagtgtcggctacccccggcaactgggaaatggaagaggccggcggcgtgttcgccgagcaggtcatccgcccgaccggcctgatcgacccgccggtcgaggtgcgccccgccaagaaccaggtcgacgatgtcgtcggcgaaatccgcgagacgaccagggccggctaccgcacgctggtgacggtgctgaccaagcgcatggccgaggatctgaccgaatacctgcatgagaacggcgtccgcgtccgctacatgcactccgacatcgacacgctggagcgcatcgagattttgcgcgatctcaggctcggggcgttcgacgtgctggtcggcatcaacctgctgcgcgaaggcctcgacattcccgaatgcggtttcgtcgccatcctcgacgccgacaaggaaggcttcctgcgttccgaaacctcgctgatccagaccatcggccgcgcggcgcgtaatgtcgacggcaaggtcatcctctacgccgaccagatcaccggctcgatggagcgcgcgatggctgagaccacgcgccgccgcgaaaagcagatggtgtggaacgaggccaacggcatcacgccggagagcgtcaagtcgcgcatctccgacatcctcgattcggtctacgagaaggaccacgtccgcgccgacatctcgcagttcaccgacgatgccggttcgatgatgggcaacaacttgaaggcccatctcgaagccctcgacaagcagatgcgcaacgccgccaccgacctcgacttcgagaaagccgcccgcatccgcgacgagatcaaacgcctgcgcgagatggagctcgccatctccgacgacccgctggcgcgcgaggtcgagggtcagagcccgtcctcgggccgcgagaagggcaagcacaacaagggcgtcgccaagcaccgcaccgccgaggagcaggagcgtttccgcaagctcgacgaggcccgcgccgccgaggaagcagccaaggccgcgcgccccaacatgttccgcaagccgacgctcgacgagatgggcgccgacggcgcggttcccgtcggcaagtcgttgttcgccaagcctcacatcgacgacatgggtcccggcaccgacatgaccacgcccgctggcgcggtgtcacgttcgctgttcaagaagcagtcggccgccgaagcccatggctccgactatggcgtccccgacgacggcaagtctctgttcaagaagaacgacctcgacgagatgaccgttcgccgtacggaaaaacccgtcgaaggcgtcaagccgatcaagcgcgccaaaatcggcgtcggctcttatgaggatcccgcagaagccgcgcgcgccaggaagaagccgggcaagtcaggacggccggggaagtaa

SEQ ID NO: 95 - DNA, 2447 bp, Aminobacter sp.
  Marker nucleic acid sequence
tgagcgaccagtccgagaaccttcccggcgcagaagccgagtatggcgcagattccatcaaggttttgaagggcttggacgccgtccgcaagcgcccgggcatgtatatcggcgacactgacgacggctcgggcctgcatcacatggtctatgaggtggtcgacaacgccatcgacgaggcgctggccggccatgccacgctggtcaccgtgtcgctcaatcccgatggatcggtgaccgtaaccgacaatggccgcggcattccgaccgacatccacccctcggaaggcgtgtccgccgccgaggtgatcatgacgcagctgcacgcaggcggtaagttcgaccagaactcctacaaggtttcgggcggcctgcatggcgtcggcgtgtcggtcgtcaacgcgctgtcggtgtggctgaagctcaggatccgccgcaagggcgagattcacgagatgaccttcaaccacggcaacgcagtggccccgctgaaggtcgtcggcgaggccggtgacgagaccggcacggaagtgaccttcctgccctcgcccgacaccttcaccatggtggagttcgactacaacacgctggagcatcgcctgcgcgagctcgccttcctgaactcgggcgtgcgcatcgtcttgtccgacaagcgccatgccgacgtgaaggtgcaggagctgctctatgacggcggcctgatcgaattcgtgaaatatctcgaccgcgccaagaagccgctgatcgcggccccgattgccatcaaggcggagcgcgacggcatcacggtcgaagtggcgatgtggtggaacgacagctaccatgagaacgtgctggccttcaccaacaacatcccgcagcgcgacggcggcacgcaccttgccggcttccgcggcgccctgacgcgtcaggtgaccggctacgccgaaacttcgggcatttccaagaaggaaaaggtctcgatcaccgccgacgacacccgcgagggcctgacggcggtgctttcggtcaaggttcccgatccgaagttctcgtcgcagaccaaggacaagctggtttcctccgaggttcgcccggtggttgaaagtctggtcaacgaagcgctcggcacctggctcgaagaacatccgacggaagccaagacgctgatcgccaaggttgtggaagcggcggccgcgcgcgaagcagcgcgtaaggcacgcgaattgacacgccgcaagggtgtgctcgacatcacatccttgcccggcaagctggccgactgccaggaacgcgatccggccaagtccgaaatcttcatcgtcgagggtgactcggcaggcggctcggccaagtccggccgctcacgccagaaccaggcgatcctgccgctacgtggcaagattctgaatgtcgagcgcgcccgcttcgaccgcatgctgtcgtccgacatgatcggcacgctgatcaccgcgctcggcacctcgatcggcaaggacgagttcaatgccgacaagctgcgctaccacaagatcatcatcatgaccgacgccgacgtcgacggcgcccacatccgcacgcttttgctgacgttcttcttccgccagatgccggaactgatcgagcgcgggcacctctatattgcccagccgccgctctacaaggtgatgcgcggcaagagctcgcagtacctgaaggacgaaagcgccttcgaggattaccttgtcgacgtcggccttgacgaggcgtcgctgacgcttgcgtccggcgaggtgcggaccggtcaggacctgcgcaccgccgttgccgatgcgcttggcgtgcggacgctgatcgacggcctgcacacgcgttactccaggccggtggtcgagcaagcggcgatcgccggcgcgctcaatcccgacatctttaacgatctcggccgcgccaaggcgatggccgaaaccatcgcccagcgcctcgacctgatctcggaagacatcgagcgcggctggagcggggaaattgcgaccagcaatgacggttcgggcggctacgtgttcgaacgcacgctgcgcagcgtcaaggagagcatccacctcgacatggcgctgatcaactctgccgacgcacgcgcactcgaccgctatgccacgcgcctcggcgaggtctacaaccagccaccggtgctgcgccgcaaggagaactcggagacactggcggggccgctggcgctgctgaacgccgtcttcgcctcgggccgcaagggcctgaccatgcagcgctacaagggcctgggcgaaatgaacgccgagcagctgtgggagacgacgctcgacccgaacgtgcgctcgctgctgcaggtcaaggtcaacgatgccaccgacgccgacgcgttgtttgcccgcctgatgggcgacgaggtcgagccgcgccgcgacttcatccaggaaaacgcactcagcgtggccaatctggacgtgtga

SEQ ID NO: 96 - DNA, 1049 bp, Aminobacter sp.
  Marker nucleic acid sequence
gtggacaaatccaaggctctggacgcggcgctgtcgcagatcgaacgcgccttcggcaagggctcgatcatgcgcctgggcgccaatgagcaggttgtcgagatcggaacggtctccaccggctcgctcggcctcgatatcgcgctgggcgtcggcggactgccgcgcggacgcatcatcgaaatctatggtccggaaagctcgggcaagacgacgctcgctttgcacaccgtggccgaagcccagaagaagggcggcatctgcgccttcgtcgacgccgaacatgcgctcgacccggtctatgcccgcaagctcggcgtcgatctcgaaaaccttttgatctcgcagcccgacacgggcgagcaggcgctggagatctgcgatacgctggtgcgttccggcgccatcgacgttctggtcgtcgactcggttgcagcgctgacgccgcgcgccgaaatcgagggcgagatgggcgagtcgctgcccggcctgcaggcccgcctgatgagccaggcgctgcgcaagctaacggcctcgatctcgcgctccaacaccatggtcatcttcatcaaccagatccgcatgaagatcggcgtcatgtatggctcgcctgaaaccaccaccggcggcaatgcgctcaagttctacgcttcggtccgcctcgacatccgccgcatcggctcggtcaaggaccgcgacgaggtcgtcggcaaccagacccgcgtcaaggtggtcaagaacaagctggccccgcccttcaagcaggtcgagttcgacatcatgtatggcgaaggcgtgtccaagactggcgagctcgtcgatctcggcgtcaaggccggcgtggtcgagaagtcgggcgcctggttttcctacaattcccagcggctcggccagggacgcgagaacgccaagctgttcctgcgcaacaatcctgacgctgcacgcgaaatcgaacagctgctcaggcagaatgccggactgatcgccgagaagttcctcgagaacggctcgcgcgaggaagacgacggtgacgaagctggcgcgatgta

SEQ ID NO: 97 - DNA, 1436 bp, Aminobacter sp.
  Marker nucleic acid sequence
tggctttcaagatcgcgataatcggccggcccaatgtcggcaaatccacccttttcaaccgcctggttggaaagaagatcgcccttgtcgacgacacgcccggcgtgacgcgcgaccgccgcgtgcacgcggccaagctctacgatctcgtcttcgacgtcatcgacacggcaggtttcgaggatgccggtgctgcaaccctgcagggccgcatgcgggcgcagaccgagatcgccatcagggaagccgatctcatcttcttcatggtcgacgccaaggcgggtctgaccccggacgaccgcaccttcgtcgagatcgtgcgccgttccggcaagcctgtcgtgctcgtcgccaacaagtccgaggcgcgcggtgcccagggcggcgcgctcgaagcctgggaactcggcatcggcgagccggtgcagatctcggccgagcatggtcttggcatgcccgacctgcgcgacgccgtcgtcgaggcgctcggcagggaagtcgtttttgccgatgacgaggacgaggccggcgacgtcgccgaaagcgacgtgctgatcggcgaggacatcgtcgatcccgacgccgaggaggcgcccgcctatgacgagaccaagccgctgcgcatcgccgtcgtcggccggccgaatgccggcaagtcgacgctgatcaatgcgctgatcggcgaggagcgcctgctcaccggacccgaggccggtatcacccgcgattcgatttcggtcgactgggactggcagggccgcagcatcaagatgttcgatacggccggcatgcgccgcaaggccaaggtccaggaaaagctcgagaagctgtcggtgcaggacggcctgcgcgccatccgtttcgccgagatcgtcatcatcgtgctcgatgcgacgatcccgttcgagaagcaggacctgcagatcgccgacctgatcatccgcgagggcagggcacccgtcattgccttcaacaagtgggatctcatcgacaacccgcaggaaacgctggccgagctgcgcgagaagaccgagcggctgttgccgcaggcgcgcggcctgcaggcggtgccgatgtcggcggagaccggccgtgggctggagaagctgatggacgctgtcatgaagacccaccgggtctggaacagccgtgtctcgaccggcaagctcaaccgctggctcgagggcatccttgcccatcatccgccgcccgccgttgccggccgccggctcaagatcaagtacatcacccaggcaaagacccggccgcccggtttcgtcctgtcctgctcgcgccccgacgtgatgccgcaatcctatgtgcgttatctcgtcaacaatctgcgcgacgccttcaacatgcccggcgtgccgatccgcatggcgctgcgcgcctccgacaatccgtttgccgggcgggccaagaagaagaaatga

SEQ ID NO: 98 - DNA, 1985 bp, Bacillus sp.
  Marker nucleic acid sequence
tgaaagatcgctttgagttagtctcgaaataccagccccagggagatcagccgaaagctattgaaaaacttgtaaaagggattcaagagggcaagaagcatcagactctgctgggtgcgacaggaacggggaaaacatttacggtgtccaatttgattaaagaagtcaataagccgacgctcgtgattgcccataacaaaaccctcgccggacagctttacagtgagttcaaggaattttttccgaacaacgctgtcgagtattttgtcagctactatgattattatcagccggaggcgtatgtgcctcaaacggatacgtttatagagaaagacgcaagtattaatgatgaaattgataaactgagacactcggccacatcgtctctgtttgagcggagagatgtcattattattgcgagtgtgtcttgtatatatggtctcggttcgcctgaagaataccgggaaatggtcgtgtcactgcggcctgaaatggaaattgagcggaacgagctgctcagaaaacttgtagacatccaatatgcccgcaacgatatcgactttcagcgcggtacatttcgtgtgcgcggagatgtagtggaaattttcccggcatcccgtgatgaacattgtatcagagttgaatttttcggtgatgaaatcgaacggatccgggaagtggatgccctgacaggagaaattctcggcgaccgcgaccatgtcgcgattttcccggcatcccacttcgtaacgcgtgccgagaaaatggagaaagcgattcaaaatatcgagaaagagcttgaggagcagctgaaggtcatgcatgaaaatggcaagctgcttgaagcgcagcgtttagagcagcggacaagatatgatcttgaaatgatgcgggaaatgggcttctgctccggcattgagaactattcaagacatttgacgcttcggccgcctggttcaacgccgtatacgcttcttgattatttcccagacgattttatgattgtggtggatgagtcgcatgtgacgattcctcaggtgcgcggcatgtttaacggagaccaggcgcggaaacaggtgctcgtggatcacggcttccgtctcccgtcagcgcttgataaccgtccgctccgttttgaagagtttgaaaagcatatgcacaatatcgtgtatgtatcagccacgccaggaccgtatgagattgagcatacggatgaaatggtcgagcagatcatccgtccgaccgggcttcttgacccgcttattgatgtgcggccgattgaagggcagattgatgatttaatcggtgaaattcatgcgagagttgaacggaatgaacgggtgcttgtgacgactttgacgaagaagatgtcagaggacctgaccgattatttaaaagaaatcggcatcaaagttaattatctgcattcagaaattaagacgcttgaacggattgagatcatccgcgacctgcgccttggcaagcatgatgtgctcattggcattaacctcctgagagaggggcttgatattccggaagtatccctggttgcgattttggatgcggataaggaaggcttcctgcgttcggagcgttcgcttattcagacgatcggacgggcggcgaggaacgcggaaggccgtgtcatcatgtacgccgataaaatgacgaagtctatggaaattgcaattaatgaaacgaaacgccgccgcgaacagcaggagcgttttaacgaagagcacggcataacgcctaaaacgataaataaagaaattcgcgatgtcattcgtgccacagcagcagctgaggataaagcggaatataaaacaaaagctgcgccgaagctgtcgaaaatgacgaagaaagaacgtcagaaagtcgttgaacagatggagcacgaaatgaaggaagccgccaaggcgcttgactttgaaagagcggcggagcttcgtgatttacttttagagctaaaagcggaaggatga

SEQ ID NO: 99 - DNA, 1046 bp, Bacillus sp.
  Marker nucleic acid sequence
tgagtgatcgtcaggcagccttagatatggctcttaaacaaatagaaaaacagttcggcaaaggttccattatgaaactgggagaaaagacagatacaagaatttctactgtcccaagcggctccctcactcttgatacagcactaggaattggcggatatccgcgcggacggattattgaagtatacggtcctgaaagctcaggtaaaacaactgtggcgcttcatgcgattgctgaggttcagcagcagggcggacaagctgcgtttatcgatgcggagcatgcgttagatccggtatacgcgcaaaagctcggtgtcaacatcgaagagcttttactgtcgcagcctgacacaggcgagcaggcgcttgaaattgcggaagcgttggttcgaagcggcgcagttgacatcgttgttgtcgactctgtggcggctctcgtcccgaaagcggaaattgaaggcgacatgggagattcgcatgtcggtttacaagcgcgcttaatgtctcaagcgcttcgtaagctctcaggggccattaacaaatcgaaaacaatcgcgattttcattaaccaaattcgtgaaaaagtcggcgttatgttcgggaacccggaaacaactcctgggggccgcgcgctgaaattctactcttccgtacgtcttgaagtgcgccgagctgaacagctgaaacaaggcaacgacgtaatggggaacaaaacgaaaatcaaagtcgtgaaaaacaaggtggctccgccgttccgtacagctgaggtggatattatgtacggagaaggcatttcaaaagaaggcgaaatcatcgatcttggaactgaacttgatatcgtgcaaaaaagcggttcatggtactcttatgaagaagagcgtcttggccaaggccgtgaaaatgcaaagcaattcttgaaagaaaataaagatatcatgctgatgatccaggagcaaattcgtgaacattacggcttggataatcacggagcagtccagaagcaagctgaagatgcgcaagaagaactcgaatttgaagaataa

SEQ ID NO: 100 - DNA, 2024 bp, Stenotrophomonas sp.
  Marker nucleic acid sequence
tgagcgacagttttgaacttgtctcgccgtattcgccggcgggcgaccagcccgatgccatcgcgaagctgaccagcaacttcgaggccggcatcgccaagcagaccctgctcggcgtgaccggctcgggcaagacctacaccatcgccaacgtcatccagaacgtgcagaagccgacgctgatcatggcgccgaacaagacgctggcggcgcagctgtatggcgagttcaaggcgttcttcccgcacaacgcggtggagtatttcgtcagctactacgactactaccagccggaagcctacgtgccgtcgtcggacaccttcatcgagaaggacagttcgatcaacgagcacatcgagcagatgcgcctggccgcgaccaagaccctgctgtcgcgcccggatgcgatcgtggtggcgacggtgtcggcgatctatggcctgggtgcaccggaagactacctgtcgctgcgcctgatcctgtccaggggcgagcgcatcgaccagcgcgacctgatcaatcacctgacccagctgcagtacacacgtaacgagtacgagctgcagcgcggtaccttccgcgtgcgcggcgaagtgatcgacgtgttcccggccgagtcggacagcgaagcactgcgcctcgaactgttcgatggcgaggtcgagaagatcaccctgttcgatccgttgaccggcgagacgctgcgcaacatgcagcgcttcacggtctacccgaagacccactatgcgaccacgcgtgagcgcgtgctggcggcgatcgagacgatcaaggtggagctgaaggaacggctggagcagctgtacgcgcagaacaagctggtggaggcgcagcgcctggcccagcgcacgcagttcgacctggaaatgatggccgaggtcggctactgcaacggcatcgagaactactcacggcacctgaccggcaagaacgcgggcgagccgccgccgaccctgttcgactacctgccggccgatgcgctgctggtgatcgacgaatcgcatgtgaccattccgcagatcggcgccatgttcaagggtgaccgctcacgcaaggagaccctggtcgagttcggcttccggctgccgtcggcgctggacaatcgtccgctgcgtttcgaggaatgggaggagcgctgcccgcgcgcgatctacgtgtctgccacgccggggccttacgaataccgcgaggccggtgacgagatcaccgagctggtggtgcgcccgaccggcctgatcgacccggtggtggagatccgcccggtgggcacccaggtcgacgacctgatgagcgaggccaacgagcgcatcaaggcgggtgaccgcgtgctggtcaccacgctgaccaagcgcatggccgagaacctcaccgaatacctgaccgagcatggcatccgcgtgcgctacctgcactcggacgtggacacggtcgagcgcgtggagatcatccgcgacctgcgcctgggcaagttcgatgtgctggtcggcatcaacctgctgcgcgagggcctggacatgcccgaggtatcgctggtggcgatcctcgacgccgacaaggagggcttcctgcgttccaccggctcgctgatccagaccatcggccgcgctgcccgcaacgtgcgtggcaaggcgatcctgtatgccgacaggatcacccgttctatgcaggcggcaatcgatgaaaccgaccgtcgccgcgccaagcaggtcgagtacaacgaagaacatggcatcgtgccgcgctcggtggcgcgcccgatcgtcgatgtcctcgaaggcgcgcgttcggatgccgcggaaaaggaatccaagaagggcaagggcaagggtcgggcgggcgtggccgaagaggcggccgactatcgttcgctgtcgccggcgcagcttggggtccgtctcaaggcgctggaacagcagatgtaccagcacgccaaggacctggaattcgaggatgccgcgcgcgtgcgtgaccagatccggcagctgaaggaagccagcctgggctga

SEQ ID NO: 101 - DNA, 512 bp, Stenotrophomonas sp.
  Marker nucleic acid sequence
atgaaagatatcgagcgccgcatcctgctgggcagggttgtcggcgcttttggtgtgcgcggcgagatcaagctcgagtcctggaccgagccacgttccgccattttccgttaccagccgtggatcttgcgcagcccgtccggcgtggaaaccacgattgaaggtgtgcgcggccgcgacagcggcaagcacctggtcgcccgtttccccggcgtcgaggaccgcgataccgtcgaagcgatgcacggcaccgagatctacgtggcccgcagtgcgttgccgccgccgaagcccgacgagtactactgggtggacctggaaggcctggacgtgaagaccaccgagggcgttgccctgggccaggtctcgcacctgttcagcaccggcgccaacgacgtggtggtggtccgtggtgaccgcgagcggatgattccgttcgtgcagccggacttcgtcaagtcggtcgacttcgaggccaacctggtcgtggtcgactgggatcccgagttctg

SEQ ID NO: 102 - DNA, 1328 bp, Stenotrophomonas sp.
  Marker nucleic acid sequence
tgcagggcgcagccctgccgagcaaccctatacttgccgccgtgactgccttccctgccctcgtcccgctggatgcgccactgctggtgggctacagcggcggtgtcgactcgaccgtgctgctgcactggctgtggcgctgcgcccaggcttccggcaccgcgctgcgtgcggtgcatatacaccatggcctgcaacccgatgcggacggctgggtgctgcattgccagcagcaatgcgcagcacggggcatcgaactggccgtgcatcgcgtgcatgtcgacaacagcgcgggccttggccctgaaggtgcggcacggcaggcgcgacgcgccgcgttcgccgccgaactgcgcgacggcgaaacgctggcgctggcgcagcacctggacgaccaggccgagaccttcctgctgcgggccctgcgcggatccggcgtcgatggccttgccgcgatgtccgcgcacagctgccttggcgaacagcctctatggcggccgctgctggcggtgccacgcagtgcactgctcgactacgcacaccagcacgcgctgcgctggatcgaagaccccagcaacgccgacgatcacgccgatcgcaacttcctgcgtctgcaggtgctgccgttgctgcgccagcgctggccgcatgccgccgcggcattggcgggcagcgccctgcattgccggcagacccgcgagctgctggacgaggaagacgccgagctgatcgcgcacctggaggtggcaccgcgcgtgctgtcgctgcagctgctgcgccaggtgtcaccgggccgcgcggcgcgcgtgctccgtgcctgggtgatcggccatgtggccgcgccactgccggccaccgtgctgcagcaggcactggacgagctgctgccggccggcaacgatcgccaggcgcgggtacgctggcacgatcatgcgatccagcaatggcgcgatcatgcctatctcctgcccgcgcagctgccggcgcttccccatgattggcaggccggatgggatggacgcgcgccattggcgctgcccgatggcggtcagctgcgcctgcagggcgccgtggccttcgaacgcccgctccaggtgcgtgcgcgtgtgggcggcgaacgcatcctgctgcccggtcgccagcattcgcatgcgctgaaggactgcctgcaacgcgaacacctcgcgccctggcgacgcgcccagctaccgctggtcttcgatggaccacagctgctcgccgccgccgacgtggtcatcgcagctcccctgcaggcctggttgcaggcccacgacgcgcagctgcagtggcgtccgggcggctggtga

SEQ ID NO: 103 - DNA, 1397 bp, Stenotrophomonas sp.
  Marker nucleic acid sequence
tgctgcctttggtcgccctggttggacggccgaatgtcggcaagtcgactattttcaatgcgcttacgcgcacccgtgacgcgctggtccatgaccagcctggcgtcacccgcgaccgcaactacggcgtctgtcgtctggacgaggacaatcatttcctcgtcgtcgacaccggcggtatcgcggaagaggaagagggactggcaggggctaccacccgccaggcccgcgccgctgcagcggaagccgacctgatcctgttcgtggtcgatgcccgtgagggtacctcggcgatggacgacgagatcctggcctggctgcgcaagctgtcgcgcccgacgctgctgctgatcaacaagatcgacggcaccgacgaggacaccgtgcgttcggagttcgcccgctacggcttcggcgagatgctgaccgtgtcggccgcgcatcgccagggcctggatgacctgctggacgaagtgatccagcgcctgccggaagaaggcagcggcgaagagctggacaacgatccgaaccgcatccgcatcgccttcgtcggccgcccgaacgtgggcaagtcgaccctggtcaaccgcatcctcggcgaggagcgcatgatcgcctccgacgtgccgggcaccacccgcgactcgatcgccgtggacctggagcgtgacggccgcgaataccggctgatcgataccgccggcctgcgccgtcgttcgcgcgtggacgaggtcgtcgagaagttctcggtggtcaagaccatgcagtcgatcgagcagtgccaggtggccgtgctgatgctcgacgccaccgaaggcgtgaccgaccaggacgccaccgtgctcggtgccgtgctcgatgccggccgcgcgctggtcattgccatcaacaagtgggatggcctgactgagtaccagcgcgagcaggctgaaaccatgctgtcgctgaagctgggcttcgtgccgtgggccgagtccgtgcgcatctcggccaagcatggctcgggcctgcgtgagctgttccgtgcggtgcaccgcgcgcacgaatcggcgaacaagaccttcaccaccagcgaagtgaacaaggcgctggaagtggcgtacgagaccaacccgccgccgaccatccgcggccacgtctccaagctgcgttacgtgcacccggctggggcaaacccgccgaccttcatcgtgcacggcacgcgcctgaaggaactgcaggaatcgtacaagcgctacctggagaacttcttccgcaagcgcttcaagctgatcggcacgccggtgagcttcatcttccgcgagggcaccaacccgtacgagggcaagaagaacgtcctcaccgagcggcaggtcaaggccaagcggcgcttgatgaagcacgtcaagggcaagtga

SEQ ID NO: 104 - DNA, 2462 bp, Carnobacterium sp.
  Marker nucleic acid sequence
atggggaaaaaacgtgttcatgagtatgctaaagagcatgatatgtctagtaagcgtgtaattgaaaaagcaaaagaactgggaattgaattaggcaatcatatgtctactatcgatgaaaatcaagttcaaaaattaaatcaagtttttacaaaaaaagaagcaggaacaaaggttgctgctaatactaaaaaatcagaccgtccacactcaagtggacaagggggtaacactacgaactctaaagaagataatcagaagcgtccagctcaaagtggtggacaaaatgcaggtaaaccaactcaaacagggaagccagcacaaaataaaacaacaaatactcgtccagctactaaacctagccaaggacaatcaaataatacaaatcgtccagctgctaaaacaggtcaaggacaatcaaacaacacaaatcgcccagcagctaaaactagtcaagggcaatcaagtaacactagtcgtccagcggctcaaggccaaggaacaacaagtggaaataatgctcaaggagcaaatcgtggaagcggcggaggacaaggcggttatcaaggtaaccgtggtggacaaggcggcaatcgcggtggctatcaaggcggttataacaatcgcaataatttcagaaagaaaaaacgtcgtagtggcgaaacaaaaccagtagcaccaccagtgccacgtaaatttaaagaattaccagaagtattagtttattctgatggtatgacagttgcggatatttctaaaaagatttaccgtgaaccagcagagattattaagaagttattcctattaggcgtaatggctactttaaatcaatctttaagcaaagaagcaattgaattattagcaacagattatggtattgaagctgaagagaaaatcgaattagatgtatctgatttagatgtctactttgaccaagagaccaacgaagaggctttagttactcgtccaccagttgttacaattatgggtcacgttgaccatggtaaaacaacacttctagattcattacgtaatacaaaagtaagtttaggagaagcaggcggaatcacacaacatattggagcttatcaaattgatgttgatggaaaaccaatcacattcttagatacaccaggacatgcggcctttacaacgatgcgtgctcgtggagcaggtatcacagatattactgtattagttgtagcagcagatgatggtgttatgccacaaactgtcgaagcaattaaccatgctaaagcagcagaagtaccgattattgttgcagttaataaagttgacaagcctggtgcaaatccagagcgtgtgatgcaagaattaactgagcatggattggttcctgaatcatggggtggagaaacaatttttgttgagatttctgctaaattccaacaaaacttagaagaactacttgaaatgattttattagttgctgaagttcaagaattaaaagctgacccaactcgtttagctattggaacagtaattgaagctcgtttagataagagtaaaggtccagttgcaacgctattagttcaagaaggaacactaaaagttggagatccaattgttgttgggaatacatttggtcgggttcgtgtgatgtctaacgaaactggtcgtcgtgtgaaaacagcgggtccagctgcaccagttgaaattactggattaaacaatgctccacaagctggcgatcgttttgttgtctttgaagatgaaaaaactgctcgttcagcaggtgaagatcgtgcgaaacgtgctgctgttgagcaacgttctgttacgaatcgtgtaacattagataacttgtttgatagcttacaagaaggcgaattaaaagaagttaacgtgattatcaaagcagatgtacaaggatctgttgaagctttagcagcaagcttacaaaaaattgaagttgaaggcgttcgtgttaaaatcattcatacggcagttggagctattaatgaaagtgatattactttagctgcagcaagtaatgcaattgtgattggatttaacgttcgtccaacacctcaagctaaacaacaagctgaacaagaagccgttgatattcgcttacaccgcattatttataatgctatcgatgaaattgaaacagcaatgaaagggatgttagatcctgaatttgaagagaaaatcactgggcaagcaattgttcgtgagaccttcaaagtgtctaaagttggaacaatcgcaggtggttatgtaactgaaggctttatcacacgtaacagtagcattcgtttaatccgtgatagcattgttatttttgaaggcgaattagctagtttgaaacgctttaaagatgatgttaaggaagttaaacttggatttgaatttggtttcatgattaaagattacaatgaagttcaaatggatgatgttattgaagcataccaaatggtagaaattaaacgtaaata

SEQ ID NO: 105 - DNA, 1148 bp, Carnobacterium sp.
  Marker nucleic acid sequence
atgactgcagcatatattcatattcctttttgtgagcatatttgtttttattgtgattttaataaagttttcttagaaggtcaaccagttgatgaatacgtggatttactggttagggaaatgaaattaacaatggaacaacaaggagaaaccgaaatcgaaacgatttatgttggtggtgggactccaacaacattaaatgaaaaacaattagatcgtttattaattggaatcaaggaaaatttaccttttaaaattgggaatgaatttacttttgaagcgaatcctggtgatttgtcagttgaaaaattacaagttttacatgatcatggagtcaatcgtttaagtatgggcgttcaatcattcaatgatgacttactaaaaaagattggacgaatccaccgtgtttccgatgtctatcaatctattagtaacgccagacaagtcggttttgaaaacatcagcattgatcttatttatcgcttgccaggtcaaactgaggctgattttgaatcaagtttaaaaaaagctttagagctagatttacctcattattctacctattcgttaattttagagaacaagacgattttttataatttaatgcggcaggggaagctgcctttaccaacagaagatgctgaggctaatatgtatcaaatggctattgatcaaatggcggacaaagggcgtaagcaatacgaaattagtaactatgctttaccaggttatgagtctaaacataacctgatttattggaaaaatgaaaaatattatggttttggtgcgggggctcatggctatttaggtggacatcgttaccaaaataatggtccaatccaacaatatttaaagcctttaagagagcataaattgccagttttaagaagccaaaaattatcacttgaggaaaaaatagaagaagaattattcttaggtttgcgcaagttgcaaggcgttagcattgaacactttttcgacaaatttcaagtttctgtttttgatatctatcaagaaacgattgaggaattagttgaagctggtttgttagaagtcagccaacagtggttacgtttaactcctagagggaaatttctaggcaacgaagtttttcaagcgttcttattgagcgatggaaaaaataaata

SEQ ID NO: 106 - DNA, 1331 bp, Carnobacterium sp.
  Marker nucleic acid sequence
atgaataaacaaacaacgtatcattttgtcggaattaaaggatcaggtatgagcgcactagcattagttttacatgacaagggatttaaagtccaagggtcagatgtagacaattactatttcactcaaaaaggaatagaagacgcgaagattactattttacctttttcaaaagaaaacattcacgatggcttaacaatcatagctggaaatgcctttccagatactcatgaagaaattaaagcagcgctagatatgggaataccagttattcgttaccataagtttattggagattttttaaatgactatacaagcgttgcaattacaggttctcatggtaaaacaagtacaactggattgcttgctcatgttttaagttctatcgagccgacaagttacatcattggagatggtacaggacacggagttcctgatgctgaattttttgttttagaagcttgcgaatatcgtcgtcatttcttagcctattcaccagactatgctattatgacaaatattgactttgatcatcctgactatttcactggtatagaagatgttttttctgcgttccaaacaatggcaaatcaagttaaaaaagctattattgcctgtggtgaagatgaggaattaaaaaaattaaaagcagatgtacccgttctttactatggtttttctgatgatcataattaccaagcaaaaaatattgttcgaaccactaaaggttctggttttgatgtctatgtagaaaaagaattctttgggcattttgaaattccaacatatggtgcgcataatgttttaaacgccttagctgttattgctgtgtgtcaattagaaggtatttcagctgaattaatcgccgagaacctaactactttccaaggggtaaaacgtcgttttagcgagaaatcaatggcagatatgattattattgatgattatgcacatcatcctcaagaaattcgtgctactttagatgctgcacgtcaaaaatatccagataaagagttgattgctgtttttcaaccccatacatttacacggacaattgctttgctaagtgattttgcagcagcattaaatttagctgatcgtgtttatttatgtgatattttcggttctgctagagaagagcaaggaaatgtaacgattcaagatttagcagataaaattaccaatggtgcattagttttacaagaagaaaatatgtcgccattattagatcatcatgatgcagttgtgattttcatgggggcaggcgatgttcaaaaatttgaatatgcttatgaaactattttaagtcgtacaattttacaaaataatta

SEQ ID NO: 107 - DNA, 1481 bp, Carnobacterium sp.
  Marker nucleic acid sequence
atgtctaactgggaaacgaaatttgctaaagagggctacacttttgatgatgttttattggtccctgctgaaagccatgtgctaccaaacgatgttgacatgagtgtcaaattggctaaaaatattacactaaatattcctttgatgagtgcgagtatggatacagtcacagattcaaaaatggcgattgcaatggcgcgtcaaggtggtttaggtgttattcataaaaatatgagtacaaaacaacaagctgacgaagttagaaaagtaaaacgttctgaaagtggcgttattattgatccattctttttaactcctgatcatttagtttcagatgcagaagaattgatgggccgttaccgtattagtggagttccaattgtcaataatatggaagatcgaattctagtaggtattttaaccaaccgcgatttacgctttgtaacagattacagtattaaaattgatgaagttatgactaaagatcaacttgtgacagctccagttggaacatctttaaaagatgctgaaaaaacattacaaaaacataaaattgaaaaattaccaattgttgacgctgaaggacgtttaagtggtctgattacaattaaagatattgaaaaagttatcgagttccctaatgccgctaaagatgagcatgggcgcttgttagttgctgcagctgttggagtaacaacagatacctttgaacgcgcagaagcgctattagacgctggtgcagatgcaattatcattgatactgctcatggacatagtgcaggtgtgattcgtaaaatcaaagaaattcgtgaacatttcccagaagcaacattaatcgctggaaacgtagcaactggcgaagcaactcgtgctctttacgatgttggtgtagatgtggttaaagttggaattggacctggttcaatttgtacaactcgagttgttgccggagttggggttcctcaactaacagctatttatgatgctgctgaagtagctcgtgaatatggacgtaccattattgcagatggaggaattaaatattctggcgatatcgttaaggcaattgctgccggtggacatgctgttatgttaggcagtatgttagccggaacagacgaatcaccaggcgaatttgaaattttccaaggccgtcgtttcaaaacataccgtggcatgggtagcttaggcgcaatggaaaaaggttcaagcgatcgctatttccaaggtggcacaaatgaagcgaacaaattagtcccagaaggaattgaaggtcgagttgcctataaaggtggcgttagtgacattattttccaaatgattggtggtctaaaagccggaatgggctatgttggagcagctgatttgaaatatctacgtgatgatgcacaatttatccgtatgagtggtgcaggtttacgcgaatctcacccacatgacgttcaaattactaaagaagcaccaaactattcattacaata

SEQ ID NO: 108 - DNA, 1091 bp, Acinetobacter sp.
  Marker nucleic acid sequence
tgggttttaattgcggtattgttggtctgccaaacgtagggaaatctacccttttcaatgcattaacgaaagcagcgattgcagcggaaaacttccctttctgtaccattgaaccaaacacaggtattgttcctgtaccagatccacgtctagacaaacttgctgcgattgttaaaccgcaacgtattttgccaactacaatggaatttgtagacattgcaggtcttgttgctggtgcatcaaaaggtgaaggcttaggtaaccaattccttgctaacattcgtgaaactgatgcaattgctcacgtggtacgttgtttcgaagatgaaaacgtgattcacgtaaatggtaaaattgatccgttagatgacatcgcaaccatcaatactgaacttgcacttgcagaccttgaaactgttgctaaagcaattttacgcttaactaaagttgctaaaggcggtgacaaagaagctgttgcaactaaagcagttttagaaaaaatccaaccattactagacgaaggtaaaccagctcgtgcggctgatttgtctgatgacgaacgtaaattagttcgtggttttggtttaatgactttaaaaccaacgatgtatattgccaacgttgctgaagacggttttgaaaataatccgcatttagatgcagtaaaaaaacttgctgctgaagaaaacgcaattgttgtccctctttgcaaccaaatcgaagctgaaatttcattattagaagatgaagatcgtgctgagttcctagaagcaatgggcatggaagaaccaggcttaaatgtggttattcgtgcaggttatgcgcttttaggtttacaaacttatttcactgctggtgtacaagaagtacgtgcatggacagtaaaagttggtgcaaccgctcctcaagcagctggtgtgattcatactgacttcgaaaaaggctttatccgtgctgaagttgttgcatatgatgacttcgtacaatacaacggtgaaaacggcgctaaagaagctggtaaatggcgtttagaaggtaaaacttacatcgttcaagacggcgatgtaatgcacttccgttttaacgtttaa

SEQ ID NO: 109 - DNA, 2021 bp, Acinetobacter sp.
  Marker nucleic acid sequence
ttgaacgaaaatcaacctttcgatttggtcactcattatcagccagcaggtgatcagccacaggcaattgaaaagctggtcaatggtatcgagcacggttttcgtaatcagttattgctaggggtaacgggctcaggtaagacctatactatggcaaatgtgatcgctcaaacacagcgacctacaattgtgatggcccacaacaaaactttggctgcccagctttatggtgagtttaaatctttcttccccaataatgcggtcgaatattttgtaagttattacgattactaccaaccagaagcttacgttccatcttcggatacgtttattgaaaaagattcagccattaatgatcacattgaccagatgcgattatcagcaacacgagctttattagaacgtcgtgatgcaattattgtggcatcggtttctgcaatttatggtttgggtgacccgaatgcttatatgcaaatgttgctgcatgtggttgagggcgaccgagtaagccgcgatgaaatcatccgccgattggttgaaatgcaatatacgcgcaatgaacttgaatttttacgtggtacttatcgtattcgcggcgaaattattgatattttcccagcggaatctgatcaacatgcgattcgtattgaactatttgatgatgaagtcgattcaatccgttggtttgacccgctaactggtaaaatggttcgtaaagtttctcggataacgatttatccgaaaagccactatgtaacaccaaaagatcatttgacccgtgccattgatacaattaaagatgaattacaagatcagcttaaattcttccgtgaacatgacaaattgcttgaagcacaacgtatcgagcaacggacacgttacgatctggaaatgatgcaacagttgggttataccaatggtatcgaaaactattcgcgtcatttgtctgggcgtccgtctggtgaagcaccgccgactttattcgactatattccagaagatgctttgctcattattgacgaatcacacgttactgttccgcaaattggagcgatgtataaaggtgaccggtcacgtaaagaaaacttagtgaattatgggttccgtttgccaagtgcgcttgataaccgtccaatgaaatttgaagagtgggagcgtattgtaccgacgaccatttttgtaagtgcaacacctgctaaatatgaattagaaaagtcagatcaaatcgttgaacaagtggtacgtccaacaggtctaatcgaccctgaaattgagattcgtcctgtcttaacccaagtagatgatgttctttctgaaattaatattcgtaaaaacttgaatgaacgggtattaattaccactttaaccaagcgtatggcagaagacctgacttcttatttaaaagaatatggcgttaaggtcgcttatctccactcagatattgatacggttgaacgcgtcaaaattattcatgaactacgtacaggtgtttttgatgttttagtcggcattaacctgctacgtgaaggtctggatatgcctgaagtttctttggtggctattttagatgcagataaagaaggctttttacgttcagaacgctcactcattcagacgattggtcgtgctgcgcgtaatgtgaaaggtaaagcaattttatatgctgaccgaattaccgattctatgcaaaaagcaatagatgagactgatcgccgtagagccaaacaaattgaatttaatgagttgcatggtattacgccacgtagtgctgttcgtcaggtcatcaaagaaattgactcgggtgaggtactttcagacgatgagattgatgaaaaagtacttgaacaagcacaagcattgagtgccgacgaaagacatattctttcagatcctaaactgtttagtaagcatattactaagcttgaaaaagatatgttaaaggcctcgaaagatttacagttcgagcaggcagcgcggatacgtgatgaaatcattcgattaaaagcgcaaatgttgcaata

SEQ ID NO: 110 - DNA, 2468 bp, Acinetobacter sp.
  Marker nucleic acid sequence
tgagttcagagtctcaatcagcctctcaaacagaacaaaccaatgaaaaggcttatgattcctctagtattaaagtattacgtggattagatgcagttcgtaaacgtccgggtatgtatattggggatacagacgacggtaccggtttacaccatatggtattcgaggtagtcgataacgcaatcgatgaagctttagctgggcattgtgatgaaattattgtcactattcatgaagatgaatcggtgagtgtttctgataatggtcgtggtattccaaccgatattcaccctgaagaaggtgtttctgcagcggaagtaattcttaccattctgcacgcaggcggtaagtttgatgacaacagttataaggtttcaggtggtttacacggtgtaggtgtttctgtagtaaacgcgctttcgagtaaattacacttaatgatttctcgtgctggtcaagtgcatgagcaagaatatcaacacggtgatccgcaatatccattacgtgtaattggtgaaacggataagagtggtacaacagtacgtttttggccaagtgaattaacatttacccaaaccatttttaacgtagaaattctagcacgccgtttacgtgagctttcattcttaaacgcgggcgtacgtattgtattacgcgatgaacgtattaaccttgaacatgtatatgactacgaaggtggattatctgagtttgtaaaatacattaacgaaggtaaaactcatctaaatgagattttccattttactgcggatacagataacggtattggtgtagaagtagccttgcagtggaatgaaagttatcaagaaaacgttcgttgttttacaaataacattcctcaaaaagatggcggtagccatttagctggtttccgtgcagctttgacgcgtggtttaaaccaatatcttgaaaatgaaaatattctcaagaaagaaaaagtcaatgtaacgggcgatgatgcacgtgaaggtttaaccgctattatttcggtaaaagtacctgatccaaaattttcatctcaaaccaaagaaaaactggtatcgagtgaagtaaaacctgctgttgagcaagcaatgaacaaagagttctctgcttacttacttgaaaacccgcaagctgcaaagtcaattgctggtaagattattgatgctgcacgtgcgcgtgatgctgcacgtaaagcgcgtgaaatgacacgtcgtaagagcgcacttgatattgctggtttgccaggtaaattggctgattgtcaggaaaaagatccagcactttctgaattatatcttgtcgagggtgactctgcgggtggtagtgccaagcaaggccgtaaccgtaaaatgcaggctattttgccgttaaaaggtaaaatcctgaacgttgaacgtgcacgctttgacaaaatgatttctagtcaggaagtaggtactttaattactgctctcggctgtggtattggccgtgaagaatataatcctgacaagctacgctatcataaaatcattatcatgaccgatgccgacgttgatggttcgcatattcgaaccttattattaacgttcttcttccgtcaaatgccagaattggttgagcgtgggtatatctatattgcacagccacctttatacaaattgaaaaaaggtaagcaggaacaatacatcaaagataatgatgcattggaaaattatttaatttcaaatgcgattgatgagctatctttgcatacgaatgctgaagcaccatcaattacaggtgaagcacttgctaaagtcattcaggattatcaagtttcgcaaaagagcttacagcgcttaacgttacgttacccagctagcttactagatgctttacttgaaatagaggcattcaagagcgatcaaaaccatgatgaagcttatgtacaacaatgggctgatcaagtaaaagacatcattacaagattacagccaagtttacgtccagaaattacactagagtcatttgagcgtgaaaatgctcagggtgaaaaatctgcgcattattggccgcgtattactgtatatgttcataaccttccacatgcatatttgcttgatgcaggtttattgaactcagctgagtatgctcgtttgctgaaaaactcgaagagttggttcagtttgcttgaagatggtgcttatttacaaaaaggtgatcgccgtattcaagtggctaacttccatcaagtttggcagcatattttgcaagattcgcgccgcggtatgatgatccaacgctataaaggtcttggtgagatgaatgctgagcagctttgggaaacgacaatggaccctgaaaaccgtcatatgttacaggtaaccattgacgatgcgattgaagcagatcgtatgttctcttgcttaatgggtgatgatgtggaaccacgtcgtgcattcattgaagagaatgctttaaatgctgatattgatgcttaa

SEQ ID NO: 111 - DNA, 1409 bp, Acinetobacter sp.
  Marker nucleic acid sequence
tgaaacccgttattgcgctcattggtcgtccgaatgtcggaaaatcaacgttatttaaccagattaccaagagtcgtgatgcacttgtagccgacttcgcgggcctgactcgtgaccgtaaatatggtgatgcgacttatcagaataagtctttcattgttgttgatactggcggtattggtgaaaatgaaggcggtattgataactacatggctgagcagtcaaaaacagcgatccatgaagcagatatcattattttcgtggttgatgcgcgtgctggcttgttggcttctgatgagcaaattgcacgggaattgcgtacgctaggtaaaaaagtttacttggttgccaataaagttgatggtgttcatgcagaagctgcgttggttgagttttacaaacttggtatgggtgaaccattacaagttgctgccagccatggacgtggtgtacagcaaatgcttgaggatgtcttggtagacgtacctgaagatgaaaatccagaagagcatgacaaagcaacaggtttacgtttagcgattattggtcgtccaaatgttggtaaatctaccttagtcaatcgcttgcttggcgaagatcgtgtagtggcatttgaccaacctggtacaactcgcgattcgatttatatcccatttgaacgtgatggtcgtcaatataccctcattgatacagcgggtgtgcgtcgtaaaggtaaagttgaagaaatgattgagaaattctcaatcgtgaaaacactacaagcaattaaagatgcacatgtgattgtagtggttcttgatgctcgtgaaggcgtagttgaacaagatttacatttgattggttacgcacttgaagctggtcgtgccatggtgatcgccattaacaaatgggacaacatgacagaatacgatcgtaaacaatgtaagttggatgttgatcgccgttttgactttattccatgggcaaaagtccatttaatctcggcattgcatggcactggtgtaggggaaatgtacccaaccatccatcgtgcttatgactcatcacatttaaaagtttcacctgcgaaattaacccaaattttgaatgatgctacagatgcacatcaaccgccaatggtgggtggtaaacgtattaaaatgcgttatgcgcatatgggtggacaaaatccaccaacgatcgtaattcacggtaataaggtcgataaaaccccagcggattatcgtcgttaccttgagaatgtgttccgcaaagtttacaaactggaaggaacgccagtgcgtgtcgattttaaaacgtcggaaaacccatttgaaggacgtaagtcacaagtggatgagcgcgttgcggcacgtaaacgtcgttatgtacagaaattcaaaaaagctgagaaaaagtttaaacgttaa

SEQ ID NO: 112 - DNA, 2012 bp, Serratia sp.
  Marker nucleic acid sequence
tgagtaaacttttcaaactgcattccgagttcaaaccggccggcgatcagccggaagccatccgcaagctggaagaggggctggaagacggcctggcgcaccagacgctgctgggggtcaccggttcgggcaaaacattcaccatcgccaacgtgatagccgatctgaatcggccgaccatggtgctggcgccgaacaagacgctggcggcgcagctgtacggcgagatgaaagcgtttttcccggagaatgcggtggaatacttcgtctcctactacgactactaccagccggaagcctacgtgcccagctccgacaccttcattgaaaaagacgcttcggtgaacgagcacatcgagcagatgcgtctttccgccaccaaggcgttgctggagcggcgcgacgtggtggtggtggcgtcggtatcggcgatctacggcctgggcgatccggatctttacctgaagatgatgctgcacctgacccagggaatgatcatcgatcagcgctccattttgcgccgcctggcggagctgcagtattcccgcaacgatcaggctttccagcgcgccaccttccgcgtgcgcggcgaggtgatcgacatcttcccggcggaatcggacgagctggcgctgcgcgtcgagctgttcgacgaagaagtggaacggctgtcgctgttcgatccgctgaccgggcagattgagcaggtggtgccgcgttttaccatttacccgaagtcgcactacgtgacgccgcgcgagcggatcatgcaggcgatggaagagatcaaggtcgatctggcggaccgccgcaaggtgctgctggccaacaacaagctgctggaagagcaacgcctgacgcagcgcacccagttcgatctggagatgatgaacgagctgggctactgctcgggcatcgaaaactactcgcgctacctttccgggcgtgccgagggcgagccgccgccgacgctgttcgactaccttccggcggacggcctgctggtggtcgacgaatcccacgtcaccattccgcagatcggcgcgatgttcaaaggcgaccgcgcgcgcaaggaaacgctggtggagtacggcttccgtctgccgtcggcgctggacaaccgcccgctgcgcttcgaggagttcgaggcgctggcgccgcagaccatctatgtttccgccacgccgggcaaatatgagctggagaaatccggcggcgatctgatcgatcaggtggtgaggccgaccgggctgctggatccgatcgtggaagtgcggccggtcgctactcaggtggacgatctgctctccgagatccgcaagcgcgcggcgatcaacgagcgcgtgctggtgaccaccctgaccaaacgcatggcggaagacctgaccgaatacctggaagagcatggcgagcgcgtgcgctacctgcactcggatatcgataccgtggagcgggtcgaaatcatccgtgacctgcgtctgggcgagtttgacgtgctggtgggcatcaacctgctgcgtgagggcctcgacatgcctgaggtgtcgctggtggcgattctggacgccgacaaggaaggcttcctgcgttccgagcgttcactgatccagaccatcggccgcgcggcgcgtaacctgaacggcaaggcgatcctgtatggcgatcgcatcaccgattcgatggccaaggcgatcggtgaaaccgagcggcgccgcggcaagcagcaggcctataacgaagccaacggcatcgtgccgcaggggctgaacaagaagatcggcgatattctgcagatcggccagccggtcaaccgcgccaaggggaaaggcaggagcaaggcggcggacggtggcgctcagctgcagaacctcacgccgaaagcgctggatcagaaaatccgcgatctggaggcgcagatgtacacccatgcgcagaacctggagttcgagcaggcagctgcgctgcgcgacgaaatccaccagctgcgcgagcagttcatcgcgatttcctga

SEQ ID NO: 113 - DNA, 2414 bp, Serratia sp.
  Marker nucleic acid sequence
tgtcgaattcttatgactcctcaagtatcaaggtattaaaagggctggatgcggtgcgcaagcgcccgggcatgtatatcggcgataccgatgacggcaccggtctgcaccacatggtattcgaggttgtggacaacgctatcgacgaagcgctcgcgggccactgtagtgacattcaggtcaccatccatgccgacaactcggtatcggtgcaggatgacggccgcggcattccgaccggcattcacccggaagaaggggtttcagccgcagaggtcatcatgaccgtgctgcacgccggcggtaaattcgacgacaactcctataaagtttccggcggcctgcacggcgtgggcgtctcggtggttaacgccctgtcggagaaactggagctggtgatccgccgcgaaggcaaagtgcacgaacaaacttacagccacggcgagccgcaggcgccgctgaacgtggtgggcgaaaccgagcagaccggcaccatggtgcgcttctggccgagtcaccagactttcaccaatgtgaccgaattcgagtacgacattctggccaagcgtctgcgcgagctgtccttcctcaactccggcgtgtctattcgcctgaaggacaagcgcaccgaccgtgaagaccacttccattacgaaggcggcatcaaggcgtttgtcgagtacctgaacaagaacaaaaccccgatccacccgaacgtgttctacttctccaccgtgaaagacgacatcggcgtggaagtggcgctgcagtggaacgacggtttccaggaaaacatctactgcttcaccaacaacattccgcagcgcgacggcggcacgcacctggtcggtttccgtaccgcgatgacccgtacgctgaacagctacatggagaaggaaggctacagcaagaaggccaaggtcagcgctaccggcgatgacgcgcgtgaaggcctgatcgccgtggtgtcggtgaaggtgccggatccgaagttctcctctcagaccaaggacaagctggtctcttccgaggtgaaaaccgcggttgaaacgctgatgaacgagaagctggtggattacctgatggagaatccgggcgacgcgaaaatcgtggtcggcaaaatcatcgacgcggcgcgcgcgcgcgaagcggcgcgtaaagcgcgtgagatgacccgccgcaaaggcgcgctggatctggccggcctgccgggcaagctggcggactgccaggaacgcgatccggcgctgtccgaactgtacctggtggaaggggactccgcgggcggctctgccaagcaggggcgcaaccgcaagaatcaggctatcttgccgctgaaggggaaaatcctcaacgtcgagaaagcgcgtttcgacaaaatgctctcttcgcaggaagtggcgacgctgatcaccgcgctgggttgcggcatcggccgcgacgagtacagcccggacaagctgcgttatcacagcatcatcatcatgaccgatgctgacgtcgacggttcgcacatccgtacgctgctgttgaccttcttctaccgccagatgccggaaatcatcgagcgtggccacgtgttcatcgcacagccgccgctgtacaaggtgaagaaaggcaagcaggagcagtacatcaaagatgacgaggcgatggatcagtaccagatcgccatcgcgatggacggcgctacgctgcacaccaacgccagcgcaccggcgctcggcggcgagcagttggaaaaactggtggccgagcactacggcgtgcagaagctgatcggccgcatggagcgccgctacccgcgcgcgctgctgaacagcctgatctatcagccgacgctgaacgaaggcgatctcagcgatgaagccaaggtgaagacctggatcgcgtcgctggtgcaggcgctgaacgacaaagagcagcacggcagcagctatgatttcgtgatcttcgagaaccgtgagcgccagatgtttgagccggcgttgcgcatccgcacccacggcgtggataccgactacaagctggacttcgacttcatccacggcggtgaataccgcaagatctgccagctgggcgagcagctgcgcgggctgatcgaagagggcgcgttcatcgagcgcggcgaacgccgtcagccggtagacagcttcgaacaggcgctggagtggctggtgaaagagtcgcgccgcggcctgtcggtgcagcgttataaaggtctgggcgagatgaacccggagcagctgtgggaaaccaccatggatccggaaagccgccgcatgctgcgcgtgaccgtcaaggacgccatcgctgccgatcagctgttcaccacgctgatgggcgatgcggttgagccgcgccgcgcctttatcgaagaaaacgccctgaaagcggcgaacatcgatatctga

SEQ ID NO: 114 - DNA, 2015 bp, Pseudomonas sp.
  Marker nucleic acid sequence
atgtctgaattccagctcgtcacccgtttcgaccccgccggcgatcaacccgaagccattcgccagatggtcgaaggcatcgaagccgggctggcgcaccagacactgctcggggtgaccgggtcgggcaagaccttcagcatcgccaacgtgatcgcccaggtgcagcgcccgaccctggtgctggcaccgaacaagaccctggccgcgcagttgtacggcgagttcaagtcgttcttcccgaacaacgctgtcgagtacttcgtttcctactacgactactaccagcccgaagcctacgtgccgtcgtccgacaccttcatcgagaaggatgcgtcgatcaacgaccatatcgaacagatgcgtctgtcggcgaccaaggcattgctcgagcgcaaggacgcgatcatcgtcaccacggtgtcgtgcatctacggtctgggcagcccggaaacctatttgaaaatggtcttgcacgtcgatcgcggcgacaagctcgaccagcgtgcgctggtgcggcgcctggccgacctgcaatacacgcgcaacgacatggatttcgcccgtgcgaccttccgggtgcgcggcgatgtgatcgacatctacccggcggaatccgatcttgaagcgatccgcatcgagttgttcgatgacgaggtggagagcatttccgcgttcgacccgctgaccggcgaagtcatccgcaaacttccacgcttcaccttctatccgaagagccactacgtgacgccccgggaaaccctgctcgacgccatcgaggggatcaaggtcgagttgcaggagcgcctcgaatacctgcgcaacaacaacaaactggtggaagcccagcgtctggagcagcgcacccggttcgacctggagatgatcctcgagctcggctactgcaacggcatcgaaaactactcgcgctatctgtcgggccgtccggctggcgcagcgccgccgacgctctacgattacctgccggccgacgccttgctggtgatcgatgagtctcacgtcagcgtgccgcaagtcggcgcgatgtataagggcgaccgttcgcgcaaggaaaccctcgtggaatacggcttccgcctgccgtcggcgctggacaaccggccgatgcgcttcgacgagtgggaaggggtgagcccacagacgattttcgtctcggccacgcccggcaactacgaagccgaacacgccgggcgggtgatcgagcaggtggtgcgcccgaccggtctggtcgacccgcaggttgaagtgcgtccggcactgacccaggtcgacgatctattgtcggaaatcaccaaacgtgtggcggtggaggagcgggtgctggtcaccacgctgaccaagcgcatggccgaagacttgaccgattacctggccgatcacggcgtgcgcgtgcgttatctgcactcggacatcgacaccgtcgagcgggtcgagatcatccgcgacctgcgtttgggcacgttcgatgtgctggtggggatcaacctgctgcgtgaaggtctggacatgccggaagtctcgctggtggcgattctcgacgcggacaaggaaggcttcctgcgttccgagcgttcgctgatccagaccatcggccgcgcggcgcgtaacctcaatggccgggcgattctctacgcggatcgcatcaccggttcgatggagcgggcgattggcgaaaccgagcgccgtcgcgacaagcagatcgcctttaacctggccaacggcatcacgccgaagggcgttttcaaggacgtcgccgacatcatggaaggcgccaccgtgcccggttcgcgcagcaagaagcgcaaaggcatggccaaggccgccgaggaaaacgccaagtacgaagccgaattgcgctcgccgagcgagatcaccaagcgcattcgccaactggaagagaagatgtaccagttggcccgcgacctggaattcgaagcggcagcacagatgcgcgacgaaatcgccaagctgcgtgagcgtttgttggccgttta

SEQ ID NO: 115 - DNA, 2417 bp, Pseudomonas sp.
  Marker nucleic acid sequence
ttgagcgaagaaaatacgtacgactcatcgagcattaaagtgctgaaaggcctggatgccgtgcgcaaacgtcccggtatgtacattggtgacaccgacgatggcagcggtctgcaccacatggtgttcgaggtggtcgacaactcgatcgacgaagccctcgccggccactgcgacgacatcagcatcatcatccacccggatgagtccatcaccgttaaagacaacggccgtggcatcccggtagacgtgcacaaagaggaaggcgtttccgccgccgaggtcatcatgaccgtcctccacgccggcggtaagttcgacgacaactcctacaaggtatccggcggtctgcacggtgtaggtgtttctgttgtgaacgcgctgtccgaagaactggtactgaccgtacgccgcagcggcaagatctgggaacagacctacgtccacggcgtgcctcaggcaccgatggcgatcgttggtgacagcgaaaccaccggtacccagattcacttcaaggcttccagcgaaaccttcaagaacattcacttcagctgggacatcctggccaagcgcattcgtgaactgtccttcctcaactccggtgtcggcatcgtcctcaaggatgagcgcagcggcaaggaagagttgttcaagtacgaaggcggcctgcgtgcgttcgttgaatacctgaacaccaacaagactgcggtcaaccaggtattccacttcaacatccagcgtgaagacggcatcggcgtggaaatcgccctgcagtggaacgacagcttcaacgagaacctgttgtgcttcaccaacaacattccgcagcgcgacggcggcacccacctggtgggcttccgttccgcactgacgcgtaacctgaacaactacatcgagcaggaaggtctggcgaagaagcacaaagtcgccaccaccggtgacgatgcccgtgaaggcctgaccgcgatcatctcggtgaaggtgccggatccgaagttcagttcccagaccaaagacaagctggtgtcttccgaagtgaagaccgcggtcgaacaggaaatgggcaagtacttctcggacttcctgctggaaaacccgaacgaagccaaactggtcgtcggcaaaatgatcgacgctgcccgtgcccgtgaagcggcgcgcaaggcccgtgagatgacccgccgcaaaggcgcgctggacatcgccggcctgccaggcaagctcgctgactgtcaggaaaaagacccggcgctgtccgaactgtacctcgtggaaggtgactccgcgggcggctctgccaagcagggacgaaaccgcaagacccaggccatcctgccgctgaagggcaagatcctcaacgtcgagaaagcccgtttcgacaagatgatctcgtcccaggaagtgggcaccctgatcaccgcgctgggctgcggtatcggtcgcgaagagtacaacatcgacaagctgcgctatcacaacatcatcatcatgaccgatgctgacgtcgacggttcgcacatccgtaccctgctgctgaccttcttcttccgtcagttgcctgagctgatcgagcgcggctacatctacatcgcccagccgccgctgtacaaggtcaagaaaggcaagcaagagcaatacatcaaagacgacgacgccatggaagagtacatgacgcagtcggccctggaagatgcgagcctgcacctgaacgaagacgcccccggcatttccggcgaagcgctggaacgtctggtgaacgacttccgcatggtaatgaagaccctcaagcgtctgtcccgcctgtacccgcaggagctgaccgagcacttcatctacctgccggccgtgagtctggaaatgctcggcgaccacgccaagatgcaagactggctggcccagtacgaagtccgtctgcgcaccgtcgagaagtcgggcctggtctacaaggccagcctgcgtgaagaccgtgaacgtggcgtgtggctgccagaggtcgaactgatctcccacggcctgtcgaactacgtcactttcaaccgcgacttcttcggcagcaacgactacaagaccgtcgtcaccctcggcgctcaactgagcaccctgctcgacgaaggcgcgtacatccagcgtggcgagcgcaagaaagcggtcaccgagttcaaggaggccctggactggttgatggctgaaagcaccaagcgccacaccattcagcgatacaaaggtctgggcgaaatgaacccggatcagctgtgggaaaccaccatggacccaagtgtgcgccgcatgctgaaagtcaccatcgaagacgccatcggcgcagaccagatcttcaacaccctgatgggtgatgcggtcgaacctcgccgtgacttcatcgagagcaatgctttggcggtttccaacctggatttctg

SEQ ID NO: 116 - DNA, 1058 bp, Pseudomonas sp.
  Marker nucleic acid sequence
tggacgacaacaagaagaaagccttggctgcggccctgggtcagatcgaacgtcaattcggcaagggtgccgtaatgcgtatgggcgatcaggaccgtcaggcgatcccggccatttccaccggctctctgggtctggacatcgcactcggcatcggcggcctgccaaaaggccgtatcgttgaaatctacggtcctgaatcttccggtaaaaccacactgacgctgtccgtgatcgcccaggctcaaaaagccggtgcgacctgcgccttcgtcgacgccgaacacgccctcgaccctgagtacgccggcaaactgggcgtcaacgtcgacgacctgctggtttcccagccggacaccggcgaacaggccctggaaatcaccgacatgctggtgcgctccaacgccgttgacgtgatcatcgtcgactccgtggccgctctggtgccaaaggctgaaatcgaaggcgaaatgggtgacatgcacgtgggcctgcaagcccgtctgatgtcccaggcgctgcgtaaaatcaccggtaacatcaagaacgccaactgcctggtgatcttcatcaaccagatccgcatgaagatcggcgtgatgttcggcagcccggaaaccaccaccggtggtaacgcgctgaagttctacgcctcggttcgtctcgacatccggcgtaccggcgcggtgaaggaaggcgacgaagtggtcggcagcgaaacccgcgtcaaggttgtgaagaacaaggtggcttcaccgttccgtcaggccgagttccagattctctacggcaagggtatctacctgaacggcgagatgatcgacctgggcgtactgcacggtttcgtcgagaagtccggcgcctggtatgcctacgaaggcaccaagatcggtcagggcaaggccaactcggccaagttcctggcggacaacccggaagtcgcggccaagctcgagaagcaactgcgtgacaaactgctgtcgccagccgtgatcgccgactccaaggcttctgcggtcaaagagaccgaagacgatctggctgacgctgacatctga

SEQ ID NO: 117 - DNA, 617 bp, Microbacterium sp.
  Marker nucleic acid sequence
tgacgaggatcatcgcaggcagcgcgagaggggcccgactcgaggttccgggctcgggaacccgacccacgagcgaccgtgtacgcgaatcactgttcggcgcactggagtccctgagcgccatcgccggtgcacgggtcctcgacctgtacgccggctcgggtgccctcggactcgagtcgctcagccgtggagcagcgagcgtcgaattcgtggaacgcggtcgccaggccgcagccatcgtccgacgcaacgcggaagtcgtcgcgaaggcggggagcctcccgctcggacgcgttcacgagagcacggcgcatgccttcctgcatcgcgccgtcggaccctacgacctcgtgttcagcgatccgccctacgacctcgacgacgcggccatgacggccgacctcgtcgcgctcgcaccgctgctgtcctcggacgcggtggtggtggtggaacgagctcgtcggtccacgcctccggacttcggcgccgcgaacctggagcagttccgcgagaagtcgtacggcgacaccaccctgtggtgggccgagccagcgacggaggacgaggaccctggccccgtgggtcagcccgcggccgagtcccagtcccgatag

SEQ ID NO: 118 - DNA, 1025 bp, Microbacterium sp.
  Marker nucleic acid sequence
tgtctgatcccctcgacgccggcgagccctccgacgagaccgaactcgccatcgaaggcgccctgcggccgacgagcctcggagagttcgtgggccagcagaaggtgcgcggtcagctgcagctgctcctcgaggccgcccgcctccagagtcgtcccgccgatcacatcctcttggcgggacctcccggcctggggaagacgacgctcgcgatgatcgtcgcacacgagagcgaacgtccgctccggctgtccagcggcccggcgatccagcacgccggtgatctcgcggccctgctctcgagcctggtccccggtgaagtgctcttcatcgacgagatccaccgcatggcgcgctccgccgaggagatgctctatctcgcgatggaggactaccgcatcgacatcatggtgggaaagggcgcgggagcgacgagcatcccgctcgaactctcgccgttcaccctggtcggtgcgacgacccgctcggggctgctgcccaacccgctccgcgaccgcttcgggttcaccggtcacctcgagttctacgaggagggcgagctggagcaggtcatcgcacgatccgcggccgtgctcggcgtcgacctccccaccgactcgctcgccgagatcgcccgccgctcccgcggcaccccgcgcatcgcgaaccgcctccttcgccgggtgcgcgattatgcgctggtgcacacggggggagttgcgaccatccgcgaggtccgtgcggccctggatctgtacgacgtcgatccgatcggtctcgaccgcctggaccgtgccgtgctcgaagcgctcgtccgacggttccgcggtggcccggtcggcctcagcacgctcgctgtcgccgtcggtgaggaagccgagaccgtggagagcgtggtcgagccgtacctcgtgcgcatcgggttcctcgggcgcacgccccgcggccgtgtggcgatgccggaggcgtacgcgcacctgggtgtcgcgcatccggacgggatgcttaagttggatgacctataa

SEQ ID NO: 119 - DNA, 1091 bp, Pseudomonas sp.
  Marker nucleic acid sequence
atgggtatcaaatgcggcatcgtcggcctgcccaacgtcggcaagtcgaccctgttcaatgcgctgaccaaggcgggtatcgccgcggcgaacttcccgttctgcaccatcgagccgaacgtcggcatcgtgccggtgccggatccgcgcctgaacgagctggcagcgatcatcaacccgcagaaggtcatcccgaccgcggtcgagttcgtcgacatcgccggcctggtcgccggtgccgccagcggtgagggcctgggcaacaagttcctggcgcacatccgcgaagtcgatgcgatcacccacgtggtgcgctgcttcgagcacggcgacatcgtgcacgtggccggcaaggtcgatccgatctcggacatcgaaaccatcgacaccgagctggcgctggccgacctggacagcgtcgagaaggcactgaaccgtgccgagcgtgcggccaagggcggcgacaaggatgcggcggcgcgcaagccggtgctggccaagctgcaggctgcactgtcggagggcaaggccggtcgctcggtcggcctggacgacgaagagaaggcgctggtgcgcgatctgttcctgctgaccctgaagccggtgatgtacatcgccaacgtgctggaagacggtttcgagaacaacccgcacctggacgccgtgcgcgcgcatgccgccgccgaaggcgcgcaggtggtgccggtgtcggccgcgatcgaagaagagctgtcccagctcgacgacgaagaccgcgataccttcctggcggacctgggcctgagcgagccgggcctgaaccgcgtgatcaacgcggcctacagcctgctgggcctgcagacctacttcaccgcaggcgtgaaggaagtccgcgcgtggaccgtgcgcaagggcgccaccgctccgcaggctgccgcggtcatccacaccgacttcgagaagggcttcatccgcgccgaaaccatcgcgtatgacgacttcatcaagtacaagggcgaagccggtgccaaggaagccggtcgcctgcgcctggaaggcaaggagtaccgcgtgcaggaaggcgacatcctgcacttccgcttcaacgtctg

SEQ ID NO: 120 - DNA, 578 bp, Pseudomonas sp.
  Marker nucleic acid sequence
gtgagcggccgtgggggcaatgacggtcaggtccgcatcatcggcgggcgctggcgcaatacccgcctgccggtgccgaccctgccagggcttcggcccagcagcgaccgcgtgcgcgagacccttttcaactggttgatgcccagactgggcggcgcgcgggtgctggacctgtttgccggcagtggcgccttgggcctggaagccgtctcgcgcggcgccgcccacgccactctggtcgagcgcgacgcgcagctgggacgcaatctgaccgcggccgtggccaagctgcaggcctacgaccaggtcaccgtcgtgcaggccgatgcgctgcgctggctgcagggcgtaccggcacagcaggccgatctggtcttcatcgacccaccgttcgcggatggcctgtggcaggacgtgctggcgcagctgccgcggcatctggccgccgatgcctggctgtacctggaatcgccggccggtcacacgccggtgctgccgccggactggctgctgcaccgcgagggcggcacccgcgaggtgcgctttgccctgtaccgccgcgccactgctacactttg

SEQ ID NO: 121 - DNA, 1844 bp, Pseudomonas sp.
  Marker nucleic acid sequence
atgaccgacgttcccgctccggtcttcgacggcaaggccttcgcggcccagctcagcaccgctcccggcgtgtaccgcatgtacgcggcggacgacaccctgctgtacgtcggcaaggcccgtgcgctgcgcaaccgcgtcggcagctatttcaacggcagcccgaagaacgcacggatcatgtcgatgatctcgcagatcacgcgcatggacgtgaccgtgacgcgctcggaagccgaggcgttgctgctggaaaaccagctgatcaagtcgctgtcgccgcgttacaacgtctcgctgcgcgacgacaagacctatccgcatgtgctgctgacccgcgaggactggccgcgcatcgccctgcatcgcggcccgcgcgccattcccggtcgctatttcgggccctatcccggcgtgaccgcggtgcgcgaaacgctgaacctgatgcacaagctgttcaagctgcgcagctgcgaagacagcgtgttccgcaaccgctcgcggccgtgcctgcagtaccagatcggccgctgcagcgcgccctgcgtggagctggtaccggcgccggactacgccgagtcggtgcgccgcgctgcgctcttcctggaaggcaagagcgacgaactgacccgcgagctgggcgagcagatgcaggccgccagcgaggcgctggagttcgagcaggccgcgcgcctgcgcgacctgatctcctcgctgcgcagtatgcagacccgccagtacgtggacggccgcgccgcggatctggacgtgctggcggtggccatgcaggggtctcaggcctgcgtgctgctgctggccttccgcgatggccgcaacctcggtacccgcccgttcttcccgcgcaccaacggcgaggagagcccggaggaagtgctggccgcgttcgtctcgcagtactacatcgagttcgagccgccgcgcgagatcctgctggaccgcgagattcccgacgccgacctgctggtcgccgcgctgtcggcctcggccgagcgcaaggtgcagctgaagtggaacgtgcgcggcgagcgtgccggctacgtggagctggccagtcgcaacgcgcagctgaccctggccaccgagctcaacagccgcaatgctcaacacgcgcgcagcgatgcactacgcgacatgctgggcctggccgagccggtcaagcgtgtcgagtgtttcgacatcagccacaccctgggcgaggccaccgtggcctcatgcgtggtgttcgacgccgccggtccggtgcgcgcgcagtaccgccgtttcaacatcagcggcatcgagccgggcgacgactatgccgccatgcgccaggccatcgatcgccgcttccgccgtgcggtggaagagcagggcgtgctgccggatgtgctgctgatcgacggcggcgccgggcagctggcacaggcgcaggccgcactggccgacctgggcgtggaaggcgtgctgctggtgggcgtggccaagggcgtggagcgccgcgccggccacgaagcgctggtgatgcctgacggccgcgagctgcgcccgggcgcagccaacccggccctgcagttcatccagcaggtgcgcgacgaggcgcaccgcttcgccatcaccggccaccgcggccgccgccagaaggcgaggatgaccagcaagctggaggacatccccggtatcggcccccgccgccgcgccagcctgctcaagcattttggtggcctggtgggcctgaaagccgctggcgaagcggaaatcgccaaggtggaaggcatcaatgacgccctcgcggcacgtatctacgctaacctgcacgggttggccacgcctgatgcggccgagta

SEQ ID NO: 122 - DNA, 1397 bp, Pseudomonas sp.
  Marker nucleic acid sequence
tgctgcctttggtcgccctggttggacggccgaatgtcggcaagtcgactattttcaatgcgcttacgcgcacccgtgacgcgctggtccatgaccagcccggcgtcacccgcgaccgcaactacggcgtctgccgcctcgacgaggacaaccatttcctcgtcgtggataccggcggtatcgcggaagaggaagaaggtctggccggggctaccacccgccaggctcgcgctgctgctgcggaagcggacctgatcctgttcgtggtcgatgcccgtgagggtacctcggcgatggacgacgagatcctggcctggctgcgcaagctgtcgcgcccgacgctgcttctgatcaacaagatcgacggcaccgacgaggacaccgtgcgttcggagttcgcccgttacggtttcggcgagatgctgaccgtgtcggctgcgcaccgccagggcctggacgacctgctggacgaagtggtccagcgcctgccggaagaaggcagcggcgaagagctggacaacgatccgaaccgcatccgcatcgccttcgtcggccgcccgaacgtgggcaagtcgacgctggtcaaccgcattcttggcgaggagcgcatgatcgcctccgacgtgccgggcaccacccgtgactcgatcgcggtggacctggagcgtgacggccgcgaataccgcctgatcgataccgccggcctgcgccgtcgttcgcgcgtggacgaggtcgtcgagaagttctcggtggtcaagaccatgcagtcgatcgagcagtgccaggtggccgtgctgatgctcgacgccaccgaaggtgtgaccgaccaggacgccaccgtgctcggtgccgtgctcgacgccggccgcgcgctggtgatcgccatcaacaagtgggatggcctgaccgagtatcagcgcgagcaggccgaaaccatgctgtcgctgaagctgggcttcgtgccgtgggccgagtccgtgcgcatctcggccaagcatggctcgggcctgcgtgagctgttccgtgcggtgcaccgcgcgtacgaatcggcgaacaagaccttcaccaccagcgaagtgaacaaggcgctggaagtggcttacgagaccaacccgccgccgaccatccgcggccacgtctccaagctgcgctacgtgcacccggccggctccaatccgccgaccttcatcgtgcacggcacccgcctgaaggagctgcaggaatcctacaagcgctacctggagaacttcttccgcaagcgtttcaagctgatcggcacgccggtgagcttcatcttccgcgagggtaccaacccgtacgagggcaagaagaacgtcctcaccgagcggcaggtcaaggccaagcggcgcttgatgaagcacgtcaagggcaagtga

SEQ ID NO: 123 - DNA, 2015 bp, Pseudomonas sp.
  Marker nucleic acid sequence
tgtccgagtttcagttggtgacccgcttcaaacctgccggcgatcagcctgaggcgatccggcagatggtcgaggggctggaggccggtctttcgcaccagaccctgctgggtgtgaccggctcgggcaagaccttctccatcgccaacgtgattgcccaggtgcagcgcccgaccctggtcctggcgccgaacaagaccctggccgcgcaactctatggcgagttcaaggcgttcttcccgaacaacgcggtggagtacttcgtctcctactacgactactaccagccggaagcctatgtgccgtcgtcggacaccttcatcgagaaggatgcctcgatcaacgaccatatcgagcagatgcgactgtcggcgaccaaggcgctgatcgagcgcaaggacgtgatcgtggtctgcaccgtgtcctcgatttacggcttgggcagccccgaggagtacctgaaaatggtgctgcacctcgaccgtggcgacaagatggatcagcgcgcgctgctgcggcgcctggccgagctgcagtacacgcgcaacgacatggacttcgcccgcgccactttccgcgtgcgcggtgatgtgatcgacgtctttccggcggaatcggatctggaagccattcgcgtcgagttgttcgatgacgaggtggagagcatcagtgccttcgatccgctgaccggcgaagtgatccagaagctgccgcgcttcaccttctaccccaagagccactacgtcaccccgcgtgaaaccttgctggaggcggttgagcagatcaaggtcgagctgcagcagcgcctggagtacctgcgtggggcgaacaagctggtggaggcgcagcgcctggagcagcgcacgcgcttcgatctggagatgatcctcgagctcggctactgcaacggcatcgagaactactcacgctacctgtcaggccggccttctggagcgccgccacccactctctatgactacctgccggacgaagcgctgctggtgatcgacgagtcccacgtctcggtgccgcaggtcggcgcgatgtacaagggcgaccgctcgcgtaaggaaaccctggtggaatacggtttccgcctgccttctgcgctggataaccgaccgatgcgtttcgaggagtgggaggcggcaagccctcagaccattttcgtttccgctacgcctgggccttacgagggcgagcatgccgggcgcgtggtggagcaattggtgcggccgaccggcctggtcgacccgcagatcgaggtgcgtccggcgcgcactcaggtcgatgatctgctctcggagatccgtttgcgtgtggcagcgggtgaccgcgtgttggtcaccaccctgaccaagcgtatggccgaggatttgaccgactacctgggcgaccatgacgtcaaggtgcgttacctgcactcggacatcgataccgttgaacgggtggagatcattcgtgatctgcgtctgggcgctttcgacgtgctggtggggatcaacctgctgcgcgaggggctggatatgcctgaggtgtcactggtggcgatcctagatgcggacaaggagggtttcttgcgcagtgagcgctcgctgatccagaccatcggtcgcgccgcacgtaacctcaacggccgagcgattctgtacgccgacaacatgaccggctcgatgcagcgcgccattggcgagaccgagcggcgccgcgccaagcagattgccttcaacgaggccaacggcatcgtgcccaagggcgtgcagaaggacgtcaaggacatcctcgaaggcgctgtggtgcctggtgcgcgcagcaacaagcgcaagggcatggccaaggcggcggaggagagtgcgcgttacgagaacgagctacgttcgccgagcgagatcaccaagcgtattcgtcagctggaagagaagatgtatgccctggcgcgcgacctggagttcgaggctgcagcgcagatgcgtgacgagattcagaagctgcgggatcggttgttgcaggtgtga

SEQ ID NO: 124 - DNA, 626 bp, Pseudomonas sp.
  Marker nucleic acid sequence
tgaacagtatggctttgcagcgcgttggcctcgtaggagcgaccctacctgaacccatccaaatactgcttcctttgcaggcgcgcctgatgagccaggcgctgcgcaagatcaccggtaacatcaagaacgccaactgcctggtgatcttcatcaaccagattcgtatgaagatcggtgtgatgttcggtagcccggaaaccaccaccggtggtaacgcgctgaagttctatgcctcggttcgcctggacatccgtcgtaccggcgcggtgaaggaaggcgaagaggtcgtgggtagcgaaacccgagtgaagatcgtcaagaacaaggtcgcgccaccgttccgtcaggccgagttccagattctgtatggcaagggcatctaccgtaatggcgagatcatcgatctcggcgtgcagcagggcctggtcgagaagtcgggtgcctggtacagctacaagggcaacaagatcggtcaaggcaaggccaatgcagccaagtacctggaagacaaccaggaagtggcgcgcgaaatcgaaggtctgattcgcgagaagttgctggtcagcagcgctccggcaaaggctccggctgccgatctggctgatgctgaagtagacttctaa

SEQ ID NO: 125 - DNA, 1091 bp, Stenotrophomonas sp.
  Marker nucleic acid sequence
tgggtatcaaatgcggcatcgtcggcctgcccaacgtcggcaagtcgaccctgttcaatgcgctgaccaaggcgggtatcgccgcggcgaacttcccgttctgcaccatcgagccgaacgtcggcatcgtgccggtgccggacccgcgcctgaacgagctggcgggcatcatcaaccctcagaaggtcatcccgaccgcggtcgagttcgtcgacatcgccggcctggtggccggtgccgccagcggtgaaggcctgggcaacaagttcctggcgcacatccgcgaagtcgacgcgatcacccacgtggtgcgctgcttcgagcacggcgacatcgtgcacgtggctggcaaggtcgatccgatctcggacatcgaaaccatcgacaccgagctggcgctggctgacctggacagtgtcgagaaggcgctgaaccgtgccgagcgtgcggccaagggcggcgacaaggacgcagcggcgcgcaagccggtgctggccaagctgcaggctgcgctgtcggacggcaaggccggtcgttcggtgggcctggacgaggaagagaaggcgctggtgcgcgacctgttcctgctgaccctgaagccggtgatgtacatcgccaacgtgctggaagacggcttcgagaacaacccgcacctggaagccgtgcgtgcgcatgctgccgccgaaggcgcgcaggtggtgccggtgtcggccgcgatcgaagaagagctgtcccagctcgatgacgaagaccgcgataccttcctggccgacctgggcctgagcgagccgggcctgaaccgcgtgatcaacgcggcctacagcctgctcggcctgcagacctacttcaccgctggcgtgaaggaagtccgcgcgtggaccgtacgcaagggcgccaccgccccgcaggctgccgcagtcatccacaccgacttcgagaagggtttcatccgcgccgaaaccatcgcgtatgacgacttcatcaagtacaagggcgaagccggcgccaaggaagccggtcgcctgcgtctggaaggcaaggaataccgcgtgcaggaaggcgacatcctgcacttccgcttcaacgtctga

SEQ ID NO: 126 - DNA, 578 bp, Stenotrophomonas sp.
  Marker nucleic acid sequence
tgagcggccgtgggagcaatgacggccaggtccgcatcatcggcggtcgctggcgcaatacccgcctgccggtgccgaccctgccggggctgcggcccagcagtgaccgcgtgcgcgagacccttttcaactggttgatgcccagactgggcggcgcgcgggtgctggacctgttcgccggcagtggcgcgctgggcctggaggcggtctcgcgcggtgccgcccacgccaccctggtcgagcgcgacgcgcagctgggacgcaatctgaccgcggccgtggccaagctccaggccgccgaccagatcaccgtggtgcaggccgatgccctgcgctggctgcagggcgcacccgcgcagcaggccgatctggtcttcatcgacccgccgttcgccgatggcctgtggcaggacgtgctggcgcagctgccgcggcacctggccgccgatgcctggctgtacctggaatccccggccggccacgtgccggtgctgccgccggactggctgctgcaccgcgagggcggtacccgcgaggtgcgctttgccctgtaccgccgcgccactgctacactttga

SEQ ID NO: 127 - DNA, 1685 bp, Stenotrophomonas sp.
  Marker nucleic acid sequence
atgaccgaatcatttgccgaactgtttgaagccagccaggccaacctggccaagctgaagccgggcgccatcgtcagcggtaccgttgttgaagtccgcggcgacgtcgtggtgatcaacgctggcctgaagtccgaaggcatcgtgccgatcgaacagttccgtaacgacgctggcgaaatcgacgtcgccgaaggcgacatcgtcaaggtcgccctcgactcgatcgagaacggcttcggcgaaaccgtcctgtcgcgcgagaaggccaagcgcgcgatggtgtgggacgagctggaagaagcgttggaaaagaacgaaaccatcaccggccgcatcagcggcaaggtcaagggtggtttcaccgtggacatcaaggatgtccgcgccttcctgccgggttccctggtcgatgtgcgcccggtgcgcgatccggcctacctggaaggcaaggaactcgagttcaagctcatcaagctggaccgcaagcgtaacaacgtcgtggtctcgcgccgcgctgtcgtcgaaagcgagcactcggaagagcgcgagcagctgatggacaagctgcaggaaggcgcgatcctgaagggtgtcgtcaagaacctgaccgattacggcgcgttcgtggacctgggcggcatcgacggcctgctgcacatcaccgacatggcgtggaagcgcgtgcgtcacccgtcggaagtcgtgaacgtcggcgacgagctggacgtccgcgtgctgaagttcgaccgcgagcgcaaccgcgtttcgctgggcctgaagcagctgggcgaggatccgtgggacaacatcggccgtcgttacccggccaacagccgcgtcttcggcaaggtctccaacgtcaccgattacggtgcgttcgttgagatcgagccgggcgtcgaaggcctggtgcacgtctccgagatggattggaccaacaagaacgtcaacccgtccaaggttgtgcaggttggtgacgaagtcgaagtgatggtgctggacgtcgatgaagagcgtcgccgtatctcgctgggcatgaagcaggttgccgccaatccgtgggaaaccttcgctgccacccacaagaagggtgacaaggtgtcgggccagatcaagtcgatcaccgacttcggcatcttcatcggcctggacggcggcatcgacggcctggtccacctgtccgacatcagctggaacaccaccggcgaagacgtcgttcgcaacttcaagaagggcgataccctggacgccgtcgtcctggctgtcgatccggagcgcgagcgcatctccctgggcgtgaagcagctggagcaggatccgttcgggcagtacatggctgccaatccgaagggctccaaggtcgaaggcgtggtgaaggaagtcgacgccaagggcgcgatcatcgagctggctgacggcatcgagggttacgtttcggctcgcgacatcgccaacgagcgcgttgacgacgccacccagcacctgaaggtcggcgacaagatcgaagccaagttcgtgggcatggaccgcaagggccgcaccctgcagctgtcgatcaaggccaaggacgacgcggaaatgcgcgaagtgctggaggaataccagtcctcttcggcttccagcggcaccacccagctgggcgcgctgctgcgtgcacagctgaacggcaacaagtccgagta

SEQ ID NO: 128 - DNA, 2054 bp, Chitinophaga sp.
  Marker nucleic acid sequence
tgtcatttaagatacatgctccatatgctcctgctggtgaccagccggaagcaataaaacaattaacccagggactcaacgaaggggagccatttcagaccctgttaggggtgactggttccggaaaaacatttacagtagcgaatgtaatccaggaagtacagcgccctaccttggtgctgacccataataaaaccctggtggcacagttatacggtgagctcaggcagtttttcccggataacgcagtggaatattatgtgtcctattacgattattaccaaccggaagcgtatatgccggtaagtgatacttatatagaaaaggatctcgccattaacgaggaattggataagctccgcctgaaagctacagccaacctgttgtctggccgcagggatattattgtggtggccagcgtatcctgtatttacggtattggtaatcctactgattatgaaaacggtattatccgtattcagaaaggacagacgctgagccgtaatactttgctgcatgggttggtaaactcgctctacagccgcaccacaggcgatttcaaccggggtaatttccgggtaaaaggggatactgtagatatcaacctttcttatgtggattatggctaccggattactttttttggagatgagatagaagagatagagacaattgatgtccagaacagtaaacgtattaccgtggtggataatgccgccattttcccggctaacatgtacctggcgcccaaagatatgatgcagcagatcatctatgaaatacaggatgaactgaaagcccaggtagagtatttcaaggccaatggcaagctgattgaagcacagcgtttatccgagcgcgtgaattatgacgtggaaatgatccgtgaactgggttactgtagtggtattgaaaactattcccggttcctggaccgccgtaagcccggcgccagacctttctgcctgctggattattttccgaaagacttcctgctggtagtagatgagagccacgtaaccattccacagataggggccatgtatgggggcgaccgttcccgtaagctgaacctggtggaattcggcttccggctgccttctgccatggataaccgtccgctgaatttctacgagtttgagaatatgctgaaccaaaccatattcgtaagcgccactcccggtgattacgaactaaagaaaacagaaggtgtggtagtagaacaggtggtacgtcctaccggcttgctggaacctcctattgaggtaaggcccagcgtgaaccaggtggatgacctgctggaggaaatagataaacgtgtacagaaaggcgaccgtgtgctcgttaccacgctcaccaagcgtatggcggaggaaatggataaatacctgggacgtattaatattaagtcgcgctacatccactccgaggtagatacgctggaacgtatagaaatcctgcgcgatctgcgattgggtaatatagatgtattagtaggggtaaacctgttgagagaggggttggacttgccggaagtgtcgctggtggctattttggatgcagataaggaaggtttcctgagggatgaacgttccctcactcaaacggctggccgtgcagcgcgtaatgtggacgggctggtgatcttttatgcagataagatgacagagagcatgcagcgtaccatcgatgaaaccgatcgtcgccgggaaaagcaggtggcttttaatatagagcataacattacgcccaaaacggtgcgtaaaagcaaggagcagatcctgggacaaacttcagtgttggaaatcaagcatttcgacgagaactcccctatggctatacatgatgaagtacagctggtggccgaggatgccatggcctgggccaaaacgtcggcagcggcggtgaaaaccatcccgcagatggagaagtcgattgccaaggtgaaaaaggacatggaaaaagcggcaagagatcttgactttatggaagcggcccgtttgagggatcagatgtttgcaatggagcgcgaattgttagaaatgaaacaataa

SEQ ID NO: 129 - DNA, 1982 bp, Chitinophaga sp.
  Marker nucleic acid sequence
atgagcgaagaattagtgcaagcacccagccccagcagcaatggatatgatgcggggagtatacaggtattggaaggcctggaagccgtacgtaagcgtccagccatgtatatcggcgatattggcatcaagggtcttcaccacctggtttacgaggtggtcgataactctatcgatgaggccctggccggatactgcaaaaatattgacgtgactatcctggaagataactccatccgggtgaaggatgatggccgtggtattccaaccggtatcattccgaaggagggtcgttctgcactggaagttgtaatgacggtactgcacgcaggtggtaaattcgacaagaacacctacaaggtatccggtggtttgcacggtgtgggtgtgagttgcgtaaacgccctgagcgaccgcctgcacgtaaccgtagaacgcgatggtaaaatatttgaacaggaatatcaccggggtgcgccgcaatatcctgtgcgtgaaataggcgccagtgacggaaccggtactaccacacacttcaaaccagatggcgaaatattcaaggaaaccacctataaccgggaaatcctggcaggtcgtttacgtgaattatcatacctgaaccgcaaaatcaagatcacattagttgacgaacgtgaaaaggatgaagaaggcaacttcttctctgaaatcttctacagcgaaggcggtatccgggaatttatacagatgctcgataagaatggccgccgtaacccgctgctgccggcccctatctatgtagaagcacatgatgcggcttccaatgtagctgtagaggtagcactggtatacaatgacgcattcagtgagaacattttctcctatgttaataatatcaacaccattgaaggaggtacacacgtagccggtttccgccgtgctattacccgtgtattcaaatcctatggtgataagaataaaatgtttgagaaatcgaaggtagaagttaccggagatgacttccgcgaaggattaagcgctatcatcagcgtgaaagttcctgagcctcagtttgaaggacaaaccaaaaccaaactcggtaactccgacgtaatgggtgtggtagatagttctgtggccgccgtattggatgccttcctggaggaaaatccacgtgaagctaaaacggtgatcaacaaggtggtattggcggcacaagcccgcgaagctgcccgcaaggcccgtcagctggtacagcgcaaaagcgtgatgaccggaagcggtttaccgggtaaactggccgactgttccgataacgatcctaccaaatgtgaactgtaccttgtcgaaggagactccgcaggtggtactgccaaacagggccgtaaccgtaactaccaggctatcctgccactccgtggtaagatcctgaacgtagaaaaagccatggagcacaaaatctacgaggataatgaaatcaaaaacatctttaccgcgctgggtgtaaccatcggtacggaagaagatgacaaggctttgaaccttagcaaactgcgctaccacaagctcattatcatgacggatgccgacgtggatggtagccacattgcgacgctgatcctgaccttcgttttccgttatatgaaagccatggtagaacagggttatgtatacattgcacaaccgccactgtatctcgtgaaaaaaggtaaggaacaaacttacgcatggacagaagaagaacgcaaggctgcaacggtaaaactcgcaggcggcggtaaagaagacagcgtaacgatacagcgttataaaggtttgggagagatgaacgcagagcagctgtgggatacaaccatgaaccctgagcaccgtaccctgaaacaggttactattgaaagtgctgcggaagcagaccgcgtattcagtatgctgatgggcgatgaggttcctccacgcagagcgtttattgaatcacatgcgaagtatgcaaaaatcgatgctta

SEQ ID NO: 130 - DNA, 1010 bp, Chitinophaga sp.
  Marker nucleic acid sequence
tgtctacagccaatgcagaaaaactgaaggccttgcgcctcacaatggacaagattgagaaagatttcggtaagggatctgtgatgatgatgggtgaaaaggcagaagccccaatggaagtaatttctacgggttccctgggtcttgatatcgcacttggaattggtggtttaccgaaaggaagaatcattgaaatatatgggcctgagtcttccggtaaaacaactattgcaatacatactattgcggaagcacagaaaaaaggcggcatttgtgccattatagatgcggagcatgcttttgacagcagctatgcaagaagactgggagttgacgtagattccctgttaatatcacaaccggatcatggtgagcaggcgctggaaatagcagatcgcctgatcctttccggagcagtagatgtagtggtaattgactccgttgctgcactggtaccaaagagcgagctggaaggtgaaatgggtgaaagcaaaatgggattacaggcccgtttgatgtcacaggcgctgcgtaagttaacagccaccatttccaaaacaaattgttgctgcatatttattaaccagttacgtgaaaagataggtgttatgtttggtaatcctgaaacgaccaccggtggtaacgcgttgaagttctacgcatccgtaagactggatatccgccgtatgagccagattaaagatggtgacgaagcagtaggtaaccgtgtgaaagtgaaagtggtgaaaaacaaggtggcgcctccattccgtcaggctgaatttgatatcatttacggtcagggcgtgtccaaaatgggagagatcattgacatgggtgttgaatatggtgttgtacagaaaagcggtagctggttcagctacgacaccaataagctaggccagggccgtgatgcagtaaaacaactgctgatcgataatcctgaactggcagcagaaatcgaagcaaaaataaaagtgaagattcaggaaaaacaggatgctgaataa

SEQ ID NO: 131 - DNA, 1313 bp, Chitinophaga sp.
  Marker nucleic acid sequence
tggcaggatttacagtagctatagtgggtcgccccaacgtaggcaaatcaacgttgttcaatcgtttactggaacaacgtaaggctatcgtggacgaccagagcggtgtaacccgtgatcgtcaatatggtattgccgactggaatggcaaaactttcaacgttgttgacaccggtggatttgtgaccaacagcgatgatgtatttgagcgggaaatccgcaaacaggcaaaagtagccatggaagaggccaacctgatcatcttcatgtgcgatgttagcaccggtattaccgacctggatgcagatgtggcagatatcctgcgccgtacctccaaaccggtttacttagtagtaaataaagtagacaataaccaacggcagctggaagcgacagagttttacagccttggttttgaacatattttcttcctttcctctatgaccggcagcggtaccggcgaattgctggatgaggtagtaagcaatatcactcctgaaatgggagaggccagcctggatacagatcagccaaagatcgccatcatcgggcaaccgaacgtgggtaagtcatccctgctgaatgcgctggtaggtgcagagcgtaatatcgtttccgatattgccggtactacccgcgataccattcacacccattataatatgttccagaaagattttatgctgatcgataccgcaggtatccggcgtaaaaccaaggtgcatgaggatctggagttttattctgtgatccgtgctatcaaagccgtagacgagtctgacgtggtgatgttgctgctggatgctgaaaaaggcgtaacagcacaggatctgagcatttttagcctggctacccgtaaaggtaaaggcatagtggtgctggtgaacaaatgggataccgttgaaaaaggtaccaacaccgcaagggattatgagaaagaactgaagaagcgcctggcgccattttccgatgttccgatcatcttcacctccgtaaccgaaaagcagcgtattttcaaggctatagaaacggcgctcgacgtatatgaaaaccgtaaacgcaggatacagacctctgtactgaacgaaatcctgctcaaagcgattgaagcttatcacccaccggtagtaagaggtattcctatccgcattaaatatgtaacacagctgcctacgcatacaccggcatttgcttttttctgtaacctgcccgacgacgtcaaaacgccgtatcgcaactacctggaaaaccagatccgttcgaactttaacttcagcggggtaccgttaaagatattctttaggaaaaaatag

SEQ ID NO: 132 - DNA, 1922 bp, Brevibacillus sp.
  Marker nucleic acid sequence
tggatgtaacaactaaagatcaaaactatgatgctagtcagatacaggtactggaaggtttagaagcggtacgtaaacgtccaggtatgtacattggttctactagcagtcgaggtctacatcatttggtatgggagattgtagacaactctatcgacgaatcacttgcgggttattgtacggaaattaacgtatttattcataaagataatacgatcaccgttgttgataacgggcgtggtattcctacaggtattcatgaaaaaacagggaaatcaacagtagagactgttctaaccgttctacatgctggcggtaaatttggcggtggcggatataaggtgtctgggggtttgcacggggtaggtagctctgttgttaatgctctttctgaatggatggatgttactgttaaacaaaatggaaacatttatttcatgcgctttaatgttggtgcaccggaagaagaccttaaaataatcggagaaacagaaataacaggaactacgattacatttaagccagatccaactatctttacggaaacgactgaatttgaatatgagctgttacaaaagcgtattcgcgaattagccttcttaaataaaggcttatgcattacgctggaggatcagcgagaaggccaagagcgtcgtgaagagttttactatgaaggcggaattaaatcctacgtagagtacctgaataaaaaccgtgagagcctgcatgaagaagtgatctactgtgaaggcgaaaaggaaggattgcaggtagaggtagcgatccaatacaacgatagctatgtaagcaacatctattcctttgcaaataacattaatacgcacgaaggtgggacacacgaagctggttttaaagcaggcttaacccgtgtaattaatgattacagccgtaaatttaattttttgaaagaaaaagatacaaacctgtctggtgatgatgttcgtgagggaattacagcgattatctccgttaaaatttctgaaccacagtttgaaggacagacaaaaaccaagctaggtaactcagaggctcgtagcatcacagaatcggtctttgtagagcgattccaagaatttatggagcagaaccctgctgtagccaaaaaggttgtagagaagtccctgatggctgctagagcgcgtgaagctgctcgtaaggcacgtgaactgactcgccgtaaaaatgccttggaagtaagtgctcttccgggtaagctggctgactgttcttccaaagatgcagcggagagcgaaatatttattgtagagggtgactctgcgggtggttctgccaaaatgggacgcgatcgtcatttccaggcgatcctgccattgcgtggtaaagtattaaacgtagagaaatcacgtctagataaaattctaggtaataatgagattcgaatgatcatcacggcactgggaacgggtattggtgaagattttgatatcagcaaagcgcgttatcataaaatcgtaatcatgaccgatgccgacgtcgatggtgctcacatccgcacgctattgttaaccttcttctatcgctatatgcgtgaactaattgaagctggatttgtgtatattgcacagccgccactctttagtattaagcaagggaaaaccatgcactatgcctttaatgatcgccaacgcgaccaaatcctaagccagctaaaagagacccctaaacctaacgttcaacgttacaaaggattaggtgaaatgaatgcagagcaattatgggagacaacaatggatccttctgttcgtactttgctacaggtagaactagaagatgctattgatgcagatatggtcttccaaactctgatgggcgatgaagtagagccgcgtcgtgactttattgaaagctactctcgtaacgtacgtaatttggatatttaa

SEQ ID NO: 133 - DNA, 521 bp, Brevibacillus sp.
  Marker nucleic acid sequence
gtgacacaaccaaaattttatacggtagggaaattggttaatacacagggattacgtggagaaatgcgtgttgtttctacaacagatttcccagatgttcgttttaaaaaaggtaataaattgcatttgttccatccaagcttgactacaccggtaatcgttgaaattgcttcgtatcgtgaacataaagattttatcatcttaacctttaaagggtactcatctattaatgacgtagaaaagtataagggtggggaattaaaggttcctgaaacggaactactagatctggaggaagacgagttttatatccatcagctcattggttgcgaagtgattacggacacaggtgaagtattaggcaagattacagaggttctacaaccaggtgccaatgacgtttgggtggtaaagggaaataatggtaaagaaatcatgctacctttcattgatgattgcattaaaaaggtagatgttcagaataaaaaagtaatttgccacatcatggatggattgctgta

SEQ ID NO: 134 - DNA, 1100 bp, Pseudomonas sp.
  Marker nucleic acid sequence
atgggattcaattgcggcatcgtcggcctgcctaacgtcggcaagtccaccctgttcaacgccctgaccaaatccgggatcgcggccgagaacttccccttctgcaccatcgagcccaacagcggtatcgtgccgatgcccgacccgcgcctggacgccctggccgccatcgtcgtgcccgagcgcgtgctgcccaccaccatggagttcgtcgacatcgccggcctggtggccggtgcttcgaaaggtgaaggcctgggcaacaagttcctggccaacatccgcgaaaccgacgccatcgcccatgtggtccgctgcttcgaagacgacaacgtgatccacgtgtccaacagcgtcgatcccaagcgcgacatcgaaatcatcgacctggaactgatctttgccgacctcgacagctgcgaaaaacagttgcagaaagtcgcccgcaacgccaagggcggtgacaaggatgccgtggcccagaaagccctgctggagcagttgatcgcccacttcaccgaaggcaagccggcccgcagcctgatgaaggacatgagcgccgacgagaagctgatcatccgtggcttccacctgctgaccaccaagccggtgatgtacatcgccaacgttgccgaagacggcttcgagaacaaccccctgctggacgtggtcaaggccattgccgaggaagaaggcgcgatcgtggtgccggtgtgcaacaagatcgaggcggaaatcgccgagctcgatgacggcgaagaaaaggacatgttcctcgaggccctgggcctggaagagcccggcctgaaccgggtgatccgcgccggctacgaaatgctcaacctgcagacctacttcaccgcaggtgtgaaggaagttcgcgcctggaccgtgcgtgtcggtgctaccgcgccccaagcggccgcggtgatccacaccgacttcgagaaaggcttcatccgtgccgaagtcatcgcctacaacgacttcatccagttcaagggtgaagccggagccaaggaagccggtaaatggcgcctggaaggcaaggaatacatcgtcaaggacggcgacgtgatgcacttccgcttcaacgtgtg

SEQ ID NO: 135 - DNA, 1382 bp, Pseudomonas sp.
  Marker nucleic acid sequence
gtgctaacgatctacaacacgctcaccaagagcaaagaagtcttcaagccgctggatggcaacaaggtgcgcatgtacgtgtgcggcatgaccgtgtacgactactgccacctggggcacggccgcagcatggtggccttcgacctgattacccgctggctgcgtttcagcgggtatgacttgacctatgtgcgcaacatcacggatatcgacgacaagatcatcaatcgtgccaatgagaacggcgaggccttcgaggccctgaccgagcgcatgatcgcggcgatgcacgaggacgaggcgcggctgaacatcaagaagccggacatggaaccgcgcgccaccgaccatatccccggcatgcacgcgatgatccagaccctgatcgacaagggctacgcctacgctccgggcaatggcgacgtgtactaccgcgtcggcaagttcatgggctacggcaagctgtcgcgcaagaagatcgaagacctgcgcatcggcgcgcggatcgaagtcgacgagtcgaaacaggacccgctggacttcgtcctgtggaaagccgccaagccgggtgagccgagctggccatcgccttggggtgacgggcgtccgggctggcacatcgagtgctcggtgatgtccacctgctgcctgggcgagaccttcgacattcatggcggcggcagcgacctggagttcccgcaccacgagaacgagatcgcccagagcgaggcggctaccggcaagacctacgccaacgcctggatgcactgcggcatgatccgcatcaatggcgagaagatgtccaagtccttgaacaacttcttcaccattcgcgacgtgctcgacaagtaccatccggaagtggtgcgttacctgctggtggccagccactaccgcagcgccatcaactactcggaagacaacctcaaggacgccaagggcgccctggagcgtttctaccatgcactcaagggcctgccgaaagtggcgccggccggtggcgaagccttcgtcgagcgtttcacccaggtgatgaacgacgacttcggtactccggaagcctgtgccgtgctgttcgagatggtgcgcgagatcaaccgcctgcgtgagagcgatatcgatgcggcggccggtctggcagcccgattgaaggaactggccagtgtgctgggcgtgctgcaactggaggccgacgacttcctgcaggccggtgccgaaggtcgggtcgatgcggcagaagtcgaggcgctgatccaggcccgcctggcggcccgtgccaacaaggattgggcagagtccgatcggatccgcgaccagatcaccgccatgggcgtgatcctggaagacggcaagggcggtacgacctggcgcctggctgactg

SEQ ID NO: 136 - DNA, 1316 bp, Pseudomonas sp.
  Marker nucleic acid sequence
tgaacgctctctccgccaaactactgcacagcctcgcgccctggcgcgactccaggagttggcgcgtggctttctccggtggcctggattccaccgtcctgttgcatctgctcgccgatctggcgcagcaccatcgccttcctccactttcggctgtgcatatccatcatgggcttcaggctgcggccgacgcctggccggagcattgtcaggccttctgtgatgcgctgggggtgccgctgcaggtgcttcgcgtccaggtgccagccggtgccagcctggagcgcgccgcccgcgaggcacgctatggcgctttcgcacggctcacggcgagcactgaagtgctgctgacggcgcagcatcgtgacgaccaggcggaaacgctgctgttccgcctgctgcgcggtgccggagtcaggggcttgtcggcaatgccgtcagcgcggccattgggcgacgggcacctgctgcgacccttgcttgatgtgtcacgtgccgaactgcagtgctacgccgtggagcatcagttgagctgggtcgaagacccatccaacgccgatcatcagtattcgcgcaattacctgcgccaccaggtcatgccggcgattaccgcgcgctggccccaggcggcactcagcatgagccgcagcgccgcgcacttgaatgaagcccagggcctgctcgatgagctggcgctcatggatctggatgcagcccgcggtgctagcgtctggccctggctggatctgccctcactggctctggcacctttgcagtctttgtcgggcgcgcgccaacgcaatgccttgagccattggttgagccggtttggccgtttgccggataccgatcactggcgtggctgggacagcctgcgagatgcgggtgccgaagggcgaccggtgtggcgtttggccgatggcgagttgcatcgtgcggcagggcgagtctggtggctgtccggcagctggctgcaaccggtcgacggatggttgcagtggctcggtccgcaaaagcccttgcatctgccgggcaacggtcaggtgtcgttccctgggccagtgccgaccgggtcgctgcatatccgctatcgccaggggggcgagatcatgcacctggccggccgcggccatcgggacttgaagcgtttgctcaatgaaagcgggctgccgggtttcgtccgtggcagattgccgctgctgtatcaggacgaccgcttgctcgcggtggcgaacctgccggcacttgcgacaaacccccagggtggctggccattgcaatggctgccaccaaccagcgatcaaggtttgagctga

SEQ ID NO: 137 - DNA, 2021 bp, Acinetobacter sp.
  Marker nucleic acid sequence
ttgaacgaaaatcaaccttttgatttggtcactcattatcagccagcaggtgatcagccacaggcaattgaaaagctggtcaatggtatcgagcatggttttcgtaatcagttattgctaggggtaactggctcaggtaagacctataccatggcaaatgtgatcgctcaaacacagcgccctacaattgtgatggcccataacaaaactttggctgcccaactttatggtgagtttaagtctttcttccccaataatgcggttgaatattttgtaagttattacgattactaccagccagaggcttacgttccatcttcggatacgttcatcgaaaaagatgcagccattaatgatcacattgaccagatgcgattatctgcaacacgagctttattagaacgccgtgatgcaattattgtggcatcggtgtctgcaatttatggtttgggcgacccaaatgcttatatgcaaatgttgctgcatgtggtcgagggtgatcgagtaagccgtgatgaaatcatccgccgtttggttgaaatgcaatatacgcgtaatgaacttgaattcttacgtggtacatatcgaattcgcggcgaaattattgatattttcccagcagaatctgatcaacatgctattcgcattgaactatttgatgatgaggtcgactcaatccgttggtttgacccgctaacaggtaaaatggttcgtaaagtttctcgagtaacgatttatccgaaaagccactatgtaacaccaaaagatcatttaacacgtgccatcgatacaattaaggatgaattacaagatcaacttaaattcttccgtgaacatgacaaattgctcgaagcacagcgtattgagcaacggacacgttacgatctggaaatgatgcaacagctgggttataccaatggtatcgaaaactattcacgtcatttgtctgggcgtccatctggtgaagctccgccgactttatttgactatattccagaagatgctttgctgattattgatgaatcacacgttaccgttccacaaattggagcgatgtataaaggtgaccgatcgcgtaaagaaaacttagtaaactatggtttccgtttaccaagtgcgcttgataaccgtcctatgaaatttgaagagtgggaacgtattgtaccgaccaccatttttgtaagtgcaacacctgctaaatatgaattagaaaagtcagaccaaatcgttgaacaggtggtacgtccaacaggtctcatcgaccctgaaattgagattcgtcctgtcttaacccaagtggatgatgttctttctgaaattaatattcgtaagagtttgaatgaaagggtattaattacgactttaacgaagcgtatggcagaagatctgacttcatatttaaaagaatatggcgttaaggtggcttatctccactcagatattgatacggttgaacgtgtcaaaattattcatgaattacgtacaggcgtatttgatgttttagtcggtattaacctgctacgtgaaggtctggatatgccagaagtttctttggtcgctattttagatgcagataaagagggctttttacgttcggaacgttcacttattcagacaattggtcgtgctgcgcgtaatgtaaaaggtaaagcaattttatatgctgaccgaattactgactctatgcaaaaggcaatagatgaaactgatcgtcgtagagccaagcaaattgaatttaatgagttgcatggtattacgcctcgtagtgcggttcgtcaggctgtcaaagaaattgactcgggtgaggtactttcagatgacgagattgatgaaaaagtacttgaacaagcacaagcattgagtgccgatgaaagacatattctttcagaccctaaattgtttagtaaacatattactaagcttgaaaaagatatgttgaaggcctcgaaagatttacagtttgagcaagcagcgcggatacgtgatgaaattcttcgattaaaagcgcagatgttgcaata

SEQ ID NO: 138 - DNA, 2468 bp, Acinetobacter sp.
  Marker nucleic acid sequence
atgagttcagagtctcaatcagcctctcaaacagaacaaaccaatgaaaaggcttatgattcctctagtattaaagtattacgtggattagatgcagttcgtaaacgtccgggtatgtatattggggatacagacgacggtaccggtttacaccatatggtatttgaggtagtcgataacgcaatcgatgaagctttagctgggcattgtgatgaaattattgtcactattcatgaagatgagtcagtcagcgtttctgataatggccgtggtattccaaccgatattcaccctgaagaaggtgtttctgcagctgaagtaattcttaccattctgcacgcaggtggtaagtttgatgacaacagctataaagtttcaggtggtttgcacggcgtaggtgtttctgtagtaaacgcactttcgcgtaaattacatttaatgatttctcgcgctggtcaagtgcatgagcaagaataccaacacggcgatccgcaatatccattacgtgtgattggtgaaacagataagagcggtacaacagtacgtttttggccaagtgcagaaacttttagccaaactatctttaacgtagaaattttagcgcgtcgtttacgtgagctttcattcttaaacgcgggcgtgcgtattgttttacgtgatgaacgaattaaccttgaacatgtgtatgactacgaaggtggcttatctgagtttgtaaaatatattaacgaaggtaaaactcatctgaatgagattttccattttactgcggatacagataacggcattgttgtagaagtagccttgcagtggaacgaaagttatcaagaaaacgttcgttgttttaccaataacattcctcaaaaagatggtggtacacatttagcaggtttccgtgcagcgttgacgcgtggtcttaaccaatatcttgaaaatgaaaatattatcaagaaagaaaaagtcaatgtaaccggtgacgacgcgcgtgaaggtttaactgctattatttcggtaaaagtacctgatccgaagttctcatctcaaaccaaagaaaaactggtatcgagtgaagtaaaaccagcagttgagcaagcaatgaacaaagagttctctgcttacttacttgaaaacccgcaagctgcaaaatcgattgcaggtaaaattattgatgctgcacgtgcgcgtgatgctgcacgtaaagcacgtgaaatgacgcgtcgtaaaagcgcacttgatatcgctggtttaccaggtaaattggcagattgtcaggaaaaagatccagcactttctgaactatatcttgtcgagggtgactcagcaggtggtagtgctaagcaaggccgtaaccgtaaaatgcaggctattttgccgttgaaaggtaaaatcctgaacgttgaacgtgcgcgctttgacaaaatgatttctagtcaggaagttggtactttaattactgctcttggctgtggtattggtcgtgaagaatataatcccgataagctacgctatcataaaattattatcatgaccgatgccgacgttgatggttcgcacattcgtaccttgttattaacgttcttcttccgtcaaatgccagaattggttgagcgtgggcatatctatattgcacagccacctttgtataaattgaaaaaagggaagcaggaacaatacatcaaagataatgatgcattggaaacttacttaatttcaaatgcgattgatgagctgtctttgcataccaatgctgaagcacctgctattacgggtgaagcacttgcaaaagtcattcaagactatcaagtttcacaaaagagcttacagcgcttaactttacgttatccagctagcttattggatgctttacttgaagtagaagcatttaagagcgatcaaaatcatgatcaggcttatgtacaacaatgggctgatcaagtaaaagctatcattacaagattacagccaagtttacgcccagaaattacactagagtcatttgagcgtgaaaatgctcaaggtgaaaaatctgcgcattattggccgcgtattacggtatatgttcataacttgccacatgcatatttgcttgatgcagctttattgaattcagctgagtatgcacgtttgttgaaaaactcgaagagttggttcagtttgcttgaagatggtgcttatttacaaaaaggtgatcgccgtattcaggtagctaacttccatcaagtttggcagcatattttgcaagattcgcgtcgcggtatgatgattcagcgctataaaggtcttggcgagatgaacgctgaacagctttgggaaacgacaatggaccctgaaaaccgtaatatgttgcaggtaactattgacgatgcgattgaagcagatcgtatgttctcttgcttgatgggtgatgatgtggaaccacgtcgtgcattcattgaagaaaatgctttaaatgcggatattgatgctta

SEQ ID NO: 139 - DNA, 1046 bp, Acinetobacter sp.
  Marker nucleic acid sequence
tggatgagaataaaagcaaagcattacaagccgcgctaagtcaaattgagaaacaatttggcaaaaatacggtgatgcgtctcggtgacaacactgttcaagctgttgaagctgtatctacaggctctttgactttagatattgcattggggattggcggtttaccaaaaggtcgtattgtcgagatttatggtccagaatcttctggtaaaaccacaatgactctacaagcaattgcacaatgtcaaaaaactggtggtacttgcgcatttatcgatgcagagcatgcattagatccacaatatgcgcgtaagcttggtgtagacattgataacctgcttgtatcgcaacctgataacggtgaacaagcactcgaaattgcagatatgctcgttcgttctggtgcaattgacttaatcgttgtcgactcagtcgctgcacttactcctaaagcagaaattgaaggtgagatgggtgactctcatatgggcttgcaagctcgtctaatgagtcaggcacttcgtaaaattacgggtaatgctaagcgttcaaactgtatggttattttcattaaccagatccgtatgaagattggagtaatgtttggtagtcctgagactacaactggtggtaacgcattgaagttctacgcttctgtacgtttagatatccgccgtattggccaagtaaaagaaggcgatgaaatcgtcggctctgaaacccgagttaaagtggttaaaaacaaaatggcccctcctttcagagaggctattttccaaatcctatatggtaaaggcacaaaccagctaggcgaacttgtagatttagctgtacaacaagatatcgtacaaaaagcaggtgcttggtattcatatcaaggtaataaaattggtcaaggtaagaacaatgttattcgccactttgaagaaaaccctcaaatggctggagaaattgaacgtaacatccgtgaacagttgttaacaacaggtactaatactgctcaagttgaagatgaagaagaagctgatcttctattagaatcttaa

SEQ ID NO: 140 - DNA, 1823 bp, Pseudomonas sp.
  Marker nucleic acid sequence
atgaccgacccgtttgatcccagtgcttttctttccacctgcagtggccgtcctggcgtgtatcgcatgttcgatggcgatgcacgtctgctctacgtcggtaaagccaagaacctgaagagccgtctggccagctactttcgcaagaccggcctggcgccgaagaccgccgccctggtggggcgtatcgcgcagatcgaaaccaccatcaccgctaacgaaaccgaggccctgctgctcgagcagacgctgatcaaggaatggcggccgccgtacaacatcctgttgcgcgacgataagtcctatccctatgtgtttttgtcggacggcgattttccacgtttcagcatccatcgcggggcgaaaaagcagaagggtaagtacttcggcccgtaccccagcgccggggccattcgcgagagcctgagcctgctgcaaaagactttcatggtccgccagtgcgaggacagctactacaagaaccgcacccggccgtgcctgcagtatcagatcaagcgctgcaaggcgccgtgcgtcggcctggtggaggcagaggtatacgccgaagatgtgcgccactcggtgatgttcctcgaaggccgcagcaatgccctgaccgatgaattgtcggccggtatggaacaggcggccagtaccctggacttcgagaaagccgccgagctgcgcgaccagatttccctgctgcggcgggtacaggaccagcagagcatggaaggcggcagcggcgacgtcgacgtggtggcggccttcgtcaacccgggcggtgcctgcgtgcacctgatcagcgtgcgtggcggccgggtgctgggcagcaagaacttctttccccaggtgggtatcgaggaagacgtggccgaggtcatgtcggcgttcctcggccagtactttctcagcagcccggagcgcgacctgcccagcgaactgatcgtcaatgtggtccatgaagacttcccgaccctgatcaccgcgatccaggccttgcgcggccgtgaactgagcatcagccaccgggtgcgtggcacgcgggcgcgctggcaacaactggcggtgaccaatgccgagcaggccctgagcgcgcgcctggccaaccgccagcatgtggcggcacgtttcgaggccctggccgacgtgctgaacctggacgaaccgccgcaacgcctggagtgctacgacatcagccactccagcggcgaggcgaccgtggcgtcctgcgtggtgttcggcccggaaggtccgctgaagtccgattatcggcgctacaacatcgaaggcgtcaccgccggcgacgactatgcggcgatgcaccaggccctgacccggcgcttcagcaagctcaaggacggcgagggcaagctgccggacatcctgctggtggacggtggcaagggccagctgtcgatggcccgcgacgtgctcaacgagctggcggtgcccgacctgatcctgctgggggtggccaagggcgcgacgcgcaaggccggtttcgagaccctctacctcaacgatgccgcccatgagttcaccctcaagggcgactcaccggcgctgcacctgatccagcagatccgcgacgaagcccaccgtttcgcgatcaccggccaccgcgcacgccgcggcaagacccgccgcacctcgaccctcgaaggcgtggccggcgtcggccccaaacgccgccgcgacctgctgaagcattttggcggattgcaggagctgtctcgcgcaagcatcgacgagatcgccaaggcaccggggatcagtaaaaagctcgctgagttgatttatgcgaacttgcatagcgagta

SEQ ID NO: 141 - DNA, 1382 bp, Pseudomonas sp.
  Marker nucleic acid sequence
gtgctaacgatttacaacacgctcaccaagagcaaagaagtcttcaagccgctcgatggcaacaaggtgcgcatgtacgtgtgcggcatgaccgtgtacgactactgccacctgggccacggccgcagcatggtcgccttcgacctggtgacccgctggctgcgtttcagcggctatgacctgacttatgtgcgcaacatcaccgacatcgacgacaagatcatcaatcgggccaacgagaacggcgagtcgttcgaggcgttgaccgagcgcatgatcgcggcgatgcacgaagacgaagcgcgcctgaacatcaagaagccggacatggaaccgcgtgccaccgaccatatcgccggcatgcacgcgatgatccagaccctgatcgacaagggctacgcctacgcgccgggcaacggcgacgtgtactaccgcgtggccaagttcatgggttacggcaagctgtcgcgcaagaagatcgaagacctgcgcatcggcgcgcgcatcgaagtcgacgagtcgaaacaggacccgctggacttcgtgctgtggaaagccgccaagccgggcgagccgagctgggagtcgccatggggcgccgggcgtccgggctggcacatcgaatgctcggtgatgtcgacctgctgcctgggcgagaccttcgacattcacggcggcggcagcgacctcgagttcccgcaccatgaaaacgagatcgcccagagcgaggcggccaccggcaagacctacgccaatgcctggatgcactgcggcatgatccgcatcaatggcgagaagatgtccaagtccttgaacaacttcttcaccattcgcgacgtgctcgacaagtaccatccggaagtggtgcgttacctgctggtgtcgagccactaccgcagcgcgatcaactactcggaagacaacctcaaggacgccaagggcgccctggagcgtttctaccacgccctcaagggcctgccgagcgtgccggccgccggtggcgaagccttcgtcgagcgtttcaccgaggtgatgaacgacgacttcggcaccccggaagcctgtgcggtgctgttcgagatggtgcgcgagatcaaccgtctgcgtgagagcgatctcaatgcggcggccggtctggcggcgcgcctgcgggagctggcggatgtgctgggtgtgctgcagctggaagccgacgacttcctgcaggccggtgccgccggccgggtcgacgcggcgcaggtcgaagcgctgatccaggcgcgcctggccgcccgtgccaacaaggactgggccgagtccgaccgcatccgcgaccagatcaccgccatgggcgtggtgctggaagatggcaagggtgggacgacctggcgcctggctgactg

SEQ ID NO: 142 - DNA, 2015 bp, Pseudomonas sp.
  Marker nucleic acid sequence
tgtctgaattccagctagtcacccgcttccagcccgccggcgatcagccggaagccatccgcctgatggtcgagggcatcgaggccgggttggcgcaccagacgctgctcggtgtgaccggctcgggcaagaccttcagcatcgccaacgtgatcgcccaggtgcagcgcccgaccctggtgctggcgccgaacaagaccctggccgcgcagctgtacggcgagttcaaggcattcttcccgaacaacgcggtggagtacttcgtttcctactacgactactaccagccggaagcctatgtaccgtcctccgacacctttatcgagaaggacgcctcgatcaacgaccatatcgagcagatgcgtctgtcggcgaccaaggcgctgctggagcgcaaggacgcgatcatcgtcaccacggtgtcctgcatctatggcctgggcagcccggaaacttatctgaagatggtgttgcacgtcgatcgcggcgacaagctcgaccagcgcgccttgctgcgccgcctggccgacctgcaatacacccgtaacgacatggattttgcccgcgccaccttccgcgtgcgtggcgatgtgatcgatatctatccggcggaatccgacctggaagcgatccgcatcgagctgttcgatgacgaggtggaaagcatctccgccttcgacccgctgaccggcgaagtgatccgcaagctgccgcgtttcaccttctatcccaagagccactacgtgacgccgcgggaaaccctgctggacgccatcgaggggatcaaggtcgagctgcaggagcgcctggaatacctgcgcagcaacaacaagctggtggaagcccagcgcctggagcagcgcacccgtttcgacctggagatgatcctcgagctgggttactgcaacggcatcgaaaactactcgcgctacctgtccgggcggcctgccggggcggcgccaccgaccctgtacgactacctgccggcggacgccttgctggtgatcgacgaatcccacgtcagcgtgccgcaggtcggcgccatgtacaaaggcgaccgttcgcgcaaggaaaccctggtggagtacggcttccgcctgccctcggcgctggacaaccggccgatgcgcttcgacgaatgggaaggggttagcccgcagacgattttcgtctcggccacgccaggcaactatgaagccgaacatgccggccgggtggtcgagcaggtggttcgccccaccggcctggtcgacccgcaggtggaagtgcgcccggcgctgacccaggtcgatgacctgctgtcggaaatcaccaagcgcgtggccgtggaagagcgggtcctggtaaccaccctgaccaagcgcatggccgaggacctgaccgattacctcgccgaccacggcgtgcgggtgcgttatctgcactcggacatcgacaccgtggagcgggtcgagatcatccgcgacctgcgcctgggcaccttcgatgtgctggtggggatcaacctgctgcgcgaaggcctggacatgccggaagtgtcgctggtggcgattctcgatgcggacaaggaaggtttcctgcgttccgagcgctcgctgatccagaccattggccgtgctgcgcgtaacctcaacggccgggcgattctctacgccgaccgcatcaccggctccatggagcgggcgattggcgaaaccgagcgccgtcgcgacaagcagatcgccttcaacctggccaatggcatcacccctaaaggcgtgttcaaggacgtcgccgatatcatggaaggcgctaccgtgcccggttcgcgcagcaagaagcgcaagggcatggccaaggccgccgaggaaaatgcccgctacgaaaacgaattgcgttcgcccagcgagatcaccaagcgcatccgccagctcgaagagaagatgtaccagctggcccgcgacctggagttcgaagccgcggcgcagatgcgcgacgaaatcggcaagctgcgcgagcgtttgctgacggtctga

SEQ ID NO: 143 - DNA, 1328 bp, Pseudomonas sp.
  Marker nucleic acid sequence
tgagcccattcgataccgtccttacgcaaaaactgctgcaacgcctcctgccctggctggatcgatcagcctggcgcatcgcgttctccggtggcctcgattccaccgtcctgctgcacctgcttgcccaactcaggcagcgacacacacttccccctttgaccgccatccatgtgcatcacggccttcaggctgtggctgacgcctggccggagcattgtcgaacccagtgcgaggccttggcggtgccgttgcaaatagtggcggtgcaggtgcaaccgggggccagcctggagcgcgcggcgcgggaagcgcgttatgccgcgttcgaagaggtaaccggcagcaacgagctgttgctgacggcccagcaccgtgacgatcaggcggaaaccctgttgtttcgtctgctgcgtggggccggggtcaggggcttgtccgccatgccggcgtcgcgtgccttggggcaggggtatttgctgcggcctttgctcgacgtctcccgcgacgaactggagcgttacgctcgtgatcaccaactgcactggatcgatgacccctccaaccaggatcggcagttctcccgcaactacttgcgtcatcaggtgttcccattgctgactgcccgctggccacaagctgccagcagcctggcgcgcagcgcggcgcatgtgcgcgaggcccgggagttgctggatgagttggcgctcatcgatctgcaagaagcggctaaggagggagagttcgagtggctgggcttgccatccctggcactggcgcctttggtcggcttgtcggaggcgcgtcagcgcaatgccctgagtcactggctgagccggttcacgacattgcccgacagcgaccactgggcgggctggcagacgctgcgcgatgccgcgggcgacagccgtccggtatggcggctgaggcagggcgagttgcagcgggcggcagggcgggtctggtggttgtcggatggctggctgcaacccgtgctgtctgcaccgagttggatcgataccgctgcgcctctgttgttgccgggtaatggtcaattgcagttcaccgggggagcgccgcaagggcccttgcaggtgcgttaccgtcaggggggcgaggtgatgcaactgcctgggcgcggccaccgcgacctcaagcgcctgctcaatgaggcgggtctgccggccttcgtccgtggcagattgccgctgctgtatcgaggcgaagaattgctcgcggtggcgaacctgccaggtcttgacgcaaatgcccatgaaggctggcaattgcattggcagccgccttcgagcgatcaaggtttgagctga

SEQ ID NO: 144 - DNA, 2024 bp, Stenotrophomonas sp.
  Marker nucleic acid sequence
tgagcgaccgttttgaactcgtctcgccgtactcgccggcgggcgaccagcctgatgccatcgccaagctgaccagcaacttcgaggcgggcatcgccaagcagaccctgctgggcgtcaccggctcgggcaagacctacaccatcgccaacgtcatccagaacgtgcagaagccgacgctgatcatggcgccgaacaagacgctggcggcgcagctgtatggcgagttcaaggcgttcttcccgcacaacgcggtcgagtatttcgtcagctattacgactactaccagccggaagcctacgtgccgtcgtcggacaccttcatcgagaaggacagttcgatcaacgagcacatcgagcagatgcgactggcggcgaccaagaccctgctgtcgcgcccggacgcgatcgtggtggcgacggtgtcggcgatctacggcctgggcgcgccggaagactacctgtcgctgcgcctgatcctgtccaagggcgagcgcatcgaccagcgcgatctgatcaaccacctcacccagctgcagtacacgcgcaacgagtacgagctgcagcgcggtacgttccgcgtgcgtggagaggtgatcgacgtattcccggccgagtcggacagcgaagcgctgcgcctggagttgttcgatggcgaggtcgagaagatcacgttgttcgatccgctcaccggcgagacgctgcgcaacatgcagcgcttcacggtctacccgaagacccactacgccaccacgcgcgagcgcgtgctggcggcggtcgagacgatcaaggtcgagttgaaggagcggctggagcagctgtacgcgcagaacaagctggtcgaagcgcagcgcctggcgcagcgcacccagttcgatctggaaatgatggccgaggtcggcttctgcaacggtatcgagaactactcccggcacctgaccggcaagaacgccggcgaaccgccgccgaccctgttcgactacctgccggccgacgcgctgctggtgatcgacgaatcgcacgtgaccattccgcagatcggcgccatgttcaagggcgaccgatcacgcaaggaaacgctggtcgagttcgggttccggctgccctcggcgctggacaaccggccgctgcgcttcgaggagtgggaagagcgctgcccgcgcagcatctacgtgtcggccacgccgggcccctatgaataccgcgaggccggtgacgagatcaccgaactggtcgtacgtcccaccggcctgatcgacccggtggtggagatccgcccggtggggacccaggtcgacgatctgatgagcgaagccaacgcgcgcatcaaggccggcgaccgggtgctggtcacgacgctgaccaagcgcatggccgagaacctcaccgaatacctgaccgagcacggcatccgcgtgcgctacctgcactcggacgtggacacggtcgagcgcgtggagatcatccgcgacctgcgcctgggcaagttcgatgtgctggtgggcatcaacctgctgcgcgaaggcctggacatgcccgaggtatcactggtggcgatcctggatgccgacaaggagggcttccttcgttccaccggctcgctgatccagaccatcggccgtgccgcgcgcaacgtgcgtggcaaggcgatcctgtatgcggacaagatcacccgttcgatgcaggccgcgatcgatgagaccgaccgccgtcgtgccaagcaggtcgagtacaacgaggagcacggcatcgtgccgcgctcggtggcgcgtccgatcaccgacgtgctggagggtgcgcgctccgatgcggccgagaaggaagcaaagaagggcaagggcaagggcaggcccggcgttgccgaggaggggggtgactatcgatcgctcagcccggcccagctggcgtcccgtctcaaggcgctggagcagcagatgtaccagcacgccaaggacctggagttcgaggatgccgcgcgcgtgcgtgaccagatccggcagctgaaggaagcgagtctgggctga

SEQ ID NO: 145 - DNA, 1100 bp, Stenotrophomonas sp.
  Marker nucleic acid sequence
tggcaaagaagacccccaaagacagcacccccaacgacacgacctccgcaaggaccaatccgatggacgagaacaagaagcgcgccctcgctgcagctctgggccagatcgaaaagcagttcggcaaaggctcggtgatgcgcatgggcgatcgcgtggtcgaacccgtcgaagccatcccgaccggttcgctgatgctcgacatcgcgctgggcattggcggtctgccgaagggccgtgtcgttgagatctacgggccggagtcctcgggcaagaccacgctcaccctgcaggccatcgccgaatgccagaagatgggcggcaccgcggccttcatcgacgccgagcacgcgctggacccgatctacgccgccaagctgggcgtgaacgtcgacgacctgctgctgtcgcagccggataccggtgagcaggcgctggaaatcgccgacatgctggtccgttcgggttcggtcgacatcctggtgatcgactcggtcgccgcgctgaccccgaaggctgaaatcgaaggcgagatgggcgatcagctgccgggcctgcaggcccgcctgatgagccaggcgctgcgcaagctgaccggcaacatcaagcgctccaacaccctggtggtcttcatcaaccagctgcgcatgaagatcggtgtgatgatgccgggccagagcccggaaaccaccaccggtggcaacgcgctgaagttctacgcctcggtgcgcctggacatccgccgtatcggcgcgatcaagaagggcgacgagatcatcggcaaccagaccaagatcaaggtcgtcaagaacaagctggcgcctccgttcaagcaggtcatcaccgagatcctgtacggcgaaggcatcagccgcgaaggcgaactgatcgacatgggtgtggatgccaagctggtcgagaaggccggcgcctggtacagctacggtgaggagcgcatcggccagggcaaggacaacgcccgtggttacctgcgcgacaacccgacggttgctgcgaagctcgaagctgagctgcgcgagaagttccagccgaccgaagccacccgtgaggaaggcgacgacgaaggcgacgacgagtaa

SEQ ID NO: 146 - DNA, 572 bp, Carnobacterium sp.
  Marker nucleic acid sequence
tgcgcgttatttcaggtgaatatggtggtagaaaaataaaggctgtcccaggaactaatacccgtccaacaactgataaaattaaagaatcaatttttaatattattggtccttattttgatggtggtatttgcttagatttatttgcaggaagtggtggattagcgattgaagctgtatctcgtggttttgataaagcgattttaattgataaagaaattcttgcaatcaaaacaattaaagaaaatattgaaatgacgaaagagcctgagaaatttcaactttatcgaaatgaagcgaatcgtgctttagatgctttaagtagacaaaatcaagcctttgatttgatttttatggaccctccttatatgaaacaagaaattgaaaaacaacttacccaaattatggataaacagttactaaacccgaatggaaaaatcatttgtgaagttgataagaaaacggatttacctgataaggttgcaggcttggtagcaacacgcagagaaagttatggaatgacaaagattgttatttatcaattagatgaagagagtgaacaagatgagtaa

SEQ ID NO: 147 - DNA, 1409 bp, Carnobacterium sp.
  Marker nucleic acid sequence
tgttaaaaatatacaatacaatgactcgtgaaaaagaagtatttgttcctttaacagaagggcaagttaaaatgtacgtttgtggtccaactgtttacaattatattcatatcggaaatgcacggagcactgttgctttcgatacggttcgtcgttactttgaattccgcgggtaccaggttcaatttgtttctaattttacggatgtagatgataaaattattcaggcagcaaaagaattgaatattacagcaccagatgttgccgatcgttttattgaggctttttatacagatgtgacagctttgggtgttgagaaagctacgaaaaacccaagagtaatggaaaatatgcctgatattattgaatttatccaagtattaatagataaaggctatgcctatgagtcagatggtgatgtctattatcgaacacgaaaatttaagaactatggtaaacttagtgatcagtcaattgatgagttagaaattggtgcgagtaaccgtttagaaaaaatagaaaatgaacgaaaagaagatcctgtcgattttgcattatggaaatcggctaaagcagatgaaattagttgggaatctccttggggtgctggacgtccaggttggcatatcgagtgttcagttttggcaacaaaatatttaggtgatacaattgatattcatggtggtggtcaagatttaacgttcccgcatcatgaaaacgaaattgctcaaagtgaagcaaaaaccggacatccatttgctaattactggatgcataatggttttgtgacaatggatgaagaaaaaatgagtaagtctttaggaaactttgttttagttcatgagctaattaagcaagttgatcctcaagtattacgttttttcatggcaacaactcattaccgacgtccgattagctacaatgaggcaactatatcagaagctaaaaataatttaaataaagttcaaatggcagcgagcaatcttcattatcgcttaagtgatgcaacgatagaattgccgacagatcaagactatttccaacaattagcagaatttaaacagacttttatcactgagatggatgatgattttaatgcggctaatggaattactgtgatttatgagttagctaaggttatgaacatttatagtgaacaagctgaagtttctaaagcagttattgaagctatgttagggctgtttaaagagttgcttcaagtttttggaatcactctagaaaatcaagctgaactattagatgaaaatattgaacaattgctaagcgaacgaactatagctagatcagagaaaaattttgctagaagcgatgagattcgggatttactaaaagcagagggcattatcattgatgatactccccaaggaacgagatggagacgagaataa

SEQ ID NO: 148 - DNA, 515 bp, Carnobacterium sp.
  Marker nucleic acid sequence
gtggctgaattatttaatgtaggtaaaattgtaaatacacaaggattaaaaggagaagtgcgtgttatttctcgaactgattttccagatgaacgttataaaaaagggaatacgttatttttagaacaagagggaaaacctttaatagaattagttgtagcgagtcatcgtaagcacaaaaattttgatattctaagtttcaaagatcatccgtcaatcaatgatgttgaaaaataccgtgatggaatgttgcaaattacttcagatcaaatgcatgatttgccagagaatgaattttatttgcatgaaatcattggcttaaaggtgattgatgaagaaggcgaagaaattggtaaaatcaaagaagtcttatcaccaggagcaaatgatgtctgggtgattcagcgtgttcaacaaaaagatttattgattccgtatattgaagaagtggtgaaaaaagtcgatatcgctgaaggggtagtcacaattcatattatggaaggattgttagacta

SEQ ID NO: 149 - DNA, 1103 bp, Ensifer sp.
  Marker nucleic acid sequence
atgggcttcaaatgcggtattgtcgggctgccgaacgtcggcaagtccacgctcttcaacgcactcaccaagacggcggccgctcaggcggccaactatcccttctgcaccatcgagccgaacaccggcgaagtggcggtgccggacccgcgcatgcgcaagcttgccgacattgccaagtcgaaggaaatcatcccgacgcgcatctccttcgtcgacatcgccggcctcgtgcgtggcgcgtcgaagggcgaaggcctcggcaatcagttcctcgccaacatccgcgaagtggatgctgtcgtgcatgtgctgcgctgcttcgaggatgatgacatcacccacgtcgaaggccgcatccacccggtcgcagatgccgagacgatcgagaccgagctgatgctcgccgacctcgacagccttgagcgccgcaccgagcagacccgcaagcgcgccggcggcaaggacaaggactccatggcgcagcttccgatcatggaagcctcgctgaagctgctgcaggacggcaagccggtacgcacgctgcttgccaagctcgatgccgaagaactgcgcattctgcagagcctcaacctcctgaccgcccatccggtgctctacgtctgcaacgtcgccgaaagcgatgcggtcgacggcaacgaacacacccgcgcggttgccgaaatggcaaaggcgcaaggggccgaaagcgtcgtgatctcggcggcgatcgaatccgaagtggcacagctgccggaagaagaatccaaggagttcctgagcgcgctcggcctagaagaagccggtctcgaccgtctgatccgcgccggctacaagctgctcgacctcatcacctacttcaccgtcggccccaaggaaacccgcgcctggacgatccagcgcggcaccaaggcaccgcaggcggccggcgtcatccacaccgacttcgagcgcggattcatccgtgccaacacgatcgcctacgaggattacatcacgctcggcggcgaagtgggcgccaaggaagccggcaaggcgcgcgacgaaggcaaggaatacgtcgtccaggacggcgacgtcatccacttccgcttcaacacgta

SEQ ID NO: 150 - DNA, 2942 bp, Ensifer sp.
  Marker nucleic acid sequence
tgaccaaaactccgaagaatgccccgccgaagccctccacgccgtccggtttcgaagaagcgccgcaggcgcccttgtccggcaccccgctcagcggcaacgtctcggattgggtgaagcagctcgaggcggatgcggaagcctccgctttcgaaacccagcgtcagatcgcctccaaggccggcaagcaccgcaagacggtagagatcgccgcctccaaggcggcgcgcaaggccgcgggcgcggatgacgaagcacagccgaagaaggccgggaagaagagcaaggtcgagatcgtcggcagcaagtccgcgcgcggcacgtcgatgggcggctcgaccgatccgaagacccgcgccgccgccggcctcaacccggtcgcgggcctcgacgtgtcgctggaggatgccgacaggctgacggccaaatccggcgtcaccgccacggtggaagcgctgtcggcgctgatcgaaagcggcaacccgctgttcaaggacggcaagctctggacgccgcaccggcctgcccggccgccgaaatccgaaggcggcgtcgagatccagatggtctccgattacgagccggccggcgaccagccgacggcgatcgccgatctggtcgagggcctgacctccggcgagcgcagccaggtgctgctcggcgtcaccggttccggcaagaccttcaccatggccaaggtaatcgaggcgacgcaacgtcccgccgtcatcctcgcgcccaacaagacgctggccgcgcagctctattccgagttcaagaacttctttcccaacaacgcggtcgagtacttcgtctcctactacgactactaccagccggaagcctacgtgccgcgctcggataccttcatcgagaaggaatcctcgatcaacgagcagatcgaccgcatgcgccactcggccacccgctcgctgctggagcgcgacgactgcatcatcgtcgcgtctgtctcgtgcatctacggtatcggctcggtcgagacctataccgccatgaccttccagatgagcgtcggcgaccggctcgaccagcggcaactgctcgccgacctggtggcgcagcagtacaagcgccgcgacatggatttccagcgcggctcttttcgcgtgcgcggggataccatcgagatttttccggcccacttggaggatgccgcctggcgcatctccatgttcggcgacgagatcgacgcgatcaccgagttcgacccgctgaccggccagaagaccggcgacctgaagtcggtgaaaatctacgccaactcgcactatgtcaccccgcgcccgacgctgaacggcgccatcaaggcgatcaaggaagagctggtgcaccggctcgccgagctggaaaagggcggccgcctgctcgaagcccagcggctggagcagcgcacccgctacgacatcgagatgctggaagcgaccggctcctgcgccggcatcgagaactattcgcgctacctcaccggccgcaaccccggcgagccgccgccgacgctgttcgaatatatccccgacaacgcgctgctgttcatcgacgagagccacgtctccgtcagccagatcggcggcatgtatcgcggcgacttccgccgcaaggcgacactggccgaatacggcttccgcctgccctcgtgcatggacaaccgtccgttgcgcttcgaggaatgggacgcgatgcgcccggacacgatcgccgtttcggcgacgcccggtagctgggaactggaacaatccggcggcgtctttgccgaacaggtcatccgcccgaccggcctgatcgacccgccggtggaagtgcgctcggcgaaaacccaggtcgacgacgtcttgggcgaaatccgcgagaccgccgccgccggctaccgcacgctcgtcaccgtgctcaccaagcgcatggccgaagacctcaccgaatacctgcacgaacagggcattcgcgtgcgctacatgcactccgacatcgacacgctggaacgcatcgagatcatccgcgacctgcgcctcggcgctttcgacgtgctcgtcggcatcaacctgttgcgcgagggcctcgacattcccgaatgcggcttcgtcgccattctcgatgccgacaaggaaggcttcctgcgctccgagacctcgctggtgcagaccatcggccgcgctgcccgtaacgtcgacggcaaggtcatcctctatgccgaccagatcaccggctcgatgcagcgcgccatggacgagaccagccgccgccgcgaaaagcagatggcctacaacgaggccaacggcatcacgccggaatcggtaaaggcgaagatctccgacatcctcgactcggtctacgagaaggaccacgtccgcgccgacatctccggcgtcgccggcaagggcttcgccgacggcggccacctcgtcggcaacaacctgcaggcccaccttaacgcgctggaaaaacagatgcgcgacgccgccgccgacctcgacttcgaaaaggccgcccgcctccgcgacgagatcaaacgcctcaaggccgccgaactggccgccttggacgacccgatggcgcgggaagaggcaaggtcgcaggaagggggcaagcggtccggctcctcgacgtcatcctcgtcctcgggttcaacccgaggatcgagccgaggatccaagaccacggacgaaaagtctctcttccagaagcccgacctcgacgacatgggccgcgacgtcgcgacacccgccggctcggacaaatcgctattccgccggaacaccctcgacgaaatgactgtcggccgcaccgaaaagccggtgatcggccatgtcccggataaaccgatcctcacccgggcgaaaccgggcgtcgggtcctatgaggatccggtggacgagaagcggcagaagggccggacgaagggcaagacgggacggccggggcggtag

SEQ ID NO: 151 - DNA, 2435 bp, Ensifer sp.
  Marker nucleic acid sequence
tgaccgacaatcctgagaccgaaaccggcgccagcgccgaatatggtgccgattccatcaaggtgctgaagggcctggatgccgtgcgcaagcggccgggcatgtatatcggcgacacggatgacggctccggtctgcaccacatggtgtatgaggtcgtcgacaacgcgatcgacgaagcgcttgccggtcatgcggatatcgtaacggtgacgcttaattctgatggatcggtaacggtcacggataacggtcgcggcatcccgaccgacatccacaaggaagaaggcgtgtcggcggccgaggtcatcatgacccagttgcacgccggcggaaaattcgaccagaactcctacaaggtttcgggcggtctgcacggcgtcggcgtgtccgtggtcaacgcgctttccgtttccctgaagctgaagattcgccgcgcaggcaaaattcacgagatgagcttcacgcacggcgtcgccgatggctctctggccgttaccggcgacgcaggcgacgcgaccggaaccgaggtgacgttcctgccgagcagcaaaaccttcaccaagaccgagttcgattacaacacgctggaacaccgccttcgcgaactcgccttcctcaattcaggcgttcatatcattctgaccgacaagcgccattcggacattcgccaggacgagatgatgtatgagggcggtctcgaggcctatgtcgcctatctcgaccgggcgaagaagccgctggtgcacaagcccgtatcgatccgcggcgaaaaggacggcatcaccgtcgaagtggcgatgtggtggaacgacagctaccacgaaaacgtgctctgctttaccaacaacatcccgcagcgcgacggcggcacccatatggcgggcttccgcggcgcgctgacgcgccaggtcacctcctacgcggattcctcgggcatcaccaagagggaaaaggtcacgctccagggcgaggactgccgcgaaggcctgacggctgttctttcggtcaaggttccagatccgaagttctcatcgcagacgaaggacaagctggtgtcttcggaagtgcgcccggttgtcgaaagcctcgtcaacgaagcgctcagcacctggttcgaagaacatccgagcgaagccaagatcctcgtcggcaaggtggtcgaagcggctgctgcccgcgaagccgcccgcaaggcccgcgaactgacgcgccgcaagggtgcgctcgatatctcgtcgctgcccggaaagcttgccgattgctccgagcgtgacccggccaagtccgaagtcttcctggtggagggtgactcggcaggtggctccgccaagcagggccgctcgcgcgaaaaccaggcgatcctgccgctgcgcggcaagatcctcaatgtcgagcgtgcgcgcttcgacaagatgctgtcgagccaggaaatcggtacgctgatcaccgcactcggcacctcgatcggcaaggacgagttcaacgccgacaagctgcgctatcacaagatcatcatcatgacggacgccgacgtcgacggcgcccatatccgcaccctgctgctcaccttcttcttccgccagatgccggaattgatcgagcgcggccatctctatatcgcccagccgccgctctacaaggtcacgcgcggcaagtcggctcaatacctcaagaacgagcaggcactggaagactacctgatcacgatgggtctggaagaagccacgctggagctggaatcaggcgaagtgcgcgccggccaggatctgcgcgaagtcatcaatgatgcactccgcctgcgcaatctgatgaacgggctgcactcgcgctacaaccgctcgatcgtcgagcaggcggcgatcgccggcgccctgaacgttgagctcaacggccagcgcgacgcctatgaagcggtcgctgcggaggttgcccgtcgcctcgacgtcatcgccgaggaaaccgaacgcggctggagcggtgcggtcacggcagaaggcggtcttcgcctcgagcgcatggtccgcggcgtcaaggaagtcgcggtgctcgatatggcgctgatcggctcgtccgatgcccgctacatcgaccagctcaccgcgaagctcaaggaagtctatgctgccccgccggttctgcggcgtcgcgacggtaccctggagatcagcggaccgcgtgcgcttctcgatgcgatcttcgcggccggtcgcaagggtctctcgatgcagcgctacaagggcctcggtgaaatgaatgccgagcagctctgggaaacgacgctcgatccgaacgtccgctcgctgctgcaggtccgcgtcaccgacgccaccgatgccgacggcctgttttcgcgcctgatgggcgacgaggtcgagccgcgccgcgacttcatccaggaaaacgcactgagcgtcgccaacctcgacatctga

SEQ ID NO: 152 - DNA, 1091 bp, Ensifer sp.
  Marker nucleic acid sequence
atggcacaaaactctttgcggctcgtagaggacaaatcggtggataaaagcaaggcacttgaagcggcactttcccagatcgaacggtcgttcggcaagggatcgatcatgaagctcggtgggaaggacagcatcatcgagatcgaaaccgtctcgacgggttctctcagcctcgacatcgcgctcggcatcggcggcctgccgaagggccgcatcgttgaaatttacggcccggaaagctcgggtaagacgacgctggcgctgcagacgatcgccgaagcacagaagaagggcggcatctgcgccttcgtcgacgccgaacacgcgctcgatccgatctatgcgcgcaagctcggtgtcgatctcgaaaacctcctgatctcgcagccggacaccggcgagcaggcgcttgagatcaccgacacgctggtgcgctccggtgcgatcgacgttctcgtcgtcgactccgtcgcagcgcttgtgccgcgcgccgaaatcgaaggcgaaatgggcgacagcctgccgggcatgcaggcacgcctgatgagccaggcgctgcgcaagctgaccgcgtcgatctccaagtcgaactgcatggtcatcttcatcaaccagatccgcatgaagatcggcgtcatgttcggttcgccggaaaccaccaccggcggcaacgcgctgaagttctacgcctcggtccgcctcgacatccgccgcatcggttcggtcaaggagcgcgaagaggtcgtcggcaaccagacccgtgtcaaggtcgtcaagaacaagatggcgccgcccttcaagcaggtcgagttcgacatcatgtatggcgaaggcgtttccaagaccggcgaactcgtcgatctcggcgtcaaggccggcatcgtcgagaaatccggtgcctggttctcctataacagccagcgcctcggccagggccgcgagaacgccaagacgttcctgcgcgacaacccggacctggcgcgtgagatcgagacggcgctgcgccagaacgccggcctgatcgccgaccggttcctcgaaaacggcggcccggaacccgacgacggcagcgacgacgccgccgaggcgtg

SEQ ID NO: 153 - DNA, 1433 bp, Ensifer sp.
  Marker nucleic acid sequence
tgagcttcaccgtcgccatcgtcgggcgtcccaatgtcggcaagtccaccttgttcaatcgcctggtcggcaagaagctggcgctcgtcgacgatacgccgggcgtcacccgcgaccgtcgtccgggcgaggcaaagctgatcgacctgcgcttccgcatcatcgacacggccggtctcgagcaatcggggcccgacacgctgcagggtcgcatgtgggcgcagacggaagccgcgatcgacgaggcggatctttcgctgttcctggtcgacgccaagggcggcctgacgccggccgacgaaaccttggccgaaatgctgcgccggcgcggcaagccggtggtgctggtggccaataagtccgaggcgcgcggctccgatggcggcttctacgatgcgtttacgctcggccttggcgaaccttgcccgatctcggccgaacacggccagggcatgctggacctgcgcgacgcgatcgtcgctgcgatcggcgaagagcgggcctatccgccggaagacgacgtggccgagaccgatatcgatctgcgcccgacgttcaaagacgaagagggcgaagaggaagaggcggagacagtctatgacgagaccaagccattgcgcgtggcgatcgtcggtcgtccgaacgccggcaagtcgacgctgatcaaccgcttcctcggcgaagaccgacttttgaccgggccggaagccggcatcacccgcgattccatctcggtcgaatgggactggcgcggccgtaccatcaagatgttcgacaccgccggcatgcgccgcaaggcgaaggtgcaggagaagctggagaagctctcggtcgccgacgcgctgcgcgccattcgctttgccgaaacggttgtcatcgtctttgacgcgaccattcccttcgagaagcaggacctgcagatcgtcgatctcgtgctgcgcgaggggcgcgcggcggtgctcgccttcaacaagtgggatctggtcgaggactggcaggcggtgctcgccgacctccgcgaaaagaccgagcggctcctgccgcaggcccgcggcatccgcgccgtgccgatttccggccacaccggctatggcctcgatcggctgatgcaggcgatcatcgaaacggacaagacctggaaccgccgcatctcgaccgcgcggctcaaccgctggctggaatcgcagacggttcagcatccgccaccggccgtgtccggtcgccgcctgaagctcaagtacatgacccaggtgaaggctcgcccgccgggcttcatgatctcctgcacacgtcccgacgcgctgccggaatcctacgtgcgctacctgatcaacggcctgcgcaacgatttcgatctgccgggtgtgccgatccgcatgcatttccgcgcgggcgacaacccctacgagtccaaggcgaagaagcgccgatag

SEQ ID NO: 154 - DNA, 1343 bp, Rahnella sp.
  Marker nucleic acid sequence
tgagttcaaaactggccgtgacatttctttcaaaacatctgctcaataaaatcggtgatgcacgaagactttgcgttgcgttcagcggcggactggattcttccgtactgctggccggactggctgagttacgcgacaaagcgttgccggatatcgaactgcgtgcattgcatatccatcatggactaagccagtttgcagatgactgggtcgcacactgccagacgttttgtcagcagcgagatattcctttctccgtcgctaaagtgcaggtagatgcacaggaaggtgggattgaagcggcggcacgaacggcacgttatcaggcatttgctgaaagtctgactgctggtgaagtgttactgacagcacaacacctgaatgaccaatgcgaaactttcatgctggcgcttaaacgtggaagcgggcctgccggtctctcggcgatgagcggggacagttcaacacatggttatcgcttgttaaggccttttctggagctcagccgtgagcagcttgaagcatacgcgaagcagttaaagctcagctggattgaagatgacagtaaccgggatgcccgctttgatcgcaattttctgcgactggatatttttcccgcactttaccagcgctggccgcactttgcgcaagccacagcccgcagcgcatcactttgcgcagagcaggagacgctgctggatgagttgctcagtgaatctctcacttcattgaccgatgagcagggcagtctgtcggttgagaggctgaaggcgttttcagccgtcagacgctctgcgctattacgtcgctggctggcatcgcgtggcgcaagaatgccttcgcgtgaacaactgcagagaatatgggatgaggtagctttgagccgcgaagatgcccaggcgcagttagtgctggggaaaatggtagtccggcgtttccgtgggcgtttgtatgtattgccagaacgccggtcgcttaaagatcaaatcctctgctgggatccggctgaaaaattaacgctacctgacggtctgggctgtctgattgccgatcatgttctcatcgcgcagggggctgactgtttgatccgccagccacgtgctggcgaaaagatcactgtgcgttttcaggtgcagggatatatcgaaaaagtcgggcgggatcgtgcgcgccaggcgaaaaaattgtggtctgagttaggtgtgccgccatgggaacgaggtcgtatccctttggtttattacaatgatcaattgatggcagccccgggttggtttattacgcgccagtcccgggctgaaaataatgaagcgcaatggcggatcatctggtcggctgcgtaa

SEQ ID NO: 155 - DNA, 1484 bp, Rahnella sp.
  Marker nucleic acid sequence
tgatacctgtcgttgcgcttgttgggcgcccgaatgtgggtaaatccactttatttaaccgtttaaccaagacgcgtgatgcgctggtggcggatttcccggggcttacgcgtgaccgcaagtatggtcgtgccgaaattgagggtaatgaattcatcatcgtggataccggcggtattgacggtaccgaagatggcgttgaaacccgcatggcagggcaatctctgctggctatcgaagaagcggatatcgtgctgttcatggtggatgcgcgcgcgggcctgatgcctgccgatcagggcattgcccagcatcttcgcagccgtgaaaaagccaccttcctggtagcgaacaaaactgacgggctggatccggacagcgcaaccggtgatttctactcactcggtttgggcgatgtgtatgctatcgcggcttcacacggccgtggcgtgacgcaactgatcgaacacgtactgatcccgttcgtgggtgaaaagccggaagaagtcgaactgaccgaggaagaagctaacgcagcttactgggccgagcaggaagccaccggtgatgaaatccctgaagacgaagaagataatttcgacccgcagtctctgccaatcaagctggctatcgtcggacgtccgaatgtaggtaagtccacactgactaaccgtattctgggtgaagagcgcgtcgtggtttatgacatgccgggcaccacccgcgacagtatttatatcccgatgaaccgagacgaccgtgaatacgttctgatcgacaccgcaggtgtgcgtaagcgcggcaaagtgactgaaaccgtggagaaattctcggtaatcaaaacccttcaggcgattgaagatgccaacgtagtgatgttggttatcgatgcccgcgaaggcatttccgatcaggatctctcgctgttaggctttatcctcaacagcgggcgctcgctggttatcgtggtcaacaagtgggatggcatgagcgaagaagcgcgtgctcaggttaaagatcagctggatctgcgtcttggtttcgttgattttgcccgcattcacttcatctctgccctgcacggcagcggcgttggtaacctgtttgagtcagtaaacgaagcgtacacctgtgcaaccacgcgtgtgagcacttccttactgacccgtatcatgcaaatggcggctgacgatcaccagccaccgctggtcaatggtcgccgcgtgaaactgaaatatgctcacgccgggggctataacccgccaatcgtggtgattcacggtaatcaggtgaaagacctggctgattcctataaacgctatctgatgaactatttccgtcgttcactgaaagtgatgggaacgccaatccgcattcagttcaaagaaggtgagaacccgtatgaaggcaagaaaaactcgctttctcctcaccagcagcgcaagcgccgacgtctgatgcagcacctgaaaaacaaatcctaa

SEQ ID NO: 156 - DNA, 2021 bp, Acinetobacter sp.
  Marker nucleic acid sequence
ttgaacgaaaatcaaccttttgatttggtcacacattatcagccagcaggtgatcagccacaggcaattgaaaagctggtcaatggtatcgagcagggttttcgtaatcagttattgctaggggtaactggctcgggtaagacctataccatggcaaatgtgatcgcccaaacacagcgacctacaattgtgatggcccataacaaaaccttggcggcccaactttatggtgagtttaaatctttctttcctaataatgcggttgaatatttcgtaagttattacgattactaccagcccgaggcttacgttccatcttcggatacgtttatcgaaaaagatgccgccatcaatgatcacattgatcagatgcgattatctgcaacacgagctttattagaacgtcgtgatgcaattattgtcgcatcggtatctgcaatttatggtttgggtgaccccaatgcttatatgcaaatgttactgcatgtggttgaaggtgaccgagtaagccgtgatgaaatcatccgccgtttggttgaaatgcaatatacgcgtaatgaacttgaatttttacgcggtacttatcgaattcgcggcgaaattattgatattttcccagctgaatccgatcaacatgccattcgcattgaactatttgatgatgaggtcgattcaatccgttggtttgacccattaacaggtaaaatggttcgtaaagtttctcgagttacgatttatccgaaaagccactatgtaacaccaaaagaccatttgacccgtgccatcgatacaattaaggatgagttacaagatcagctcaaattcttccgtgaacatgacaaattgctcgaagcacagcgtattgaacaacggacacgttacgatctggaaatgatgcaacagttgggttataccaatggtatcgaaaactattcacgtcatttgtctggacgtccgtctggtgaagcaccaccgactttattcgactatattccagaagatgctttgctcattattgacgaatcacatgttacggttccgcaaattggagcgatgtataaaggtgaccgctcacgtaaagaaaacttagtgaattatgggttccgtttgccaagtgcgcttgataaccgcccaatgaagtttgaagaatgggagcgtattgtaccgactaccatttttgtaagtgcaacacctgctaaatatgaattagaaaagtcagaccaaattgttgaacaagtggtacgtccaacaggtctaatcgaccctgaaattgaaattcgtcctgttttaacccaagtggatgatgttctttctgaaattaatattcgtaagaatttgaatgaaagggtgttgattaccacattaacaaaacgtatggcagaagacctgacttcttatttgaaagagtatggcgttaaggtcgcttatctccactcagatattgatacggttgaacgtgtcaaaattattcatgaattacgtacaggtgtatttgatgttttagtcggtattaacctgctccgtgaaggtctggatatgccagaagtttctttagttgctattttagatgcagataaagagggttttttacgttctgaacgttcactcattcagacgattggtcgtgcggcgcgtaatgtgaaaggtaaagcaattttatatgctgaccgaattaccgactctatgcaaaaagcaatagatgaaactgatcgccgtagagctaaacaaatcgaatttaatgagctacatggtattacgccgcgtagtgctgttcgtcaggctatcaaagaaattgactcgggtgaggtactttcagacgacgagattgatgaaaaagtacttgaacaagcacaagcattgagtgccgatgaaagacatattctttcagatccaaaattgtttagtaaacatatcactaagcttgaaaaagatatgttaaaggcctcgaaagatttacagttcgagcaagcagcgcggatacgtgatgaaatcattcgattaaaagcgcaaatgttgcaata

SEQ ID NO: 157 - DNA, 2468 bp, Acinetobacter sp.
  Marker nucleic acid sequence
tgagttcagagtctcaatcagcctctcaaacagaacaaaccaatgaaaaggcttatgattcctctagtattaaagtattacgcggattagatgcagttcgtaaacgtccgggtatgtatattggggatacagacgacggtaccggtttacaccatatggtattcgaggtagtcgataacgccatcgatgaagctttagctgggcattgtgatgaaattattgtcactattcatgaagatgaatcagtgagtgtttcggataatggccgtggtattccaactgatattcaccctgaagaaggtgtttctgcagcggaagtaattcttaccattctgcacgcaggcggtaagtttgatgacaacagctataaggtttcaggtggtttacacggcgtaggtgtttctgtagtaaacgcactttcaagtaaattacatttaatgatttctcgtgctggtctagtgcatgagcaagaatatcaacacggtgatccgcaatatccattacgtgtgattggtgaaacggataagagtggtacaacagtacgtttttggccaagtgaattaacatttactcaaaccatttttaacgtagaaattctagcacgccgtttacgtgagctttcattcttaaacgcgggcgtacgtattgtattgcgcgatgagcgtattaaccttgagcatgtatatgactacgaaggtggattatctgagtttgtaaaatacattaacgaaggtaaaactcatcttaatgagattttccattttactgcagatacagataacggtattggtgtagaagtagccttgcagtggaatgaaagttatcaagaaaacgttcgttgctttaccaataacattcctcaaaaagatggcggtagccatttagctggtttccgtgcagctttgacgcgtggtttaaaccaatatcttgaaaatgaaaatattctcaagaaagaaaaagttaatgtaaccggcgacgatgcacgtgaaggtttaaccgctattatttcggtaaaagtacctgatccaaaattttcatctcaaaccaaagaaaaactggtatcgagtgaagtaaaacctgctgttgagcaagcaatgaacaaagagttctctgcttacttgcttgaaaacccacaagctgcaaagtcaattgccggtaagattattgatgctgcacgtgcacgtgatgctgcacgtaaagcgcgtgaaatgacacgtcgtaagagcgcacttgatatcgctggtttaccaggtaaattagcagattgtcaggaaaaagatcctgcactttctgaattgtatcttgtcgagggtgactctgcgggtggtagtgctaagcaaggccgtaaccgtaaaatgcaggctattttaccgttgaaaggtaaaatcctgaacgttgaacgtgcgcgctttgacaaaatgatttctagtcaggaagttggtactttaattactgctctcggctgtggtattggtcgtgaagaatataatcctgacaaactacgctatcataaaatcattatcatgaccgatgctgacgttgatggttcgcacattcgaaccttgttattaacgttcttcttccgtcaaatgccagaattggttgagcgtgggcacatctacattgctcagccccctttatacaaattgaaaaaaggtaagcaggaacaatacatcaaagataatgatgcattagaaacttatctgatttcaaatgcgattgatgagctgtcgttacatattagtgctgatgctcctgcaattactggcgaagcacttgcaaaagtgatcaaggattatcaagtttcacaaaagagcttgcaacgtttaaccttacgttatccagctagcttactagatgctttactggaagtagaagctttcaaagccgatcaaaatcatgatcaggcttatgtacagcaatgggctgatcaggttcgtgaaacagtcactaaattgcaaccaagtttacgtccggaaattaccctagagtcatttgaacgtgaaaatgctcagggtgaaaaatctgcacattattggccacgtattaccgtatatgttcataacttgccgcatgcctatttgcttgatgctggtttattgaattcagcggaatatgctcgtttattaaaaaactcgaagagttggttcagcttaattgaagagggtgcttatttacaaaaaggtgaccgtcgtattcaagtggctaacttccatcaagtttggcagcacattttgcaagattcgcgccgcggtatgatgatccagcgctataaaggtcttggtgagatgaatgctgaacagctttgggaaacgacaatggaccctgacaaccgtaatatgttgcaggtgaccattgatgatgcgattgaagcagatcgtatgttctcttgcttaatgggtgatgacgttgaaccacgtcgtgcattcattgaagagaatgctttaaatgcagatattgatgcttaa

SEQ ID NO: 158 - DNA, 1046 bp, Acinetobacter sp.
  Marker nucleic acid sequence
atggatgagaataaaagcaaagcattacaagccgcgctaagtcaaattgagaaacaatttggcaaaaatacggtgatgcgtctcggtgacaacactgttcaagctgttgaagctgtgtctacaggctctttgaccttagatattgcattggggattggcggtttaccaaaaggtcgtattgtcgagatttatggtccagaatcttctggtaaaaccacaatgaccctacaagcgattgcacaatgtcaaaaaactggcggtacttgtgcatttatcgatgcagagcatgcattagatccacaatatgcacgtaaacttggtgtagacattgataatttacttgtatcacagcctgataacggtgaacaagcacttgaaattgcggatatgctagttcgttctggcgcaattgatatgattgttgtcgactcagtcgctgcacttacacctaaagcagaaattgaaggtgagatgggtgactctcatatgggcttacaagctcgtttaatgagtcaggcacttcgtaaaattacgggtaatgctaaacgctcaaactgtatggttattttcattaaccagatccgtatgaagattggggtaatgtttggtagccctgagaccacgactggtggtaacgcattgaagttctacgcttctgtccgtttagatatccgccgtattggtcaagtaaaagaaggtgatgaaatcgtcggttctgaaactcgagttaaagttgttaaaaacaaaatggctcctcctttcagagaagctattttccaaatcctatatggtaaaggcacaaaccaactaggcgaacttgtagatttagctgtacagcaagatatcgtacaaaaagcgggtgcttggtattcatatcaaggtaacaaaattggtcaaggtaagaacaatgttattcgccactttgaagagaaccctcaaatggctggagagattgaacgtaatatccgtgaacagctgttaacaacaggtacgaatactgctcaagttgaagatgaagaagaagctgatcttctattagaatctta

SEQ ID NO: 159 - DNA, 1094 bp, Paraburkholderia sp.
  Marker nucleic acid sequence
atgagcctcaaatgcgggatcgtcggcttgcccaacgtcggcaagtccaccctgttcaacgcgctgaccaaggccggcatcgcggccgagaactatccgttctgcacgatcgagccgaacgtcggcatcgtcgaggtgccggacgcgcgcttgaaggcgctcggcgagatcgtgaagcccgagcgcgtggtgccggccgtcgtcgagttcgtcgacatcgcgggcctcgtcgccggcgcgagcaagggcgagggcctcggcaaccagttcctcgcgaacatccgcgaaaccgacgcgatcacgcacgtcgtgcggtgcttcgaggacgacaacgtcatccacgtcgccggcaaggtgagcccgatcgacgacatcgaagtgatcaacaccgagctcgcgctcgccgacctcggcacggtcgagaaggcgctcgcgcgctactcgaaggccgcgaagtcgggcaacgacaaggaagcggcgaagctcgtcgcggtgctcgacaaggtgcgcgcgcagctcgaccagggcaacgccgtgcgcggcctcgcgctgtcggacgacgagcgcgcgctgctgaagccgttctgcctcatcaccgcgaagccggcgatgtacgtcgcgaacgtgaaggacgacggcttcgagaacaacccgcacctcgacgcggtgcgcaagtacgcggaaggcgagaactcgcccgtggtcgcggtgtgcgcggcgatcgaggcggaaatcgccgatctcgacgacgaggacaaggaagcgttcctcgccgacatgggcatggacgagccgggcctcgaccgcgtgatccgcgcgggcttcaagctgctcggcctgcagacgtacttcaccgcgggcgtgaaggaagtgcgcgcgtggacgatccatatcggcgacacggcgccgcaggcggccggcgtgatccacaccgacttcgagcgcggcttcatccgcgcgcagacgatcgcgttcgacgactacatcacgtacaagggcgagcaaggcgcgaaggaagccggcaagatgcgcgcggaaggcaaggaatacgtcgtgcacgacggagacgtcatgaacttcctgttcaacgtctg

SEQ ID NO: 160 - DNA, 2090 bp, Paraburkholderia sp.
  Marker nucleic acid sequence
atgtccgatcatccagccgaagccgccgacgcgctcgacgaatcgaaattcgtgaccttcgacggctcgccgttccagctctatcaaccgtatccgcccgccggcgatcagccgaccgcgatcgacacgctcgccgaaggcgtcgaggacggcctgtcgttccagacgctgctcggtgtcacgggttccggcaagacgttcacgatggcgaacacgatcgcgcggctcggccgcccggcgatcgtgttcgcgccaaacaagacgctcgccgcgcagctctattcggagttccgcgagttctttccgcgcaacgcggtcgagtacttcgtgtcgtactacgattactaccagcccgaagcgtacgtgccgcagcgcgacctgttcatcgagaaggactcgtcgatcaacgagcacatcgagcagatgcggctgtccgcgacgaagagcctgatggagcggcgcgacgtcgtgatcgtcgcgaccgtgtcggcgatctacggcatcggcaatccgtccgagtatcaccagatgatcctgacgctgcgccacggcgacaagatcggccagcgcgacgtgatcgcgcggctgatcgcgatgcagtacacgcgcaacgagcaggacttccagcgcggcacgttccgcgtgcgcggcgacacgatcgacatcttcccggccgagcacgcggagatggcggtgcgcgtcgagctgttcgacgacgaggtcgacacgctgcagctcttcgatccgctgaccggccgcgtgcggcagaagatcgcgcgcttcaccgtgtatccgtcgtcgcactacgtgacgccgcgcgacaccgtgatgcgcgcggtcgagacgatcaaggacgagctgcgcgagcgcctcgagttcttccatcgcgagaacaagctcgtcgaggtgcagcggctcgagcagcgcacgcgcttcgatctcgagatgctgcaggagctcggcttctgcaagggcatcgagaactactcgcggcacttctcgggcgccgcgcccggcgagccgccgccgacgctcgtcgactatctgccgaccgacgcgctgatgttcctcgacgagtcgcacgtgctgatcggccagttgaacggcatgtacaacggcgaccgcgcgcgcaaggaaaacctcgtcgactacggcttccggctgccgtccgcgctcgacaaccggccgctgaagtttcccgaattcgagcgcaagatgcgtcaggtcgtgttcgtgtccgcgacgccggccgactacgagcagcgcgtgtcgggccaggtcgccgagcaggtcgtgcggccgacgggcctcgtcgacccggagatcgaggtgcggcccgcgcgctcgcaggtcgacgacgtgctcgccgagatccacgagcgcgtgaaggccggcgagcgcgtgctgatcaccgtgctcacgaagcggatggcggagcagctgaccgagttcctcgccgatcacggcgtgaaggtccgctatctgcacagcgacatcgacacggtcgagcgcgtcgagatcatccgcgacctgcggctcggcacgttcgacgtgctggtcgggatcaacctgctgcgcgaggggctcgacattccggaggtgtcgctcgtcgcgatcctcgacgcggacaaggaaggcttcctgcgcgcggagcgctcgctgatccagacgatcggccgcgcggcgcgcaacgtgaacggcaaggcgatcctgtacgcggacacgatgaccgagtcgatgaagcgcgcgatcggcgaaaccgagcggcgccgcgcgaagcagatcgcgcacaacgagcggctcggcatcacgccgcgcggcgtcgtcaagcggatcaaggacatcatcgacggcgtgtacaacgccgacgaggcgcgcgcggagctgaaggaggcgcagcagcgcgcgaagctcgaggacatgtccgagaagcagatcgcgaaggagatcaagcgcctcgagaagcagatggccgattacgcgaagaacctcgaattcgagaaggccgcgcagacgcgcgaccagctcgcgctcctgcgcgagcgcgtgttcggcgcgaacgtcggcgatcacgtgcccggcggccggtg

SEQ ID NO: 161 - DNA, 2471 bp, Paraburkholderia sp.
  Marker nucleic acid sequence
atgactgaacagcacaattcgcagcccgacaccagctacggcgcgtcgtcgatccagatcctcgaagggctggaggcggtgcgcaagcgccccgggatgtacatcggcgacacgtcggacggcaccggtctgcaccacctcgtgttcgaggtgctcgacaactcgatcgacgaggcgctcgccggatactgcaacgacatccacgtgacgatccacgcggacaactcgatctccatcaccgacaacggacgcggcattccgaccgacgtgaagatggacgacaagcacgagccgaagcgctcggccgccgagatcgtgatgaccgagctgcacgcgggcggcaagttcgaccagaacagctacaaggtgtcgggcgggctgcacggcgtgggcgtgtcgtgcgtgaatgcgctgtcgagctggctgcgcctcgtcgtgcgccgcaacggcaagaagcacttcatggagttccatcgcggcatcgtgcagaaccgcgagatcgagacgcgcgacggcgtcgacgtgtcgccgatgcaggtgacgggcgacaccgagcagcgcggcaccgaagtgcatttcatggccgatccgacgatcttcggcacggtcgagtatcactacgacatcctcgcgaagcggattcgcgagctgtccttcctgaacaacggcgtgcgcatccgcctgaccgaccagcgcaccggcaaggaagacgatttcgcgttcgtcggcggcgtgaagggcttcgtcgagtacatcaacaagacgaagaccgtgctgcatccgacgattttccacatcgtcggcgagaaggacggcgtcggcgtcgagatcgcgatgcagtggaacgacagctacaacgagaacgtgctgtgcttcacgaacaacatcccgcagcgcgacggcggcacgcacctcaccggcctgcgcgcggcgatgacgcgcgtgatcaacaagtacatcgccgacaacgagatcgcgaagaaggcgaaggtcgagacgtcgggcgacgacatgcgtgaagggctgtcgtgcgtgctgtcggtgaaggtgccggagccgaagttcagctcgcagacgaaggacaagctcgtttcgtcggaggtgcgcgcgccggtggaagacgtggtggcgaaggcgctcgaggagttcctgctcgagacgccgaacgacgcgaagatcatctgcggcaagatcgtcgaggccgcgcgcgcgcgcgacgccgcgcgcaaggcgcgcgagatgacgcgccgcaagggcgtgctcgacggcgtcgggctgccgggcaagctcgcggactgccaggaaaaggatccggcgaagtcggagatctacatcgtcgagggcgactcggcaggcgggtcggcgaagcaggggcgcgatcgcaagttccaggctattctgccgctgcgcgggaaggtgctgaacgtcgagaaggcgcgctacgacaagctgctgtcgtccgagcagatcgtcacgctcgtgaccgcgctcgggtgtgggatcggcaaggacgattacaacctcgacaagctgcgttatcaccggatcatcatcatgaccgacgcggatgtcgatggtgcgcacatccgcacgctgctgctcacgttcttctatcggcagatgccggacatcatcgagcgcgggtatgtttatatcgcgcagccgccgctttacaaggtgaaggcgggcaaggacgagcgctatctgaaggacgacgcggagctcaacgcgcatatgctgcggctcgcgttgctggggtccgagttgctgcctagtacgaacggcacgccgattacgggggatgcgcttggggagctggcgcggtcttatctgttgtcgcagggcgtggtcgacaggttgagccggctttatgatccggcagcgctcggggccatcatggatggggtggcgatcgatctttccagcgaggaatcgacggaggcttctgcgaaggcactgcaggccgcgctcgaggatgatgcgctcaagcaggacgtgcgggtggtgccttcgtatgacccggtgcgcgaggtgcgatcgctgcgggtcgagcggccgcatcacgggaatgtgcgggtttccgtcattgacgaggagtttcggctgactgcggattatcagcagttggtgaatacggcgaatctgttcaagggactcattggcgaaggggcggtgatcaagcgcggtgaacgaagcatggccgtgacggacttcaagggcgccatgaagtggctcatcgccgatgcggagcggaatgtttccaagcagcggtataaggggctgggcgagatgaaccccgagcagctttgggacaccacgatgaacccggaagtgcgacggatgctgagggtgcagatcgaggatgctattgctgcggatgggatctttactaccctgatgggggatgaggtcgagccgcggagagcgtttatcgagtcgaatgcgcttagggcggggaatattgatgtttg

SEQ ID NO: 162 - DNA, 1073 bp, Paraburkholderia sp.
  Marker nucleic acid sequence
atggaagaaagcaagaaaggctccgggatgactgcggagaagagcaaggcgctgtccgctgcgctcgcgcagatcgagaagcagttcggcaaggggtcgatcatgcggatgggcgccggcgaggcggtcgaggacatccaggtggtgtcgaccggatcgctgggtctcgatatcgcgctcggcgtcggcgggctgccgcgcggccgcgtggtcgagatctacggccccgaatcgtccggtaaaacgacgctgacgctgcaggtgatcgccgaattgcagaagctcggcggcaccgcggcgttcatcgacgccgagcatgcgctcgacgtgcagtacgcggggaagctcggcgtcaacgtgccggaactgctgatctcgcagccggacaccggcgagcaggcgctcgaaatcaccgatgcgctggtgcgctcgggctcggtcgacatgatcgtgatcgactcggtcgcggcgctcgtgccgaaggccgaaatcgagggcgagatgggcgattcgctgccgggcctgcaggcgcgcctgatgtcgcaggcgctgcgcaagctgacgggcacgatcaagcgcacgaactgcctcgtgatcttcatcaaccagatccggatgaagatcggcgtgatgttcgggaacccggaaaccacgacgggcggcaacgcgctgaagttctactcgtcggtgcgtctcgacatccgccggatcggctcgatcaagaagaacgacgaggtgatcggcaacgaaacgcgcgtgaaggtcgtgaagaacaaggtgtcgccgccgttccgcgaagcgatcttcgacatcctgtacggcgaaggcatttcgcgccagggcgagatcatcgatctcggcgtgcaggcgaagatcgtcgacaaggcgggcgcgtggtacagctacaagggcgagaagatcggccagggcaaggacaacgcgcgcgagttcctgcgcgagaatccggagatcgcgcgggagatcgagaaccgcatccgcgaatcgctcggcgtggccgcgatgccgcaaggcgtcgatgctgacgaaatcgaggcgatgggcgaagaagagtg

SEQ ID NO: 163 - DNA, 1337 bp, Paraburkholderia sp.
  Marker nucleic acid sequence
tgaaaccggtcattgccctcgttgggcgccccaatgtggggaaatccacgctgttcaaccggctcacgcgctcgcgcgatgcgctggtggccgatctgcccggcctcacgcgcgatcgccactacggcgaggggcgcgttggcgagcgtccgtatctcgtcgtcgacacgggcggcttcgagccggtcgcgaaggacgggatcctgcacgagatggcgaggcagacgcggcaggcggtcgaggaggccgacgtcgtcgtgttcatcgtcgacggccgcaacgggctcgcgccgcaggacaagtcgatcgccgactatctgcgcaagacgggccggccgatcttcctcgtcgtgaacaaggcggaggggatgaagtacacggcggtcgcgacggacttctacgagctcgggctcggcgatccgcgcgcgatctcggccgcccacggcgacggcgtgaccgacatgatcaacgaggcgctcgaggtcgcatacgcggggcagccggaggaaagcgaagacgacgacgcgaagcgcggcgtcaagatcgcgatcgtcggccggccgaacgtcggcaagtcgacgctgatcaacacgctggtcggcgaggaccgcgtgatcgcgttcgacatgccgggcacgacgcgcgattcgatctacgtcgacttcgagcgcaacggcaagcagtacacgctgatcgacaccgcgggcctgcgccggcgcggcaaggtgttcgaggcgatcgaaaagttctcggtcgtgaagacgctgcagtcgatcgccgacgcgaacgtcgtgatcatgctgctcgacgcgcgccaggacatctcggaccaggacgcgcacatcgcgggcttcgtcgtcgagcaggggcgcgcgctcgtcgtcgcggtcaacaagtgggacggcctcgattcgcatgtgcgcgagcgcacgaaggccgatttgacccgcaagctaaaattcctcgaattcgcgaagttccacttcatctccgcgctcgagaagaccggcatcggcgcgctgatgcggtcggtcgacgaagcgtatgcggccgcgatggcgaagctgccgacgccgaagctcacgcgcgcgctgatcgaggcggtcgaattccagcagccgcgtcgccgcgggccggtgcgtccgaagctgcgctatgcgcaccagggcgggcagaatccgccgatcatcgtgattcacggcaatgcgctcgatgcggttaccgacacgtacaagcgctatctggaaaaccgcttccgcgaaactttctcgctgaccgggactccattgcgcatagagttccgttcctcgacgaatccgtacgcggacaaggactga

SEQ ID NO: 164 - DNA, 611 bp, Serratia sp.
  Marker nucleic acid sequence
tgataggtcgtctcagaggaaatattctggaaaagcagccgccgctggtgttactggaggcgaacggcgtgggttatgaagtgcacatgccgatgacctgcttttacgagctgccggaactggggcaagaggccatcgtgtttacccacttcgtggtgcgtgaagacgcgcagctgctgtacggtttcaacgacaagcaggagcgcgcgctgttccgcgagctgatcaaggtcaacggcgtcgggccgaagctggcgctggccatcctgtccggcatgtccgcgcagcagttcgtcagcgcggtggagcgtgaagagatcaccgcgctggtcaagttgccgggcgtgggcaagaaaaccgccgagcgcctggtggtagagatgaaagaccgcttcaaggggctcaacggcgatctgttcaacagcagcgagatcagcctgccggccgccgccgacaaaacgccggaagcggacgcagaagccgaagctgtctcggcgctggtggcgctgggctataagccgcaggaagccagccgcatggtcagcaagattgccaagccgggcgccgactgcgaaaccctgatccgcgacgcgctgcgcgccgccctgtaa

SEQ ID NO: 165 - DNA, 1034 bp, Serratia sp.
  Marker nucleic acid sequence
tgatccgcatttatccggaacaacttgccgcgcagctccgagaggggctgcgcgcttgttatttattgagcggcaatgaaccgctgctgctgcaggaaagccaggatttactccggcaggccgcgcaacagcagcagttcagcgagcactacagcatttccctcgacgcccatactgactgggacgccatcttcggcatctgccaggcgatgagcctgttcgccagccgccagacgctgctgctgatcttcccggaaaacggcccgacggcgccgatcggcgaacagctgaccaagctcgccacgctgctgcacgaggatattttgctgatcctgcgcggcccgcgcctcaccaaagcgcaggaaaacagcgcctggttcacggcgctcagcccgcacggcgctctcgtcagttgccagacacctgaacaggcgcagctgccgcgctgggtcgccgcgcgcgccaaagcgatgaagctggagttggacgacgcggccaatcagctgctgtgttactgctacgaaggcaacctgctggcgctgtcgcaggcgctggagcggctgtcgctgctgcacccggacggcaagctcacgctgccgcgcgtcgagcaggcggtgaacgacgccgcccacttcacgccgttccactggctggatgccctgctggccggcaagagcaagcgcgcctggcatattttgcagcagttgcagcaagaggacgtggagccggtgatcctgctgcgcaccctgcagcgcgaactgctgctgctgctgacgctgcagcgccgcatggcttccgcgccgctgcgctcgctgttcgatcagcacaaggtctggcagaaccgccgcccgttggtgacccaggcgttgcaacgtctgtccggcccccagctgcagcaggcggtacagctgctgacgcaaatcgagctgaccctcaagcaggactatggccagtcggtctggccggaactggaaaccctgtcgatgctgctgtgcggcaaacccctggccacgagttttagcgatgcccactaa

SEQ ID NO: 166 - DNA, 1091 bp, Chryseobacterium sp.
  Marker nucleic acid sequence
tgaaatgtggaatcgtaggcctgccgaatgtaggtaaatcaactctttttaactgtttgagtaatgcaaaagctcaatcagcaaactatcctttctgtactattgaaccgaaccttggaacggtttctgtaccggatcagagattgtttgaactggagaaaatcgtaaaacctgagagagttttaccagctgtagttgagattgttgatattgcaggtcttgtaaaaggagccagcaaaggagaaggattggggaaccagttcttggctaatatccgtgagtgtgaagcaattattcacgttttaagatgctttgataatggaaatatcatccacgttgaaggttctgtagatcctttaagagataaagaaattattgatatcgagctgcaattgaaagaccttgaaacagtagggaaagcggttgaaaaggctaagaaattcatcaagtcagggaagaaagaagatatccttacttacgaaacacttcagaatttacagaaattccttgaagatggtaaaaacgcaagagaatttgctaccgatgattttacaaaagcaattattggagaagttcagttgttaaccaataaacctgtactttacgtttgtaatgtagacgaaaattctatcaaaaatggaaatgactggattggtaagattgaagagatggctaagaatgaaggtgcagaaatagtagtattagctgcacagatagaagctgatatcaatgagcttgaaacttttgaagagagagaaattttccttgaagaattaggtcttactgagccgggggttaaccgtttgatcagaaaagcttacgatcttttaaaacttcagacttactttactgctggagtgaaagaagtaagagcctggactatcggacagggatggactgcgccacaggctgcaggagtaatccacactgattttgagaaaggatttatccgtgcagaagtaatcaagtataacgattatattacgtacggttctgaagtaaaaatcaaagaagccggaaaactttctgtagaaggtaaagaatatgtagtacaggatggagacatcatgcacttcagattcaatgtataa

SEQ ID NO: 167 - DNA, 2012 bp, Chryseobacterium sp.
  Marker nucleic acid sequence
tgttgtatacattcttttctatggattttaagcttcaatcagaatataaacctaccggagaccagccccaagcaattgaaaaacttaccgaagggatagagatcggtgagaaatatcaaacgctactcggagttacaggctccggaaaaacctttactgttgcgaatgttgtaaaaaacgttcagcgcccaaccttagttctggctcataataaaactcttgcggcacagcttttcatggagtttaaagagttttttccggaaaatgccgtggaatactttgtaagctactatgattactaccagcctgaggcgtatattgctacaacaggaacttatattgaaaaagacctgagcatcaatgaagaggtggaaaagctgcgtctttctgcaactgccagccttctttcaggaagaagggatgttttgattgtagcctctgtttcctgtatttacggtatcggaaaccctacagaatttcataagtctttaatttccattgctattggtgaaaaagtgacaagaacagcacttctccattcactagttaatgcattatatgccagaacattaaatgaattccaacgaggaacattccgtgtaaaaggagatgtgattgatgtatttcctgcttatactgataatgccatcagaattcagttttttggagatgaaattgaaaaaattcaaagctttgatcctgttacagggaatgtggaagataattttgatcagatacaaatttatcctgccaatctttttgtcacatcaaaggaaaccttaaacggtgccatcaaagaaattcaggatgatatggtaaaacaggttgatttctttagctctgttggaaagccattagaagccaagcgacttcaggaaagaactgaattggaccttgagatgatcaaagaattgggctattgttcaggaattgagaactattcgagatatcttgacggccgtcttccgggcacaaggcctttctgtctgatcgactatttccctaaagactttttaatggttattgatgaaagccacgtaactgttccacaggttcacgccatgtacggaggtgaccgaagcagaaaagaagcattagtggagtacggcttcagacttccggctgctatggacaacagacctttaaaatttgaagaatttgaagctattcagaatcaggtaatctatgtatcagcaactcctgcagattatgaacttgagaaaaccggaggagcttatatagagcagattattcgtccgacaggacttcttgaccctattattgaggtaagacctaccatcaaccagattgatgatttgatggaggaaatccataaaagatcggatgctgatgaaagagtactggtaacgactttaacgaagaaaatggctgaagaacttacgaaatactttaccaaattcggaatcagaacaagatatattcactctgatgttgaaacgctggaacgtattcagatcatgcaggatcttcgtctgggactttttgacgtgcttattggagtcaacttattgcgagaaggattggatttaccagaagtttcattagtagctattctggatgctgataaagaaggaatgctgagaagcagaagatcaatgattcaaactgtgggacgtgctgcaaggaatgtgaacggaaaagcaatcatgtatgctgataaaatcacaaaatctatgcaggctaccctggatgaaaccgaataccgccgtgctaaacaaatgcagtacaatgaagatcacggcctgaagccaaaagcattaaataaaaagatttctgaaaaccttgtcggaagaagcaaagacttcccggatgagaaatatacccaaaaagaaattcttcagaaagttgctgaaacaaaagcaacctatgcaagtgaagatatagagaaaatgattactcagaagcagaaagaaatggaagcggctgcgaaaaaccttgatttcataaaagcggccaagctgagagatgaaattgcagctttgaaagcttaa

SEQ ID NO: 168 - DNA, 1934 bp, Chryseobacterium sp.
  Marker nucleic acid sequence
tgagtcaaaaacaatatacagctagtagtattcaggcattggaaggaatggagcacgttcgtatgcgtccttcaatgtacattggtgatgtaggaacaagaggtcttcaccatttggtttatgaagtagtagataactctattgacgaggcattggcaggatactgcgacacgatcttcgttagcatcaaggaaggaaacggaattgaagtaagtgataacggtagaggtatcccggttgatttccacgaaaaagagcagaaatcagctcttgaagttgtaatgacaaaaattggagccggtggtaagtttgataaagactcttacaaagtttcaggaggtcttcacggagttggggtatcatgtgtgaatgcactttccaacgaaatggtaactactgtttacagagacggaaatatttatcagcagatttattctaaaggaaaagcacagactcaggttgaagagatcggacatagcgagaaaagaggtacaaagcagtttttccagccggatgatactatttttacggaactgatttataactatgatacattagcaagccgtttaagagaactttcttaccttaataaaggaattaccattacgcttaccgatgaaagagaaaaattggaagacggttctttccgttcagaagtttttcattctgaaggagggttgaaagaattcgttgcttatatcgatggtaaccgtgaatctattatggaacacgtgattttcatggaaggcgaaagagatgatattcctgttgaagtggcgatgcgttataacacgtcattcaacgaaaatcttcactcttacgttaataatattaatacccacgaaggaggtactcaccttgcaggtttcagaagagctttaacaagaactttaaagaagtatgcagacgaattgggacttccggcaaaagaaaaagtagaaattaccggtgatgacttccgtgaaggattaacagctgtaatttctgtaaaagtaatggaacctcagtttgaaggacagaccaaaacaaaattaggaaactctgaagtttctggtgctgttgataaaattgtaggagagatgcttacaaacttcctggaagagaaccctaatgaagctaaggtaatcgttcaaaaagttgttttagctgctaaggcaagacaggctgcgaaaaaagctcgtgaaatggttcagagaaaatctccaatgggaggttctggtcttcccgggaaactttctgactgttcatctaaagacccggctgaatctgaactattccttgtagagggagattccgcaggtggaacagctaaacagggaagagacagacacttccaggctattcttccgttaagaggtaaaattttgaacgtagagaaatctatgcttcataaagtatatgacaacgaggaaatcagaaatatctacactgcccttggagtatctgtagggactgaagaagacagcaaagctttgaatatggcgaagcttagatatcataaaattgtgattatgaccgatgctgatatcgatggttctcacatttcaactctgattcttactttcttcttcagatatatgaaggaacttattgagaacggatatatctatattgctcaaccacctttatacttattaaagagaggtaacaaaaaagtatatgcgtacaatgaaaaagaacgtgaagagtttactttagaaatgtctccggacggaaaaggagtagaagtacaacgttacaaaggtcttggggaaatgaaccctgaacagctttgggaaacaaccctgaatcctgaacacagaattctgaaacaggtaactattgataatgcagtggaagcagacagtgttttctcaatgttgatgggagatgaagttccgccaagaagagagtttatcgagaaaaatgcgaaatatgcaaaaattgatgcataa

SEQ ID NO: 169 - DNA, 1001 bp, Chryseobacterium sp.
  Marker nucleic acid sequence
atgagtaacattgatgataagaaaaaagcacttgctctagtgcttgacaaattagataaaacatacggaaagggaacagtaatgactttaggtgatgaatctatagacaatacaatagaagtaattccttccggttctttaggattagatatcgcattaggtatcggcggatatccaaaaggaagaatcattgaaatatacggtcctgaatcttcaggtaaaacaacattaacccttcatgctattgctgaagctcaaaaagcaggtggtattgcagcatttattgatgcagagcacgctttcgacagaacctatgctgcgaaactaggaattgatctggaaaaccttatcatttctcagccggataacggagaacaggctttagaaattgcagataaccttatccgttcaggagctatcgatatcgttgtgattgactctgttgctgctcttacaccaaaagcagagattgaaggtgaaatgggagattctaaaatgggtcttcacgcaagattgatgtctcaggcattaagaaagcttacagctactatttcaagaacgaaatgtacagtgatcttcattaaccagttgagagaaaaaatcggggtaatgttcggaaatcctgaaactactaccggaggtaacgctcttaagttctatgcttctgtaagaattgatatcagaaaagcaagtgcacctatcaaacaaggtgatgaggctatcggtagccgtgtgaaagtgaagattgtgaaaaacaaagtagcacctcctttcaaacaggcagaattcgatatcatgtacggagaaggagtttctaaagtaggtgaaattcttgataccgcagtagacatgggaatcgtgaagaaaagcggatcttggttcagctacgaagagaccaaacttggtcagggacgtgatgctgtaaaagatgttttaaaagacaacccggatctttctgaggaattggagaacaagatcaaggaagaaatgaaaaacaaata

SEQ ID NO: 170 - DNA, 1310 bp, Chryseobacterium sp.
  Marker nucleic acid sequence
tgtcaaatattgtcgcaatcgttggacgtcccaacgtaggaaaatccacgctttttaatcgtttattagaaagaagagaagctattgtagattctacagccggtgttaccagagaccgtcactacggaaaatcagactggaacggggtagactttactgttattgataccggaggttatgatgtaggtacagatgatatctttgaagaagaaatccgtaagcaggtacaattagctgtagaagaggctacttctattatctttatgatgaatgtagaagaaggacttactgatacagattacgaaatttacaatttactaagaagatccaataaaccgatttatattgttatcaataaagtagattctgccaaagaagagcttccggcaacagaattttaccagttaggaattgataaatattacactatttcttctgcgaccggttcaggaacaggagaaatcttagacgatatcgttaaagactttccaacaacagaatataaagatcctttcgaaggattacctaaaattactattgcaggccgtccaaacgtagggaaatctacaatgaccaatgctttattggatgttgaaagaaatatcgtaacggatattgcaggaactaccagagacagtatccagactctgtataacaaattcgggcatgagtttgttttggtagatacagcaggaatgagaagaaagtccaaggtaaacgaagaccttgaattctattctgtaatgagatctatccgttctattgagtattctgacgtagttatcattatggtagatgctacacaaggatgggaatctcaggatatgaatatcttcggactggctcagaaaaacagaaaaggaattgtaatccttgttaataaatgggatttgatcgaagacaagcagaccaatacaatgcgtgatttcgaaaaatcaatcaaagataaaatcggtcagttccaggatattccaatcctgtttgtttcagctttaacgaagcagagaatcttaaaagctgtagaagtggcaatgcaggtttatgaagaccgtaagaagaaaattaaaacttcaaaactgaatgaagtaatgcttccgatcttcgaaaatactccgccaccggcattgaaaggaaaatatatcaagatcaaatattgtgtgcagcttccaacgccttcaccacagtttgtattcttctgtaaccttccacagtatgtgaaggaaccttataaaagatttactgaaaaccagctgagaaaagaattcgggtttaccggagttccgattgaagtatatttcagacaaaaataa

SEQ ID NO: 171 - DNA, 1091 bp, Variovorax sp.
  Marker nucleic acid sequence
atgagtttgcaatgcggcatcgtcggcctgcccaacgtcggtaaatccacccttttcaatgcgttgaccaaggccggcatcgcagcggaaaactatccgttctgcaccatcgagcccaacgtgggcgtggtggaagtgcccgacccccggctcgcgcaactgagcgagatcgtcaagcccgagcgcgtggtgcccgccatcgtcgagttcgtcgacatcgccggcctggtggccggtgccagcaccggcgaaggcctgggcaacaagttcctggcgcacatccgcgagaccgacgccaccgtcaacgtggtgcgctgcttcgacgacgagaacgtgatccacgtggccggcaaggtcgacccgatctccgacatcgaagtgatccagaccgaactctgcctggccgacctggccacggtcgagaaggcgctgcaccgtcacaccaaggtggcccgttccggcgacaaggacgcgcagaagctcgtcggcctgctcgagcgctgccaggccgcgctgaacgagaacacgcccgtgcgtgcgctcgacttcaccaaggaagagctgccgctggtcaagagcttcacgctgatcaccgccaagcccgcgatgttcgtgggcaacgtggcggaagacggcttcgagaacaacccgtatctcgaccgcctgcgcgaatacgcggccaagcagggcgcgcccgtggtcgccatctgcgccaagatcgaagccgacctggccgagatggacgacgaagacaagaagatgttcctcgccgagatcggccaggaagagccgggcctgaaccgcctgatccgcgcggccttcaagctgctgggcctgcagacctacttcaccgccggcgtgaaggaagtgcgtgcatggaccatccacatcggcgacaccggcccgcaggcggccggcgtgatccacggcgacttcgagaagggctacatccgcgcccagaccatcgcgttcgaggactacatcgccttcaagggcgagcagggcgcgaaggacgcggggaagatgcgttctgaaggcaaggaatacgtcgtgaaagatggcgacgtgatgaacttcctgttcagctcctg

SEQ ID NO: 172 - DNA, 2117 bp, Variovorax sp.
  Marker nucleic acid sequence
atgccagagaacaacgaagccatagcagacctgaagaaactggacccgatcgggccggcgaaacagggggaattcatccgctatccggactcgcctttcgagcttttccagccctatccgccggccggcgaccagccgaaagcgatcgaggggctggtcgagggcgtgatcgatggcgaagtgttccagaccctgctgggcgtgaccggttcgggcaagaccttcaccatggccaacgtgatcgcccggctgggccgcccggccatcgtgtttgcgcccaacaagacgctggcggcgcagctctacagcgaattccgcgagttcttcccgaagaacgccgtcgagtacttcgtgagctactacgactactaccagcccgaggcctatgtgccccagcgcgacttgttcatcgagaaagattcatcgatcaacgagcacatcgagcagatgcggctatcggccaccaagagcgtgctggagcggcgcgacacggtcatcgtggccacggtgagcgccatctacggcatcggcacgcccgaggactacatggagatgcggctgattgcccgcgtcggcgacaaggtcggccagcgcgacctgatttcgcggctcatccgcatgcagtacctgcgcaacgagcaggactttgcccgcggcaccttccgcgtgcggggcgacaccatcgacgtgtttccggcggaacacagcgagctggcgatccgcttcgagctgttcgacgacgagatcgagtcgctgcagctcttcgacccgctgaccggccgcatccgccagaaggtgccgcgcttcaccgtgtatccgtcgagccattacgtcacgccgcgcgacaaggtcatggcggcggtggaaaccatcaagatcgagcttggcgatcggctcaaggaactggtgggggcgggcaagctggtcgaggcgcagcggctggagcagcgcacccgtttcgacctcgaaatgctcgccgaaatcggccattgcaagggcattgagaactacacgcgccacctctcgggcgctgcgccaggcgacccgccggcaacgctgaccgattacctgccgaaggacgcgctgatgttcctcgacgaaagtcaccagatgatcggccagctcagcgccatgtacaacggcgaccgggcgcgcaagaccacgctggtcgagtacggctttcgactgccgagtgcgctggacaaccggccgctcaagatggaagagttcgaaaagcgcatgcgccagtgcatcttcgtttcggccacgccggcgcaatacgagaaggaccatgccggcaacgtggtcgaacagctggtgcggcccaccggactgatcgaccccgaggtcgaggtgcggcccgccacgcatcaggtggacgacgtgctgggcgagatccgcgtgcgcgtggacaagaacgagcgcgtgctgatcaccacgctgaccaagcgcatggccgagcagctgaccgactacctgggcgacaacggggtgaaggtgcgctacctgcacagtgacgtcgacacggtcgagcgggtggaaatcctgcgcgatctgcgcctgggcaccttcgacgtgctggtgggcatcaatctgctgcgcgaggggctggatattcccgaggtatcgctggtggcgatcctcgacgccgacaaggaaggctttttgcgcgcggagcgctcgctgatccagaccatcggccgcgcagcgcggaacctgaacggcaaggccatcctctacgccgaccggatgaccgagtcgatgaagaaggccatcggcgaaaccgaacgccgccgtgcccggcagattgcctacaacgaagccaacggcatcacgccgcgcagcatcgtcaagcaggtgcgcgacctgatcgacggcgtctacagcgagaaaatgggcaaggaaatggccaagctcgacctggaacgcgccaaggtcgaggacatgagcgaaaaggacatcgcccgggagatcaagcggctggaaaagcagatgctggaacacgcccgcaacctcgaattcgagaaggcagcccgggtgcgcgaccagttggcgctgctgcgggagcaggcgttcggggcgtcgggcggcgacaacatcgcgatcccgccgcagta

SEQ ID NO: 173 - DNA, 2606 bp, Variovorax sp.
  Marker nucleic acid sequence
tgagtgctgaagaaaacaagcccgacgtgccccacaccgagcccgtctacatccccgagatcgacaccgtggcccccgacaccagctacggcgaaggctccatcacgatcctcgaagggcttgaagccgtgcgcaagcgccccggcatgtacatcggcgacacctccgacggcaccggcctgcaccacctggtgttcgaagtcgtcgacaactccatcgacgaggccctggctggccattgcgacgacatcgtcgtgaccatccactccgacaactcgatcagcgttaccgacaacggccgcggcatcccgaccggcgtgaagatggacgacaagcacgagcccaagcgctcggctgcggaaattgcgctcaccgaactgcacgccggcggcaagttcaaccagaacagctacaaggtgtcgggcggcctgcacggcgtgggcgtgagttgcgtgaacgcgctgagcgtgatgctgcgtttggtggtgcgccgcgaaggcaagatccacgaactcgaattcagccgcggcttcgtgcagaaccgcctgctcgagacggtgaacggcgtcgaagtctcgcccatgaagatcatcggcgacaccgacaagcgcggcaccgaggtgcactttctgcctgacacccagatcttcaaggagaactcggacttccactacgagattctcagcaagcgcctgcgcgaactgagtttcctgaacaacggcgtgcgcatccgcctgaaggacgagcgcaccggcaaggaagacgacttctccggcgccggcggcgtgcgcggcttcgtcgagttcatcaacaagggcaagaccgtcctgcatccgaactcgttctacgccgagggcgagcgcccggccgacacctacggcggcatccccggcacgcacatcggcgtcgaagtggcgatgcagtggaacagcggctacaacgagcaggtgctgtgcttcaccaacaacatcccgcagcgtgacggcggcacccacctcaccggcctgcgcgccgcgatgacccgcgtcatcaacaagtacatcgaagagaacgagttcgcgaagaaggcgaaagtcgaagtcaccggcgacgacatgcgcgaaggcctgtgctgcgtgctgagcgtgaaggtgcccgagcccaagttctcgagccagaccaaggacaagctggtgtccagcgaagtgcgcgcgccggtggaggacatcgtcggcaagctgctcaccgactacctgcaggagcgcccgaacgacgccaagatcatctgcggcaagatcgtcgaggccgcccgcgcccgcgaagccgcccgcaaggcccgcgaaatgacgcgccgcaagggcgtgctcgacggcatgggcctgcccggcaagctggccgactgccaggagaaagatccggcgctgtgcgaggtctacctggtggaaggtgactccgccggcggctcggccaagcagggccgcgaccggaagttccaggccatcctgccgctgcgcggcaagatcctgaacgtcgagaaggcacgctacgagaagctgctcaccagcaacgaaattctcacgatgatcaccgcgctgggcaccggcatcggccgtgccggcgcaaccaccgcgggcggcggcgcggacgacttcaacgtcgccaagctgcgctaccaccgcatcatcatcatgaccgacgccgacgtcgacggcgcccacatccgcacgctgctgctgacgttcttctatcgccagatgcccgagctggtcgagcgcggccacatctacatcgcgcagcctccgctgtacaaggtgaaggtcggcaaggaagagcagtacctgaaggacggccccgcgctcgacgccttcctgctcaaggtggcgctgaaagacgcgagcctgcagaccggcggcgccaactccaccacgctgagcggcgacaccctgggcgagctggcacgcaagcaccaggtggccgaggccgtgatcgcgcgcctgcgcaacttcatggacgccgaggcactgcgcgccatcgccgacggcgtggcgctcgacctcgacaccacgcccgccgccgaggcatcggccgttgcgctgcaggccaagctgcgtgagctcaacaccaccggcattccggccgaggtgagcagcgagttcgacgcgcgcaccgacaagccgctgctgcgcatcagccgccgccaccacggcaacatcaagagcagcgtgctcacgcaagacttcgtgcacggcgccgactacgccgcgctcgccgaagcagccaacaccttccgcggcctgctgagccccgaaggcgcgctggtgaagcgcggcgaaggcgaacgcgccaaggaagaaaaggtcgacgacttccgccaggccatgaagtggctcatcggcgaagccgagcgcgccacctcgcgccagcgctacaagggcctgggcgagatgaaccccgagcagctgtgggaaaccacgatggacccgaccgtgcggcgcctgctgcgcgtgcagatcgacgatgcgatcgaggccgatcgggtgttcacgatgctgatgggcgacgaggtggagccgcgccgcgagttcatcgagcagaacgcgctgcgggcggcgaatatcgacgtgtaa

SEQ ID NO: 174 - DNA, 1106 bp, Variovorax sp.
  Marker nucleic acid sequence
atggacgcagtcgtcaagggcgcaagcatctcggtcgccaacagtgaaaaagccaaggcgctgcaagccgcgctggcccagatcgaaaagcagttcggcaagggcacgatcatgcggctcggcgaaggcgaggcgctcgaagacatccaggtggtttccaccggctcgctgggcctggacatcgcgctgggcgtcggcggcctgccgcgcggccgcgtgatcgaaatctacggcccggaatcgtcgggcaagaccacgctcacgctgcaagtcattgccgccatgcagaagcaggccggcacctgcgccttcgtcgatgccgagcacgccctcgacgtgcaatacgcccagaagctcggcgtgaacctgtccgacctgctgatcagccagcccgacaccggcgagcaggcccttgaaatcgtcgactcgctggtgcgctccggcgccgtcgacctgatcgtcatcgactcggtcgccgcgctcacgcccaaggctgaaatcgaaggcgaaatgggcgactcgctgcccggcctgcaggcccgcctgatgagccaggcgctgcgcaagctcacggccacgatcaagaagaccaactgcatggtcatcttcatcaaccagatccgcatgaagatcggcgtgatgttcggctcgcccgaaaccactaccggcggcaacgcgctgaagttctacgcctcggtgcgcctggacatccgccgcatcggcaccatcaagaagggcgacgaggccatcggcaacgaaaccaaggtgaaggtggtgaagaacaaggtctcgcccccgttcaagacggcggagttcgacatcctgttcggcgaaggcatcagccgcgaaggcgagatcatcgacatgggcgtgaccgccaagatcgtcgacaagtcgggcgcctggtacgcctacaacggcgagaagatcggccagggccgcgacaacgcccgcgagttcctgcgcgagaaccccgagctgtcgcgcgagatcgagaacaaggtgcgcgagtcgctgggcatcccgctgctggccgccgatgccggcgccacgcccgagaagccgcagaaggctgccaaggccgagaaggccgacaagggcgaata

SEQ ID NO: 175 - DNA, 1343 bp, Variovorax sp.
  Marker nucleic acid sequence
atgaagccggtcgtggccctggtcgggcggcccaacgttggcaaatcgaccctcttcaaccgcctgacccagacgcgcgacgccatcgtcgccgacttcgccggactcacgcgcgaccgtcactacggcaacggccgcctgggcaagcacgagttcatcgtcatcgacaccggcggcttcgagcctgacgcgggcagcggcatctacaaggaaatggccaagcagactcggcaggccgtggccgaggccgacgtggtgatcttcgtggtcgacgcccgtgagggcctctcggctcaagaccacgacattgccaacgaactgcgccgccttggcaagccctgcgtgctggcggccaacaaggccgagggcatgcacgacggcaccaagctggtcgacttctacgaactcggcttcggcgacgtgcatggcgtgtcggctgcgcacgggcagggcatgcgcgaccttgtcgagcttgccctcgagccgctgaacctgcctgatcccgacgacgagacggacgaggacgaggtcaacaagccgatcaagctggccgtggccggccggcccaatgtgggcaagtccacgctgatcaacacctggctgggtgaagagcgcctggtggccttcgacatgccgggcacgacgcgcgacgccatctccgtgcctttcgagcgaaacggccagcgcttcgagctgatcgacacggccggcctgcgtcgcaagggcagggtgttcgaggcgatcgagaagttctcggtggtcaagacgctgcaggccatcgagtcggccaatgtcgtgctgctgctgctcgacgccacgcagggcgtgaccgaccaggatgcgcacattgccggctacatcctcgagagtggccgcgcggtggtgatcgccatcaacaagtgggacgcggtcgacagctaccagcgtgagcagatccagcggcagatcgagacccggctgccgttcctgaagttcgcggcgctgcatttcatctcggccatcaagcgccagggcctgggccctgtgtggcaggcgattgcgcaggcccacaagtcggccacgcgcaagatgtcgaccccggtgctgacacgcctgctgctggaggcggtgcagttccaggcgcccaagcgtggcggtatgtaccgccccaagctgcgctatgcgcatcaggggggtatgaacccgccggtgatcgtgatccacgggaattcgctggagcatgtcaccgacgcttacaagcgcttcctcgaggggcgcttccgcaaggaattcgaccttgtgggtacccccttgcgcatcgagttcaagagctcgcacaatccatttgccgacaaggacgattg

SEQ ID NO: 176 - DNA, 20 bp, Artificial Sequence
  misc_feature (12)..(12): n is a, c, g, or t
  Primer
agagtttgatcntggctcag

SEQ ID NO: 177 - DNA, 22 bp, Artificial Sequence
  misc_feature (6)..(6): n is a, c, g, or t
  Primer
tacggntaccttgttacgactt

SEQ ID NO: 178 - DNA, 19 bp, Artificial Sequence
  misc_feature (9)..(9): n is a, c, g, or t
  Primer
gtgccagcngccgcggtaa

SEQ ID NO: 179 - DNA, 20 bp, Artificial Sequence
  misc_feature (11)..(11): n is a, c, g, or t
  misc_feature (15)..(15): n is a, c, g, or t
  Primer
ccgtcaattcntttnagttt